(19) United States
(12) Patent Application Publication
(10) Pub. No.: US 2010/0035239 A1
(43) Pub. Date: Feb. 11, 2010

(54) Compositions for use in identification of bacteria

(75) Inventors: Rangarajan Sampath, San Diego, CA (US); Thomas A. Hall, Oceanside, CA (US); David J. Ecker, Encinitas, CA (US); Mark W. Eshoo, Solana Beach, CA (US); Christian Massire, Carlsbad, CA (US)

Correspondence Address: Casimir Jones, S.C., 2275 Deming Way, Suite 310, Madison, WI, 53562, US

(73) Assignee: ISIS Pharmaceuticals, Inc., Carlsbad, CA (US)

(21) Appl. No.: 11/060,135

(22) Filed: Feb. 17, 2005

Related U.S. Application Data
(63) Continuation-in-part of application No. 10/728,486, filed on Dec. 5, 2003.
(60) Provisional application No. 60/501,926, filed on Sep. 11, 2003.
(60) Provisional application No. 60/545,425, filed on Feb. 18, 2004.
(60) Provisional application No. 60/559,754, filed on Apr. 5, 2004.
(60) Provisional application No. 60/632,862, filed on Dec. 3, 2004.
(60) Provisional application No. 60/639,068, filed on Dec. 22, 2004.
(60) Provisional application No. 60/648,188, filed on Jan. 28, 2005.

(51) Int. Cl. C12Q 1/68

(52) U.S. Cl. 435/6

(57) ABSTRACT

The present invention provides oligonucleotide primers and compositions and kits containing the same for rapid identification of bacteria by amplification of a segment of bacterial nucleic acid followed by molecular mass analysis.

### CROSS-REFERENCE TO RELATED APPLICATIONS

The present application is 1) a continuation-in-part of U.S. application Ser. No. 10/728,486, filed Dec. 5, 2003, which claims the benefit of priority to U.S. Provisional Application Ser. No. 60/501,926, filed Sep. 11, 2003, and 2) claims the benefit of priority to: U.S. Provisional Application Ser. No. 60/545,425 filed Feb. 18, 2004, U.S. Provisional Application Ser. No. 60/559,754, filed Apr. 5, 2004, U.S. Provisional Application Ser. No. 60/632,862, filed Dec. 3, 2004, U.S. Provisional Application Ser. No. 60/639,068, filed Dec. 22, 2004, and U.S. Provisional Application Ser. No. 60/648,188, filed Jan. 28, 2005, each of which is incorporated herein by reference in its entirety.

### STATEMENT OF GOVERNMENT SUPPORT

This invention was made with United States Government support under DARPA/SPO contract BAA00-09. The United States Government may have certain rights in the invention.

### FIELD OF THE INVENTION

The present invention relates generally to the field of genetic identification of bacteria and provides nucleic acid compositions and kits useful for this purpose when combined with molecular mass analysis.

### BACKGROUND OF THE INVENTION

A problem in determining the cause of a natural infectious outbreak or a bioterrorist attack is the sheer variety of organisms that can cause human disease. There are over 1400 organisms infectious to humans; many of these have the potential to emerge suddenly in a natural epidemic or to be used in a malicious attack by bioterrorists (Taylor et al. Philos. Trans. R. Soc. London B. Biol. Sci., 2001, 356, 983-989). This number does not include numerous strain variants, bioengineered versions, or pathogens that infect plants or animals.

Much of the new technology being developed for detection of biological weapons incorporates a polymerase chain reaction (PCR) step based upon the use of highly specific primers and probes designed to selectively detect certain pathogenic organisms. Although this approach is appropriate for the most obvious bioterrorist organisms, like smallpox and anthrax, experience has shown that it is very difficult to predict which of hundreds of possible pathogenic organisms might be employed in a terrorist attack. Likewise, naturally emerging human disease that has caused devastating consequence in public health has come from unexpected families of bacteria, viruses, fungi, or protozoa. Plants and animals also have their natural burden of infectious disease agents and there are equally important biosafety and security concerns for agriculture.

A major conundrum in public health protection, biodefense, and agricultural safety and security is that these disciplines need to be able to rapidly identify and characterize infectious agents, while there is no existing technology with the breadth of function to meet this need. Currently used methods for identification of bacteria rely upon culturing the bacterium to effect isolation from other organisms and to obtain sufficient quantities of nucleic acid followed by sequencing of the nucleic acid, both processes which are time and labor intensive.

Mass spectrometry provides detailed information about the molecules being analyzed, including high mass accuracy. It is also a process that can be easily automated. DNA chips with specific probes can only determine the presence or absence of specifically anticipated organisms. Because there are hundreds of thousands of species of benign bacteria, some very similar in sequence to threat organisms, even arrays with 10,000 probes lack the breadth needed to identify a particular organism.

There is a need for a method for identification of bioagents which is both specific and rapid, and in which no culture or nucleic acid sequencing is required. Disclosed in U.S. patent application Ser. Nos. 09/798,007, 09/891,793, 10/405,756, 10/418,514, 10/660,997, 10/660,122, 10/660,996, 10/728,486, 10/754,415 and 10/829,826, each of which is commonly owned and incorporated herein by reference in its entirety, are methods for identification of bioagents (any organism, cell, or virus, living or dead, or a nucleic acid derived from such an organism, cell or virus) in an unbiased manner by molecular mass and base composition analysis of “bioagent identifying amplicons” which are obtained by amplification of segments of essential and conserved genes which are involved in, for example, translation, replication, recombination and repair, transcription, nucleotide metabolism, amino acid metabolism, lipid metabolism, energy generation, uptake, secretion and the like. Examples of these proteins include, but are not limited to, ribosomal RNAs, ribosomal proteins, DNA and RNA polymerases, elongation factors, tRNA synthetases, protein chain initiation factors, heat shock protein groEL, phosphoglycerate kinase, NADH dehydrogenase, DNA ligases, DNA gyrases and DNA topoisomerases, metabolic enzymes, and the like.

To obtain bioagent identifying amplicons, primers are selected to hybridize to conserved sequence regions which bracket variable sequence regions to yield a segment of nucleic acid which can be amplified and which is amenable to methods of molecular mass analysis. The variable sequence regions provide the variability of molecular mass which is used for bioagent identification. Upon amplification by PCR or other amplification methods with the specifically chosen primers, an amplification product that represents a bioagent identifying amplicon is obtained. The molecular mass of the amplification product, obtained by mass spectrometry for example, provides the means to uniquely identify the bioagent without a requirement for prior knowledge of the possible identity of the bioagent. The molecular mass of the amplification product or the corresponding base composition (which can be calculated from the molecular mass of the amplification product) is compared with a database of molecular masses or base compositions and a match indicates the identity of the bioagent. Furthermore, the method can be applied to rapid parallel analyses (for example, in a multi-well plate format) the results of which can be employed in a triangulation identification strategy which is amenable to rapid throughput and does not require nucleic acid sequencing of the amplified target sequence for bioagent identification.

The result of determination of a previously unknown base composition of a previously unknown bioagent (for example, a newly evolved and heretofore unobserved bacterium or virus) has downstream utility by providing new bioagent indexing information with which to populate base composition databases. The process of subsequent bioagent identification analyses is thus greatly improved as more base composition data for bioagent identifying amplicons becomes available.

The present invention provides oligonucleotide primers and compositions and kits containing the oligonucleotide primers, which define bacterial bioagent identifying amplicons and, upon amplification, produce corresponding amplification products whose molecular masses provide the means to identify bacteria, for example, at and below the species taxonomic level.

### SUMMARY OF THE INVENTION

The present invention provides primers and compositions comprising pairs of primers, and kits containing the same for use in identification of bacteria. The primers are designed to produce bacterial bioagent identifying amplicons of DNA encoding genes essential to life such as, for example, 16S and 23S rRNA, DNA-directed RNA polymerase subunits (rpoB and rpoC), valyl-tRNA synthetase (valS), elongation factor EF-Tu (TufB), ribosomal protein L2 (rplB), protein chain initiation factor (infB), and spore protein (sspE). The invention further provides drill-down primers, compositions comprising pairs of primers and kits containing the same, which are designed to provide sub-species characterization of bacteria.

In particular, the present invention provides an oligonucleotide primer 16 to 35 nucleobases in length comprising 80% to 100% sequence identity with SEQ ID NO: 26, or a composition comprising the same; an oligonucleotide primer 20 to 27 nucleobases in length comprising at least a 20 nucleobase portion of SEQ ID NO: 388, or a composition comprising the same; a composition comprising both primers; and a composition comprising a first oligonucleotide primer 15 to 35 nucleobases in length comprising between 70% to 100% sequence identity of SEQ ID NO: 26, and a second oligonucleotide primer 16 to 35 nucleobases in length comprising between 70% to 100% sequence identity of SEQ ID NO: 388.

The present invention also provides an oligonucleotide primer 22 to 35 nucleobases in length comprising SEQ ID NO: 29, or a composition comprising the same; an oligonucleotide primer 18 to 35 nucleobases in length comprising SEQ ID NO: 391, or a composition comprising the same; a composition comprising both primers; and a composition comprising a first oligonucleotide primer 16 to 35 nucleobases in length comprising between 70% to 100% sequence identity of SEQ ID NO: 29, and a second oligonucleotide primer 13 to 35 nucleobases in length comprising between 70% to 100% sequence identity of SEQ ID NO: 391.

The present invention also provides an oligonucleotide primer 22 to 26 nucleobases in length comprising SEQ ID NO: 37, or a composition comprising the same; an oligonucleotide primer 20 to 30 nucleobases in length comprising SEQ ID NO: 362, or a composition comprising the same; a composition comprising both primers; and a composition comprising a first oligonucleotide primer 16 to 35 nucleobases in length comprising between 70% to 100% sequence identity of SEQ ID NO: 37, and a second oligonucleotide primer 14 to 35 nucleobases in length comprising between 70% to 100% sequence identity of SEQ ID NO: 362.

The present invention also provides an oligonucleotide primer 13 to 35 nucleobases in length comprising 70% to 100% sequence identity with SEQ ID NO: 48, or a composition comprising the same; an oligonucleotide primer 19 to 35 nucleobases in length comprising SEQ ID NO: 404, or a composition comprising the same; a composition comprising both primers; and a composition comprising a first oligonucleotide primer 13 to 35 nucleobases in length comprising between 70% to 100% sequence identity of SEQ ID NO: 48, and a second oligonucleotide primer 14 to 35 nucleobases in length comprising between 70% to 100% sequence identity of SEQ ID NO: 404.

The present invention also provides an oligonucleotide primer 21 to 35 nucleobases in length comprising 70% to 100% sequence identity with SEQ ID NO: 160, or a composition comprising the same; an oligonucleotide primer 21 to 35 nucleobases in length comprising at least a 16 nucleobase portion of SEQ ID NO: 515, or a composition comprising the same; a composition comprising both primers; and a composition comprising a first oligonucleotide primer 21 to 35 nucleobases in length comprising between 70% to 100% sequence identity of SEQ ID NO: 160, and a second oligonucleotide primer 21 to 35 nucleobases in length comprising between 70% to 100% sequence identity of SEQ ID NO: 515.

The present invention also provides an oligonucleotide primer 17 to 35 nucleobases in length comprising 70% to 100% sequence identity with SEQ ID NO: 261, or a composition comprising the same; an oligonucleotide primer 18 to 35 nucleobases in length comprising at least a 16 nucleobase portion of SEQ ID NO: 624, or a composition comprising the same; a composition comprising both primers; and a composition comprising a first oligonucleotide primer 17 to 35 nucleobases in length comprising between 70% to 100% sequence identity of SEQ ID NO: 261, and a second oligonucleotide primer 18 to 35 nucleobases in length comprising between 70% to 100% sequence identity of SEQ ID NO: 624.

The present invention also provides an oligonucleotide primer 21 to 35 nucleobases in length comprising 70% to 100% sequence identity with SEQ ID NO: 231, or a composition comprising the same; an oligonucleotide primer 17 to 35 nucleobases in length comprising 70% to 100% sequence identity with SEQ ID NO: 591, or a composition comprising the same; a composition comprising both primers; and a composition comprising a first oligonucleotide primer 21 to 35 nucleobases in length comprising between 70% to 100% sequence identity of SEQ ID NO: 231, and a second oligonucleotide primer 17 to 35 nucleobases in length comprising between 70% to 100% sequence identity of SEQ ID NO: 591.

The present invention also provides an oligonucleotide primer 14 to 35 nucleobases in length comprising 70% to 100% sequence identity with SEQ ID NO: 349, or a composition comprising the same; an oligonucleotide primer 17 to 35 nucleobases in length comprising 70% to 100% sequence identity with SEQ ID NO: 711, or a composition comprising the same; a composition comprising both primers; and a composition comprising a first oligonucleotide primer 14 to 35 nucleobases in length comprising between 70% to 100% sequence identity of SEQ ID NO: 349, and a second oligonucleotide primer 17 to 35 nucleobases in length comprising between 70% to 100% sequence identity of SEQ ID NO: 711.

The present invention also provides an oligonucleotide primer 16 to 35 nucleobases in length comprising 70% to 100% sequence identity with SEQ ID NO: 240, or a composition comprising the same; an oligonucleotide primer 15 to 35 nucleobases in length comprising 70% to 100% sequence identity with SEQ ID NO: 596, or a composition comprising the same; a composition comprising both primers; and a composition comprising a first oligonucleotide primer 16 to 35 nucleobases in length comprising between 70% to 100% sequence identity of SEQ ID NO: 240, and a second oligonucleotide primer 15 to 35 nucleobases in length comprising between 70% to 100% sequence identity of SEQ ID NO: 596.

The present invention also provides an oligonucleotide primer 16 to 35 nucleobases in length comprising 70% to 100% sequence identity with SEQ ID NO: 58, or a composition comprising the same; an oligonucleotide primer 21 to 35 nucleobases in length comprising at least a 16 nucleobase portion of SEQ ID NO:414, or a composition comprising the same; a composition comprising both primers; and a composition comprising a first oligonucleotide primer 16 to 35 nucleobases in length comprising between 70% to 100% sequence identity of SEQ ID NO: 58, and a second oligonucleotide primer 15 to 35 nucleobases in length comprising between 70% to 100% sequence identity of SEQ ID NO: 414.

The present invention also provides an oligonucleotide primer 16 to 35 nucleobases in length comprising at least a 16 nucleobase portion of SEQ ID NO: 6, or a composition comprising the same; an oligonucleotide primer 16 to 35 nucleobases in length comprising at least a 16 nucleobase portion of SEQ ID NO:369, or a composition comprising the same; a composition comprising both primers; and a composition comprising a first oligonucleotide primer 16 to 35 nucleobases in length comprising between 70% to 100% sequence identity of SEQ ID NO: 6, and a second oligonucleotide primer 15 to 35 nucleobases in length comprising between 70% to 100% sequence identity of SEQ ID NO: 369.

The present invention also provides an oligonucleotide primer 16 to 35 nucleobases in length comprising 70% to 100% sequence identity with SEQ ID NO: 246, or a composition comprising the same; an oligonucleotide primer 19 to 35 nucleobases in length comprising 70% to 100% sequence identity with SEQ ID NO: 602, or a composition comprising the same; a composition comprising both primers; and a composition comprising a first oligonucleotide primer 16 to 35 nucleobases in length comprising between 70% to 100% sequence identity of SEQ ID NO: 246, and a second oligonucleotide primer 19 to 35 nucleobases in length comprising between 70% to 100% sequence identity of SEQ ID NO: 602.

The present invention also provides an oligonucleotide primer 21 to 35 nucleobases in length comprising 70% to 100% sequence identity with SEQ ID NO: 256, or a composition comprising the same; an oligonucleotide primer 14 to 35 nucleobases in length comprising 70% to 100% sequence identity with SEQ ID NO: 620, or a composition comprising the same; a composition comprising both primers; and a composition comprising a first oligonucleotide primer 21 to 35 nucleobases in length comprising between 70% to 100% sequence identity of SEQ ID NO: 256, and a second oligonucleotide primer 14 to 35 nucleobases in length comprising between 70% to 100% sequence identity of SEQ ID NO: 620.

The present invention also provides an oligonucleotide primer 16 to 35 nucleobases in length comprising 70% to 100% sequence identity with SEQ ID NO: 344, or a composition comprising the same; an oligonucleotide primer 18 to 35 nucleobases in length comprising 70% to 100% sequence identity with SEQ ID NO: 700, or a composition comprising the same; a composition comprising both primers; and a composition comprising a first oligonucleotide primer 16 to 35 nucleobases in length comprising between 70% to 100% sequence identity of SEQ ID NO: 344, and a second oligonucleotide primer 18 to 35 nucleobases in length comprising between 70% to 100% sequence identity of SEQ ID NO: 700.

The present invention also provides an oligonucleotide primer 16 to 35 nucleobases in length comprising 70% to 100% sequence identity with SEQ ID NO: 235, or a composition comprising the same; an oligonucleotide primer 16 to 35 nucleobases in length comprising 70% to 100% sequence identity with SEQ ID NO: 587, or a composition comprising the same; a composition comprising both primers; and a composition comprising a first oligonucleotide primer 16 to 35 nucleobases in length comprising between 70% to 100% sequence identity of SEQ ID NO: 235, and a second oligonucleotide primer 16 to 35 nucleobases in length comprising between 70% to 100% sequence identity of SEQ ID NO: 587.

The present invention also provides an oligonucleotide primer 16 to 35 nucleobases in length comprising 70% to 100% sequence identity with SEQ ID NO: 322, or a composition comprising the same; an oligonucleotide primer 19 to 35 nucleobases in length comprising 70% to 100% sequence identity with SEQ ID NO: 686, or a composition comprising the same; a composition comprising both primers; and a composition comprising a first oligonucleotide primer 16 to 35 nucleobases in length comprising between 70% to 100% sequence identity of SEQ ID NO: 322, and a second oligonucleotide primer 19 to 35 nucleobases in length comprising between 70% to 100% sequence identity of SEQ ID NO: 686.

The present invention also provides an oligonucleotide primer 21 to 35 nucleobases in length comprising 70% to 100% sequence identity with SEQ ID NO: 97, or a composition comprising the same; an oligonucleotide primer 20 to 35 nucleobases in length comprising 70% to 100% sequence identity with SEQ ID NO: 451, or a composition comprising the same; a composition comprising both primers; and a composition comprising a first oligonucleotide primer 21 to 35 nucleobases in length comprising between 70% to 100% sequence identity of SEQ ID NO: 97, and a second oligonucleotide primer 20 to 35 nucleobases in length comprising between 70% to 100% sequence identity of SEQ ID NO: 451.

The present invention also provides an oligonucleotide primer 19 to 35 nucleobases in length comprising 70% to 100% sequence identity with SEQ ID NO: 127, or a composition comprising the same; an oligonucleotide primer 14 to 35 nucleobases in length comprising 70% to 100% sequence identity with SEQ ID NO: 482, or a composition comprising the same; a composition comprising both primers; and a composition comprising a first oligonucleotide primer 19 to 35 nucleobases in length comprising between 70% to 100% sequence identity of SEQ ID NO: 127, and a second oligonucleotide primer 14 to 35 nucleobases in length comprising between 70% to 100% sequence identity of SEQ ID NO: 482.

The present invention also provides an oligonucleotide primer 19 to 35 nucleobases in length comprising 70% to 100% sequence identity with SEQ ID NO: 174, or a composition comprising the same; an oligonucleotide primer 21 to 35 nucleobases in length comprising 70% to 100% sequence identity with SEQ ID NO: 530, or a composition comprising the same; a composition comprising both primers; and a composition comprising a first oligonucleotide primer 19 to 35 nucleobases in length comprising between 70% to 100% sequence identity of SEQ ID NO: 174, and a second oligonucleotide primer 21 to 35 nucleobases in length comprising between 70% to 100% sequence identity of SEQ ID NO: 530.

The present invention also provides an oligonucleotide primer 21 to 35 nucleobases in length comprising 70% to 100% sequence identity with SEQ ID NO: 310, or a composition comprising the same; an oligonucleotide primer 19 to 35 nucleobases in length comprising 70% to 100% sequence identity with SEQ ID NO: 668, or a composition comprising the same; a composition comprising both primers; and a composition comprising a first oligonucleotide primer 21 to 35 nucleobases in length comprising between 70% to 100% sequence identity of SEQ ID NO: 310, and a second oligonucleotide primer 19 to 35 nucleobases in length comprising between 70% to 100% sequence identity of SEQ ID NO: 668.

The present invention also provides an oligonucleotide primer 21 to 35 nucleobases in length comprising 70% to 100% sequence identity with SEQ ID NO: 313, or a composition comprising the same; an oligonucleotide primer 21 to 35 nucleobases in length comprising 70% to 100% sequence identity with SEQ ID NO: 670, or a composition comprising the same; a composition comprising both primers; and a composition comprising a first oligonucleotide primer 21 to 35 nucleobases in length comprising between 70% to 100% sequence identity of SEQ ID NO: 313, and a second oligonucleotide primer 21 to 35 nucleobases in length comprising between 70% to 100% sequence identity of SEQ ID NO: 670.

The present invention also provides an oligonucleotide primer 17 to 35 nucleobases in length comprising 70% to 100% sequence identity with SEQ ID NO: 277, or a composition comprising the same; an oligonucleotide primer 21 to 35 nucleobases in length comprising 70% to 100% sequence identity with SEQ ID NO: 632, or a composition comprising the same; a composition comprising both primers; and a composition comprising a first oligonucleotide primer 17 to 35 nucleobases in length comprising between 70% to 100% sequence identity of SEQ ID NO: 277, and a second oligonucleotide primer 21 to 35 nucleobases in length comprising between 70% to 100% sequence identity of SEQ ID NO: 632.

The present invention also provides an oligonucleotide primer 21 to 35 nucleobases in length comprising 70% to 100% sequence identity with SEQ ID NO: 285, or a composition comprising the same; an oligonucleotide primer 19 to 35 nucleobases in length comprising 70% to 100% sequence identity with SEQ ID NO: 640, or a composition comprising the same; a composition comprising both primers; and a composition comprising a first oligonucleotide primer 21 to 35 nucleobases in length comprising between 70% to 100% sequence identity of SEQ ID NO: 285, and a second oligonucleotide primer 19 to 35 nucleobases in length comprising between 70% to 100% sequence identity of SEQ ID NO: 640.

The present invention also provides an oligonucleotide primer 21 to 35 nucleobases in length comprising 70% to 100% sequence identity with SEQ ID NO: 301, or a composition comprising the same; an oligonucleotide primer 21 to 35 nucleobases in length comprising 70% to 100% sequence identity with SEQ ID NO: 656, or a composition comprising the same; a composition comprising both primers; and a composition comprising a first oligonucleotide primer 21 to 35 nucleobases in length comprising between 70% to 100% sequence identity of SEQ ID NO: 301, and a second oligonucleotide primer 21 to 35 nucleobases in length comprising between 70% to 100% sequence identity of SEQ ID NO: 656.

The present invention also provides an oligonucleotide primer 18 to 35 nucleobases in length comprising 70% to 100% sequence identity with SEQ ID NO: 308, or a composition comprising the same; an oligonucleotide primer 18 to 35 nucleobases in length comprising 70% to 100% sequence identity with SEQ ID NO: 663, or a composition comprising the same; a composition comprising both primers; and a composition comprising a first oligonucleotide primer 18 to 35 nucleobases in length comprising between 70% to 100% sequence identity of SEQ ID NO: 308, and a second oligonucleotide primer 18 to 35 nucleobases in length comprising between 70% to 100% sequence identity of SEQ ID NO: 663.

The present invention also provides compositions, such as those described herein, wherein either or both of the first and second oligonucleotide primers comprise at least one modified nucleobase, a non-templated T residue on the 5′-end, at least one non-template tag, or at least one molecular mass modifying tag, or any combination thereof.

The present invention also provides kits comprising any of the compositions described herein. The kits can comprise at least one calibration polynucleotide, or at least one ion exchange resin linked to magnetic beads, or both.

The present invention also provides methods for identification of an unknown bacterium. Nucleic acid from the bacterium is amplified using any of the compositions described herein to obtain an amplification product. The molecular mass of the amplification product is determined. Optionally, the base composition of the amplification product is determined from the molecular mass. The base composition or molecular mass is compared with a plurality of base compositions or molecular masses of known bacterial bioagent identifying amplicons, wherein a match between the base composition or molecular mass and a member of the plurality of base compositions or molecular masses identifies the unknown bacterium. The molecular mass can be measured by mass spectrometry. In addition, the presence or absence of a particular clade, genus, species, or sub-species of a bioagent can be determined by the methods described herein.

The present invention also provides methods for determination of the quantity of an unknown bacterium in a sample. The sample is contacted with any of the compositions described herein and a known quantity of a calibration polynucleotide comprising a calibration sequence. Concurrently, nucleic acid from the bacterium in the sample is amplified with any of the compositions described herein and nucleic acid from the calibration polynucleotide in the sample is amplified with any of the compositions described herein to obtain a first amplification product comprising a bacterial bioagent identifying amplicon and a second amplification product comprising a calibration amplicon. The molecular mass and abundance for the bacterial bioagent identifying amplicon and the calibration amplicon is determined. The bacterial bioagent identifying amplicon is distinguished from the calibration amplicon based on molecular mass, wherein comparison of bacterial bioagent identifying amplicon abundance and calibration amplicon abundance indicates the quantity of bacterium in the sample. The method can also comprise determining the base composition of the bacterial bioagent identifying amplicon.

### BRIEF DESCRIPTION OF THE DRAWINGS

FIG. 1 is a representative pseudo-four dimensional plot of base compositions of bioagent identifying amplicons of enterobacteria obtained with a primer pair targeting the rpoB gene (primer pair no 14 (SEQ ID NOs: 37:362). The quantity each of the nucleobases A, G and C are represented on the three axes of the plot while the quantity of nucleobase T is represented by the diameter of the spheres. Base composition probability clouds surrounding the spheres are also shown.

FIG. 2 is a representative diagram illustrating the primer selection process.

FIG. 3 lists common pathogenic bacteria and primer pair coverage. The primer pair number in the upper right hand corner of each polygon indicates that the primer pair can produce a bioagent identifying amplicon for all species within that polygon.

FIG. 4 is a representative 3D diagram of base composition (axes A, G and C) of bioagent identifying amplicons obtained with primer pair number 14 (a precursor of primer pair number 348 which targets 16S rRNA). The diagram indicates that the experimentally determined base compositions of the clinical samples (labeled NHRC samples) closely match the base compositions expected for Streptococcus pyogenes and are distinct from the expected base compositions of other organisms.

FIG. 5 is a representative mass spectrum of amplification products representing bioagent identifying amplicons of Streptococcus pyogenes, Neisseria meningitidis, and Haemophilus influenzae obtained from amplification of nucleic acid from a clinical sample with primer pair number 349 which targets 23S rRNA. Experimentally determined molecular masses and base compositions for the sense strand of each amplification product are shown.

FIG. 6 is a representative mass spectrum of amplification products representing a bioagent identifying amplicon of Streptococcus pyogenes, and a calibration amplicon obtained from amplification of nucleic acid from a clinical sample with primer pair number 356 which targets rplB. The experimentally determined molecular mass and base composition for the sense strand of the Streptococcus pyogenes amplification product is shown.

FIG. 7 is a representative process diagram for identification and determination of the quantity of a bioagent in a sample.

FIG. 8 is a representative mass spectrum of an amplified nucleic acid mixture which contained the Ames strain of Bacillus anthracis, a known quantity of combination calibration polynucleotide (SEQ ID NO: 741), and primer pair number 350 which targets the capC gene on the virulence plasmid pX02 of Bacillus anthracis. Calibration amplicons produced in the amplification reaction are visible in the mass spectrum as indicated and abundance data (peak height) are used to calculate the quantity of the Ames strain of Bacillus anthracis.

### DESCRIPTION OF EMBODIMENTS

The present invention provides oligonucleotide primers which hybridize to conserved regions of nucleic acid of genes encoding, for example, proteins or RNAs necessary for life which include, but are not limited to: 16S and 23S rRNAs, RNA polymerase subunits, t-RNA synthetases, elongation factors, ribosomal proteins, protein chain initiation factors, cell division proteins, chaperonin groEL, chaperonin dnaK, phosphoglycerate kinase, NADH dehydrogenase, DNA ligases, metabolic enzymes and DNA topoisomerases. These primers provide the functionality of producing, for example, bacterial bioagent identifying amplicons for general identification of bacteria at the species level, for example, when contacted with bacterial nucleic acid under amplification conditions.

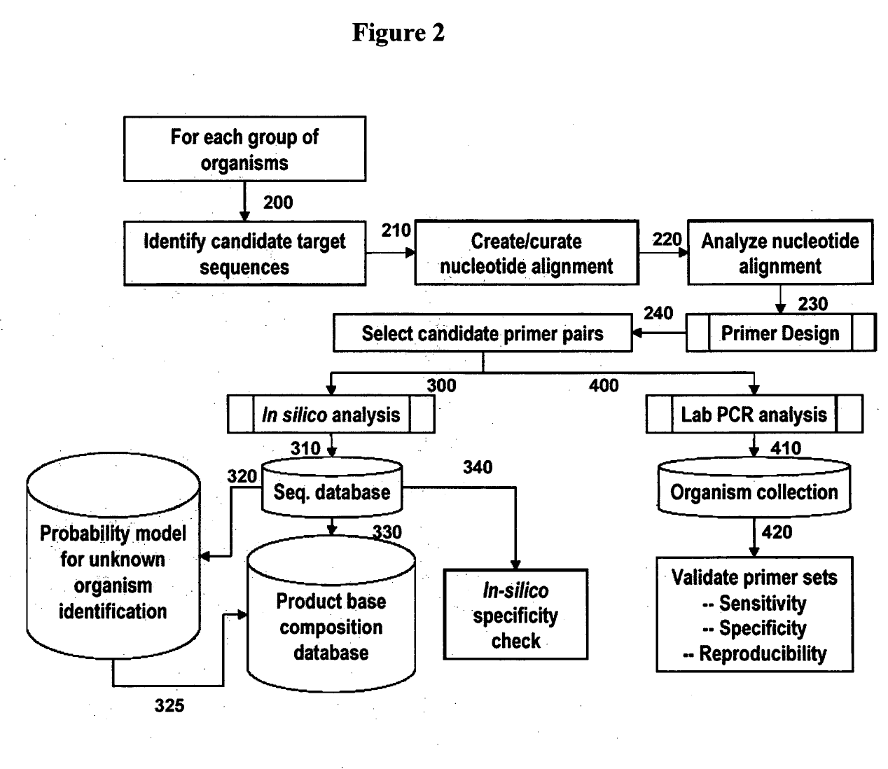

Referring to FIG. 2, primers are designed as follows: for each group of organisms, candidate target sequences are identified (200) from which nucleotide alignments are created (210) and analyzed (220). Primers are designed by selecting appropriate priming regions (230) which allows the selection of candidate primer pairs (240). The primer pairs are subjected to in silico analysis by electronic PCR (ePCR) (300) wherein bioagent identifying amplicons are obtained from sequence databases such as, for example, GenBank or other sequence collections (310), and checked for specificity in silico (320). Bioagent identifying amplicons obtained from GenBank sequences (310) can also be analyzed by a probability model which predicts the capability of a particular amplicon to identify unknown bioagents such that the base compositions of amplicons with favorable probability scores are stored in a base composition database (325). Alternatively, base compositions of the bioagent identifying amplicons obtained from the primers and GenBank sequences can be directly entered into the base composition database (330). Candidate primer pairs (240) are validated by in vitro amplification by a method such as, for example, PCR analysis (400) of nucleic acid from a collection of organisms (410). Amplification products that are obtained are optionally analyzed to confirm the sensitivity, specificity and reproducibility of the primers used to obtain the amplification products (420).

Synthesis of primers is well known and routine in the art. The primers may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed.

The primers can be employed as compositions for use in, for example, methods for identification of bacterial bioagents as follows. In some embodiments, a primer pair composition is contacted with nucleic acid of an unknown bacterial bioagent. The nucleic acid is amplified by a nucleic acid amplification technique, such as PCR for example, to obtain an amplification product that represents a bioagent identifying amplicon. The molecular mass of one strand or each strand of the double-stranded amplification product is determined by a molecular mass measurement technique such as, for example, mass spectrometry wherein the two strands of the double-stranded amplification product are separated during the ionization process. In some embodiments, the mass spectrometry is electrospray Fourier transform ion cyclotron resonance mass spectrometry (ESI-FTICR-MS) or electrospray time of flight mass spectrometry (ESI-TOF-MS). A list of possible base compositions can be generated for the molecular mass value obtained for each strand and the choice of the correct base composition from the list is facilitated by matching the base composition of one strand with a complementary base composition of the other strand. The molecular mass or base composition thus determined is compared with a database of molecular masses or base compositions of analogous bioagent identifying amplicons for known bacterial bioagents. A match between the molecular mass or base composition of the amplification product from the unknown bacterial bioagent and the molecular mass or base composition of an analogous bioagent identifying amplicon for a known bacterial bioagent indicates the identity of the unknown bioagent.

In some embodiments, the primer pair used is one of the primer pairs of Table 1. In some embodiments, the method is repeated using a different primer pair to resolve possible ambiguities in the identification process or to improve the confidence level for the identification assignment.

In some embodiments, a bioagent identifying amplicon may be produced using only a single primer (either the forward or reverse primer of any given primer pair), provided an appropriate amplification method is chosen, such as, for example, low stringency single primer PCR (LSSP-PCR). Adaptation of this amplification method in order to produce bioagent identifying amplicons can be accomplished by one with ordinary skill in the art without undue experimentation.

In some embodiments, the oligonucleotide primers are “broad range survey primers” which hybridize to conserved regions of nucleic acid encoding RNA, such as ribosomal RNA (rRNA), of all, or at least 70%, at least 80%, at least 85%, at least 90%, or at least 95% of known bacteria and produce bacterial bioagent identifying amplicons. As used herein, the term “broad range survey primers” refers to primers that bind to nucleic acid encoding rRNAs of all, or at least 70%, at least 80%, at least 85%, at least 90%, or at least 95% known species of bacteria. In some embodiments, the rRNAs to which the primers hybridize are 16S and 23S rRNAs. In some embodiments, the broad range survey primer pairs comprise oligonucleotides ranging in length from 13 to 35 nucleobases, each of which have from 70% to 100% sequence identity with primer pair numbers 3, 10, 11, 14, 16, and 17 which consecutively correspond to SEQ ID NOs: 6:369, 26:388, 29:391, 37:362, 48:404, and 58:414.

In some cases, the molecular mass or base composition of a bacterial bioagent identifying amplicon defined by a broad range survey primer pair does not provide enough resolution to unambiguously identify a bacterial bioagent at the species level. These cases benefit from further analysis of one or more bacterial bioagent identifying amplicons generated from at least one additional broad range survey primer pair or from at least one additional “division-wide” primer pair (vide infra). The employment of more than one bioagent identifying amplicon for identification of a bioagent is herein referred to as “triangulation identification” (vide infra).

In other embodiments, the oligonucleotide primers are “division-wide” primers which hybridize to nucleic acid encoding genes of broad divisions of bacteria such as, for example, members of the Bacillus/Clostridia group or members of the α-, β-, γ-, and ε-proteobacteria. In some embodiments, a division of bacteria comprises any grouping of bacterial genera with more than one genus represented. For example, the β-proteobacteria group comprises members of the following genera: Eikenella, Neisseria, Achromobacter, Bordetella, Burkholderia, and Raltsonia. Species members of these genera can be identified using bacterial bioagent identifying amplicons generated with primer pair 293 (SEQ ID NOs: 344:700) which produces a bacterial bioagent identifying amplicon from the tufB gene of β-proteobacteria. Examples of genes to which division-wide primers may hybridize to include, but are not limited to: RNA polymerase subunits such as rpoB and rpoC, tRNA synthetases such as valyl-tRNA synthetase (valS) and aspartyl-tRNA synthetase (aspS), elongation factors such as elongation factor EF-Tu (tufB), ribosomal proteins such as ribosomal protein L2 (rplB), protein chain initiation factors such as protein chain initiation factor infB, chaperonins such as groL and dnaK, and cell division proteins such as peptidase ftsH (hflB). In some embodiments, the division-wide primer pairs comprise oligonucleotides ranging in length from 13 to 35 nucleobases, each of which have from 70% to 100% sequence identity with primer pair numbers 34, 52, 66, 67, 71, 72, 289, 290 and 293 which consecutively correspond to SEQ ID NOs: 160:515, 261:624, 231:591, 235:587, 349:711, 240:596, 246:602, 256:620, 344:700.

In other embodiments, the oligonucleotide primers are designed to enable the identification of bacteria at the clade group level, which is a monophyletic taxon referring to a group of organisms which includes the most recent common ancestor of all of its members and all of the descendants of that most recent common ancestor. The Bacillus cereus clade is an example of a bacterial clade group. In some embodiments, the clade group primer pairs comprise oligonucleotides ranging in length from 13 to 35 nucleobases, each of which have from 70% to 100% sequence identity with primer pair number 58 which corresponds to SEQ ID NOs: 322:686.

In other embodiments, the oligonucleotide primers are “drill-down” primers which enable the identification of species or “sub-species characteristics.” Sub-species characteristics are herein defined as genetic characteristics that provide the means to distinguish two members of the same bacterial species. For example, Escherichia coli O157:H7 and Escherichia coli K12 are two well known members of the species Escherichia coli. Escherichia coli O157:H7, however, is highly toxic due to the its Shiga toxin gene which is an example of a sub-species characteristic. Examples of sub-species characteristics may also include, but are not limited to: variations in genes such as single nucleotide polymorphisms (SNPs), variable number tandem repeats (VNTRs). Examples of genes indicating sub-species characteristics include, but are not limited to, housekeeping genes, toxin genes, pathogenicity markers, antibiotic resistance genes and virulence factors. Drill-down primers provide the functionality of producing bacterial bioagent identifying amplicons for drill-down analyses such as strain typing when contacted with bacterial nucleic acid under amplification conditions. Identification of such sub-species characteristics is often critical for determining proper clinical treatment of bacterial infections. Examples of pairs of drill-down primers include, but are not limited to, a trio of primer pairs for identification of strains of Bacillus anthracis. Primer pair 24 (SEQ ID NOs: 97:451) targets the capC gene of virulence plasmid pX02, primer pair 30 (SEQ ID NOs: 127:482) targets the cyA gene of virulence plasmid pX02, and primer pair 37 (SEQ ID NOs: 174:530) targets the lef gene of virulence plasmid pX02. Additional examples of drill-down primers include, but are not limited to, six primer pairs that are used for determining the strain type of group A Streptococcus. Primer pair 80 (SEQ ID NOs: 310:668) targets the gki gene, primer pair 81 (SEQ ID NOs: 313:670) targets the gtr gene, primer pair 86 (SEQ ID NOs: 227:632) targets the murI gene, primer pair 90 (SEQ ID NOs: 285:640) targets the mutS gene, primer pair 96 (SEQ ID NOs: 301:656) targets the xpt gene, and primer pair 98 (SEQ ID NOs: 308:663) targets the yqiL gene.

In some embodiments, the primers used for amplification hybridize to and amplify genomic DNA, DNA of bacterial plasmids, or DNA of DNA viruses.

In some embodiments, the primers used for amplification hybridize directly to ribosomal RNA or messenger RNA (mRNA) and act as reverse transcription primers for obtaining DNA from direct amplification of bacterial RNA or rRNA. Methods of amplifying RNA using reverse transcriptase are well known to those with ordinary skill in the art and can be routinely established without undue experimentation.

One with ordinary skill in the art of design of amplification primers will recognize that a given primer need not hybridize with 100% complementarity in order to effectively prime the synthesis of a complementary nucleic acid strand in an amplification reaction. Moreover, a primer may hybridize over one or more segments such that intervening or adjacent segments are not involved in the hybridization event (e.g., a loop structure or a hairpin structure). The primers of the present invention may comprise at least 70%, at least 75%, at least 80%, at least 85%, at least 90%, at least 95% or at least 99% sequence identity with any of the primers listed in Table 1. Thus, in some embodiments of the present invention, an extent of variation of 70% to 100%, or any range therewithin, of the sequence identity is possible relative to the specific primer sequences disclosed herein. Determination of sequence identity is described in the following example: a primer 20 nucleobases in length which is otherwise identical to another 20 nucleobase primer but having two non-identical residues has 18 of 20 identical residues (18/20=0.9 or 90% sequence identity). In another example, a primer 15 nucleobases in length having all residues identical to a 15 nucleobase segment of primer 20 nucleobases in length would have 15/20=0.75 or 75% sequence identity with the 20 nucleobase primer.

Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). In some embodiments, homology, sequence identity, or complementarity of primers with respect to the conserved priming regions of bacterial nucleic acid, is at least 70%, at least 80%, at least 90%, at least 92%, at least 94%, at least 95%, at least 96%, at least 97%, at least 98%, at least 99%, or is 100%.

In some embodiments, the primers described herein comprise at least 70%, at least 75%, at least 80%, at least 85%, at least 90%, at least 92%, at least 94%, at least 95%, at least 96%, at least 98%, or at least 99%, or 100% (or any range therewithin) sequence identity with the primer sequences specifically disclosed herein. Thus, for example, a primer may have between 70% and 100%, between 75% and 100%, between 80% and 100%, and between 95% and 100% sequence identity with SEQ ID NO: 26. Likewise, a primer may have similar sequence identity with any other primer whose nucleotide sequence is disclosed herein.

One with ordinary skill is able to calculate percent sequence identity or percent sequence homology and able to determine, without undue experimentation, the effects of variation of primer sequence identity on the function of the primer in its role in priming synthesis of a complementary strand of nucleic acid for production of an amplification product of a corresponding bioagent identifying amplicon.

In some embodiments of the present invention, the oligonucleotide primers are between 13 and 35 nucleobases in length (13 to 35 linked nucleotide residues). These embodiments comprise oligonucleotide primers 13, 14, 15, 16, 17, 18, 19, 20, 21, 22, 23, 24, 25, 26, 27, 28, 29, 30, 31, 32, 33, 34 or 35 nucleobases in length, or any range therewithin.

In some embodiments, any given primer comprises a modification comprising the addition of a non-templated T residue to the 5′ end of the primer (i.e., the added T residue does not necessarily hybridize to the nucleic acid being amplified). The addition of a non-templated T residue has an effect of minimizing the addition of non-templated A residues as a result of the non-specific enzyme activity of Taq polymerase (Magnuson et al. Biotechniques, 1996, 21, 700-709), an occurrence which may lead to ambiguous results arising from molecular mass analysis.

In some embodiments of the present invention, primers may contain one or more universal bases. Because any variation (due to codon wobble in the 3^{rd} position) in the conserved regions among species is likely to occur in the third position of a DNA triplet, oligonucleotide primers can be designed such that the nucleotide corresponding to this position is a base which can bind to more than one nucleotide, referred to herein as a “universal nucleobase.” For example, under this “wobble” pairing, inosine (I) binds to U, C or A; guanine (G) binds to U or C, and uridine (U) binds to U or C. Other examples of universal nucleobases include nitroindoles such as 5-nitroindole or 3-nitropyrrole (Loakes et al., Nucleosides and Nucleotides, 1995, 14, 1001-1003), the degenerate nucleotides dP or dK (Hill et al.), an acyclic nucleoside analog containing 5-nitroindazole (Van Aerschot et al., Nucleosides and Nucleotides, 1995, 14, 1053-1056) or the purine analog 1-(2-deoxy-β-D-ribofuranosyl)-imidazole-4-carboxamide (Sala et al., Nucl. Acids Res., 1996, 24, 3302-3306).

In some embodiments, to compensate for the somewhat weaker binding by the “wobble” base, the oligonucleotide primers are designed such that the first and second positions of each triplet are occupied by nucleotide analogs which bind with greater affinity than the unmodified nucleotide. Examples of these analogs include, but are not limited to, 2,6-diaminopurine which binds to thymine, 5-propynyluracil which binds to adenine and 5-propynylcytosine and phenoxazines, including G-clamp, which binds to G. Propynylated pyrimidines are described in U.S. Pat. Nos. 5,645,985, 5,830,653 and 5,484,908, each of which is commonly owned and incorporated herein by reference in its entirety. Propynylated primers are described in U.S. Ser. No. 10/294,203 which is also commonly owned and incorporated herein by reference in entirety. Phenoxazines are described in U.S. Pat. Nos. 5,502,177, 5,763,588, and 6,005,096, each of which is incorporated herein by reference in its entirety. G-clamps are described in U.S. Pat. Nos. 6,007,992 and 6,028,183, each of which is incorporated herein by reference in its entirety.

In some embodiments, non-template primer tags are used to increase the melting temperature (Tₘ) of a primer-template duplex in order to improve amplification efficiency. A non-template tag is at least three consecutive A or T nucleotide residues on a primer which are not complementary to the template. In any given non-template tag, A can be replaced by C or G and T can also be replaced by C or G. Although Watson-Crick hybridization is not expected to occur for a non-template tag relative to the template, the extra hydrogen bond in a G-C pair relative to a A-T pair confers increased stability of the primer-template duplex and improves amplification efficiency for subsequent cycles of amplification when the primers hybridize to strands synthesized in previous cycles.

In other embodiments, propynylated tags may be used in a manner similar to that of the non-template tag, wherein two or more 5-propynylcytidine or 5-propynyluridine residues replace template matching residues on a primer. In other embodiments, a primer contains a modified internucleoside linkage such as a phosphorothioate linkage, for example.

In some embodiments, the primers contain mass-modifying tags. Reducing the total number of possible base compositions of a nucleic acid of specific molecular weight provides a means of avoiding a persistent source of ambiguity in determination of base composition of amplification products. Addition of mass-modifying tags to certain nucleobases of a given primer will result in simplification of de novo determination of base composition of a given bioagent identifying amplicon (vide infra) from its molecular mass.

In some embodiments of the present invention, the mass modified nucleobase comprises one or more of the following: for example, 7-deaza-2′-deoxyadenosine-5-triphosphate, 5-iodo-2′-deoxyuridine-5′-triphosphate, 5-bromo-2′-deoxyuridine-5′-triphosphate, 5-bromo-2′-deoxycytidine-5′-triphosphate, 5-iodo-2′-deoxycytidine-5′-triphosphate, 5-hydroxy-2′-deoxyuridine-5′-triphosphate, 4-thiothymidine-5′-triphosphate, 5-aza-2′-deoxyuridine-5′-triphosphate, 5-fluoro-2′-deoxyuridine-5′-triphosphate, O6-methyl-2′-deoxyguanosine-5′-triphosphate, N2-methyl-2′-deoxyguanosine-5′-triphosphate, 8-oxo-2′-deoxyguanosine-5′-triphosphate or thiothymidine-5′-triphosphate. In some embodiments, the mass-modified nucleobase comprises ¹⁵N or ¹³C or both ¹⁵N and ¹³C.

In some embodiments of the present invention, at least one bacterial nucleic acid segment is amplified in the process of identifying the bioagent. Thus, the nucleic acid segments that can be amplified by the primers disclosed herein and that provide enough variability to distinguish each individual bioagent and whose molecular masses are amenable to molecular mass determination are herein described as “bioagent identifying amplicons.” The term “amplicon” as used herein, refers to a segment of a polynucleotide which is amplified in an amplification reaction. In some embodiments of the present invention, bioagent identifying amplicons comprise from about 45 to about 200 nucleobases (i.e. from about 45 to about 200 linked nucleosides), from about 60 to about 150 nucleobases, from about 75 to about 125 nucleobases. One of ordinary skill in the art will appreciate that the invention embodies compounds of 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, and 200 nucleobases in length, or any range therewithin. It is the combination of the portions of the bioagent nucleic acid segment to which the primers hybridize (hybridization sites) and the variable region between the primer hybridization sites that comprises the bioagent identifying amplicon. Since genetic data provide the underlying basis for identification of bioagents by the methods of the present invention, it is prudent to select segments of nucleic acids which ideally provide enough variability to distinguish each individual bioagent and whose molecular mass is amenable to molecular mass determination.

In some embodiments, bioagent identifying amplicons amenable to molecular mass determination which are produced by the primers described herein are either of a length, size or mass compatible with the particular mode of molecular mass determination or compatible with a means of providing a predictable fragmentation pattern in order to obtain predictable fragments of a length compatible with the particular mode of molecular mass determination. Such means of providing a predictable fragmentation pattern of an amplification product include, but are not limited to, cleavage with restriction enzymes or cleavage primers, for example. Methods of using restriction enzymes and cleavage primers are well known to those with ordinary skill in the art.

In some embodiments, amplification products corresponding to bacterial bioagent identifying amplicons are obtained using the polymerase chain reaction (PCR) which is a routine method to those with ordinary skill in the molecular biology arts. Other amplification methods may be used such as ligase chain reaction (LCR), low-stringency single primer PCR, and multiple strand displacement amplification (MDA) which are also well known to those with ordinary skill.

In the context of this invention, a “bioagent” is any organism, cell, or virus, living or dead, or a nucleic acid derived from such an organism, cell or virus. Examples of bioagents include, but are not limited, to cells, (including but not limited to human clinical samples, bacterial cells and other pathogens), viruses, fungi, protists, parasites, and pathogenicity markers (including but not limited to: pathogenicity islands, antibiotic resistance genes, virulence factors, toxin genes and other bioregulating compounds). Samples may be alive or dead or in a vegetative state (for example, vegetative bacteria or spores) and may be encapsulated or bioengineered. In the context of this invention, a “pathogen” is a bioagent which causes a disease or disorder.

In the context of this invention, the term “unknown bioagent” may mean either: (i) a bioagent whose existence is known (such as the well known bacterial species Staphylococcus aureus for example) but which is not known to be in a sample to be analyzed, or (ii) a bioagent whose existence is not known (for example, the SARS coronavirus was unknown prior to April 2003). For example, if the method for identification of coronaviruses disclosed in commonly owned U.S. patent Ser. No. 10/829,826 (incorporated herein by reference in its entirety) was to be employed prior to April 2003 to identify the SARS coronavirus in a clinical sample, both meanings of “unknown” bioagent are applicable since the SARS coronavirus was unknown to science prior to April, 2003 and since it was not known what bioagent (in this case a coronavirus) was present in the sample. On the other hand, if the method of U.S. patent Ser. No. 10/829,826 was to be employed subsequent to April 2003 to identify the SARS coronavirus in a clinical sample, only the first meaning (i) of “unknown” bioagent would apply since the SARS coronavirus became known to science subsequent to April 2003 and since it was not known what bioagent was present in the sample.

The employment of more than one bioagent identifying amplicon for identification of a bioagent is herein referred to as “triangulation identification.” Triangulation identification is pursued by analyzing a plurality of bioagent identifying amplicons selected within multiple core genes. This process is used to reduce false negative and false positive signals, and enable reconstruction of the origin of hybrid or otherwise engineered bioagents. For example, identification of the three part toxin genes typical of B. anthracis (Bowen et al., J. Appl. Microbiol., 1999, 87, 270-278) in the absence of the expected signatures from the B. anthracis genome would suggest a genetic engineering event.

In some embodiments, the triangulation identification process can be pursued by characterization of bioagent identifying amplicons in a massively parallel fashion using the polymerase chain reaction (PCR), such as multiplex PCR where multiple primers are employed in the same amplification reaction mixture, or PCR in multi-well plate format wherein a different and unique pair of primers is used in multiple wells containing otherwise identical reaction mixtures. Such multiplex and multi-well PCR methods are well known to those with ordinary skill in the arts of rapid throughput amplification of nucleic acids.

In some embodiments, the molecular mass of a particular bioagent identifying amplicon is determined by mass spectrometry. Mass spectrometry has several advantages, not the least of which is high bandwidth characterized by the ability to separate (and isolate) many molecular peaks across a broad range of mass to charge ratio (m/z). Thus, mass spectrometry is intrinsically a parallel detection scheme without the need for radioactive or fluorescent labels, since every amplification product is identified by its molecular mass. The current state of the art in mass spectrometry is such that less than femtomole quantities of material can be readily analyzed to afford information about the molecular contents of the sample. An accurate assessment of the molecular mass of the material can be quickly obtained, irrespective of whether the molecular weight of the sample is several hundred, or in excess of one hundred thousand atomic mass units (amu) or Daltons.

In some embodiments, intact molecular ions are generated from amplification products using one of a variety of ionization techniques to convert the sample to gas phase. These ionization methods include, but are not limited to, electrospray ionization (ES), matrix-assisted laser desorption ionization (MALDI) and fast atom bombardment (FAB). Upon ionization, several peaks are observed from one sample due to the formation of ions with different charges. Averaging the multiple readings of molecular mass obtained from a single mass spectrum affords an estimate of molecular mass of the bioagent identifying amplicon. Electrospray ionization mass spectrometry (ESI-MS) is particularly useful for very high molecular weight polymers such as proteins and nucleic acids having molecular weights greater than 10 kDa, since it yields a distribution of multiply-charged molecules of the sample without causing a significant amount of fragmentation.

The mass detectors used in the methods of the present invention include, but are not limited to, Fourier transform ion cyclotron resonance mass spectrometry (FT-ICR-MS), time of flight (TOF), ion trap, quadrupole, magnetic sector, Q-TOF, and triple quadrupole.

In some embodiments, conversion of molecular mass data to a base composition is useful for certain analyses. As used herein, a “base composition” is the exact number of each nucleobase (A, T, C and G). For example, amplification of nucleic acid of Neisseria meningitidis with a primer pair that produces an amplification product from nucleic acid of 23S rRNA that has a molecular mass (sense strand) of 28480.75124, from which a base composition of A25 G27 C22 T18 is assigned from a list of possible base compositions calculated from the molecular mass using standard known molecular masses of each of the four nucleobases.

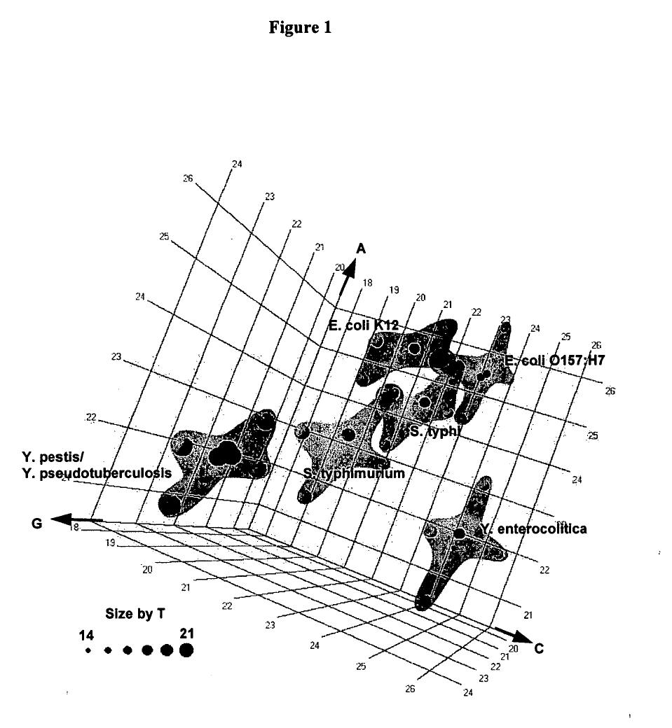

In some embodiments, assignment of base compositions to experimentally determined molecular masses is accomplished using “base composition probability clouds.” Base compositions, like sequences, vary slightly from isolate to isolate within species. It is possible to manage this diversity by building “base composition probability clouds” around the composition constraints for each species. This permits identification of organisms in a fashion similar to sequence analysis. A “pseudo four-dimensional plot” (FIG. 1) can be used to visualize the concept of base composition probability clouds. Optimal primer design requires optimal choice of bioagent identifying amplicons and maximizes the separation between the base composition signatures of individual bioagents. Areas where clouds overlap indicate regions that may result in a misclassification, a problem which is overcome by a triangulation identification process using bioagent identifying amplicons not affected by overlap of base composition probability clouds.

In some embodiments, base composition probability clouds provide the means for screening potential primer pairs in order to avoid potential misclassifications of base compositions. In other embodiments, base composition probability clouds provide the means for predicting the identity of a bioagent whose assigned base composition was not previously observed and/or indexed in a bioagent identifying amplicon base composition database due to evolutionary transitions in its nucleic acid sequence. Thus, in contrast to probe-based techniques, mass spectrometry determination of base composition does not require prior knowledge of the composition or sequence in order to make the measurement.

The present invention provides bioagent classifying information similar to DNA sequencing and phylogenetic analysis at a level sufficient to identify a given bioagent. Furthermore, the process of determination of a previously unknown base composition for a given bioagent (for example, in a case where sequence information is unavailable) has downstream utility by providing additional bioagent indexing information with which to populate base composition databases. The process of future bioagent identification is thus greatly improved as more BCS indexes become available in base composition databases.

In one embodiment, a sample comprising an unknown bioagent is contacted with a pair of primers which provide the means for amplification of nucleic acid from the bioagent, and a known quantity of a polynucleotide that comprises a calibration sequence. The nucleic acids of the bioagent and of the calibration sequence are amplified and the rate of amplification is reasonably assumed to be similar for the nucleic acid of the bioagent and of the calibration sequence. The amplification reaction then produces two amplification products: a bioagent identifying amplicon and a calibration amplicon. The bioagent identifying amplicon and the calibration amplicon should be distinguishable by molecular mass while being amplified at essentially the same rate. Effecting differential molecular masses can be accomplished by choosing as a calibration sequence, a representative bioagent identifying amplicon (from a specific species of bioagent) and performing, for example, a 2 to 8 nucleobase deletion or insertion within the variable region between the two priming sites. The amplified sample containing the bioagent identifying amplicon and the calibration amplicon is then subjected to molecular mass analysis by mass spectrometry, for example. The resulting molecular mass analysis of the nucleic acid of the bioagent and of the calibration sequence provides molecular mass data and abundance data for the nucleic acid of the bioagent and of the calibration sequence. The molecular mass data obtained for the nucleic acid of the bioagent enables identification of the unknown bioagent and the abundance data enables calculation of the quantity of the bioagent, based on the knowledge of the quantity of calibration polynucleotide contacted with the sample.

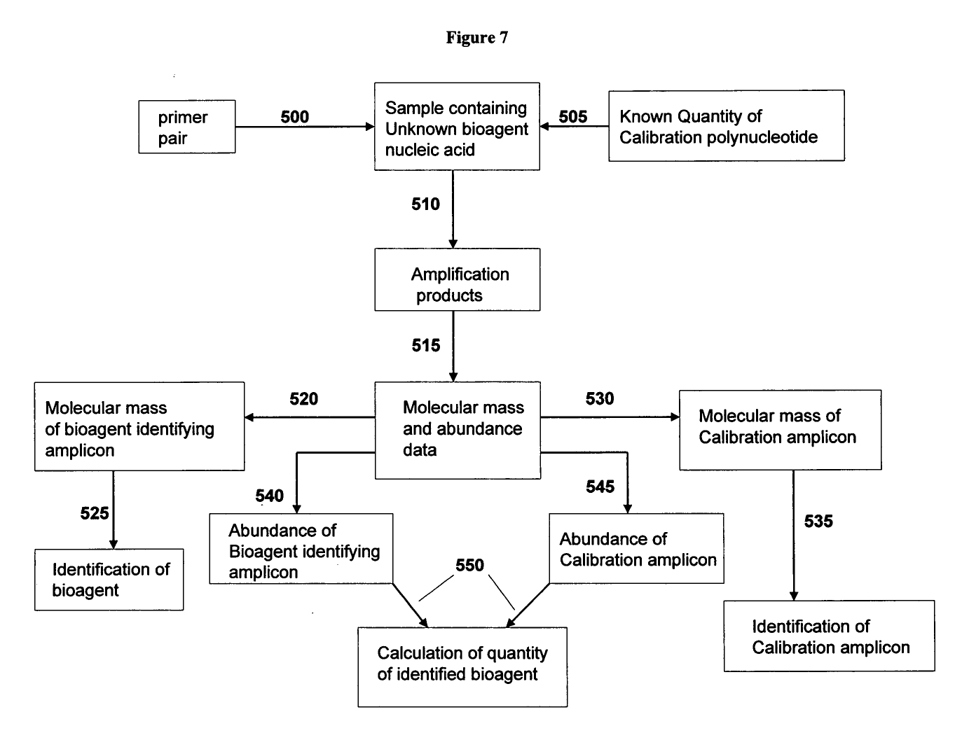

In some embodiments, the identity and quantity of a particular bioagent is determined using the process illustrated in FIG. 7. For instance, to a sample containing nucleic acid of an unknown bioagent are added primers (500) and a known quantity of a calibration polynucleotide (505). The total nucleic acid in the sample is subjected to an amplification reaction (510) to obtain amplification products. The molecular masses of amplification products are determined (515) from which are obtained molecular mass and abundance data. The molecular mass of the bioagent identifying amplicon (520) provides the means for its identification (525) and the molecular mass of the calibration amplicon obtained from the calibration polynucleotide (530) provides the means for its identification (535). The abundance data of the bioagent identifying amplicon is recorded (540) and the abundance data for the calibration data is recorded (545), both of which are used in a calculation (550) which determines the quantity of unknown bioagent in the sample.

In some embodiments, construction of a standard curve where the amount of calibration polynucleotide spiked into the sample is varied, provides additional resolution and improved confidence for the determination of the quantity of bioagent in the sample. The use of standard curves for analytical determination of molecular quantities is well known to one with ordinary skill and can be performed without undue experimentation.

In some embodiments, multiplex amplification is performed where multiple bioagent identifying amplicons are amplified with multiple primer pairs which also amplify the corresponding standard calibration sequences. In this or other embodiments, the standard calibration sequences are optionally included within a single vector which functions as the calibration polynucleotide. Multiplex amplification methods are well known to those with ordinary skill and can be performed without undue experimentation.

In some embodiments, the calibrant polynucleotide is used as an internal positive control to confirm that amplification conditions and subsequent analysis steps are successful in producing a measurable amplicon. Even in the absence of copies of the genome of a bioagent, the calibration polynucleotide should give rise to a calibration amplicon. Failure to produce a measurable calibration amplicon indicates a failure of amplification or subsequent analysis step such as amplicon purification or molecular mass determination. Reaching a conclusion that such failures have occurred is in itself, a useful event.

In some embodiments, the calibration sequence is inserted into a vector which then itself functions as the calibration polynucleotide. In some embodiments, more than one calibration sequence is inserted into the vector that functions as the calibration polynucleotide. Such a calibration polynucleotide is herein termed a “combination calibration polynucleotide.” The process of inserting polynucleotides into vectors is routine to those skilled in the art and can be accomplished without undue experimentation. Thus, it should be recognized that the calibration method should not be limited to the embodiments described herein. The calibration method can be applied for determination of the quantity of any bioagent identifying amplicon when an appropriate standard calibrant polynucleotide sequence is designed and used. The process of choosing an appropriate vector for insertion of a calibrant is also a routine operation that can be accomplished by one with ordinary skill without undue experimentation.

The present invention also provides kits for carrying out, for example, the methods described herein. In some embodiments, the kit may comprise a sufficient quantity of one or more primer pairs to perform an amplification reaction on a target polynucleotide from a bioagent to form a bioagent identifying amplicon. In some embodiments, the kit may comprise from one to fifty primer pairs, from one to twenty primer pairs, from one to ten primer pairs, or from two to five primer pairs. In some embodiments, the kit may comprise one or more primer pairs recited in Table 1.

In some embodiments, the kit may comprise one or more broad range survey primer(s), division wide primer(s), clade group primer(s) or drill-down primer(s), or any combination thereof. A kit may be designed so as to comprise particular primer pairs for identification of a particular bioagent. For example, a broad range survey primer kit may be used initially to identify an unknown bioagent as a member of the Bacillus/Clostridia group. Another example of a division-wide kit may be used to distinguish Bacillus anthracis, Bacillus cereus and Bacillus thuringiensis from each other. A clade group primer kit may be used, for example, to identify an unknown bacterium as a member of the Bacillus cereus clade group. A drill-down kit may be used, for example, to identify genetically engineered Bacillus anthracis. In some embodiments, any of these kits may be combined to comprise a combination of broad range survey primers and division-wide primers, clade group primers or drill-down primers, or any combination thereof, for identification of an unknown bacterial bioagent.

In some embodiments, the kit may contain standardized calibration polynucleotides for use as internal amplification calibrants. Internal calibrants are described in commonly owned U.S. Patent Application Ser. No. 60/545,425 which is incorporated herein by reference in its entirety.

In some embodiments, the kit may also comprise a sufficient quantity of reverse transcriptase (if an RNA virus is to be identified for example), a DNA polymerase, suitable nucleoside triphosphates (including any of those described above), a DNA ligase, and/or reaction buffer, or any combination thereof, for the amplification processes described above. A kit may further include instructions pertinent for the particular embodiment of the kit, such instructions describing the primer pairs and amplification conditions for operation of the method. A kit may also comprise amplification reaction containers such as microcentrifuge tubes and the like. A kit may also comprise reagents or other materials for isolating bioagent nucleic acid or bioagent identifying amplicons from amplification, including, for example, detergents, solvents, or ion exchange resins which may be linked to magnetic beads. A kit may also comprise a table of measured or calculated molecular masses and/or base compositions of bioagents using the primer pairs of the kit.

In order that the invention disclosed herein may be more efficiently understood, examples are provided below. It should be understood that these examples are for illustrative purposes only and are not to be construed as limiting the invention in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1

### Selection of Primers that Define Bioagent Identifying Amplicons

For design of primers that define bacterial bioagent identifying amplicons, relevant sequences from, for example, GenBank are obtained, aligned and scanned for regions where pairs of PCR primers would amplify products of about 45 to about 200 nucleotides in length and distinguish species from each other by their molecular masses or base compositions. A typical process shown in FIG. 2 is employed.

A database of expected base compositions for each primer region is generated using an in silico PCR search algorithm, such as (ePCR). An existing RNA structure search algorithm (Macke et al., Nuc. Acids Res., 2001, 29, 4724-4735, which is incorporated herein by reference in its entirety) has been modified to include PCR parameters such as hybridization conditions, mismatches, and thermodynamic calculations (SantaLucia, Proc. Natl. Acad. Sci. U.S.A., 1998, 95, 1460-1465, which is incorporated herein by reference in its entirety). This also provides information on primer specificity of the selected primer pairs.

Table 1 represents a collection of primers (sorted by forward primer name) designed to identify bacteria using the methods herein described. The forward or reverse primer name indicates the gene region of bacterial genome to which the primer hybridizes relative to a reference sequence eg: the forward primer name 16S_EC_{—}1077_{—}1106 indicates that the primer hybridizes to residues 1077-1106 of the gene encoding 16S ribosomal RNA in an E. coli reference sequence represented by a sequence extraction of coordinates 4033120.4034661 from GenBank gi number 16127994 (as indicated in Table 2). As an additional example: the forward primer name BONTA_X52066_{—}450_{—}473 indicates that the primer hybridizes to residues 450-437 of the gene encoding Clostridium botulinum neurotoxin type A (BoNT/A) represented by GenBank Accession No. X52066 (primer pair name codes appearing in Table 1 are defined in Table 2). In Table 1, U^{a}=5-propynyluracil; C^{a}=5-propynylcytosine; *=phosphorothioate linkage. The primer pair number is an in-house database index number.

TABLE 1
Primer Pairs for Identification of Bacterial Bioagents

| | For. | | For. | | | Rev. |
|---|---|---|---|---|---|---|
| Primer pair number | primer name | Forward sequence | SEQ ID NO: | Rev. primer name | Reverse sequence | SEQ ID NO: |
| 1 | 16S_EC_1077_1106_F | GTGAGATGTTGGGTTAA | 1 | 16S_EC_1175_1195_R | GACGTCATCCCCACCTTCC | 368 |
| | | GTCCCGTAACGAG | | | TC | |
| 266 | 16S_EC_1082_1100_F | ATGTTGGGTTAAGTCCC | 2 | 16S_EC_1177_1196_10G_11G_R | TGACGTCATGGCCACCTTCC | 372 |
| | | GC | | | | |
| 265 | 16S_EC_1082_1100_F | ATGTTGGGTTAAGTCCC | 2 | 16S_EC_1177_1196_10G_R | TGACGTCATGCCCACCTTCC | 373 |
| | | GC | | | | |
| 230 | 16S_EC_1082_1100_F | ATGTTGGGTTAAGTCCC | 2 | 16S_EC_1177_1196_R | TGACGTCATCCCCACCTTCC | 374 |
| | | GC | | | | |
| 263 | 16S_EC_1082_1100_F | ATGTTGGGTTAAGTCCC | 2 | 16S_EC_1525_1541_R | AAGGAGGTGATCCAGCC | 382 |
| | | GC | | | | |
| 2 | 16S_EC_1082_1106_F | ATGTTGGGTTAAGTCCC | 3 | 16S_EC_1175_1197_R | TTGACGTCATCCCCACCTT | 371 |
| | | GCAACGAG | | | CCTC | |
| 278 | 16S_EC_1090_1111_2_F | TTAAGTCCCGCAACGAG | 4 | 16S_EC_1175_1196_R | TGACGTCATCCCCACCTTC | 369 |
| | | CGCAA | | | CTC | |
| 361 | 16S_EC_1090_1111_2_TMOD_F | TTTAAGTCCCGCAACGA | 5 | 16S_EC_1175_1196_TMOD_R | TTGACGTCATCCCCACCTT | 370 |
| | | GCGCAA | | | CCTC | |
| 3 | 16S_EC_1090_1111_F | TTAAGTCCCGCAACGAT | 6 | 16S_EC_1175_1196_R | TGACGTCATCCCCACCTTC | 369 |
| | | CGCAA | | | CTC | |
| 256 | 16S_EC_1092_1109_F | TAGTCCCGCAACGAGCGC | 7 | 16S_EC_1174_1195_R | GACGTCATCCCCACCTTCC | 367 |
| | | | | | TCC | |
| 159 | 16S_EC_1100_1116_F | CAACGAGCGCAACCCTT | 8 | 16S_EC_1174_1188_R | TCCCCACCTTCCTCC | 366 |
| 247 | 16S_EC_1195_1213_F | CAAGTCATCATGGCCCT | 9 | 16S_EC_1525_1541_R | AAGGAGGTGATCCAGCC | 382 |
| | | TA | | | | |
| 4 | 16S_EC_1222_1241_F | GCTACACACGTGCTACA | 10 | 16S_EC_1303_1323_R | CGAGTTGCAGACTGCGATC | 376 |
| | | ATG | | | CG | |
| 232 | 16S_EC_1303_1323_F | CGGATTGGAGTCTGCAA | 11 | 16S_EC_1389_1407_R | GACGGGCGGTGTGTACAAG | 378 |
| | | CTCG | | | | |
| 5 | 16S_EC_1332_1353_F | AAGTCGGAATCGCTAGT | 12 | 16S_EC_1389_1407_R | GACGGGCGGTGTGTACAAG | 378 |
| | | AATCG | | | | |
| 252 | 16S_EC_1367_1387_F | TACGGTGAATACGTTCC | 13 | 16S_EC_1485_1506_R | ACCTTGTTACGACTTCACC | 379 |
| | | CGGG | | | CCA | |
| 250 | 16S_EC_1387_1407_F | GCCTTGTACACACCTCC | 14 | 16S_EC_1494_1513_R | CACGGCTACCTTGTTACGAC | 381 |
| | | CGTC | | | | |
| 231 | 16S_EC_1389_1407_F | CTTGTACACACCGCCCG | 15 | 16S_EC_1525_1541_R | AAGGAGGTGATCCAGCC | 382 |
| | | TC | | | | |
| 251 | 16S_EC_1390_1411_F | TTGTACACACCGCCCGT | 16 | 16S_EC_1486_1505_R | CCTTGTTACGACTTCACCCC | 380 |
| | | CATAC | | | | |
| 6 | 16S_EC_30_54_F | TGAACGCTGGTGGCATG | 17 | 16S_EC_105_126_R | TACGCATTACTCACCCGTC | 361 |
| | | CTTAACAC | | | CGC | |
| 243 | 16S_EC_314_332_F | CACTGGAACTGAGACAC | 18 | 16S_EC_556_575_R | CTTTACGCCCAGTAATTCCG | 385 |
| | | GG | | | | |
| 7 | 16S_EC_38_64_F | GTGGCATGCCTAATACA | 19 | 16S_EC_101_120_R | TTACTCACCCGTCCGCCGCT | 357 |
| | | TGCAAGTCG | | | | |
| 279 | 16S_EC_405_432_F | TGAGTGATGAAGGCCTT | 20 | 16S_EC_507_527_R | CGGCTGCTGGCACGAAGTT | 384 |
| | | AGGGTTGTAAA | | | AG | |
| 8 | 16S_EC_49_68_F | TAACACATGCAAGTCGA | 21 | 16S_EC_104_120_R | TTACTCACCCGTCCGCC | 359 |
| | | ACG | | | | |
| 275 | 16S_EC_49_68_F | TAACACATGCAAGTCGA | 21 | 16S_EC_1061_1078_R | ACGACACGAGCTGACGAC | 364 |
| | | ACG | | | | |
| 274 | 16S_EC_49_68_F | TAACACATGCAAGTCGA | 21 | 16S_EC_880_894_R | CGTACTCCCCAGGCG | 390 |
| | | ACG | | | | |
| 244 | 16S_EC_518_536_F | CCAGCAGCCGCGGTAAT | 22 | 16S_EC_774_795_R | GTATCTAATCCTGTTTGCT | 387 |
| | | AC | | | CCC | |
| 226 | 16S_EC_556_575_F | CGGAATTACTGGGCGTA | 23 | 16S_EC_683_700_R | CGCATTTCACCGCTACAC | 386 |
| | | AAG | | | | |
| 264 | 16S_EC_556_575_F | CGGAATTACTGGGCGTA | 23 | 16S_EC_774_795_R | GTATCTAATCCTGTTTGCT | 387 |
| | | AAG | | | CCC | |
| 273 | 16S_EC_683_700_F | GTGTAGCGGTGAAATGCG | 24 | 16S_EC_1303_1323_R | CGAGTTGCAGACTGCGATC | 377 |
| | | | | | CG | |
| 9 | 16S_EC_683_700_F | GTGTAGCGGTGAAATGCG | 24 | 16S_EC_774_795_R | GTATCTAATCCTGTTTGCT | 387 |
| | | | | | CCC | |
| 158 | 16S_EC_683_700_F | GTGTAGCGGTGAAATGCG | 24 | 16S_EC_880_894_R | CGTACTCCCCAGGCG | 390 |
| 245 | 16S_EC_683_700_F | GTGTAGCGGTGAAATGCG | 24 | 16S_EC_967_985_R | GGTAAGGTTCTTCGCGTTG | 396 |
| 294 | 16S_EC_7_33_F | GAGAGTTTGATCCTGGC | 25 | 16S_EC_101_122_R | TGTTACTCACCCGTCTGCC | 358 |
| | | TCAGAACGAA | | | ACT | |
| 10 | 16S_EC_713_732_F | AGAACACCGATGGCGAA | 26 | 16S_EC_789_809_R | CGTGGACTACCAGGGTATC | 388 |
| | | GGC | | | TA | |
| 346 | 16S_EC_713_732_TMOD_F | TAGAACACCGATGGCGA | 27 | 16S_EC_789_809_TMOD_R | TCGTGGACTACCAGGGTAT | 389 |
| | | AGGC | | | CTA | |
| 228 | 16S_EC_774_795_F | GGGAGCAAACAGGATTA | 28 | 16S_EC_880_894_R | CGTACTCCCCAGGCG | 390 |
| | | GATAC | | | | |
| 11 | 16S_EC_785_806_F | GGATTAGAGACCCTGGT | 29 | 16S_EC_880_897_R | GGCCGTACTCCCCAGGCG | 391 |
| | | AGTCC | | | | |
| 347 | 16S_EC_785_806_TMOD_F | TGGATTAGAGACCCTGG | 30 | 16S_EC_880_897_TMOD_R | TGGCCGTACTCCCCAGGCG | 392 |
| | | TAGTCC | | | | |
| 12 | 16S_EC_785_810_F | GGATTAGATACCCTGGT | 31 | 16S_EC_880_897_2_R | GGCCGTACTCCCCAGGCG | 391 |
| | | AGTCCACGC | | | | |
| 13 | 16S_EC_789_810_F | TAGATACCCTGGTAGTC | 32 | 16S_EC_880_894_R | CGTACTCCCCAGGCG | 390 |
| | | CACGC | | | | |
| 255 | 16S_EC_789_810_F | TAGATACCCTGGTAGTC | 32 | 16S_EC_882_899_R | GCGACCGTACTCCCCAGG | 393 |
| | | CACGC | | | | |
| 254 | 16S_EC_791_812_F | GATACCCTGGTAGTCCA | 33 | 16S_EC_886_904_R | GCCTTGCGACCGTACTCCC | 394 |
| | | CACCG | | | | |
| 248 | 16S_EC_8_27_F | AGAGTTTGATCATGGCT | 34 | 16S_EC_1525_1541_R | AAGGAGGTGATCCAGCC | 382 |
| | | CAG | | | | |
| 242 | 16S_EC_8_27_F | AGAGTTTGATCATGGCT | 34 | 16S_EC_342_358_R | ACTGCTGCCTCCCGTAG | 383 |
| | | CAG | | | | |
| 253 | 16S_EC_804_822_F | ACCACGCCGTAAACGAT | 35 | 16S_EC_909_929_R | CCCCCGTCAATTCCTTTGA | 395 |
| | | GA | | | GT | |
| 246 | 16S_EC_937_954_F | AAGCGGTGGAGCATGTGG | 36 | 16S_EC_1220_1240_R | ATTGTAGCACGTGTGTAGC | 375 |
| | | | | | CC | |
| 14 | 16S_EC_960_981_F | TTCGATGCAACGCGAAG | 37 | 16S_EC_1054_1073_R | ACGAGCTGACGACAGCCATG | 362 |
| | | AACCT | | | | |
| 348 | 16S_EC_960_981_TMOD_F | TTTCGATGCAACGCGAA | 38 | 16S_EC_1054_1073_TMOD_R | TACGAGCTGACGACAGCCA | 363 |
| | | GAACCT | | | TG | |
| 119 | 16S_EC_969_985_1P_F | ACGCGAAGAACCTTA | 39 | 16S_EC_1061_1078_2P_R | ACGACACGAGU^{a}C^{a}GACGAC | 364 |
| | | U^{a}C | | | | |
| 15 | 16S_EC_969_985_F | ACGCGAAGAACCTTACC | 39 | 16S_EC_1061_1078_R | ACGACACGAGCTGACGAC | 364 |
| 272 | 16S_EC_969_985_F | ACGCGAAGAACCTTACC | 40 | 16S_EC_1389_1407_R | GACGGGCGGTGTGTACAAG | 378 |
| 344 | 16S_EC_971_990_F | GCGAAGAACCTTACCAG | 41 | 16S_EC_1043_1062_R | ACAACCATGCACCACCTGTC | 360 |
| | | GTC | | | | |
| 120 | 16S_EC_972_985_2P_F | CGAAGAAU^{a}U^{a}TTACC | 42 | 16S_EC_1064_1075_2P_R | ACACGAGU^{a}C^{a}GAC | 365 |
| 121 | 16S_EC_972_985_F | CGAAGAACCTTACC | 42 | 16S_EC_1064_1075_R | ACACGAGCTGAC | 365 |
| 1073 | 23S_BRM_1110_1129_F | TGCGCGGAAGATGTAAC | 43 | 23S_BRM_1176_1201_R | TCGCAGGCTTACAGAACGC | 397 |
| | | GGG | | | TCTCCTA | |
| 1074 | 23S_BRM_515_536_F | TGCATACAAACAGTCGG | 44 | 23S_BRM_616_635_R | TCGGACTCGCTTTCGCTACG | 398 |
| | | AGCCT | | | | |
| 241 | 23S_BS_- | AAACTAGATAACAGTAG | 45 | 23S_BS_5_21_R | GTGCGCCCTTTCTAACTT | 399 |
| | 68_-44_F | ACATCAC | | | | |
| 235 | 23S_EC_1602_1620_F | TACCCCAAACCGACACA | 46 | 23S_EC_1686_1703_R | CCTTCTCCCGAAGTTACG | 402 |
| | | GG | | | | |
| 236 | 23S_EC_1685_1703_F | CCGTAACTTCGGGAGAA | 47 | 23S_EC_1828_1842_R | CACCGGGCAGGCGTC | 403 |
| | | GG | | | | |
| 16 | 23S_EC_1826_1843_F | CTGACACCTGCCCGGTGC | 48 | 23S_EC_1906_1924_R | GACCGTTATAGTTACGGCC | 404 |
| 349 | 23S_EC_1826_1843_TMOD_F | TCTGACACCTGCCCGGT | 49 | 23S_EC_1906_1924_TMOD_R | TGACCGTTATAGTTACGGCC | 405 |
| | | GC | | | | |
| 237 | 23S_EC_1827_1843_F | GACGCCTGCCCGGTGC | 50 | 23S_EC_1929_1949_R | CCGACAAGGAATTTCGCTA | 407 |
| | | | | | CC | |
| 249 | 23S_EC_1831_1849_F | ACCTGCCCAGTGCTGGA | 51 | 23S_EC_1919_1936_R | TCGCTACCTTAGGACCGT | 406 |
| | | AG | | | | |
| 234 | 23S_EC_187_207_F | GGGAACTGAAACATCTA | 52 | 23S_EC_242_256_R | TTCGCTCGCCGCTAC | 408 |
| | | AGTA | | | | |
| 233 | 23S_EC_23_37_F | GGTGGATGCCTTGGC | 53 | 23S_EC_115_130_R | GGGTTTCCCCATTCGG | 401 |
| 238 | 23S_EC_2434_2456_F | AAGGTACTCCGGGGATA | 54 | 23S_EC_2490_2511_R | AGCCGACATCGAGGTGCCA | 409 |
| | | ACAGGC | | | AAC | |
| 257 | 23S_EC_2586_2607_F | TAGAACGTCGCGAGACA | 55 | 23S_EC_2658_2677_R | AGTCCATCCCGGTCCTCTCG | 411 |
| | | GTTCG | | | | |
| 239 | 23S_EC_2599_2616_F | GACAGTTCGGTCCCTATC | 56 | 23S_EC_2653_2669_R | CCGGTCCTCTCGTACTA | 410 |
| 18 | 23S_EC_2645_2669_2_F | CTGTCCCTAGTACGAGA | 57 | 23S_EC_2751_2767_R | GTTTCATGCTTAGATGCTT | 417 |
| | | GGACCGG | | | TCAGC | |
| 17 | 23S_EC_2645_2669_F | TCTGTCCCTAGTACGAG | 58 | 23S_EC_2744_2761_R | TGCTTAGATGCTTTCAGC | 414 |
| | | AGGACCGG | | | | |
| 118 | 23S_EC_2646_2667_F | CTGTTCTTAGTACGAGA | 59 | 23S_EC_2745_2765_R | TTCGTGCTTAGATGCTTTC | 415 |
| | | GGACC | | | AG | |
| 360 | 23S_EC_2646_2667_TMOD_F | TCTGTTCTTAGTACGAG | 60 | 23S_EC_2745_2765_TMOD_R | TTTCGTGCTTAGATGCTTT | 416 |
| | | AGGACC | | | CAG | |
| 147 | 23S_EC_2652_2669_F | CTAGTACGAGAGGACCGG | 61 | 23S_EC_2741_2760_R | ACTTAGATGCTTTCAGCGGT | 413 |
| 240 | 23S_EC_2653_2669_F | TAGTACGAGAGGACCGG | 62 | 23S_EC_2737_2758_R | TTAGATGCTTTCAGCACTT | 412 |
| | | | | | ATC | |
| 20 | 23S_EC_493_518_2_F | GGGGAGTGAAAGAGATC | 63 | 23S_EC_551_571_2_R | ACAAAAGGCACGCCATCAC | 418 |
| | | CTGAAACCG | | | CC | |
| 19 | 23S_EC_493_518_F | GGGGAGTGAAAGAGATC | 63 | 23S_EC_551_571_R | ACAAAAGGTACGCCGTCAC | 419 |
| | | CTGAAACCG | | | CC | |
| 21 | 23S_EC_971_992_F | CGAGAGGGAAACAACCC | 64 | 23S_EC_1059_1077_R | TGGCTGCTTCTAAGCCAAC | 400 |
| | | AGACC | | | | |
| 1158 | AB_MLST- | TCGTGCCCGCAATTTGC | 65 | AB_MLST-11- | TAATGCCGGGTAGTGCAAT | 420 |
| | 11- | ATAAAGC | | OIF007_1266_1296_R | CCATTCTTCTAG | |
| | OIF007_1202_1225_F | | | | | |
| 1159 | AB_MLST- | TCGTGCCCGCAATTTGC | 65 | AB_MLST-11- | TGCACCTGCGGTCGAGCG | 421 |
| | 11- | ATAAAGC | | OIF007_1299_1316_R | | |
| | OIF007_1202_1225_F | | | | | |
| 1160 | AB_MLST- | TTGTAGCACAGCAAGGC | 66 | AB_MLST-11- | TGCCATCCATAATCACGCC | 422 |
| | 11- | AAATTTCCTGAAAC | | OIF007_1335_1362_R | ATACTGACG | |
| | OIF007_1234_1264_F | | | | | |
| 1161 | AB_MLST- | TAGGTTTACGTCAGTAT | 67 | AB_MLST-11- | TGCCAGTTTCCACATTTCA | 423 |
| | 11- | GGCGTGATTATGG | | OIF007_1422_1448_R | CGTTCGTG | |
| | OIF007_1327_1356_F | | | | | |
| 1162 | AB_MLST- | TCGTGATTATGGATGGC | 68 | AB_MLST-11- | TCGCTTGAGTGTAGTCATG | 424 |
| | 11- | AACGTGAA | | OIF007_1470_1494_R | ATTGCG | |
| | OIF007_1345_1369_F | | | | | |
| 1163 | AB_MLST- | TTATGGATGGCAACGTG | 69 | AB_MLST-11- | TCGCTTGAGTGTAGTCATG | 424 |
| | 11- | AAACGCGT | | OIF007_1470_1494_R | ATTGCG | |
| | OIF007_1351_1375_F | | | | | |
| 1164 | AB_MLST- | TCTTTGCCATTGAAGAT | 70 | AB_MLST-11- | TCGCTTGAGTGTAGTCATG | 424 |
| | 11- | GACTTAAGC | | OIF007_1470_1494_R | ATTGCG | |
| | OIF007_1387_1412_F | | | | | |
| 1165 | AB_MLST- | TACTAGCGGTAAGCTTA | 71 | AB_MLST-11- | TGAGTCGGGTTCACTTTAC | 425 |
| | 11- | AACAAGATTGC | | OIF007_1656_1680_R | CTGGCA | |
| | OIF007_1542_1569_F | | | | | |
| 1166 | AB_MLST- | TTGCCAATGATATTCGT | 72 | AB_MLST-11- | TGAGTCGGGTTCACTTTAC | 425 |
| | 11- | TGGTTAGCAAG | | OIF007_1656_1680_R | CTGGCA | |
| | OIF007_1566_1593_F | | | | | |
| 1167 | AB_MLST- | TCGGCGAAATCCGTATT | 73 | AB_MLST-11- | TACCGGAAGCACCAGCGAC | 427 |
| | 11- | CCTGAAAATGA | | OIF007_1731_1757_R | ATTAATAG | |
| | OIF007_1611_1638_F | | | | | |
| 1168 | AB_MLST- | TACCACTATTAATGTCG | 74 | AB_MLST-11- | TGCAACTGAATAGATTGCA | 428 |
| | 11- | CTGGTGCTTC | | OIF007_1790_1821_R | GTAAGTTATAAGC | |
| | OIF007_1726_1752_F | | | | | |
| 1169 | AB_MLST- | TTATAACTTACTGCAAT | 75 | AB_MLST-11- | TGAATTATGCAAGAAGTGA | 429 |
| | 11- | CTATTCAGTTGCTTGGTG | | OIF007_1876_1909_R | TCAATTTTCTCACGA | |
| | OIF007_1792_1826_F | | | | | |
| 1170 | AB_MLST- | TTATAACTTACTGCAAT | 75 | AB_MLST-11- | TGCCGTAACTAACATAAGA | 430 |
| | 11- | CTATTCAGTTGCTTGGTG | | OIF007_1895_1927_R | GAATTATGCAAGAA | |
| | OIF007_1792_1826_F | | | | | |
| 1152 | AB_MLST- | TATTGTTTCAAATGTAC | 76 | AB_MLST-11- | TCACAGGTTCTACTTCATC | 432 |
| | 11- | AAGGTGAAGTGCG | | OIF007_291_324_R | AATAATTTCCATTGC | |
| | OIF007_185_214_F | | | | | |
| 1171 | AB_MLST- | TGGTTATGTACCAAATA | 77 | AB_MLST-11- | TGACGGCATCGATACCACC | 431 |
| | 11- | CTTTGTCTGAAGATGG | | OIF007_2097_2118_R | GTC | |
| | OIF007_1970_2002_F | | | | | |
| 1154 | AB_MLST- | TGAAGTGCGTGATGATA | 78 | AB_MLST-11- | TCCGCCAAAAACTCCCCTT | 433 |
| | 11- | TCGATGCACTTGATGTA | | OIF007_318_344_R | TTCACAGG | |
| | OIF007_206_239_F | | | | | |
| 1153 | AB_MLST- | TGGAACGTTATCAGGTG | 79 | AB_MLST-11- | TTGCAATCGACATATCCAT | 434 |
| | 11- | CCCCAAAAATTCG | | OIF007_364_393_R | TTCACCATGCC | |
| | OIF007_260_289_F | | | | | |
| 1155 | AB_MLST- | TCGGTTTAGTAAAAGAA | 80 | AB_MLST-11- | TTCTGCTTGAGGAATAGTG | 435 |
| | 11- | CGTATTGCTCAACC | | OIF007_587_610_R | CGTGG | |
| | OIF007_522_552_F | | | | | |
| 1156 | AB_MLST- | TCAACCTGACTGCGTGA | 81 | AB_MLST-11- | TACGTTCTACGATTTCTTC | 436 |
| | 11- | ATGGTTGT | | OIF007_656_686_R | ATCAGGTACATC | |
| | OIF007_547_571_F | | | | | |
| 1157 | AB_MLST- | TCAAGCAGAAGCTTTGG | 82 | AB_MLST-11- | TACAACGTGATAAACACGA | 437 |
| | 11- | AAGAAGAAGG | | OIF007_710_736_R | CCAGAAGC | |
| | OIF007_601_627_F | | | | | |
| 1151 | AB_MLST- | TGAGATTGCTGAACATT | 83 | AB_MLST-11- | TTGTACATTTGAAACAATA | 426 |
| | 11- | TAATGCTGATTGA | | OIF007_169_203_R | TGCATGACATGTGAAT | |
| | OIF007_62_91_F | | | | | |
| 1100 | ASD_FRT_1_29_F | TTGCTTAAAGTTGGTTT | 84 | ASD_FRT_86_116_R | TGAGATGTCGAAAAAAACG | 439 |
| | | TATTGGTTGGCG | | | TTGGCAAAATAC | |
| 1101 | ASD_FRT_43_76_F | TCAGTTTTAATGTCTCG | 85 | ASD_FRT_129_156_R | TCCATATTGTTGCATAAAA | 438 |
| | | TATGATCGAATCAAAAG | | | CCTGTTGGC | |
| 291 | ASPS_EC_405_422_F | GCACAACCTGCGGCTGCG | 86 | ASPS_EC_521_538_R | ACGGCACGAGGTAGTCGC | 440 |
| 485 | BONTA_X52066_450_473_F | TCTAGTAATAATAGGAC | 87 | BONTA_X52066_517_539_R | TAACCATTTCGCGTAAGAT | 441 |
| | | CCTCAGC | | | TCAA | |
| 486 | BONTA_X52066_450_473P_F | T*U^{a}*C^{a}AGTAATAATAG | 87 | BONTA_X52066_517_539P_R | TAACCA*C^{a}*C^{a}*C^{a}*U^{a}*GC | 441 |
| | | GA*U^{a}*U^{a}*U^{a}*C^{a}*U^{a}AGC | | | GTAAGA*C^{a}*C^{a}*U^{a}AA | |
| 481 | BONTA_X52066_538_552_F | TATGGCTCTACTCAA | 88 | BONTA_X52066_647_660_R | TGTTACTGCTGGAT | 443 |
| 482 | BONTA_X52066_538_552P_F | TA*C^{a}GGC*C^{a}*U^{a}*C^{a}A | 88 | BONTA_X52066_647_660P_R | TG*C^{a}*C^{a}A*U^{a}*C^{a}G*U^{a}*C^{a} | 443 |
| | | *U^{a}*C^{a}*U^{a}AA | | | GGAT | |
| 487 | BONTA_X52066_591_620_F | TGAGTCACTTGAAGTTG | 89 | BONTA_X52066_644_671_R | TCATGTGCTAATGTTACTG | 442 |
| | | ATACAAATCCTCT | | | CTGGATCTG | |
| 483 | BONTA_X52066_701_720_F | GAATAGCAATTAATCCA | 90 | BONTA_X52066_759_775_R | TTACTTCTAACCCACTC | 444 |
| | | AAT | | | | |
| 484 | BONTA_X52066_701_720P_F | GAA*C^{a}AG*U^{a}AA*C^{a}*C^{a} | 90 | BONTA_X52066_759_775P_R | TTA*U^{a}*C^{a}*C^{a}*U^{a}*C^{a}AA* | 444 |
| | | AA*C^{a}*U^{a}*U^{a}AAAT | | | U^{a}*U^{a}*U^{a}A*U^{a}*C^{a}C | |
| 774 | CAF1_AF053947_33407_33430_F | TCAGTTCCGTTATCGCC | 91 | CAF1_AF053947_33494_33514_R | TGCGGGCTGGTTCAACAAG | 445 |
| | | ATTGCAT | | | AG | |
| 776 | CAF1_AF053947_33435_33457_F | TGGAACTATTGCAACTG | 92 | CAF1_AF053947_33499_33517_R | TGATGCGGGCTGGTTCAAC | 446 |
| | | CTAATG | | | | |
| 775 | CAF1_AF053947_33515_33541_F | TCACTCTTACATATAAG | 93 | CAF1_AF053947_33595_33621_R | TCCTGTTTTATAGCCGCCA | 447 |
| | | GAAGGCGCTC | | | AGAGTAAG | |
| 777 | CAF1_AF053947_33687_33716_F | TCAGGATGGAAATAACC | 94 | CAF1_AF053947_33755_33782_R | TCAAGGTTCTCACCGTTTA | 448 |
| | | ACCAATTCACTAC | | | CCTTAGGAG | |
| 22 | CAPC_BA_104_131_F | GTTATTTAGCACTCGTT | 95 | CAPC_BA_180_205_R | TGAATCTTGAAACACCATA | 449 |
| | | TTTAATCAGCC | | | CGTAACG | |
| 23 | CAPC_BA_114_133_F | ACTCGTTTTTAATCAGC | 96 | CAPC_BA_185_205_R | TGAATCTTGAAACACCATA | 450 |
| | | CCG | | | CG | |
| 24 | CAPC_BA_274_303_F | GATTATTGTTATCCTGT | 97 | CAPC_BA_349_376_R | GTAACCCTTGTCTTTGAAT | 451 |
| | | TATGCCATTTGAG | | | TGTATTTGC | |
| 350 | CAPC_BA_274_303_TMOD_F | TGATTATTGTTATCCTG | 98 | CAPC_BA_349_376_TMOD_R | TGTAACCCTTGTCTTTGAA | 452 |
| | | TTATGCCATTTGAG | | | TTGTATTTGC | |
| 25 | CAPC_BA_276_296_F | TTATTGTTATCCTGTTA | 99 | CAPC_BA_358_377_R | GGTAACCCTTGTCTTTGAAT | 453 |
| | | TGCC | | | | |
| 26 | CAPC_BA_281_301_F | GTTATCCTGTTATGCCA | 100 | CAPC_BA_361_378_R | TGGTAACCCTTGTCTTTG | 454 |
| | | TTTG | | | | |
| 27 | CAPC_BA_315_334_F | CCGTGGTATTGGAGTTA | 101 | CAPC_BA_361_378_R | TGGTAACCCTTGTCTTTG | 454 |
| | | TTG | | | | |
| 1053 | CJST_CJ_1080_1110_F | TTGAGGGTATGCACCGT | 102 | CJST_CJ_1166_1198_R | TCCCCTCATGTTTAAATGA | 456 |
| | | CTTTTTGATTCTTT | | | TCAGGATAAAAAGC | |
| 1063 | CJST_CJ_1268_1299_F | AGTTATAAACACGGCTT | 103 | CJST_CJ_1349_1379_R | TCGGTTTAAGCTCTACATG | 457 |
| | | TCCTATGGCTTATCC | | | ATCGTAAGGATA | |
| 1050 | CJST_CJ_1290_1320_F | TGGCTTATCCAAATTTA | 104 | CJST_CJ_1406_1433_R | TTTGCTCATGATCTGCATG | 458 |
| | | GATCGTGGTTTTAC | | | AAGCATAAA | |
| 1058 | CJST_CJ_1643_1670_F | TTATCGTTTGTGGAGCT | 105 | CJST_CJ_1724_1752_R | TGCAATGTGTGCTATGTCA | 459 |
| | | AGTGCTTATGC | | | GCAAAAAGAT | |
| 1045 | CJST_CJ_1668_1700_F | TGCTCGAGTGATTGACT | 106 | CJST_CJ_1774_1799_R | TGAGCGTGTGGAAAAGGAC | 460 |
| | | TTGCTAAATTTAGAGA | | | TTGGATG | |
| 1064 | CJST_CJ_1680_1713_F | TGATTTTGCTAAATTTA | 107 | CJST_CJ_1795_1822_R | TATGTGTAGTTGAGCTTAC | 461 |
| | | GAGAAATTGCGGATGAA | | | TACATGAGC | |
| 1056 | CJST_CJ_1880_1910_F | TCCCAATTAATTCTGCC | 108 | CJST_CJ_1981_2011_R | TGGTTCTTACTTGCTTTGC | 462 |
| | | ATTTTTCCAGGTAT | | | ATAAACTTTCCA | |
| 1054 | CJST_CJ_2060_2090_F | TCCCGGACTTAATATCA | 109 | CJST_CJ_2148_2174_R | TCGATCCGCATCACCATCA | 463 |
| | | ATGAAAATTGTGGA | | | AAAGCAAA | |
| 1059 | CJST_CJ_2165_2194_F | TGCGGATCGTTTGGTGG | 110 | CJST_CJ_2247_2278_R | TCCACACTGGATTGTAATT | 464 |
| | | TTGTAGATGAAAA | | | TACCTTGTTCTTT | |
| 1046 | CJST_CJ_2171_2197_F | TCGTTTGGTGGTGGTAG | 111 | CJST_CJ_2283_2313_R | TCTCTTTCAAAGCACCATT | 465 |
| | | ATGAAAAAGG | | | GCTCATTATAGT | |
| 1057 | CJST_CJ_2185_2212_F | TAGATGAAAAGGGCGAA | 112 | CJST_CJ_2283_2316_R | TGAATTCTTTCAAAGCACC | 466 |
| | | GTGGCTAATGG | | | ATTGCTCATTATAGT | |
| 1049 | CJST_CJ_2636_2668_F | TGCCTAGAAGATCTTAA | 113 | CJST_CJ_2753_2777_R | TTGCTGCCATAGCAAAGCC | 467 |
| | | AAATTTCCGCCAACTT | | | TACAGC | |
| 1062 | CJST_CJ_2678_2703_F | TCCCCAGGACACCCTGA | 114 | CJST_CJ_2760_2787_R | TGTGCTTTTTTTGCTGCCA | 468 |
| | | AATTTCAAC | | | TAGCAAAGC | |
| 1065 | CJST_CJ_2857_2887_F | TGGCATTTCTTATGAAG | 115 | CJST_CJ_2965_2998_R | TGCTTCAAAACGCATTTTT | 469 |
| | | CTTGTTCTTTAGCA | | | ACATTTTCGTTAAAG | |
| 1055 | CJST_CJ_2869_2895_F | TGAAGCTTGTTCTTTAG | 116 | CJST_CJ_2979_3007_R | TCCTCCTTGTGCCTCAAAA | 470 |
| | | CAGGACTTCA | | | CGCATTTTTA | |
| 1051 | CJST_CJ_3267_3293_F | TTTGATTTTACGCCGTC | 117 | CJST_CJ_3356_3385_R | TCAAAGAACCCGCACCTAA | 471 |
| | | CTCCAGGTCG | | | TTCATCATTTA | |
| 1061 | CJST_CJ_360_393_F | TCCTGTTATCCCTGAAG | 118 | CJST_CJ_443_477_R | TACAACTGGTTCAAAAACA | 473 |
| | | TAGTTAATCAAGTTTGT | | | TTAAGCTGTAATTGTC | |
| 1048 | CJST_CJ_360_394_F | TCCTGTTATCCCTGAAG | 119 | CJST_CJ_442_476_R | TCAACTGGTTCAAAAACAT | 472 |
| | | TAGTTAATCAAGTTTGTT | | | TAAGTTGTAATTGTCC | |
| 1052 | CJST_CJ_5_39_F | TAGGCGAAGATATACAA | 120 | CJST_CJ_104_137_R | TCCCTTATTTTTCTTTCTA | 455 |
| | | AGAGTATTAGAAGCTAGA | | | CTACCTTCGGATAAT | |
| 1047 | CJST_CJ_584_616_F | TCCAGGACAAATGTATG | 121 | CJST_CJ_663_692_R | TTCATTTTCTGGTCCAAAG | 474 |
| | | AAAAATGTCCAAGAAG | | | TAAGCAGTATC | |
| 1060 | CJST_CJ_599_632_F | TGAAAAATGTCCAAGAA | 122 | CJST_CJ_711_743_R | TCCCGAACAATGAGTTGTA | 475 |
| | | GCATAGCAAAAAAAGCA | | | TCAACTATTTTTAC | |
| 1096 | CTXA_VBC_117_142_F | TCTTATGCCAAGAGGAC | 123 | CTXA_VBC_194_218_R | TGCCTAACAAATCCCGTCT | 476 |
| | | AGAGTGAGT | | | GAGTTC | |
| 1097 | CTXA_VBC_351_377_F | TGTATTAGGGGCATACA | 124 | CTXA_VBC_441_466_R | TGTCATCAAGCACCCCAAA | 477 |
| | | GTCCTCATCC | | | ATGAACT | |
| 28 | CYA_BA_1055_1072_F | GAAAGAGTTCGGATTGGG | 125 | CYA_BA_1112_1130_R | TGTTGACCATGCTTCTTAG | 479 |
| 277 | CYA_BA_1349_1370_F | ACAACGAAGTACAATAC | 126 | CYA_BA_1426_1447_R | CTTCTACATTTTTAGCCAT | 480 |
| | | AAGAC | | | CAC | |
| 30 | CYA_BA_1353_1379_F | CGAAGTACAATACAAGA | 127 | CYA_BA_1448_1467_R | TGTTAACGGCTTCAAGACCC | 482 |
| | | CAAAAGAAGG | | | | |
| 351 | CYA_BA_1353_1379_TMOD_F | TCGAAGTACAATACAAG | 128 | CYA_BA_1448_1467_TMOD_R | TTGTTAACGGCTTCAAGAC | 483 |
| | | ACAAAAGAAGG | | | CC | |
| 31 | CYA_BA_1359_1379_F | ACAATACAAGACAAAAG | 129 | CYA_BA_1447_1461_R | CGGCTTCAAGACCCC | 481 |
| | | AAGG | | | | |
| 32 | CYA_BA_914_937_F | CAGGTTTAGTACCAGAA | 130 | CYA_BA_999_1026_R | ACCACTTTTAATAAGGTTT | 484 |
| | | CATGCAG | | | GTAGCTAAC | |
| 33 | CYA_BA_916_935_F | GGTTTAGTACCAGAACA | 131 | CYA_BA_1003_1025_R | CCACTTTTAATAAGGTTTG | 478 |
| | | TGC | | | TAGC | |
| 115 | DNAK_EC_428_449_F | CGGCGTACTTCAACGAC | 132 | DNAK_EC_503_522_R | CGCGGTCGGCTCGTTGATGA | 485 |
| | | AGCCA | | | | |
| 1102 | GALE_FRT_168_199_F | TTATCAGCTAGACCTTT | 133 | GALE_FRT_241_269_R | TCACCTACAGCTTTAAAGC | 486 |
| | | TAGGTAAAGCTAAGC | | | CAGCAAAATG | |
| 1104 | GALE_FRT_308_339_F | TCCAAGGTACACTAAAC | 134 | GALE_FRT_390_422_R | TCTTCTGTAAAGGGTGGTT | 487 |
| | | TTACTTGAGCTAATG | | | TATTATTCATCCCA | |
| 1103 | GALE_FRT_834_865_F | TCAAAAAGCCCTAGGTA | 135 | GALE_FRT_901_925_R | TAGCCTTGGCAACATCAGC | 488 |
| | | AAGAGATTCCATATC | | | AAAACT | |
| 1092 | GLTA_RKP_1023_1055_F | TCCGTTCTTACAAATAG | 136 | GLTA_RKP_1129_1156_R | TTGGCGACGGTATACCCAT | 489 |
| | | CAATAGAACTTGAAGC | | | AGCTTTATA | |
| 1093 | GLTA_RKP_1043_1072_2_F | TGGAGCTTGAAGCTATC | 137 | GLTA_RKP_1138_1162_R | TGAACATTTGCGACGGTAT | 490 |
| | | GCTCTTAAAGATG | | | ACCCAT | |
| 1094 | GLTA_RKP_1043_1072_3_F | TGGAACTTGAAGCTCTC | 138 | GLTA_RKP_1138_1164_R | TGTGAACATTTGCGACGGT | 492 |
| | | GCTCTTAAAGATG | | | ATACCCAT | |
| 1090 | GLTA_RKP_1043_1072_F | TGGGACTTGAAGCTATC | 139 | GLTA_RKP_1138_1162_R | TGAACATTTGCGACGGTAT | 491 |
| | | GCTCTTAAAGATG | | | ACCCAT | |
| 1091 | GLTA_RKP_400_428_F | TCTTCTCATCCTATGGC | 140 | GLTA_RKP_499_529_R | TGGTGGGTATCTTAGCAAT | 493 |
| | | TATTATGCTTGC | | | CATTCTAATAGC | |
| 1095 | GLTA_RKP_400_428_F | TCTTCTCATCCTATGGC | 140 | GLTA_RKP_505_534_R | TGCGATGGTAGGTATCTTA | 494 |
| | | TATTATGCTTGC | | | GCAATCATTCT | |
| 224 | GROL_EC_219_242_F | GGTGAAAGAAGTTGCCT | 141 | GROL_EC_328_350_R | TTCAGGTCCATCGGGTTCA | 496 |
| | | CTAAAGC | | | TGCC | |
| 280 | GROL_EC_496_518_F | ATGGACAAGGTTGGCAA | 142 | GROL_EC_577_596_R | TAGCCGCGGTCGAATTGCAT | 498 |
| | | GGAAGG | | | | |
| 281 | GROL_EC_511_536_F | AAGGAAGGCGTGATCAC | 143 | GROL_EC_571_593_R | CCGCGGTCGAATTGCATGC | 497 |
| | | CGTTGAAGA | | | CTTC | |
| 220 | GROL_EC_941_959_F | TGGAAGATCTGGGTCAG | 144 | GROL_EC_1039_1060_R | CAATCTGCTGACGGATCTG | 495 |
| | | GC | | | AGC | |
| 924 | GYRA_AF100557_4_23_F | TCTGCCCGTGTCGTTGG | 145 | GYRA_AF100557_119_142_R | TCGAACCGAAGTTACCCTG | 499 |
| | | TGA | | | ACCAT | |
| 925 | GYRA_AF100557_70_94_F | TCCATTGTTCGTATGGC | 146 | GYRA_AF100557_178_201_R | TGCCAGCTTAGTCATACGG | 500 |
| | | TCAAGACT | | | ACTTC | |
| 926 | GYRB_AB008700_19_40_F | TCAGGTGGCTTACACGG | 147 | GYRB_AB008700_111_140_R | TATTGCGGATCACCATGAT | 501 |
| | | CGTAG | | | GATATTCTTGC | |
| 927 | GYRB_AB008700_265_292_F | TCTTTCTTGAATGCTGG | 148 | GYRB_AB008700_369_395_R | TCGTTGAGATGGTTTTTAC | 502 |
| | | TGTACGTATCG | | | CTTCGTTG | |
| 928 | GYRB_AB008700_368_394_F | TCAACGAAGGTAAAAAC | 149 | GYRB_AB008700_466_494_R | TTTGTGAAACAGCGAACAT | 503 |
| | | CATCTCAACG | | | TTTCTTGGTA | |
| 929 | GYRB_AB008700_477_504_F | TGTTCGCTGTTTCACAA | 150 | GYRB_AB008700_611_632_R | TCACGCGCATCATCACCAG | 504 |
| | | ACAACATTCCA | | | TCA | |
| 949 | GYRB_AB008700_760_787_F | TACTTACTTGAGAATCC | 151 | GYRB_AB008700_862_888_2_R | TCCTGCAATATCTAATGCA | 505 |
| | | ACAAGCTGCAA | | | CTCTTACG | |
| 930 | GYRB_AB008700_760_787_F | TACTTACTTGAGAATCC | 151 | GYRB_AB008700_862_888_R | ACCTGCAATATCTAATGCA | 506 |
| | | ACAAGCTGCAA | | | CTCTTACG | |
| 222 | HFLB_EC_1082_1102_F | TGGCGAACCTGGTGAAC | 152 | HFLB_EC_1144_1168_R | CTTTCGCTTTCTCGAACTC | 507 |
| | | GAAGC | | | AACCAT | |
| 1128 | HUPB_CJ_113_134_F | TAGTTGCTCAAACAGCT | 153 | HUPB_CJ_157_188_R | TCCCTAATAGTAGAAATAA | 509 |
| | | GGGCT | | | CTGCATCAGTAGC | |
| 1130 | HUPB_CJ_76_102_F | TCCCGGAGCTTTTATGA | 154 | HUPB_CJ_114_135_R | TAGCCCAGCTGTTTGAGCA | 508 |
| | | CTAAAGCAGAT | | | ACT | |
| 1129 | HUPB_CJ_76_102_F | TCCCGGAGCTTTTATGA | 154 | HUPB_CJ_157_188_R | TCCCTAATAGTAGAAATAA | 510 |
| | | CTAAAGCAGAT | | | CTGCATCAGTAGC | |
| 1079 | ICD_CXB_176_198_F | TCGCCGTGGAAAAATCC | 155 | ICD_CXB_224_247_R | TAGCCTTTTCTCCGGCGTA | 512 |
| | | TACGCT | | | GATCT | |
| 1078 | ICD_CXB_92_120_F | TTCCTGACCGACCCATT | 156 | ICD_CXB_172_194_R | TAGGATTTTTCCACGGCGG | 510 |
| | | ATTCCCTTTATC | | | CATC | |
| 1077 | ICD_CXB_93_120_F | TCCTGACCGACCCATTA | 157 | ICD_CXB_172_194_R | TAGGATTTTTCCACGGCGG | 511 |
| | | TTCCCTTTATC | | | CATC | |
| 221 | INFB_EC_1103_1124_F | GTCGTGAAAACGAGCTG | 158 | INFB_EC_1174_1191_R | CATGATGGTCACAACCGG | 513 |
| | | GAAGA | | | | |
| 964 | INFB_EC_1347_1367_F | TGCGTTTACCGCAATGC | 159 | INFB_EC_1414_1432_R | TCGGCATCACGCCGTCGTC | 514 |
| | | GTGC | | | | |
| 34 | INFB_EC_1365_1393_F | TGCTCGTGGTGCACAAG | 160 | INFB_EC_1439_1467_R | TGCTGCTTTCGCATGGTTA | 515 |
| | | TAACGGATATTA | | | ATTGCTTCAA | |
| 352 | INFB_EC_1365_1393_TMOD_F | TTGCTCGTGGTGCACAA | 161 | INFB_EC_1439_1467_TMOD_R | TTGCTGCTTTCGCATGGTT | 516 |
| | | GTAACGGATATTA | | | AATTGCTTCAA | |
| 223 | INFB_EC_1969_1994_F | CGTCAGGGTAAATTCCG | 162 | INFB_EC_2038_2058_R | AACTTCGCCTTCGGTCATG | 517 |
| | | TGAAGTTAA | | | TT | |
| 781 | INV_U22457_1558_1581_F | TGGTAACAGAGCCTTAT | 163 | INV_U22457_1619_1643_R | TTGCGTTGCAGATTATCTT | 518 |
| | | AGGCGCA | | | TACCAA | |
| 778 | INV_U22457_515_539_F | TGGCTCCTTGGTATGAC | 164 | INV_U22457_571_598_R | TGTTAAGTGTGTTGCGGCT | 519 |
| | | TCTGCTTC | | | GTCTTTATT | |
| 779 | INV_U22457_699_724_F | TGCTGAGGCCTGGACCG | 165 | INV_U22457_753_776_R | TCACGCGACGAGTGCCATC | 520 |
| | | ATTATTTAC | | | CATTG | |
| 780 | INV_U22457_834_858_F | TTATTTACCTGCACTCC | 166 | INV_U22457_942_966_R | TGACCCAAAGCTGAAAGCT | 521 |
| | | CACAACTG | | | TTACTG | |
| 1106 | IPAH_SGF_113_134_F | TCCTTGACCGCCTTTCC | 167 | IPAH_SGF_172_191_R | TTTTCCAGCCATGCAGCGAC | 522 |
| | | GATAC | | | | |
| 1105 | IPAH_SGF_258_277_F | TGAGGACCGTGTCGCGC | 168 | IPAH_SGF_301_327_R | TCCTTCTGATGCCTGATGG | 523 |
| | | TCA | | | ACCAGGAG | |
| 1107 | IPAH_SGF_462_486_F | TCAGACCATGCTCGCAG | 169 | IPAH_SGF_522_540_R | TGTCACTCCCGACACGCCA | 524 |
| | | AGAAACTT | | | | |
| 1080 | IS1111A_NC002971_6866_6891_F | TCAGTATGTATCCACCG | 170 | IS1111A_NC002971_6928_6954_R | TAAACGTCCGATACCAATG | 525 |
| | | TAGCCAGTC | | | GTTCGCTC | |
| 1081 | IS1111A_NC002971_7456_7483_F | TGGGTGACATTCATCAA | 171 | IS1111A_NC002971_7529_7554_R | TCAACAACACCTCCTTATT | 526 |
| | | TTTCATCGTTC | | | CCCACTC | |
| 35 | LEF_BA_1033_1052_F | TCAAGAAGAAAAAGAGC | 172 | LEF_BA_1119_1135_R | GAATATCAATTTGTAGC | 527 |
| 36 | LEF_BA_1036_1066_F | CAAGAAGAAAAAGAGCT | 173 | LEF_BA_1119_1149_R | AGATAAAGAATCACGAATA | 528 |
| | | TCTAAAAAGAATAC | | | TCAATTTGTAGC | |
| 37 | LEF_BA_756_781_F | AGCTTTTGCATATTATA | 174 | LEF_BA_843_872_R | TCTTCCAAGGATAGATTTA | 530 |
| | | TCGAGCCAC | | | TTTCTTGTTCG | |
| 353 | LEF_BA_756_781_TMOD_F | TAGCTTTTGCATATTAT | 175 | LEF_BA_843_872_TMOD_R | TTCTTCCAAGGATAGATTT | 531 |
| | | ATCGAGCCAC | | | ATTTCTTGTTCG | |
| 38 | LEF_BA_758_778_F | CTTTTGCATATTATATC | 176 | LEF_BA_843_865_R | AGGATAGATTTATTTCTTG | 529 |
| | | GAGC | | | TTCG | |
| 39 | LEF_BA_795_813_F | TTTACAGCTTTATGCAC | 177 | LEF_BA_883_900_R | TCTTGACAGCATCCGTTG | 532 |
| | | CG | | | | |
| 40 | LEF_BA_883_899_F | CAACGGATGCTGGCAAG | 178 | LEF_BA_939_958_R | CAGATAAAGAATCGCTCCAG | 533 |
| 782 | LL_NC003143_2366996_2367019_F | TGTAGCCGCTAAGCACT | 179 | LL_NC003143_2367073_2367097_R | TCTCATCCCGATATTACCG | 534 |
| | | ACCATCC | | | CCATGA | |
| 783 | LL_NC003143_2367172_2367194_F | TGGACGGCATCACGATT | 180 | LL_NC003143_2367249_2367271_R | TGGCAACAGCTCAACACCT | 535 |
| | | CTCTAC | | | TTGG | |
| 878 | MECA_Y14051_3645_3670_F | TGAAGTAGAAATGACTG | 181 | MECA_Y14051_3690_3719_R | TGATCCTGAATGTTTATAT | 536 |
| | | AACGTCCGA | | | CTTTAACGCCT | |
| 877 | MECA_Y14051_3774_3802_F | TAAAACAAACTACGGTA | 182 | MECA_Y14051_3828_3854_R | TCCCAATCTAACTTCCACA | 537 |
| | | ACATTGATCGCA | | | TACCATCT | |
| 879 | MECA_Y14051_4507_4530_F | TCAGGTACTGCTATCCA | 183 | MECA_Y14051_4555_4581_R | TGGATAGACGTCATATGAA | 538 |
| | | CCCTCAA | | | GGTGTGCT | |
| 880 | MECA_Y14051_4510_4530_F | TGTACTGCTATCCACCC | 184 | MECA_Y14051_4586_4610_R | TATTCTTCGTTACTCATGC | 539 |
| | | TCAA | | | CATACA | |
| 882 | MECA_Y14051_4520_4530P_F | TU^{a}U^{a}AU^{a}U^{a}U^{a}C^{a}U^{a}AA | 185 | MECA_Y14051_4590_4600P_R | C^{a}AU^{a}C^{a}U^{a}AC^{a}GU^{a}U^{a}A | 540 |
| 883 | MECA_Y14051_4520_4530P_F | TU^{a}U^{a}AU^{a}U^{a}U^{a}C^{a}U^{a}AA | 185 | MECA_Y14051_4600_4610P_R | C^{a}AC^{a}C^{a}U^{a}C^{a}C^{a}U^{a}GC^{a}T | 541 |
| 881 | MECA_Y14051_4669_4698_F | TCACCAGGTTCAACTCA | 186 | MECA_Y14051_4765_4793_R | TAACCACCCCAAGATTTAT | 542 |
| | | AAAAATATTAACA | | | CTTTTTGCCA | |
| 876 | MECIA_Y14051_3315_3341_F | TTACACATATCGTGAGC | 187 | MECIA_Y14051_3367_3393_R | TGTGATATGGAGGTGTAGA | 543 |
| | | AATGAACTGA | | | AGGTGTTA | |
| 914 | OMPA_AY485227_272_301_F | TTACTCCATTATTGCTT | 188 | OMPA_AY485227_364_388_R | GAGCTGCGCCAACGAATAA | 544 |
| | | GGTTACACTTTCC | | | ATCGTC | |
| 916 | OMPA_AY485227_311_335_F | TACACAACAATGGCGGT | 189 | OMPA_AY485227_424_453_R | TACGTCGCCTTTAACTTGG | 545 |
| | | AAAGATGG | | | TTATATTCAGC | |
| 915 | OMPA_AY485227_379_401_F | TGCGCAGCTCTTGGTAT | 190 | OMPA_AY485227_492_519_R | TGCCGTAACATAGAAGTTA | 546 |
| | | CGAGTT | | | CCGTTGATT | |
| 917 | OMPA_AY485227_415_441_F | TGCCTCGAAGCTGAATA | 191 | OMPA_AY485227_514_546_R | TCGGGCGTAGTTTTTAGTA | 547 |
| | | TAACCAAGTT | | | ATTAAATCAGAAGT | |
| 918 | OMPA_AY485227_494_520_F | TCAACGGTAACTTCTAT | 192 | OMPA_AY485227_569_596_R | TCGTCGTATTTATAGTGAC | 548 |
| | | GTTACTTCTG | | | CAGCACCTA | |
| 919 | OMPA_AY485227_551_577_F | TCAAGCCGTACGTATTA | 193 | OMPA_AY485227_658_680_R | TTTAAGCGCCAGAAAGCAC | 550 |
| | | TTAGGTGCTG | | | CAAC | |
| 920 | OMPA_AY485227_555_581_F | TCCGTACGTATTATTAG | 194 | OMPA_AY485227_635_662_R | TCAACACCAGCGTTACCTA | 549 |
| | | GTGCTGGTCA | | | AAGTACCTT | |
| 921 | OMPA_AY485227_556_583_F | TCGTACGTATTATTAGG | 195 | OMPA_AY485227_659_683_R | TCGTTTAAGCGCCAGAAAG | 551 |
| | | TGCTGGTCACT | | | CACCAA | |
| 922 | OMPA_AY485227_657_679_F | TGTTGGTGCTTTCTGGC | 196 | OMPA_AY485227_739_765_R | TAAGCCAGCAAGAGCTGTA | 552 |
| | | GCTTAA | | | TAGTTCCA | |
| 923 | OMPA_AY485227_660_683_F | TGGTGCTTTCTGGCGCT | 197 | OMPA_AY485227_786_807_R | TACAGGAGCAGCAGGCTTC | 553 |
| | | TAAACGA | | | AAG | |
| 1088 | OMPB_RKP_192_1221_F | TCTACTGATTTTGGTAA | 198 | OMPB_RKP_1288_1315_R | TAGCAGCAAAAGTTATCAC | 554 |
| | | TCTTGCAGCACAG | | | ACCTGCAGT | |
| 1089 | OMPB_RKP_3417_3440_F | TGCAAGTGGTACTTCAA | 199 | OMPB_RKP_3520_3550_R | TGGTTGTAGTTCCTGTAGT | 555 |
| | | CATGGGG | | | TGTTGCATTAAC | |
| 1087 | OMPB_RKP_860_890_F | TTACAGGAAGTTTAGGT | 200 | OMPB_RKP_972_996_R | TCCTGCAGCTCTACCTGCT | 556 |
| | | GGTAATCTAAAAGG | | | CCATTA | |
| 41 | PAG_BA_122_142_F | CAGAATCAAGTTCCCAG | 201 | PAG_BA_190_209_R | CCTGTAGTAGAAGAGGTAAC | 558 |
| | | GGG | | | | |
| 42 | PAG_BA_123_145_F | AGAATCAAGTTCCCAGG | 202 | PAG_BA_187_210_R | CCCTGTAGTAGAAGAGGTA | 557 |
| | | GGTTAC | | | ACCAC | |
| 43 | PAG_BA_269_287_F | AATCTGCTATTTGGTCA | 203 | PAG_BA_326_344_R | TGATTATCAGCGGAAGTAG | 559 |
| | | GG | | | | |
| 44 | PAG_BA_655_675_F | GAAGGATATACGGTTGA | 204 | PAG_BA_755_772_R | CCGTGCTCCATTTTTCAG | 560 |
| | | TGTC | | | | |
| 45 | PAG_BA_753_772_F | TCCTGAAAAATGGAGCA | 205 | PAG_BA_849_868_R | TCGGATAAGCTGCCACAAGG | 561 |
| | | CGG | | | | |
| 46 | PAG_BA_763_781_F | TGGAGCACGGCTTCTGA | 206 | PAG_BA_849_868_R | TCGGATAAGCTGCCACAAGG | 562 |
| | | TC | | | | |
| 912 | PARC_X95819_123_147_F | GGCTCAGCCATTTAGTT | 207 | PARC_X95819_232_260_R | TCGCTCAGCAATAATTCAC | 566 |
| | | ACCGCTAT | | | TATAAGCCGA | |
| 913 | PARC_X95819_43_63_F | TCAGCGCGTACAGTGGG | 208 | PARC_X95819_143_170_R | TTCCCCTGACCTTCGATTA | 563 |
| | | TGAT | | | AAGGATAGC | |
| 911 | PARC_X95819_87_110_F | TGGTGACTCGGCATGTT | 209 | PARC_X95819_192_219_R | GGTATAACGCATCGCAGCA | 564 |
| | | ATGAAGC | | | AAAGATTTA | |
| 910 | PARC_X95819_87_110_F | TGGTGACTCGGCATGTT | 209 | PARC_X95819_201_222_R | TTCGGTATAACGCATCGCA | 565 |
| | | ATGAAGC | | | GCA | |
| 773 | PLA_AF053945_7186_7211_F | TTATACCGGAAACTTCC | 210 | PLA_AF053945_7257_7280_R | TAATGCGATACTGGCCTGC | 567 |
| | | CGAAAGGAG | | | AAGTC | |
| 770 | PLA_AF053945_7377_7402_F | TGACATCCGGCTCACGT | 211 | PLA_AF053945_7434_7462_R | TGTAAATTCCGCAAAGACT | 568 |
| | | TATTATGGT | | | TTGGCATTAG | |
| 771 | PLA_AF053945_7382_7404_F | TCCGGCTCACGTTATTA | 212 | PLA_AF053945_7482_7502_R | TGGTCTGAGTACCTCCTTT | 569 |
| | | TGGTAC | | | GC | |
| 772 | PLA_AF053945_7481_7503_F | TGCAAAGGAGGTACTCA | 213 | PLA_AF053945_7539_7562_R | TATTGGAAATACCGGCAGC | 570 |
| | | GACCAT | | | ATCTC | |
| 909 | RECA_AF251469_169_190_F | TGACATGCTTGTCCGTT | 214 | RECA_AF251469_277_300_R | TGGCTCATAAGACGCGCTT | 572 |
| | | CAGGC | | | GTAGA | |
| 908 | RECA_AF251469_43_68_F | TGGTACATGTGCCTTCA | 215 | RECA_AF251469_140_163_R | TTCAAGTGCTTGCTCACCA | 571 |
| | | TTGATGCTG | | | TTGTC | |
| 1072 | RNASEP_BDP_574_592_F | TGGCACGGCCATCTCCG | 216 | RNASEP_BDP_616_635_R | TCGTTTCACCCTGTCATGC | 573 |
| | | TG | | | CG | |
| 1070 | RNASEP_BKM_580_599_F | TGCGGGTAGGGAGCTTG | 217 | RNASEP_BKM_665_686_R | TCCGATAAGCCGGATTCTG | 574 |
| | | AGC | | | TGC | |
| 1071 | RNASEP_BKM_616_637_F | TCCTAGAGGAATGGCTG | 218 | RNASEP_BKM_665_687_R | TGCCGATAAGCCGGATTCT | 575 |
| | | CCACG | | | GTGC | |
| 1112 | RNASEP_BRM_325_347_F | TACCCCAGGGAAAGTGC | 219 | RNASEP_BRM_402_428_R | TCTCTTACCCCACCCTTTC | 576 |
| | | CACAGA | | | ACCCTTAC | |
| 1172 | RNASEP_BRM_461_488_F | TAAACCCCATCGGGAGC | 220 | RNASEP_BRM_542_561_2_R | TGCCTCGTGCAACCCACCCG | 577 |
| | | AAGACCGAATA | | | | |
| 1111 | RNASEP_BRM_461_488_F | TAAACCCCATCGGGAGC | 220 | RNASEP_BRM_542_561_R | TGCCTCGCGCAACCTACCCG | 578 |
| | | AAGACCGAATA | | | | |
| 258 | RNASEP_BS_43_61_F | GAGGAAAGTCCATGCTC | 221 | RNASEP_BS_363_384_R | GTAAGCCATGTTTTGTTCC | 579 |
| | | GC | | | ATC | |
| 259 | RNASEP_BS_43_61_F | GAGGAAAGTCCATGCTC | 221 | RNASEP_BS_363_384_R | GTAAGCCATGTTTTGTTCC | 578 |
| | | GC | | | ATC | |
| 258 | RNASEP_BS_43_61_F | GAGGAAAGTCCATGCTC | 221 | RNASEP_EC_345_362_R | ATAAGCCGGGTTCTGTCG | 581 |
| | | GC | | | | |
| 258 | RNASEP_BS_43_61_F | GAGGAAAGTCCATGCTC | 221 | RNASEP_SA_358_379_R | ATAAGCCATGTTCTGTTCC | 584 |
| | | GC | | | ATC | |
| 1076 | RNASEP_CLB_459_487_F | TAAGGATAGTGCAACAG | 222 | RNASEP_CLB_498_522_R | TTTACCTCGCCTTTCCACC | 579 |
| | | AGATATACCGCC | | | CTTACC | |
| 1075 | RNASEP_CLB_459_487_F | TAAGGATAGTGCAACAG | 222 | RNASEP_CLB_498_526_R | TGCTCTTACCTCACCGTTC | 580 |
| | | AGATATACCGCC | | | CACCCTTACC | |
| 258 | RNASEP_EC_61_77_F | GAGGAAAGTCCGGGCTC | 223 | RNASEP_BS_363_384_R | GTAAGCCATGTTTTGTTCC | 578 |
| | | | | | ATC | |
| 258 | RNASEP_EC_61_77_F | GAGGAAAGTCCGGGCTC | 223 | RNASEP_EC_345_362_R | ATAAGCCGGGTTCTGTCG | 581 |
| 260 | RNASEP_EC_61_77_F | GAGGAAAGTCCGGGCTC | 223 | RNASEP_EC_345_362_R | ATAAGCCGGGTTCTGTCG | 581 |
| 258 | RNASEP_EC_61_77_F | GAGGAAAGTCCGGGCTC | 223 | RNASEP_SA_358_379_R | ATAAGCCATGTTCTGTTCC | 584 |
| | | | | | ATC | |
| 1085 | RNASEP_RKP_264_287_F | TCTAAATGGTCGTGCAG | 224 | RNASEP_RKP_295_321_R | TCTATAGAGTCCGGACTTT | 582 |
| | | TTGCGTG | | | CCTCGTGA | |
| 1082 | RNASEP_RKP_419_448_F | TGGTAAGAGCGCACCGG | 225 | RNASEP_RKP_542_565_R | TCAAGCGATCTACCCGCAT | 583 |
| | | TAAGTTGGTAACA | | | TACAA | |
| 1083 | RNASEP_RKP_422_443_F | TAAGAGCGCACCGGTAA | 226 | RNASEP_RKP_542_565_R | TCAAGCGATCTACCCGCAT | 583 |
| | | GTTGG | | | TACAA | |
| 1086 | RNASEP_RKP_426_448_F | TGCATACCGGTAAGTTG | 227 | RNASEP_RKP_542_565_R | TCAAGCGATCTACCCGCAT | 583 |
| | | GCAACA | | | TACAA | |
| 1084 | RNASEP_RKP_466_491_F | TCCACCAAGAGCAAGAT | 228 | RNASEP_RKP_542_565_R | TCAAGCGATCTACCCGCAT | 583 |
| | | CAAATAGGC | | | TACAA | |
| 258 | RNASEP_SA_31_49_F | GAGGAAAGTCCATGCTC | 229 | RNASEP_BS_363_384_R | GTAAGCCATGTTTTGTTCC | 578 |
| | | AC | | | ATC | |
| 258 | RNASEP_SA_31_49_F | GAGGAAAGTCCATGCTC | 229 | RNASEP_EC_345_362_R | ATAAGCCGGGTTCTGTCG | 581 |
| | | AC | | | | |
| 258 | RNASEP_SA_31_49_F | GAGGAAAGTCCATGCTC | 229 | RNASEP_SA_358_379_R | ATAAGCCATGTTCTGTTCC | 584 |
| | | AC | | | ATC | |
| 262 | RNASEP_SA_31_49_F | GAGGAAAGTCCATGCTC | 229 | RNASEP_SA_358_379_R | ATAAGCCATGTTCTGTTCC | 584 |
| | | AC | | | ATC | |
| 1098 | RNASEP_VBC_331_349_F | TCCGCGGAGTTGACTGG | 230 | RNASEP_VBC_388_414_R | TGACTTTCCTCCCCCTTAT | 585 |
| | | GT | | | CAGTCTCC | |
| 66 | RPLB_EC_650_679_F | GACCTACAGTAAGAGGT | 231 | RPLB_EC_739_762_R | TCCAAGTGCTGGTTTACCC | 591 |
| | | TCTGTAATGAACC | | | CATGG | |
| 356 | RPLB_EC_650_679_TMOD_F | TGACCTACAGTAAGAGG | 232 | RPLB_EC_739_762_TMOD_R | TTCCAAGTGCTGGTTTACC | 592 |
| | | TTCTGTAATGAACC | | | CCATGG | |
| 73 | RPLB_EC_669_698_F | TGTAATGAACCCTAATG | 233 | RPLB_EC_735_761_R | CCAAGTGCTGGTTTACCCC | 586 |
| | | ACCATCCACACGG | | | ATGGAGTA | |
| 74 | RPLB_EC_671_700_F | TAATGAACCCTAATGAC | 234 | RPLB_EC_737_762_R | TCCAAGTGCTGGTTTACCC | 590 |
| | | CATCCACACGGTG | | | CATGGAG | |
| 67 | RPLB_EC_688_710_F | CATCCACACGGTGGTGG | 235 | RPLB_EC_736_757_R | GTGCTGGTTTACCCCATGG | 587 |
| | | TGAAGG | | | AGT | |
| 70 | RPLB_EC_688_710_F | CATCCACACGGTGGTGG | 235 | RPLB_EC_743_771_R | TGTTTTGTATCCAAGTGCT | 593 |
| | | TGAAGG | | | GGTTTACCCC | |
| 357 | RPLB_EC_688_710_TMOD_F | TCATCCACACGGTGGTG | 236 | RPLB_EC_736_757_TMOD_R | TGTGCTGGTTTACCCCATG | 588 |
| | | GTGAAGG | | | GAGT | |
| 449 | RPLB_EC_690_710_F | TCCACACGGTGGTGGTG | 237 | RPLB_EC_737_758_R | TGTGCTGGTTTACCCCATG | 589 |
| | | AAGG | | | GAG | |
| 113 | RPOB_EC_1336_1353_F | GACCACCTCGGCAACCGT | 238 | RPOB_EC_1438_1455_R | TTCGCTCTCGGCCTGGCC | 594 |
| 963 | RPOB_EC_1527_1549_F | TCAGCTGTCGCAGTTCA | 239 | RPOB_EC_1630_1649_R | TCGTCGCGGACTTCGAAGCC | 595 |
| | | TGGACC | | | | |
| 72 | RPOB_EC_1845_1866_F | TATCGCTCAGGCGAACT | 240 | RPOB_EC_1909_1929_R | GCTGGATTCGCCTTTGCTA | 596 |
| | | CCAAC | | | CG | |
| 359 | RPOB_EC_1845_1866_TMOD_F | TTATCGCTCAGGCGAAC | 241 | RPOB_EC_1909_1929_TMOD_R | TGCTGGATTCGCCTTTGCT | 597 |
| | | TCCAAC | | | ACG | |
| 962 | RPOB_EC_2005_2027_F | TCGTTCCTGGAACACGA | 242 | RPOB_EC_2041_2064_R | TTGACGTTGCATGTTCGAG | 598 |
| | | TGACGC | | | CCCAT | |
| 69 | RPOB_EC_3762_3790_F | TCAACAACCTCTTGGAG | 243 | RPOB_EC_3836_3865_R | TTTCTTGAAGAGTATGAGC | 600 |
| | | GTAAAGCTCAGT | | | TGCTCCGTAAG | |
| 111 | RPOB_EC_3775_3803_F | CTTGGAGGTAAGTCTCA | 244 | RPOB_EC_3829_3858_R | CGTATAAGCTGCACCATAA | 599 |
| | | TTTTGGTGGGCA | | | GCTTGTAATGC | |
| 940 | RPOB_EC_3798_3821_F | TGGGCAGCGTTTCGGCG | 245 | RPOB_EC_3862_3889_2_R | TGTCCGACTTGACGGTTAG | 604 |
| | | AAATGGA | | | CATTTCCTG | |
| 939 | RPOB_EC_3798_3821_F | TGGGCAGCGTTTCGGCG | 245 | RPOB_EC_3862_3889_R | TGTCCGACTTGACGGTCAG | 605 |
| | | AAATGGA | | | CATTTCCTG | |
| 289 | RPOB_EC_3799_3821_F | GGGCAGCGTTTCGGCGA | 246 | RPOB_EC_3862_3888_R | GTCCGACTTGACGGTCAAC | 602 |
| | | AATGGA | | | ATTTCCTG | |
| 362 | RPOB_EC_3799_3821_TMOD_F | TGGGCAGCGTTTCGGCG | 245 | RPOB_EC_3862_3888_TMOD_R | TGTCCGACTTGACGGTCAA | 603 |
| | | AAATGGA | | | CATTTCCTG | |
| 288 | RPOB_EC_3802_3821_F | CAGCGTTTCGGCGAAAT | 247 | RPOB_EC_3862_3885_R | CGACTTGACGGTTAACATT | 601 |
| | | GGA | | | TCCTG | |
| 48 | RPOC_EC_1018_1045_2_F | CAAAACTTATTAGGTAA | 248 | RPOC_EC_1095_1124_2_R | TCAAGCGCCATCTCTTTCGF | 610 |
| | | GCGTGTTGACT | | | GTAATCCACAT | |
| 47 | RPOC_EC_1018_1045_F | CAAAACTTATTAGGTAA | 248 | RPOC_EC_1095_1124_R | TCAAGCGCCATTTCTTTTG | 611 |
| | | GCGTGTTGACT | | | GTAAACCACAT | |
| 68 | RPOC_EC_1036_1060_F | CGTGTTGACTATTCGGG | 249 | RPOC_EC_1097_1126_R | ATTCAAGAGCCATTTCTTT | 612 |
| | | GCGTTCAG | | | TGGTAAACCAC | |
| 49 | RPOC_EC_114_140_F | TAAGAAGCCGGAAACCA | 250 | RPOC_EC_213_232_R | GGCGCTTGTACTTACCGCAC | 617 |
| | | TCAACTACCG | | | | |
| 227 | RPOC_EC_1256_1277_F | ACCCAGTGCTGCTGAAC | 251 | RPOC_EC_1295_1315_R | GTTCAAATGCCTGGATACC | 613 |
| | | CGTGC | | | CA | |
| 292 | RPOC_EC_1374_1393_F | CGCCGACTTCGACGGTG | 252 | RPOC_EC_1437_1455_R | GAGCATCAGCGTGCGTGCT | 614 |
| | | ACC | | | | |
| 364 | RPOC_EC_1374_1393_TMOD_F | TCGCCGACTTCGACGGT | 253 | RPOC_EC_1437_1455_TMOD_R | TGAGCATCAGCGTGCGTGCT | 615 |
| | | GACC | | | | |
| 229 | RPOC_EC_1584_1604_F | TGGCCCGAAAGAAGCTG | 254 | RPOC_EC_1623_1643_R | ACGCGGGCATGCAGAGATG | 616 |
| | | AGCG | | | CC | |
| 978 | RPOC_EC_2145_2175_F | TCAGGAGTCGTTCAACT | 255 | RPOC_EC_2228_2247_R | TTACGCCATCAGGCCACGCA | 622 |
| | | CGATCTACATGATG | | | | |
| 290 | RPOC_EC_2146_2174_F | CAGGAGTCGTTCAACTC | 256 | RPOC_EC_2227_2245_R | ACGCCATCAGGCCACGCAT | 620 |
| | | GATCTACATGAT | | | | |
| 363 | RPOC_EC_2146_2174_TMOD_F | TCAGGAGTCGTTCAACT | 257 | RPOC_EC_2227_2245_TMOD_R | TACGCCATCAGGCCACGCAT | 621 |
| | | CGATCTACATGAT | | | | |
| 51 | RPOC_EC_2178_2196_2_F | TGATTCCGGTGCCCGTG | 258 | RPOC_EC_2225_2246_2_R | TTGGCCATCAGACCACGCA | 618 |
| | | GT | | | TAC | |
| 50 | RPOC_EC_2178_2196_F | TGATTCTGGTGCCCGTG | 259 | RPOC_EC_2225_2246_R | TTGGCCATCAGGCCACGCA | 619 |
| | | GT | | | TAC | |
| 53 | RPOC_EC_2218_2241_2_F | CTTGCTGGTATGCGTGG | 260 | RPOC_EC_2313_2337_2_R | CGCACCATGCGTAGAGATG | 623 |
| | | TCTGATG | | | AAGTAC | |
| 52 | RPOC_EC_2218_2241_F | CTGGCAGGTATGCGTGG | 261 | RPOC_EC_2313_2337_R | CGCACCGTGGGTTGAGATG | 624 |
| | | TCTGATG | | | AAGTAC | |
| 354 | RPOC_EC_2218_2241_TMOD_F | TCTGGCAGGTATGCGTG | 262 | RPOC_EC_2313_2337_TMOD_R | TCGCACCGTGGGTTGAGAT | 625 |
| | | GTCTGATG | | | GAAGTAC | |
| 958 | RPOC_EC_2223_2243_F | TGGTATGCGTGGTCTGA | 263 | RPOC_EC_2329_2352_R | TGCTAGACCTTTACGTGCA | 626 |
| | | TGGC | | | CCGTG | |
| 960 | RPOC_EC_2334_2357_F | TGCTCGTAAGGGTCTGG | 264 | RPOC_EC_2380_2403_R | TACTAGACGACGGGTCAGG | 627 |
| | | CGGATAC | | | TAACC | |
| 55 | RPOC_EC_808_833_2_F | CGTCGTGTAATTAACCG | 265 | RPOC_EC_865_891_R | ACGTTTTTCGTTTTGAACG | 629 |
| | | TAACAACCG | | | ATAATGCT | |
| 54 | RPOC_EC_808_833_F | CGTCGGGTGATTAACCG | 266 | RPOC_EC_865_889_R | GTTTTTCGTTGCGTACGAT | 628 |
| | | TAACAACCG | | | GATGTC | |
| 961 | RPOC_EC_917_938_F | TATTGGACAACGGTCGT | 267 | RPOC_EC_1009_1034_R | TTACCGAGCAGGTTCTGAC | 607 |
| | | CGCGG | | | GGAAACG | |
| 959 | RPOC_EC_918_938_F | TCTGGATAACGGTCGTC | 268 | RPOC_EC_1009_1031_R | TCCAGCAGGTTCTGACGGA | 606 |
| | | GCGG | | | AACG | |
| 57 | RPOC_EC_993_1019_2_F | CAAAGGTAAGCAAGGAC | 269 | RPOC_EC_1036_1059_2_R | CGAACGGCCAGAGTAGTCA | 608 |
| | | GTTTCCGTCA | | | ACACG | |
| 56 | RPOC_EC_993_1019_F | CAAAGGTAAGCAAGGTC | 270 | RPOC_EC_1036_1059_R | CGAACGGCCTGAGTAGTCA | 609 |
| | | GTTTCCGTCA | | | ACACG | |
| 75 | SP101_SPET11_1_29_F | AACCTTAATTGGAAAGA | 271 | SP101_SPET11_92_116_R | CCTACCCAACGTTCACCAA | 676 |
| | | AACCCAAGAAGT | | | GGGCAG | |
| 446 | SP101_SPET11_1_29_TMOD_F | TAACCTTAATTGGAAAG | 272 | SP101_SPET11_92_116_TMOD_R | TCCTACCCAACGTTCACCA | 677 |
| | | AAACCCAAGAAGT | | | AGGGCAG | |
| 85 | SP101_SPET11_1154_1179_F | CAATACCGCAACAGCGG | 273 | SP101_SPET11_1251_1277_R | GACCCCAACCTGGCCTTTT | 630 |
| | | TGGCTTGGG | | | GTCGTTGA | |
| 424 | SP101_SPET11_1154_1179_TMOD_F | TCAATACCGCAACAGCG | 274 | SP101_SPET11_1251_1277_TMOD_R | TGACCCCAACCTGGCCTTT | 631 |
| | | GTGGCTTGGG | | | TGTCGTTGA | |
| 76 | SP101_SPET11_118_147_F | GCTGGTGAAAATAACCC | 275 | SP101_SPET1 | TGTGGCCGATTTCACCACC | 644 |
| | | AGATGTCGTCTTC | | 1_213_238_R | TGCTCCT | |
| 425 | SP101_SPET11_118_147_TMOD_F | TGCTGGTGAAAATAACC | 276 | SP101_SPET11_213_238_TMOD_R | TTGTGGCCGATTTCACCAC | 645 |
| | | CAGATGTCGTCTTC | | | CTGCTCCT | |
| 86 | SP101_SPET11_1314_1336_F | CGCAAAAAAATCCAGCT | 277 | SP101_SPET11_1403_1431_R | AAACTATTTTTTTAGCTAT | 632 |
| | | ATTAGC | | | ACTCGAACAC | |
| 426 | SP101_SPET11_1314_1336_TMOD_F | TCGCAAAAAAATCCAGC | 278 | SP101_SPET11_1403_1431_TMOD_R | TAAACTATTTTTTTAGCTA | 633 |
| | | TATTAGC | | | TACTCGAACAC | |
| 87 | SP101_SPET11_1408_1437_F | CGAGTATAGCTAAAAAA | 279 | SP101_SPET11_1486_1515_R | GGATAATTGGTCGTAACAA | 634 |
| | | ATAGTTTATGACA | | | GGGATAGTGAG | |
| 427 | SP101_SPET11_1408_1437_TMOD_F | TCGAGTATAGCTAAAAA | 280 | SP101_SPET11_1486_1515_TMOD_R | TGGATAATTGGTCGTAACA | 635 |
| | | AATAGTTTATGACA | | | AGGGATAGTGAG | |
| 88 | SP101_SPET11_1688_1716_F | CCTATATTAATCGTTTA | 281 | SP101_SPET11_1783_1808_R | ATATGATTATCATTGAACT | 636 |
| | | CAGAAACTGGCT | | | GCGGCCG | |
| 428 | SP101_SPET11_1688_1716_TMOD_F | TCCTATATTAATCGTTT | 282 | SP101_SPET11_1783_1808_TMOD_R | TATATGATTATCATTGAAC | 637 |
| | | ACAGAAACTGGCT | | | TGCGGCCG | |
| 89 | SP101_SPET11_1711_1733_F | CTGGCTAAAACTTTGGC | 283 | SP101_SPET11_1808_1835_R | GCGTGACGACCTTCTTGAA | 638 |
| | | AACGGT | | | TTGTAATCA | |
| 429 | SP101_SPET11_1711_1733_TMOD_F | TCTGGCTAAAACTTTGG | 284 | SP101_SPET11_1808_1835_TMOD_R | TGCGTGACGACCTTCTTGA | 639 |
| | | CAACGGT | | | ATTGTAATCA | |
| 90 | SP101_SPET11_1807_1835_F | ATGATTACAATTCAAGA | 285 | SP101_SPET11_1901_1927_R | TTGGACCTGTAATCAGCTG | 640 |
| | | AGGTCGTCACGC | | | AATACTGG | |
| 430 | SP101_SPET11_1807_1835_TMOD_F | TATGATTACAATTCAAG | 286 | SP101_SPET11_1901_1927_TMOD_R | TTTGGACCTGTAATCAGCT | 641 |
| | | AAGGTCGTCACGC | | | GAATACTGG | |
| 91 | SP101_SPET11_1967_1991_F | TAACGGTTATCATGGCC | 287 | SP101_SPET11_2062_2083_R | ATTGCCCAGAAATCAAATC | 642 |
| | | CAGATGGG | | | ATC | |
| 431 | SP101_SPET11_1967_1991_TMOD_F | TTAACGGTTATCATGGC | 288 | SP101_SPET11_2062_2083_TMOD_R | TATTGCCCAGAAATCAAAT | 643 |
| | | CCAGATGGG | | | CATC | |
| 77 | SP101_SPET11_216_243_F | AGCAGGTGGTGAAATCG | 289 | SP101_SPET11_308_333_R | TGCCACTTTGACAACTCCT | 654 |
| | | GCCACATGATT | | | GTTGCTG | |
| 432 | SP101_SPET11_216_243_TMOD_F | TAGCAGGTGGTGAAATC | 290 | SP101_SPET11_308_333_TMOD_R | TTGCCACTTTGACAACTCC | 655 |
| | | GGCCACATGATT | | | TGTTGCTG | |
| 92 | SP101_SPET11_2260_2283_F | CAGAGACCGTTTTATCC | 291 | SP101_SPET11_2375_2397_R | TCTGGGTGACCTGGTGTTT | 656 |
| | | TATCAGC | | | TAGA | |
| 433 | SP101_SPET11_2260_2283_TMOD_F | TCAGAGACCGTTTTATC | 292 | SP101_SPET11_2375_2397_TMOD_R | TTCTGGGTGACCTGGTGTT | 647 |
| | | CTATCAGC | | | TTAGA | |
| 93 | SP101_SPET11_2375_2399_F | TCTAAAACACCAGGTCA | 293 | SP101_SPET11_2470_2497_R | AGCTGCTAGATGAGCTTCT | 648 |
| | | CCCAGAAG | | | GCCATGGCC | |
| 434 | SP101_SPET11_2375_2399_TMOD_F | TTCTAAAACACCAGGTC | 294 | SP101_SPET11_2470_2497_TMOD_R | TAGCTGCTAGATGAGCTTC | 649 |
| | | ACCCAGAAG | | | TGCCATGGCC | |
| 94 | SP101_SPET11_2468_2487_F | ATGGCCATGGCAGAAGC | 295 | SP101_SPET11_2543_2570_R | CCATAAGGTCACCGTCACC | 650 |
| | | TCA | | | ATTCAAAGC | |
| 435 | SP101_SPET11_2468_2487_TMOD_F | TATGGCCATGGCAGAAG | 296 | SP101_SPET11_2543_2570_TMOD_R | TCCATAAGGTCACCGTCAC | 651 |
| | | CTCA | | | CATTCAAAGC | |
| 78 | SP101_SPET11_266_295_F | CTTGTACTTGTGGCTCA | 297 | SP101_SPET11_355_380_R | GCTGCTTTGATGGCTGAAT | 661 |
| | | CACGGCTGTTTGG | | | CCCCTTC | |
| 436 | SP101_SPET11_266_295_TMOD_F | TCTTGTACTTGTGGCTC | 298 | SP101_SPET11_355_380_TMOD_R | TGCTGCTTTGATGGCTGAA | 662 |
| | | ACACGGCTGTTTGG | | | TCCCCTTC | |
| 95 | SP101_SPET11_2961_2984_F | ACCATGACAGAAGGCAT | 299 | SP101_SPET11_3023_3045_R | GGAATTTACCAGCGATAGA | 652 |
| | | TTTGACA | | | CACC | |
| 437 | SP101_SPET11_2961_2984_TMOD_F | TACCATGACAGAAGGCA | 300 | SP101_SPET11_3023_3045_TMOD_R | TGGAATTTACCAGCGATAG | 653 |
| | | TTTTGACA | | | ACACC | |
| 96 | SP101_SPET11_3075_3103_F | GATGACTTTTTAGCTAA | 301 | SP101_SPET11_3168_3196_R | AATCGACGACCATCTTGGA | 656 |
| | | TGGTCAGGCAGC | | | AAGATTTCTC | |
| 438 | SP101_SPET11_3075_3103_TMOD_F | TGATGACTTTTTAGCTA | 302 | SP101_SPET11_3168_3196_TMOD_R | TAATCGACGACCATCTTGG | 657 |
| | | ATGGTCAGGCAGC | | | AAAGATTTCTC | |
| 448 | SP101_SPET11_3085_3104_F | TAGCTAATGGTCAGGCA | 303 | SP101_SPET11_3170_3194_R | TCGACGACCATCTTGGAAA | 658 |
| | | GCC | | | GATTTC | |
| 79 | SP101_SPET11_322_344_F | GTCAAAGTGGCACGTTT | 304 | SP101_SPET11_423_441_R | ATCCCCTGCTTCTGCTGCC | 665 |
| | | ACTGGC | | | | |
| 439 | SP101_SPET11_322_344_TMOD_F | TGTCAAAGTGGCACGTT | 305 | SP101_SPET11_423_441_TMOD_R | TATCCCCTGCTTCTGCTGCC | 666 |
| | | TACTGGC | | | | |
| 97 | SP101_SPET11_3386_3403_F | AGCGTAAAGGTGAACCTT | 306 | SP101_SPET11_3480_3506_R | CCAGCAGTTACTGTCCCCT | 659 |
| | | | | | CATCTTTG | |
| 440 | SP101_SPET11_3386_3403_TMOD_F | TAGCGTAAAGGTGAACC | 307 | SP101_SPET11_3480_3506_TMOD_R | TCCAGCAGTTACTGTCCCC | 660 |
| | | TT | | | TCATCTTTG | |
| 98 | SP101_SPET11_3511_3535_F | GCTTCAGGAATCAATGA | 308 | SP101_SPET11_3605_3629_R | GGGTCTACACCTGCACTTG | 663 |
| | | TGGAGCAG | | | CATAAC | |
| 441 | SP101_SPET11_3511_3535_TMOD_F | TGCTTCAGGAATCAATG | 309 | SP101_SPET11_3605_3629_TMOD_R | TGGGTCTACACCTGCACTT | 664 |
| | | ATGGAGCAG | | | GCATAAC | |
| 80 | SP101_SPET11_358_387_F | GGGGATTCAGCCATCAA | 310 | SP101_SPET11_448_473_R | CCAACCTTTTCCACAACAG | 668 |
| | | AGCAGCTATTGAC | | | AATCAGC | |
| 442 | SP101_SPET11_358_387_TMOD_F | TGGGGATTCAGCCATCA | 311 | SP101_SPET11_448_473_TMOD_R | TCCAACCTTTTCCACAACA | 669 |
| | | AAGCAGCTATTGAC | | | GAATCAGC | |
| 447 | SP101_SPET11_364_385_F | TCAGCCATCAAAGCAGC | 312 | SP101_SPET11_448_471_R | TACCTTTTCCACAACAGAA | 667 |
| | | TATTG | | | TCAGC | |
| 81 | SP101_SPET11_600_629_F | CCTTACTTCGAACTATG | 313 | SP101_SPET11_686_714_R | CCCATTTTTTCACGCATGC | 670 |
| | | AATCTTTTGGAAG | | | TGAAAATATC | |
| 443 | SP101_SPET11_600_629_TMOD_F | TCCTTACTTCGAACTAT | 314 | SP101_SPET11_686_714_TMOD_R | TCCCATTTTTTCACGCATG | 671 |
| | | GAATCTTTTGGAAG | | | CTGAAAATATC | |
| 82 | SP101_SPET11_658_684_F | GGGGATTGATATCACCG | 315 | SP101_SPET11_756_784_R | GATTGGCGATAAAGTGATA | 672 |
| | | ATAAGAAGAA | | | TTTTCTAAAA | |
| 444 | SP101_SPET11_658_684_TMOD_F | TGGGGATTGATATCACC | 316 | SP101_SPET11_756_784_TMOD_R | TGATTGGCGATAAAGTGAT | 673 |
| | | GATAAGAAGAA | | | ATTTTCTAAAA | |
| 83 | SP101_SPET11_776_801_F | TCGCCAATCAAAACTAA | 317 | SP101_SPET11_871_896_R | GCCCACCAGAAAGACTAGC | 674 |
| | | GGGAATGGC | | | AGGATAA | |
| 445 | SP101_SPET11_776_801_TMOD_F | TTCGCCAATCAAAACTA | 318 | SP101_SPET11_871_896_TMOD_R | TGCCCACCAGAAAGACTAG | 675 |
| | | AGGGAATGGC | | | CAGGATAA | |
| 84 | SP101_SPET11_893_921_F | GGGCAACAGCAGCGGAT | 319 | SP101_SPET11_988_1012_R | CATGACAGCCAAGACCTCA | 678 |
| | | TGCGATTGCGCG | | | CCCACC | |
| 423 | SP101_SPET11_893_921_TMOD_F | TGGGCAACAGCAGCGGA | 320 | SP101_SPET11_988_1012_TMOD_R | TCATGACAGCCAAGACCTC | 679 |
| | | TTGCGATTGCGCG | | | ACCCACC | |
| 706 | SSPE_BA_114_137_F | TCAAGCAAACGCACAAT | 321 | SSPE_BA_196_222_R | TTGCACGTCTGTTTCAGTT | 683 |
| | | CAGAAGC | | | GCAAATTC | |
| 612 | SSPE_BA_114_137P_F | TCAAGCAAACGCACAAC | 321 | SSPE_BA_196_222P_R | TTGCACGTU^{a}C^{a}GTTTCAGT | 684 |
| | | ^{a}U^{a}AGAAGC | | | TGCAAATTC | |
| 58 | SSPE_BA_115_137_F | CAAGCAAACGCACAATC | 322 | SSPE_BA_197_222_R | TGCACGTCTGTTTCAGTTG | 686 |
| | | AGAAGC | | | CAAATTC | |
| 355 | SSPE_BA_115_137_TMOD_F | TCAAGCAAACGCACAAT | 321 | SSPE_BA_197_222_TMOD_R | TTGCACGTCTGTTTCAGTT | 687 |
| | | CAGAAGC | | | GCAAATTC | |
| 215 | SSPE_BA_121_137_F | AACGCACAATCAGAAGC | 323 | SSPE_BA_197_216_R | TCTGTTTCAGTTGCAAATTC | 685 |
| 699 | SSPE_BA_123_153_F | TGCACAATCAGAAGCTA | 324 | SSPE_BA_202_231_R | TTTCACAGCATGCACGTCT | 688 |
| | | AGAAAGCGCAAGCT | | | GTTTCAGTTGC | |
| 704 | SSPE_BA_146_168_F | TGCAAGCTTCTGGTGCT | 325 | SSPE_BA_242_267_R | TTGTGATTGTTTTGCAGCT | 689 |
| | | AGCATT | | | GATTGTG | |
| 702 | SSPE_BA_150_168_F | TGCTTCTGGTGCTAGCA | 326 | SSPE_BA_243_264_R | TGATTGTTTTGCAGCTGAT | 691 |
| | | TT | | | TGT | |
| 610 | SSPE_BA_150_168P_F | TGCTTCTGGC^{a}GU^{a}C^{a}AG | 326 | SSPE_BA_243_264P_R | TGATTGTTTTGU^{a}AGU^{a}TGA | 691 |
| | | U^{a}ATT | | | C^{a}C^{a}GT | |
| 700 | SSPE_BA_156_168_F | TGGTGCTAGCATT | 327 | SSPE_BA_243_255_R | TGCAGCTGATTGT | 690 |
| 608 | SSPE_BA_156_168P_F | TGGC^{a}GU^{a}C^{a}AGU^{a}ATT | 327 | SSPE_BA_243_255P_R | TGU^{a}AGU^{a}TGAC^{a}C^{a}GT | 690 |
| 705 | SSPE_BA_6389_F | TGCTAGTTATGGTACAG | 328 | SSPE_BA_163_191_R | TCATAACTAGCATTTGTGC | 682 |
| | | AGTTTGCGAC | | | TTTGAATGCT | |
| 703 | SSPE_BA_72_89_F | TGGTACAGAGTTTGCGAC | 329 | SSPE_BA_163_182_R | TCATTTGTGCTTTGAATGCT | 681 |
| 611 | SSPE_BA_72_89P_F | TGGTAU^{a}AGAGC^{a}C^{a}C^{a}G | 329 | SSPE_BA_163_182P_R | TCATTTGTGCC^{a}C^{a}C^{a}GAAC | 681 |
| | | U^{a}GAC | | | ^{a}GU^{a}T | |
| 701 | SSPE_BA_75_89_F | TACAGAGTTTGCGAC | 330 | SSPE_BA_163_177_R | TGTGCTTTGAATGCT | 680 |
| 609 | SSPE_BA_75_89P_F | TAU^{a}AGAGC^{a}C^{a}C^{a}CGU^{a}G | 330 | SSPE_BA_163_177P_R | TGTGCC^{a}C^{a}C^{a}GAAC^{a}GU^{a}T | 680 |
| | | AC | | | | |
| 1099 | TOXR_VBC_135_158_F | TCGATTAGGCAGCAACG | 331 | TOXR_VBC_221_246_R | TTCAAAACCTTGCTCTCGC | 692 |
| | | AAAGCCG | | | CAAACAA | |
| 905 | TRPE_AY094355_1064_1086_F | TCGACCTTTGGCAGGAA | 332 | TRPE_AY094355_1171_1196_R | TACATCGTTTCGCCCAAGA | 693 |
| | | CTAGAC | | | TCAATCA | |
| 904 | TRPE_AY094355_1278_1303_F | TCAAATGTACAAGGTGA | 333 | TRPE_AY094355_1392_1418_R | TCCTCTTTTCACAGGCTCT | 694 |
| | | AGTGCGTGA | | | ACTTCATC | |
| 903 | TRPE_AY094355_1445_1471_F | TGGATGGCATGGTGAAA | 334 | TRPE_AY094355_1551_1580_R | TATTTGGGTTTCATTCCAC | 695 |
| | | TGGATATGTC | | | TCAGATTCTGG | |
| 902 | TRPE_AY094355_1467_1491_F | ATGTCGATTGCAATCCG | 335 | TRPE_AY094355_1569_1592_R | TGCGCGAGCTTTTATTTGG | 696 |
| | | TACTTGTG | | | GTTTC | |
| 906 | TRPE_AY094355_666_688_F | GTGCATGCGGATACAGA | 336 | TRPE_AY094355_769_791_R | TTCAAAATGCGGAGGCGTA | 697 |
| | | GCAGAG | | | TGTG | |
| 907 | TRPE_AY094355_757_776_F | TGCAAGCGCGACCACAT | 337 | TRPE_AY094355_864_883_R | TGCCCAGGTACAACCTGCAT | 698 |
| | | ACG | | | | |
| 114 | TUFB_EC_225_251_F | GCACTATGCACACGTAG | 338 | TUFB_EC_284_309_R | TATAGCACCATCCATCTGA | 706 |
| | | ATTGTCCTGG | | | GCGGCAC | |
| 60 | TUFB_EC_239_259_2_F | TTGACTGCCCAGGTCAC | 339 | TUFB_EC_283_303_2_R | GCCGTCCATTTGAGCAGCA | 704 |
| | | GCTG | | | CC | |
| 59 | TUFB_EC_239_259_F | TAGACTGCCCAGGACAC | 340 | TUFB_EC_283_303_R | GCCGTCCATCTGAGCAGCA | 705 |
| | | GCTG | | | CC | |
| 942 | TUFB_EC_251_278_F | TGCACGCCGACTATGTT | 341 | TUFB_EC_337_360_R | TATGTGCTCACGAGTTTGC | 707 |
| | | AAGAACATGAT | | | GGCAT | |
| 941 | TUFB_EC_275_299_F | TGATCACTGGTGCTGCT | 342 | TUFB_EC_337_362_R | TGGATGTGCTCACGAGTCT | 708 |
| | | CAGATGGA | | | GTGGCAT | |
| 117 | TUFB_EC_757_774_F | AAGACGACCTGCACGGGC | 343 | TUFB_EC_849_867_R | GCGCTCCACGTCTTCACGC | 709 |
| 293 | TUFB_EC_957_979_F | CCACACGCCGTTCTTCA | 344 | TUFB_EC_1034_1058_R | GGCATCACCATTTCCTTGT | 700 |
| | | ACAACT | | | CCTTCG | |
| 367 | TUFB_EC_957_979_TMOD_F | TCCACACGCCGTTCTTC | 345 | TUFB_EC_1034_1058_TMOD_R | TGGCATCACCATTTCCTTG | 701 |
| | | AACAACT | | | TCCTTCG | |
| 62 | TUFB_EC_976_1000_2_F | AACTACCGTCCTCAGTT | 346 | TUFB_EC_1045_1068_2_R | GTTGTCACCAGGCATTACC | 702 |
| | | CTACTTCC | | | ATTTC | |
| 61 | TUFB_EC_976_1000_F | AACTACCGTCCGCAGTT | 347 | TUFB_EC_1045_1068_R | GTTGTCGCCAGGCATAACC | 703 |
| | | CTACTTCC | | | ATTTC | |
| 63 | TUFB_EC_985_1012_F | CCACAGTTCTACTTCCG | 348 | TUFB_EC_1033_1062_R | TCCAGGCATTACCATTTCT | 699 |
| | | TACTACTGACG | | | ACTCCTTCTGG | |
| 225 | VALS_EC_1105_1124_F | CGTGGCGGCGTGGTTAT | 349 | VALS_EC_1195_1214_R | ACGAACTGCATGTCGCCGTT | 710 |
| | | CGA | | | | |
| 71 | VALS_EC_1105_1124_F | CGTGGCGGCGTGGTTAT | 349 | VALS_EC_1195_1218_R | CGGTACGAACTGGATGTCG | 711 |
| | | CGA | | | CCGTT | |
| 358 | VALS_EC_1105_1124_TMOD_F | TCGTGGCGGCGTGGTTA | 350 | VALS_EC_1195_1218_TMOD_R | TCGGTACGAACTGGATGTC | 712 |
| | | TCGA | | | GCCGTT | |
| 965 | VALS_EC_1128_1151_F | TATGCTGACCGACCAGT | 351 | VALS_EC_1231_1257_R | TTCGCGCATCCAGGAGAAG | 713 |
| | | GGTACGT | | | TACATGTT | |
| 112 | VALS_EC_1833_1850_F | CGACGCGCTGCGCTTCAC | 352 | VALS_EC_1920_1943_R | GCGTTCCACAGCTTGTTGC | 714 |
| | | | | | AGAAG | |
| 116 | VALS_EC_1920_1943_F | CTTCTGCAACAAGCTGT | 353 | VALS_EC_1948_1970_R | TCGCAGTTCATCAGCACGA | 715 |
| | | GGAACGC | | | AGCG | |
| 295 | VALS_EC_610_649_F | ACCGAGCAAGGAGACCA | 354 | VALS_EC_705_727_R | TATAACGCACATCGTCAGG | 716 |
| | | GC | | | GTGA | |
| 931 | WAAA_Z96925_2_29_F | TCTTGCTCTTTCGTGAG | 355 | WAAA_Z96925_115_138_R | CAAGCGGTTTGCCTCAAAT | 717 |
| | | TTCAGTAAATG | | | AGTCA | |
| 932 | WAAA_Z96925_286_311_F | TCGATCTGGTTTCATGC | 356 | WAAA_Z96925_394_412_R | TGGCACGAGCCTGACCTGT | 718 |
| | | TGTTTCAGT | | | | |

Primer pair name codes and reference sequences are shown in Table 2. The primer name code typically represents the gene to which the given primer pair is targeted. The primer pair name includes coordinates with respect to a reference sequence defined by an extraction of a section of sequence or defined by a GenBank gi number, or the corresponding complementary sequence of the extraction, or the entire GenBank gi number as indicated by the label “no extraction.” Where “no extraction” is indicated for a reference sequence, the coordinates of a primer pair named to the reference sequence are with respect to the GenBank gi listing. Gene abbreviations are shown in bold type in the “Gene Name” column.

TABLE 2
Primer Name Codes and Reference Sequences

| | | | | | Extraction |
|---|---|---|---|---|---|
| Primer | | | Reference | Extracted gene | or entire |
| name | | | GenBank | coordinates of gi | gene |
| code | Gene Name | Organism | gi number | number | SEQ ID NO: |
| 16S_EC | 16S rRNA (16S | Escherichia | 16127994 | 4033120 . . . 4034661 | 719 |
| | ribosomal RNA | coli | | | |
| | gene) | | | | |
| 23S_EC | 23S rRNA (23S | Escherichia | 16127994 | 4166220 . . . 4169123 | 720 |
| | ribosomal RNA | coli | | | |
| | gene) | | | | |
| CAPC_BA | capC (capsule | Bacillus | 6470151 | Complement | 721 |
| | biosynthesis gene) | anthracis | | (55628 . . . 56074) | |
| CYA_BA | cya (cyclic AMP | Bacillus | 4894216 | Complement | 722 |
| | gene) | anthracis | | (154288 . . . 156626) | |
| DNAK_EC | dnaK (chaperone | Escherichia | 16127994 | 12163 . . . 14079 | 723 |
| | dnaK gene) | coli | | | |
| GROL_EC | groL (chaperonin | Escherichia | 16127994 | 4368603 . . . 4370249 | 724 |
| | groL) | coli | | | |
| HFLB_EC | hflb (cell | Escherichia | 16127994 | Complement | 725 |
| | division protein | coli | | (3322645 . . . 3324576) | |
| | peptidase ftsH) | | | | |
| INFB_EC | infB (protein | Escherichia | 16127994 | Complement | 726 |
| | chain initiation | coli | | (3310983 . . . 3313655) | |
| | factor infB gene) | | | | |
| LEF_BA | lef (lethal | Bacillus | 21392688 | Complement | 727 |
| | factor) | anthracis | | (149357 . . . 151786) | |
| PAG_BA | pag (protective | Bacillus | 21392688 | 143779 . . . 146073 | 728 |
| | antigen) | anthracis | | | |
| RPLB_EC | rplB (50S | Escherichia | 16127994 | 3449001 . . . 3448180 | 729 |
| | ribosomal protein | coli | | | |
| | L2) | | | | |
| RPOB_EC | rpoB (DNA-directed | Escherichia | 6127994 | Complement | 730 |
| | RNA polymerase | coli | | 4178823 . . . 4182851 | |
| | beta chain) | | | | |
| RPOC_EC | rpoC (DNA-directed | Escherichia | 16127994 | 4182928 . . . 4187151 | 731 |
| | RNA polymerase | coli | | | |
| | beta′ chain) | | | | |
| SP101ET_SPET_11 | Concatenation | Artificial | 15674250 | | 732 |
| | comprising: | Sequence* - | | | |
| | gki (glucose | partial gene | | Complement | |
| | kinase) | sequences of | | (1258294 . . . 1258791) | |
| | gtr (glutamine | Streptococcus | | complement | |
| | transporter | pyogenes | | (1236751 . . . 1237200) | |
| | protein) | | | | |
| | murI (glutamate | | | 312732 . . . 313169 | |
| | racemase) | | | | |
| | mutS (DNA mismatch | | | Complement | |
| | repair protein) | | | (1787602 . . . 1788007) | |
| | xpt (xanthine | | | 930977 . . . 931425 | |
| | phosphoribosyl | | | | |
| | transferase) | | | | |
| | yqiL (acetyl-CoA- | | | 129471 . . . 129903 | |
| | acetyl | | | | |
| | transferase) | | | | |
| | tkt | | | 1391844 . . . 1391386 | |
| | (transketolase) | | | | |
| SSPE_BA | sspE (small acid- | Bacillus | 30253828 | 226496 . . . 226783 | 733 |
| | soluble spore | anthracis | | | |
| | protein) | | | | |
| TUFB_EC | tufB (Elongation | Escherichia | 16127994 | 4173523 . . . 4174707 | 734 |
| | factor Tu) | coli | | | |
| VALS_EC | valS (Valyl-tRNA | Escherichia | 16127994 | Complement | 735 |
| | synthetase) | coli | | (4481405 . . . 4478550) | |
| ASPS_EC | aspS (Aspartyl- | Escherichia | 16127994 | complement (1946777 . . . 1948546) | 736 |
| | tRNA synthetase) | coli | | | |
| CAF1_AF053947 | caf1 (capsular | Yersinia | 2996286 | No extraction - | — |
| | protein caf1) | pestis | | GenBank coordinates | |
| | | | | used | |
| INV_U22457 | inv (invasin) | Yersinia | 1256565 | 74 . . . 3772 | 737 |
| | | pestis | | | |
| LL_NC003143 | Y. pestis specific | Yersinia | 16120353 | No extraction - | — |
| | chromosomal genes - | pestis | | GenBank coordinates | |
| | difference | | | used | |
| | region | | | | |
| BONTA_X52066 | BoNT/A (neurotoxin | Clostridium | 40381 | 77 . . . 3967 | 738 |
| | type A) | botulinum | | | |
| MECA_Y14051 | mecA methicillin | Staphylococcus | 2791983 | No extraction - | 739 |
| | resistance gene | aureus | | GenBank coordinates | |
| | | | | used | |
| TRPE_AY094355 | trpE (anthranilate | Acinetobacter | 20853695 | No extraction - | 740 |
| | synthase (large | baumanii | | GenBank coordinates | |
| | component)) | | | used | |
| RECA_AF251469 | recA (recombinase | Acinetobacter | 9965210 | No extraction - | 741 |
| | A) | baumanii | | GenBank coordinates | |
| | | | | used | |
| GYRA_AF100557 | gyrA (DNA gyrase | Acinetobacter | 4240540 | No extraction - | 742 |
| | subunit A) | baumanii | | GenBank coordinates | |
| | | | | used | |
| GYRB_AB008700 | gyrB (DNA gyrase | Acinetobacter | 4514436 | No extraction - | 743 |
| | subunit B) | baumanii | | GenBank coordinates | |
| | | | | used | |
| WAAA_Z96925 | waaA (3-deoxy-D- | Acinetobacter | 2765828 | No extraction - | 744 |
| | manno-octulosonic- | baumanii | | GenBank coordinates | |
| | acid transferase) | | | used | |
| CJST_CJ | Concatenation | Artificial | 15791399 | | 745 |
| | comprising: | Sequence* - | | | |
| | tkt | partial gene | | 1569415 . . . 1569873 | |
| | (transketolase) | sequences of | | | |
| | glyA (serine | Campylobacter | | 367573 . . . 368079 | |
| | hydroxymethyltransferase) | jejuni | | | |
| | gltA (citrate | | | complement | |
| | synthase) | | | (1604529 . . . 1604930) | |
| | aspA (aspartate | | | 96692 . . . 97168 | |
| | ammonia lyase) | | | | |
| | glnA (glutamine | | | complement | |
| | synthase) | | | (657609 . . . 658065) | |
| | pgm | | | 327773 . . . 328270 | |
| | (phosphoglycerate | | | | |
| | mutase) | | | | |
| | uncA (ATP | | | 112163 . . . 112651 | |
| | synthetase alpha | | | | |
| | chain) | | | | |
| RNASEP_BDP | RNase P | Bordetella | 33591275 | Complement | 746 |
| | (ribonuclease P) | pertussis | | (3226720 . . . 3227933) | |
| RNASEP_BKM | RNase P | Burkholderia | 53723370 | Complement | 747 |
| | (ribonuclease P) | mallei | | (2527296 . . . 2528220) | |
| RNASEP_BS | RNase P | Bacillus | 16077068 | Complement | 748 |
| | (ribonuclease p) | subtilis | | (2330250 . . . 2330962) | |
| RNASEP_CLB | RNase P | Clostridium | 18308982 | Complement | 749 |
| | (ribonuclease P) | perfringens | | (2291757 . . . 2292584) | |
| RNASEP_EC | RNase P | Escherichia | 16127994 | Complement | 750 |
| | (ribonuclease P) | coli | | (3267457 . . . 3268233 | |
| RNASEP_RKP | RNase P | Rickettsia | 15603881 | complement (605276 . . . 606109) | 751 |
| | (ribonuclease P) | prowazekii | | | |
| RNASEP_SA | RNase P | Staphylococcus | 15922990 | complement (1559869 . . . 1560651) | 752 |
| | (ribonuclease P) | aureus | | | |
| RNASEP_VBC | RNase P | Vibrio | 15640032 | complement (2580367 . . . 2581452) | 753 |
| | (ribonuclease P) | cholerae | | | |
| ICD_CXB | icd (isocitrate | Coxiella | 29732244 | complement (1143867 . . . 1144235) | 754 |
| | dehydrogenase) | burnetii | | | |
| IS1111A | multi-locus | Acinetobacter | 29732244 | No extraction | — |
| | IS1111A insertion | baumannii | | | |
| | element | | | | |
| OMPA_AY485227 | ompA (outer | Rickettsia | 40287451 | No extraction | 755 |
| | membrane protein | prowazekii | | | |
| | A) | | | | |
| OMPB_RKP | ompB (outer | Rickettsia | 15603881 | complement (881264 . . . 886195) | 756 |
| | membrane protein | prowazekii | | | |
| | B) | | | | |
| GLTA_RKP | gltA (citrate | Vibrio | 15603881 | complement (1062547 . . . 1063857) | 757 |
| | synthase) | cholerae | | | |
| TOXR_VBC | toxR | Francisella | 15640032 | complement (1047143 . . . 1048024) | 758 |
| | (transcription | tularensis | | | |
| | regulator toxR) | | | | |
| ASD_FRT | asd (Aspartate | Francisella | 56707187 | complement (438608 . . . 439702) | 759 |
| | semialdehyde | tularensis | | | |
| | dehydrogenase) | | | | |
| GALE_FRT | galE (UDP-glucose | Shigella | 56707187 | 809039 . . . 810058 | 760 |
| | 4-epimerase) | flexneri | | | |
| IPAH_SGF | ipaH (invasion | Campylobacter | 30061571 | 2210775 . . . 2211614 | 761 |
| | plasmid antigen) | jejuni | | | |
| HUPB_CJ | hupB (DNA-binding | Coxiella | 15791399 | complement (849317 . . . 849819) | 762 |
| | protein Hu-beta) | burnetii | | | |
| AB_MLST | Concatenation | Artificial | — | Sequenced in-house | 763 |
| | comprising: | Sequence* - | | | |
| | trpE (anthranilate | partial gene | | | |
| | synthase component | sequences of | | | |
| | I)) | Acinetobacter | | | |
| | adk (adenylate | baumannii | | | |
| | kinase) | | | | |
| | mutY (adenine | | | | |
| | glycosylase) | | | | |
| | fumC (fumarate | | | | |
| | hydratase) | | | | |
| | efp (elongation | | | | |
| | factor p) | | | | |
| | ppa (pyrophosphate | | | | |
| | phospho- | | | | |
| | hydratase | | | | |

*Note:
These artificial reference sequences represent concatenations of partial gene extractions from the indicated reference gi number. Partial sequences were used to create the concatenated sequence because complete gene sequences were not necessary for primer design. The stretches of arbitrary residues “N”s were added for the convenience of separation of the partial gene extractions (100N for SP101_SPET11 (SEQ ID NO: 732); 50N for CJST_CJ (SEQ ID NO: 745); and 40N for AB_MLST (SEQ ID NO: 763)).

### Example 2

### DNA Isolation and Amplification

Genomic materials from culture samples or swabs were prepared using the DNeasy® 96 Tissue Kit (Qiagen, Valencia, Calif.). All PCR reactions are assembled in 50 μl reactions in the 96 well microtiter plate format using a Packard MPII liquid handling robotic platform and MJ Dyad® thermocyclers (MJ research, Waltham, Mass.). The PCR reaction consisted of 4 units of Amplitaq Gold®, 1× buffer II (Applied Biosystems, Foster City, Calif.), 1.5 mM MgCl₂, 0.4 M betaine, 800 μM dNTP mix, and 250 nM of each primer.

The following PCR conditions were used to amplify the sequences used for mass spectrometry analysis: 95 C for 10 minutes followed by 8 cycles of 95 C for 30 seconds, 48 C for 30 seconds, and 72 C for 30 seconds, with the 48 C annealing temperature increased 0.9 C after each cycle. The PCR was then continued for 37 additional cycles of 95 C for 15 seconds, 56 C for 20 seconds, and 72 C for 20 seconds.

### Example 3

### Solution Capture Purification of PCR Products for Mass Spectrometry with Ion Exchange Resin-Magnetic Beads

For solution capture of nucleic acids with ion exchange resin linked to magnetic beads, 25 μl of a 2.5 mg/mL suspension of BioClon amine terminated supraparamagnetic beads were added to 25 to 50 μl of a PCR reaction containing approximately 10 pM of a typical PCR amplification product. The above suspension was mixed for approximately 5 minutes by vortexing or pipetting, after which the liquid was removed after using a magnetic separator. The beads containing bound PCR amplification product were then washed 3× with 50 mM ammonium bicarbonate/50% MeOH or 100 mM ammonium bicarbonate/50% MeOH, followed by three more washes with 50% MeOH. The bound PCR amplicon was eluted with 25 mM piperidine, 25 mM imidazole, 35% MeOH, plus peptide calibration standards.

### Example 4

### Mass Spectrometry and Base Composition Analysis

The ESI-FTICR mass spectrometer is based on a Bruker Daltonics (Billerica, Mass.) Apex II 70e electrospray ionization Fourier transform ion cyclotron resonance mass spectrometer that employs an actively shielded 7 Tesla superconducting magnet. The active shielding constrains the majority of the fringing magnetic field from the superconducting magnet to a relatively small volume. Thus, components that might be adversely affected by stray magnetic fields, such as CRT monitors, robotic components, and other electronics, can operate in close proximity to the FTICR spectrometer. All aspects of pulse sequence control and data acquisition were performed on a 600 MHz Pentium II data station running Bruker's Xmass software under Windows NT 4.0 operating system. Sample aliquots, typically 15 μl, were extracted directly from 96-well microtiter plates using a CTC HTS PAL autosampler (LEAP Technologies, Carrboro, N.C.) triggered by the FTICR data station. Samples were injected directly into a 10 μl sample loop integrated with a fluidics handling system that supplies the 100 μl/hr flow rate to the ESI source. Ions were formed via electrospray ionization in a modified Analytica (Branford, Conn.) source employing an off axis, grounded electrospray probe positioned approximately 1.5 cm from the metalized terminus of a glass desolvation capillary. The atmospheric pressure end of the glass capillary was biased at 6000 V relative to the ESI needle during data acquisition. A counter-current flow of dry N₂ was employed to assist in the desolvation process. Ions were accumulated in an external ion reservoir comprised of an rf-only hexapole, a skimmer cone, and an auxiliary gate electrode, prior to injection into the trapped ion cell where they were mass analyzed. Ionization duty cycles >99% were achieved by simultaneously accumulating ions in the external ion reservoir during ion detection. Each detection event consisted of 1M data points digitized over 2.3 s. To improve the signal-to-noise ratio (S/N), 32 scans were co-added for a total data acquisition time of 74 s.

The ESI-TOF mass spectrometer is based on a Bruker Daltonics MicroTOF™. Ions from the ESI source undergo orthogonal ion extraction and are focused in a reflectron prior to detection. The TOF and FTICR are equipped with the same automated sample handling and fluidics described above. Ions are formed in the standard MicroTOF™ ESI source that is equipped with the same off-axis sprayer and glass capillary as the FTICR ESI source. Consequently, source conditions were the same as those described above. External ion accumulation was also employed to improve ionization duty cycle during data acquisition. Each detection event on the TOF was comprised of 75,000 data points digitized over 75 μs.

The sample delivery scheme allows sample aliquots to be rapidly injected into the electrospray source at high flow rate and subsequently be electrosprayed at a much lower flow rate for improved ESI sensitivity. Prior to injecting a sample, a bolus of buffer was injected at a high flow rate to rinse the transfer line and spray needle to avoid sample contamination/carryover. Following the rinse step, the autosampler injected the next sample and the flow rate was switched to low flow. Following a brief equilibration delay, data acquisition commenced. As spectra were co-added, the autosampler continued rinsing the syringe and picking up buffer to rinse the injector and sample transfer line. In general, two syringe rinses and one injector rinse were required to minimize sample carryover. During a routine screening protocol a new sample mixture was injected every 106 seconds. More recently a fast wash station for the syringe needle has been implemented which, when combined with shorter acquisition times, facilitates the acquisition of mass spectra at a rate of just under one spectrum/minute.

Raw mass spectra were post-calibrated with an internal mass standard and deconvoluted to monoisotopic molecular masses. Unambiguous base compositions were derived from the exact mass measurements of the complementary single-stranded oligonucleotides. Quantitative results are obtained by comparing the peak heights with an internal PCR calibration standard present in every PCR well at 500 molecules per well for the ribosomal DNA-targeted primers and 100 molecules per well for the protein-encoding gene targets. Calibration methods are commonly owned and disclosed in U.S. Provisional Patent Application Ser. No. 60/545,425.

### Example 5

### De Novo Determination of Base Composition of Amplification Products Using Molecular Mass Modified Deoxynucleotide Triphosphates

Because the molecular masses of the four natural nucleobases have a relatively narrow molecular mass range (A=313.058, G=329.052, C=289.046, T=304.046—See Table 3), a persistent source of ambiguity in assignment of base composition can occur as follows: two nucleic acid strands having different base composition may have a difference of about 1 Da when the base composition difference between the two strands is G [symbol] A (−15.994) combined with C [symbol] T (+15.000). For example, one 99-mer nucleic acid strand having a base composition of A₂₇G₃₀C₂₁T₂₁ has a theoretical molecular mass of 30779.058 while another 99-mer nucleic acid strand having a base composition of A₂₆G₃₁C₂₂T₂₀ has a theoretical molecular mass of 30780.052. A 1 Da difference in molecular mass may be within the experimental error of a molecular mass measurement and thus, the relatively narrow molecular mass range of the four natural nucleobases imposes an uncertainty factor.

The present invention provides for a means for removing this theoretical 1 Da uncertainty factor through amplification of a nucleic acid with one mass-tagged nucleobase and three natural nucleobases. The term “nucleobase” as used herein is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP).

Addition of significant mass to one of the 4 nucleobases (dNTPs) in an amplification reaction, or in the primers themselves, will result in a significant difference in mass of the resulting amplification product (significantly greater than 1 Da) arising from ambiguities arising from the G [symbol] A combined with C [symbol] T event (Table 3). Thus, the same the G [symbol] A (−15.994) event combined with 5-Iodo-C [symbol] T (−110.900) event would result in a molecular mass difference of 126.894. If the molecular mass of the base composition A₂₇G₃₀ 5-Iodo-C₂₁T₂₁ (33422.958) is compared with A₂₆G₃₁5-Iodo-C₂₂T₂₀, (33549.852) the theoretical molecular mass difference is +126.894. The experimental error of a molecular mass measurement is not significant with regard to this molecular mass difference. Furthermore, the only base composition consistent with a measured molecular mass of the 99-mer nucleic acid is A₂₇G₃₀5-Iodo-C₂₁T₂₁. In contrast, the analogous amplification without the mass tag has 18 possible base compositions.

TABLE 3
Molecular Masses of Natural Nucleobases and the
Mass-Modified Nucleobase 5-Iodo-C and Molecular
Mass Differences Resulting from Transitions

| Nucleobase | Molecular Mass | Transition | Δ Molecular Mass |
|---|---|---|---|
| A | 313.058 | A-->T | −9.012 |
| A | 313.058 | A-->C | −24.012 |
| A | 313.058 | A-->5-Iodo-C | 101.888 |
| A | 313.058 | A-->G | 15.994 |
| T | 304.046 | T-->A | 9.012 |
| T | 304.046 | T-->C | −15.000 |
| T | 304.046 | T-->5-Iodo-C | 110.900 |
| T | 304.046 | T-->G | 25.006 |
| C | 289.046 | C-->A | 24.012 |
| C | 289.046 | C-->T | 15.000 |
| C | 289.046 | C-->G | 40.006 |
| 5-Iodo-C | 414.946 | 5-Iodo-C-->A | −101.888 |
| 5-Iodo-C | 414.946 | 5-Iodo-C-->T | −110.900 |
| 5-Iodo-C | 414.946 | 5-Iodo-C-->G | −85.894 |
| G | 329.052 | G-->A | −15.994 |
| G | 329.052 | G-->T | −25.006 |
| G | 329.052 | G-->C | −40.006 |
| G | 329.052 | G-->5-Iodo-C | 85.894 |

### Example 6

### Data Processing

Mass spectra of bioagent identifying amplicons are analyzed independently using a maximum-likelihood processor, such as is widely used in radar signal processing. This processor, referred to as GenX, first makes maximum likelihood estimates of the input to the mass spectrometer for each primer by running matched filters for each base composition aggregate on the input data. This includes the GenX response to a calibrant for each primer.

The algorithm emphasizes performance predictions culminating in probability-of-detection versus probability-of-false-alarm plots for conditions involving complex backgrounds of naturally occurring organisms and environmental contaminants. Matched filters consist of a priori expectations of signal values given the set of primers used for each of the bioagents. A genomic sequence database is used to define the mass base count matched filters. The database contains the sequences of known bacterial bioagents and includes threat organisms as well as benign background organisms. The latter is used to estimate and subtract the spectral signature produced by the background organisms. A maximum likelihood detection of known background organisms is implemented using matched filters and a running-sum estimate of the noise covariance. Background signal strengths are estimated and used along with the matched filters to form signatures which are then subtracted. the maximum likelihood process is applied to this “cleaned up” data in a similar manner employing matched filters for the organisms and a running-sum estimate of the noise-covariance for the cleaned up data.

The amplitudes of all base compositions of bioagent identifying amplicons for each primer are calibrated and a final maximum likelihood amplitude estimate per organism is made based upon the multiple single primer estimates. Models of all system noise are factored into this two-stage maximum likelihood calculation. The processor reports the number of molecules of each base composition contained in the spectra. The quantity of amplification product corresponding to the appropriate primer set is reported as well as the quantities of primers remaining upon completion of the amplification reaction.

### Example 7

### Use of Broad Range Survey and Division Wide Primer Pairs for Identification of Bacteria in an Epidemic Surveillance Investigation

This investigation employed a set of 16 primer pairs which is herein designated the “surveillance primer set” and comprises broad range survey primer pairs, division wide primer pairs and a single Bacillus clade primer pair. The surveillance primer set is shown in Table 4 and consists of primer pairs originally listed in Table 1. This surveillance set comprises primers with T modifications (note TMOD designation in primer names) which constitutes a functional improvement with regard to prevention of non-templated adenylation (vide supra) relative to originally selected primers which are displayed below in the same row. Primer pair 449 (non-T modified) has been modified twice. Its predecessors are primer pairs 70 and 357, displayed below in the same row. Primer pair 360 has also been modified twice and its predecessors are primer pairs 17 and 118.

TABLE 4
Bacterial Primer Pairs of the Surveillance Primer Set

| | | Forward | | Reverse | |
|---|---|---|---|---|---|
| Primer | | Primer | | Primer | |
| Pair | | (SEQ ID | | (SEQ ID | |
| No. | Forward Primer Name | NO:) | Reverse Primer Name | NO:) | Target Gene |
| 346 | 16S_EC_713_732_TMOD_F | 27 | 16S_EC_789_809_TMOD_R | 389 | 16S rRNA |
| 10 | 16S_EC_713_732_F | 26 | 16S_EC_789_809 | 388 | 16S rRNA |
| 347 | 16S_EC_785_806_TMOD_F | 30 | 16S_EC_880_897_TMOD_R | 392 | 16S rRNA |
| 11 | 16S_EC_785_806_F | 29 | 16S_EC_880_897_R | 391 | 16S rRNA |
| 348 | 16S_EC_960_981_TMOD_F | 38 | 16S_EC_1054_1073_TMOD_R | 363 | 16S rRNA |
| 14 | 16S_EC_960_981_F | 37 | 16S_EC_1054_1073_R | 362 | 16S rRNA |
| 349 | 23S_EC_1826_1843_TMOD_F | 49 | 23S_EC_1906_1924_TMOD_R | 405 | 23S rRNA |
| 16 | 23S_EC_1826_1843_F | 48 | 23S_EC_1906_1924_R | 404 | 23S rRNA |
| 352 | INFB_EC_1365_1393_TMOD_F | 161 | INFB_EC_1439_1467_TMOD_R | 516 | infB |
| 34 | INFB_EC_1365_1393_F | 160 | INFB_EC_1439_1467_R | 515 | infB |
| 354 | RPOC_EC_2218_2241_TMOD_F | 262 | RPOC_EC_2313_2337_TMOD_R | 625 | rpoC |
| 52 | RPOC_EC_2218_2241_F | 261 | RPOC_EC_2313_2337_R | 624 | rpoC |
| 355 | SSPE_BA_115_137_TMOD_F | 321 | SSPE_BA_197_222_TMOD_R | 687 | sspE |
| 58 | SSPE_BA_115_137_F | 322 | SSPE_BA_197_222_R | 686 | sspE |
| 356 | RPLB_EC_650_679_TMOD_F | 232 | RPLB_EC_739_762_TMOD_R | 592 | rplB |
| 66 | RPLB_EC_650_679_F | 231 | RPLB_EC_739_762_R | 591 | rplB |
| 358 | VALS_EC_1105_1124_TMOD_F | 350 | VALS_EC_1195_1218_TMOD_R | 712 | valS |
| 71 | VALS_EC_1105_1124_F | 349 | VALS_EC_1195_1218_R | 711 | valS |
| 359 | RPOB_EC_1845_1866_TMOD_F | 241 | RPOB_EC_1909_1929_TMOD_R | 597 | rpoB |
| 72 | RPOB_EC_1845_1866_F | 240 | RPOB_EC_1909_1929_R | 596 | rpoB |
| 360 | 23S_EC_2646_2667_TMOD_F | 60 | 23S_EC_2745_2765_TMOD_R | 416 | 23S rRNA |
| 118 | 23S_EC_2646_2667_F | 59 | 23S_EC_2745_2765_R | 415 | 23S rRNA |
| 17 | 23S_EC_2645_2669_F | 58 | 23S_EC_2744_2761_R | 414 | 23S rRNA |
| 361 | 16S_EC_1090_1111_2_TMOD_F | 5 | 16S_EC_1175_1196_TMOD_R | 370 | 16S rRNA |
| 3 | 16S_EC_1090_1111_2_F | 6 | 16S_EC_1175_1196_R | 369 | 16S rRNA |
| 362 | RPOB_EC_3799_3821_TMOD_F | 245 | RPOB_EC_3862_3888_TMOD_R | 603 | rpoB |
| 289 | RPOB_EC_3799_3821_F | 246 | RPOB_EC_3862_3888_R | 602 | rpoB |
| 363 | RPOC_EC_2146_2174_TMOD_F | 257 | RPOC_EC_2227_2245_TMOD_R | 621 | rpoC |
| 290 | RPOC_EC_2146_2174_F | 256 | RPOC_EC_2227_2245_R | 620 | rpoC |
| 367 | TUFB_EC_957_979_TMOD_F | 345 | TUFB_EC_1034_1058_TMOD_R | 701 | tufB |
| 293 | TUFB_EC_957_979_F | 344 | TUFB_EC_1034_1058_R | 700 | tufB |
| 449 | RPLB_EC_690_710_F | 237 | RPLB_EC_737_758_R | 589 | rplB |
| 357 | RPLB_EC_688_710_TMOD_F | 236 | RPLB_EC_736_757_TMOD_R | 588 | rplB |
| 67 | RPLB_EC_688_710_F | 235 | RPLB_EC_736_757_R | 587 | rplB |

The 16 primer pairs of the surveillance set are used to produce bioagent identifying amplicons whose base compositions are sufficiently different amongst all known bacteria at the species level to identify, at a reasonable confidence level, any given bacterium at the species level. As shown in Tables 6A-E, common respiratory bacterial pathogens can be distinguished by the base compositions of bioagent identifying amplicons obtained using the 16 primer pairs of the surveillance set. In some cases, triangulation identification improves the confidence level for species assignment. For example, nucleic acid from Streptococcus pyogenes can be amplified by nine of the sixteen surveillance primer pairs and Streptococcus pneumoniae can be amplified by ten of the sixteen surveillance primer pairs. The base compositions of the bioagent identifying amplicons are identical for only one of the analogous bioagent identifying amplicons and differ in all of the remaining analogous bioagent identifying amplicons by up to four bases per bioagent identifying amplicon. The resolving power of the surveillance set was confirmed by determination of base compositions for 120 isolates of respiratory pathogens representing 70 different bacterial species and the results indicated that natural variations (usually only one or two base substitutions per bioagent identifying amplicon) amongst multiple isolates of the same species did not prevent correct identification of major pathogenic organisms at the species level.

Bacillus anthracis is a well known biological warfare agent which has emerged in domestic terrorism in recent years. Since it was envisioned to produce bioagent identifying amplicons for identification of Bacillus anthracis, additional drill-down analysis primers were designed to target genes present on virulence plasmids of Bacillus anthracis so that additional confidence could be reached in positive identification of this pathogenic organism. Three drill-down analysis primers were designed and are listed in Tables 1 and 5. In Table 5 the drill-down set comprises primers with T modifications (note TMOD designation in primer names) which constitutes a functional improvement with regard to prevention of non-templated adenylation (vide supra) relative to originally selected primers which are displayed below in the same row.

TABLE 5
Drill-Down Primer Pairs for Confirmation of Identification of Bacillus anthracis

| | | Forward | | Reverse | |
|---|---|---|---|---|---|
| Primer | | Primer | | Primer | |
| Pair | | (SEQ ID | | (SEQ ID | |
| No. | Forward Primer Name | NO:) | Reverse Primer Name | NO:) | Target Gene |
| 350 | CAPC_BA_274_303_TMOD_F | 98 | CAPC_BA_349_376_TMOD_R | 452 | capC |
| 24 | CAPC_BA_274_303_F | 97 | CAPC_BA_349_376_R | 451 | capC |
| 351 | CYA_BA_1353_1379_TMOD_F | 128 | CYA_BA_1448_1467_TMOD_R | 483 | cyA |
| 30 | CYA_BA_1353_1379_F | 127 | CYA_BA_1448_1467_R | 482 | cyA |
| 353 | LEF_BA_756_781_TMOD_F | 175 | LEF_BA_843_872_TMOD_R | 531 | lef |
| 37 | LEF_BA_756_781_F | 174 | LEF_BA_843_872_R | 530 | lef |

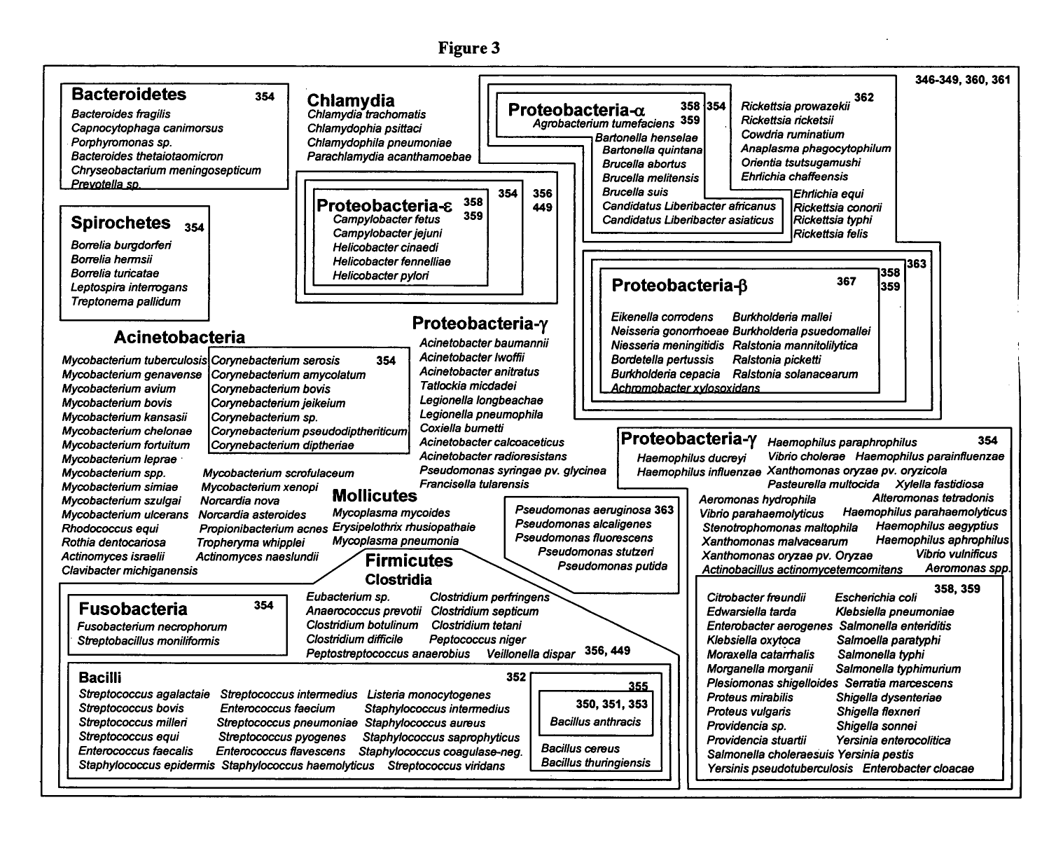

Phylogenetic coverage of bacterial space of the sixteen surveillance primers of Table 4 and the three Bacillus anthracis drill-down primers of Table 5 is shown in FIG. 3 which lists common pathogenic bacteria. FIG. 3 is not meant to be comprehensive in illustrating all species identified by the primers. Only pathogenic bacteria are listed as representative examples of the bacterial species that can be identified by the primers and methods of the present invention. Nucleic acid of groups of bacteria enclosed within the polygons of FIG. 3 can be amplified to obtain bioagent identifying amplicons using the primer pair numbers listed in the upper right hand corner of each polygon. Primer coverage for polygons within polygons is additive. As an illustrative example, bioagent identifying amplicons can be obtained for Chlamydia trachomatis by amplification with, for example, primer pairs 346-349, 360 and 361, but not with any of the remaining primers of the surveillance primer set. On the other hand, bioagent identifying amplicons can be obtained from nucleic acid originating from Bacillus anthracis (located within 5 successive polygons) using, for example, any of the following primer pairs: 346-349, 360, 361 (base polygon), 356, 449 (second polygon), 352 (third polygon), 355 (fourth polygon), 350, 351 and 353 (fifth polygon). Multiple coverage of a given organism with multiple primers provides for increased confidence level in identification of the organism as a result of enabling broad triangulation identification.

In Tables 6A-E, base compositions of respiratory pathogens for primer target regions are shown. Two entries in a cell, represent variation in ribosomal DNA operons. The most predominant base composition is shown first and the minor (frequently a single operon) is indicated by an asterisk (*). Entries with NO DATA mean that the primer would not be expected to prime this species due to mismatches between the primer and target region, as determined by theoretical PCR.

TABLE 6A
Base Compositions of Common Respiratory Pathogens for Bioagent
Identifying Amplicons Corresponding to Primer Pair Nos: 346, 347 and 348

| | | Primer 346 | Primer 347 | Primer 348 |
|---|---|---|---|---|
| Organism | Strain | [A G C T] | [A G C T] | [A G C T] |
| Klebsiella | MGH78578 | [29 32 25 13] | [23 38 28 26] | [26 32 28 30] |
| pneumoniae | | [29 31 25 13]* | [23 37 28 26]* | [26 31 28 30]* |
| Yersinia pestis | CO-92 Biovar | [29 32 25 13] | [22 39 28 26] | [29 30 28 29] |
| | Orientalis | | | [30 30 27 29]* |
| Yersinia pestis | KIM5 P12 (Biovar | [29 32 25 13] | [22 39 28 26] | [29 30 28 29] |
| | Mediaevalis) | | | |
| Yersinia pestis | 91001 | [29 32 25 13] | [22 39 28 26] | [29 30 28 29] |
| | | | | [30 30 27 29]* |
| Haemophilus | KW20 | [28 31 23 17] | [24 37 25 27] | [29 30 28 29] |
| influenzae | | | | |
| Pseudomonas | PAO1 | [30 31 23 15] | [26 36 29 24] | [26 32 29 29] |
| aeruginosa | | | [27 36 29 23]* | |
| Pseudomonas | Pf0-1 | [30 31 23 15] | [26 35 29 25] | [28 31 28 29] |
| fluorescens | | | | |
| Pseudomonas | KT2440 | [30 31 23 15] | [28 33 27 27] | [27 32 29 28] |
| putida | | | | |
| Legionella | Philadelphia-1 | [30 30 24 15] | [33 33 23 27] | [29 28 28 31] |
| pneumophila | | | | |
| Francisella | schu 4 | [32 29 22 16] | [28 38 26 26] | [25 32 28 31] |
| tularensis | | | | |
| Bordetella | Tohama I | [30 29 24 16] | [23 37 30 24] | [30 32 30 26] |
| pertussis | | | | |
| Burkholderia | J2315 | [29 29 27 14] | [27 32 26 29] | [27 36 31 24] |
| cepacia | | | | [20 42 35 19]* |
| Burkholderia | K96243 | [29 29 27 14] | [27 32 26 29] | [27 36 31 24] |
| pseudomallei | | | | |
| Neisseria | FA 1090, ATCC | [29 28 24 18] | [27 34 26 28] | [24 36 29 27] |
| gonorrhoeae | 700825 | | | |
| Neisseria | MC58 (serogroup B) | [29 28 26 16] | [27 34 27 27] | [25 35 30 26] |
| meningitidis | | | | |
| Neisseria | serogroup C, FAM18 | [29 28 26 16] | [27 34 27 27] | [25 35 30 26] |
| meningitidis | | | | |
| Neisseria | Z2491 (serogroup A) | [29 28 26 16] | [27 34 27 27] | [25 35 30 26] |
| meningitidis | | | | |
| Chlamydophila | TW-183 | [31 27 22 19] | NO DATA | [32 27 27 29] |
| pneumoniae | | | | |
| Chlamydophila | AR39 | [31 27 22 19] | NO DATA | [32 27 27 29] |
| pneumoniae | | | | |
| Chlamydophila | CWL029 | [31 27 22 19] | NO DATA | [32 27 27 29] |
| pneumoniae | | | | |
| Chlamydophila | J138 | [31 27 22 19] | NO DATA | [32 27 27 29] |
| pneumoniae | | | | |
| Corynebacterium | NCTC13129 | [29 34 21 15] | [22 38 31 25] | [22 33 25 34] |
| diphtheriae | | | | |
| Mycobacterium | k10 | [27 36 21 15] | [22 37 30 28] | [21 36 27 30] |
| avium | | | | |
| Mycobacterium | 104 | [27 36 21 15] | [22 37 30 28] | [21 36 27 30] |
| avium | | | | |
| Mycobacterium | CSU#93 | [27 36 21 15] | [22 37 30 28] | [21 36 27 30] |
| tuberculosis | | | | |
| Mycobacterium | CDC 1551 | [27 36 21 15] | [22 37 30 28] | [21 36 27 30] |
| tuberculosis | | | | |
| Mycobacterium | H37Rv (lab strain) | [27 36 21 15] | [22 37 30 28] | [21 36 27 30] |
| tuberculosis | | | | |
| Mycoplasma | M129 | [31 29 19 20] | NO DATA | NO DATA |
| pneumoniae | | | | |
| Staphylococcus | MRSA252 | [27 30 21 21] | [25 35 30 26] | [30 29 30 29] |
| aureus | | | | [29 31 30 29]* |
| Staphylococcus | MSSA476 | [27 30 21 21] | [25 35 30 26] | [30 29 30 29] |
| aureus | | | | [30 29 29 30]* |
| Staphylococcus | COL | [27 30 21 21] | [25 35 30 26] | [30 29 30 29] |
| aureus | | | | [30 29 29 30]* |
| Staphylococcus | Mu50 | [27 30 21 21] | [25 35 30 26] | [30 29 30 29] |
| aureus | | | | [30 29 29 30]* |
| Staphylococcus | MW2 | [27 30 21 21] | [25 35 30 26] | [30 29 30 29] |
| aureus | | | | [30 29 29 30]* |
| Staphylococcus | N315 | [27 30 21 21] | [25 35 30 26] | [30 29 30 29] |
| aureus | | | | [30 29 29 30]* |
| Staphylococcus | NCTC 8325 | [27 30 21 21] | [25 35 30 26] | [30 29 30 29] |
| aureus | | | [25 35 31 26]* | [30 29 29 30] |
| Streptococcus | NEM316 | [26 32 23 18] | [24 36 31 25] | [25 32 29 30] |
| agalactiae | | | [24 36 30 26]* | |
| Streptococcus | NC_002955 | [26 32 23 18] | [23 37 31 25] | [29 30 25 32] |
| equi | | | | |
| Streptococcus | MGAS8232 | [26 32 23 18] | [24 37 30 25] | [25 31 29 31] |
| pyogenes | | | | |
| Streptococcus | MGAS315 | [26 32 23 18] | [24 37 30 25] | [25 31 29 31] |
| pyogenes | | | | |
| Streptococcus | SSI-1 | [26 32 23 18] | [24 37 30 25] | [25 31 29 31] |
| pyogenes | | | | |
| Streptococcus | MGAS10394 | [26 32 23 18] | [24 37 30 25] | [25 31 29 31] |
| pyogenes | | | | |
| Streptococcus | Manfredo (M5) | [26 32 23 18] | [24 37 30 25] | [25 31 29 31] |
| pyogenes | | | | |
| Streptococcus | SF370 (M1) | [26 32 23 18] | [24 37 30 25] | [25 31 29 31] |
| pyogenes | | | | |
| Streptococcus | 670 | [26 32 23 18] | [25 35 28 28] | [25 32 29 30] |
| pneumoniae | | | | |
| Streptococcus | R6 | [26 32 23 18] | [25 35 28 28] | [25 32 29 30] |
| pneumoniae | | | | |
| Streptococcus | TIGR4 | [26 32 23 18] | [25 35 28 28] | [25 32 30 29] |
| pneumoniae | | | | |
| Streptococcus | NCTC7868 | [25 33 23 18] | [24 36 31 25] | [25 31 29 31] |
| gordonii | | | | |
| Streptococcus | NCTC 12261 | [26 32 23 18] | [25 35 30 26] | [25 32 29 30] |
| mitis | | | | [24 31 35 29]* |
| Streptococcus | UA159 | [24 32 24 19] | [25 37 30 24] | [28 31 26 31] |
| mutans | | | | |

TABLE 6B
Base Compositions of Common Respiratory Pathogens for Bioagent
Identifying Amplicons Corresponding to Primer Pair Nos: 349, 360, and 356

| | | Primer 349 | Primer 360 | Primer 356 |
|---|---|---|---|---|
| Organism | Strain | [A G C T] | [A G C T] | [A G C T] |
| Klebsiella | MGH78578 | [25 31 25 22] | [33 37 25 27] | NO DATA |
| pneumoniae | | | | |
| Yersinia pestis | CO-92 Biovar | [25 31 27 20] | [34 35 25 28] | NO DATA |
| | Orientalis | [25 32 26 20]* | | |
| Yersinia pestis | KIM5 P12 (Biovar | [25 31 27 20] | [34 35 25 28] | NO DATA |
| | Mediaevalis) | [25 32 26 20]* | | |
| Yersinia pestis | 91001 | [25 31 27 20] | [34 35 25 28] | NO DATA |
| Haemophilus | KW20 | [28 28 25 20] | [32 38 25 27] | NO DATA |
| influenzae | | | | |
| Pseudomonas | PAO1 | [24 31 26 20] | [31 36 27 27] | NO DATA |
| aeruginosa | | | [31 36 27 28]* | |
| Pseudomonas | Pf0-1 | NO DATA | [30 37 27 28] | NO DATA |
| fluorescens | | | [30 37 27 28] | |
| Pseudomonas | KT2440 | [24 31 26 20] | [30 37 27 28] | NO DATA |
| putida | | | | |
| Legionella | Philadelphia-1 | [23 30 25 23] | [30 39 29 24] | NO DATA |
| pneumophila | | | | |
| Francisella | schu 4 | [26 31 25 19] | [32 36 27 27] | NO DATA |
| tularensis | | | | |
| Bordetella | Tohama I | [21 29 24 18] | [33 36 26 27] | NO DATA |
| pertussis | | | | |
| Burkholderia | J2315 | [23 27 22 20] | [31 37 28 26] | NO DATA |
| cepacia | | | | |
| Burkholderia | K96243 | [23 27 22 20] | [31 37 28 26] | NO DATA |
| pseudomallei | | | | |
| Neisseria | FA 1090, ATCC 700825 | [24 27 24 17] | [34 37 25 26] | NO DATA |
| gonorrhoeae | | | | |
| Neisseria | MC58 (serogroup B) | [25 27 22 18] | [34 37 25 26] | NO DATA |
| meningitidis | | | | |
| Neisseria | serogroup C, FAM18 | [25 26 23 18] | [34 37 25 26] | NO DATA |
| meningitidis | | | | |
| Neisseria | Z2491 (serogroup A) | [25 26 23 18] | [34 37 25 26] | NO DATA |
| meningitidis | | | | |
| Chlamydophila | TW-183 | [30 28 27 18] | NO DATA | NO DATA |
| pneumoniae | | | | |
| Chlamydophila | AR39 | [30 28 27 18] | NO DATA | NO DATA |
| pneumoniae | | | | |
| Chlamydophila | CWL029 | [30 28 27 18] | NO DATA | NO DATA |
| pneumoniae | | | | |
| Chlamydophila | J138 | [30 28 27 18] | NO DATA | NO DATA |
| pneumoniae | | | | |
| Corynebacterium | NCTC13129 | NO DATA | [29 40 28 25] | NO DATA |
| diphtheriae | | | | |
| Mycobacterium | k10 | NO DATA | [33 35 32 22] | NO DATA |
| avium | | | | |
| Mycobacterium | 104 | NO DATA | [33 35 32 22] | NO DATA |
| avium | | | | |
| Mycobacterium | CSU#93 | NO DATA | [30 36 34 22] | NO DATA |
| tuberculosis | | | | |
| Mycobacterium | CDC 1551 | NO DATA | [30 36 34 22] | NO DATA |
| tuberculosis | | | | |
| Mycobacterium | H37Rv (lab strain) | NO DATA | [30 36 34 22] | NO DATA |
| tuberculosis | | | | |
| Mycoplasma | M129 | [28 30 24 19] | [34 31 29 28] | NO DATA |
| pneumoniae | | | | |
| Staphylococcus | MRSA252 | [26 30 25 20] | [31 38 24 29] | [33 30 31 27] |
| aureus | | | | |
| Staphylococcus | MSSA476 | [26 30 25 20] | [31 38 24 29] | [33 30 31 27] |
| aureus | | | | |
| Staphylococcus | COL | [26 30 25 20] | [31 38 24 29] | [33 30 31 27] |
| aureus | | | | |
| Staphylococcus | Mu50 | [26 30 25 20] | [31 38 24 29] | [33 30 31 27] |
| aureus | | | | |
| Staphylococcus | MW2 | [26 30 25 20] | [31 38 24 29] | [33 30 31 27] |
| aureus | | | | |
| Staphylococcus | N315 | [26 30 25 20] | [31 38 24 29] | [33 30 31 27] |
| aureus | | | | |
| Staphylococcus | NCTC 8325 | [26 30 25 20] | [31 38 24 29] | [33 30 31 27] |
| aureus | | | | |
| Streptococcus | NEM316 | [28 31 22 20] | [33 37 24 28] | [37 30 28 26] |
| agalactiae | | | | |
| Streptococcus | NC_002955 | [28 31 23 19] | [33 38 24 27] | [37 31 28 25] |
| equi | | | | |
| Streptococcus | MGAS8232 | [28 31 23 19] | [33 37 24 28] | [38 31 29 23] |
| pyogenes | | | | |
| Streptococcus | MGAS315 | [28 31 23 19] | [33 37 24 28] | [38 31 29 23] |
| pyogenes | | | | |
| Streptococcus | SSI-1 | [28 31 23 19] | [33 37 24 28] | [38 31 29 23] |
| pyogenes | | | | |
| Streptococcus | MGAS10394 | [28 31 23 19] | [33 37 24 28] | [38 31 29 23] |
| pyogenes | | | | |
| Streptococcus | Manfredo (M5) | [28 31 23 19] | [33 37 24 28] | [38 31 29 23] |
| pyogenes | | | | |
| Streptococcus | SF370 (M1) | [28 31 23 19] | [33 37 24 28] | [38 31 29 23] |
| pyogenes | | [28 31 22 20]* | | |
| Streptococcus | 670 | [28 31 22 20] | [34 36 24 28] | [37 30 29 25] |
| pneumoniae | | | | |
| Streptococcus | R6 | [28 31 22 20] | [34 36 24 28] | [37 30 29 25] |
| pneumoniae | | | | |
| Streptococcus | TIGR4 | [28 31 22 20] | [34 36 24 28] | [37 30 29 25] |
| pneumoniae | | | | |
| Streptococcus | NCTC7868 | [28 32 23 20] | [34 36 24 28] | [36 31 29 25] |
| gordonii | | | | |
| Streptococcus | NCTC 12261 | [28 31 22 20] | [34 36 24 28] | [37 30 29 25] |
| mitis | | [29 30 22 20]* | | |
| Streptococcus | UA159 | [26 32 23 22] | [34 37 24 27] | NO DATA |
| mutans | | | | |

TABLE 6C
Base Compositions of Common Respiratory Pathogens for Bioagent
Identifying Amplicons Corresponding to Primer Pair Nos: 449, 354, and 352

| | | Primer 449 | Primer 354 | Primer 352 |
|---|---|---|---|---|
| Organism | Strain | [A G C T] | [A G C T] | [A G C T] |
| Klebsiella | MGH78578 | NO DATA | [27 33 36 26] | NO DATA |
| pneumoniae | | | | |
| Yersinia pestis | CO-92 Biovar | NO DATA | [29 31 33 29] | [32 28 20 25] |
| | Orientalis | | | |
| Yersinia pestis | KIM5 P12 (Biovar | NO DATA | [29 31 33 29] | [32 28 20 25] |
| | Mediaevalis) | | | |
| Yersinia pestis | 91001 | NO DATA | [29 31 33 29] | NO DATA |
| Haemophilus | KW20 | NO DATA | [30 29 31 32] | NO DATA |
| influenzae | | | | |
| Pseudomonas | PAO1 | NO DATA | [26 33 39 24] | NO DATA |
| aeruginosa | | | | |
| Pseudomonas | Pf0-1 | NO DATA | [26 33 34 29] | NO DATA |
| fluorescens | | | | |
| Pseudomonas | KT2440 | NO DATA | [25 34 36 27] | NO DATA |
| putida | | | | |
| Legionella | Philadelphia-1 | NO DATA | NO DATA | NO DATA |
| pneumophila | | | | |
| Francisella | schu 4 | NO DATA | [33 32 25 32] | NO DATA |
| tularensis | | | | |
| Bordetella | Tohama I | NO DATA | [26 33 39 24] | NO DATA |
| pertussis | | | | |
| Burkholderia | J2315 | NO DATA | [25 37 33 27] | NO DATA |
| cepacia | | | | |
| Burkholderia | K96243 | NO DATA | [25 37 34 26] | NO DATA |
| pseudomallei | | | | |
| Neisseria | FA 1090, ATCC 700825 | [17 23 22 10] | [29 31 32 30] | NO DATA |
| gonorrhoeae | | | | |
| Neisseria | MC58 (serogroup B) | NO DATA | [29 30 32 31] | NO DATA |
| meningitidis | | | | |
| Neisseria | serogroup C, FAM18 | NO DATA | [29 30 32 31] | NO DATA |
| meningitidis | | | | |
| Neisseria | Z2491 (serogroup A) | NO DATA | [29 30 32 31] | NO DATA |
| meningitidis | | | | |
| Chlamydophila | TW-183 | NO DATA | NO DATA | NO DATA |
| pneumoniae | | | | |
| Chlamydophila | AR39 | NO DATA | NO DATA | NO DATA |
| pneumoniae | | | | |
| Chlamydophila | CWL029 | NO DATA | NO DATA | NO DATA |
| pneumoniae | | | | |
| Chlamydophila | J138 | NO DATA | NO DATA | NO DATA |
| pneumoniae | | | | |
| Corynebacterium | NCTC13129 | NO DATA | NO DATA | NO DATA |
| diphtheriae | | | | |
| Mycobacterium | k10 | NO DATA | NO DATA | NO DATA |
| avium | | | | |
| Mycobacterium | 104 | NO DATA | NO DATA | NO DATA |
| avium | | | | |
| Mycobacterium | CSU#93 | NO DATA | NO DATA | NO DATA |
| tuberculosis | | | | |
| Mycobacterium | CDC 1551 | NO DATA | NO DATA | NO DATA |
| tuberculosis | | | | |
| Mycobacterium | H37Rv (lab strain) | NO DATA | NO DATA | NO DATA |
| tuberculosis | | | | |
| Mycoplasma | M129 | NO DATA | NO DATA | NO DATA |
| pneumoniae | | | | |
| Staphylococcus | MRSA252 | [17 20 21 17] | [30 27 30 35] | [36 24 19 26] |
| aureus | | | | |
| Staphylococcus | MSSA476 | [17 20 21 17] | [30 27 30 35] | [36 24 19 26] |
| aureus | | | | |
| Staphylococcus | COL | [17 20 21 17] | [30 27 30 35] | [35 24 19 27] |
| aureus | | | | |
| Staphylococcus | Mu50 | [17 20 21 17] | [30 27 30 35] | [36 24 19 26] |
| aureus | | | | |
| Staphylococcus | MW2 | [17 20 21 17] | [30 27 30 35] | [36 24 19 26] |
| aureus | | | | |
| Staphylococcus | N315 | [17 20 21 17] | [30 27 30 35] | [36 24 19 26] |
| aureus | | | | |
| Staphylococcus | NCTC 8325 | [17 20 21 17] | [30 27 30 35] | [35 24 19 27] |
| aureus | | | | |
| Streptococcus | NEM316 | [22 20 19 14] | [26 31 27 38] | [29 26 22 28] |
| agalactiae | | | | |
| Streptococcus | NC_002955 | [22 21 19 13] | NO DATA | NO DATA |
| equi | | | | |
| Streptococcus | MGAS8232 | [23 21 19 12] | [24 32 30 36] | NO DATA |
| pyogenes | | | | |
| Streptococcus | MGAS315 | [23 21 19 12] | [24 32 30 36] | NO DATA |
| pyogenes | | | | |
| Streptococcus | SSI-1 | [23 21 19 12] | [24 32 30 36] | NO DATA |
| pyogenes | | | | |
| Streptococcus | MGAS10394 | [23 21 19 12] | [24 32 30 36] | NO DATA |
| pyogenes | | | | |
| Streptococcus | Manfredo (M5) | [23 21 19 12] | [24 32 30 36] | NO DATA |
| pyogenes | | | | |
| Streptococcus | SF370 (M1) | [23 21 19 12] | [24 32 30 36] | NO DATA |
| pyogenes | | | | |
| Streptococcus | 670 | [22 20 19 14] | [25 33 29 35] | [30 29 21 25] |
| pneumoniae | | | | |
| Streptococcus | R6 | [22 20 19 14] | [25 33 29 35] | [30 29 21 25] |
| pneumoniae | | | | |
| Streptococcus | TIGR4 | [22 20 19 14] | [25 33 29 35] | [30 29 21 25] |
| pneumoniae | | | | |
| Streptococcus | NCTC7868 | [21 21 19 14] | NO DATA | [29 26 22 28] |
| gordonii | | | | |
| Streptococcus | NCTC 12261 | [22 20 19 14] | [26 30 32 34] | NO DATA |
| mitis | | | | |
| Streptococcus | UA159 | NO DATA | NO DATA | NO DATA |
| mutans | | | | |

TABLE 6D
Base Compositions of Common Respiratory Pathogens for Bioagent
Identifying Amplicons Corresponding to Primer Pair Nos: 355, 358, and 359

| | | Primer 355 | Primer 358 | Primer 359 |
|---|---|---|---|---|
| Organism | Strain | [A G C T] | [A G C T] | [A G C T] |
| Klebsiella | MGH78578 | NO DATA | [24 39 33 20] | [25 21 24 17] |
| pneumoniae | | | | |
| Yersinia pestis | CO-92 Biovar | NO DATA | [26 34 35 21] | [23 23 19 22] |
| | Orientalis | | | |
| Yersinia pestis | KIM5 P12 (Biovar | NO DATA | [26 34 35 21] | [23 23 19 22] |
| | Mediaevalis) | | | |
| Yersinia pestis | 91001 | NO DATA | [26 34 35 21] | [23 23 19 22] |
| Haemophilus | KW20 | NO DATA | NO DATA | NO DATA |
| influenzae | | | | |
| Pseudomonas | PAO1 | NO DATA | NO DATA | NO DATA |
| aeruginosa | | | | |
| Pseudomonas | Pf0-1 | NO DATA | NO DATA | NO DATA |
| fluorescens | | | | |
| Pseudomonas | KT2440 | NO DATA | [21 37 37 21] | NO DATA |
| putida | | | | |
| Legionella | Philadelphia-1 | NO DATA | NO DATA | NO DATA |
| pneumophila | | | | |
| Francisella | schu 4 | NO DATA | NO DATA | NO DATA |
| tularensis | | | | |
| Bordetella | Tohama I | NO DATA | NO DATA | NO DATA |
| pertussis | | | | |
| Burkholderia | J2315 | NO DATA | NO DATA | NO DATA |
| cepacia | | | | |
| Burkholderia | K96243 | NO DATA | NO DATA | NO DATA |
| pseudomallei | | | | |
| Neisseria | FA 1090, ATCC 700825 | NO DATA | NO DATA | NO DATA |
| gonorrhoeae | | | | |
| Neisseria | MC58 (serogroup B) | NO DATA | NO DATA | NO DATA |
| meningitidis | | | | |
| Neisseria | serogroup C, FAM18 | NO DATA | NO DATA | NO DATA |
| meningitidis | | | | |
| Neisseria | Z2491 (serogroup A) | NO DATA | NO DATA | NO DATA |
| meningitidis | | | | |
| Chlamydophila | TW-183 | NO DATA | NO DATA | NO DATA |
| pneumoniae | | | | |
| Chlamydophila | AR39 | NO DATA | NO DATA | NO DATA |
| pneumoniae | | | | |
| Chlamydophila | CWL029 | NO DATA | NO DATA | NO DATA |
| pneumoniae | | | | |
| Chlamydophila | J138 | NO DATA | NO DATA | NO DATA |
| pneumoniae | | | | |
| Corynebacterium | NCTC13129 | NO DATA | NO DATA | NO DATA |
| diphtheriae | | | | |
| Mycobacterium | k10 | NO DATA | NO DATA | NO DATA |
| avium | | | | |
| Mycobacterium | 104 | NO DATA | NO DATA | NO DATA |
| avium | | | | |
| Mycobacterium | CSU#93 | NO DATA | NO DATA | NO DATA |
| tuberculosis | | | | |
| Mycobacterium | CDC 1551 | NO DATA | NO DATA | NO DATA |
| tuberculosis | | | | |
| Mycobacterium | H37Rv (lab strain) | NO DATA | NO DATA | NO DATA |
| tuberculosis | | | | |
| Mycoplasma | M129 | NO DATA | NO DATA | NO DATA |
| pneumoniae | | | | |
| Staphylococcus | MRSA252 | NO DATA | NO DATA | NO DATA |
| aureus | | | | |
| Staphylococcus | MSSA476 | NO DATA | NO DATA | NO DATA |
| aureus | | | | |
| Staphylococcus | COL | NO DATA | NO DATA | NO DATA |
| aureus | | | | |
| Staphylococcus | Mu50 | NO DATA | NO DATA | NO DATA |
| aureus | | | | |
| Staphylococcus | MW2 | NO DATA | NO DATA | NO DATA |
| aureus | | | | |
| Staphylococcus | N315 | NO DATA | NO DATA | NO DATA |
| aureus | | | | |
| Staphylococcus | NCTC 8325 | NO DATA | NO DATA | NO DATA |
| aureus | | | | |
| Streptococcus | NEM316 | NO DATA | NO DATA | NO DATA |
| agalactiae | | | | |
| Streptococcus | NC_002955 | NO DATA | NO DATA | NO DATA |
| equi | | | | |
| Streptococcus | MGAS8232 | NO DATA | NO DATA | NO DATA |
| pyogenes | | | | |
| Streptococcus | MGAS315 | NO DATA | NO DATA | NO DATA |
| pyogenes | | | | |
| Streptococcus | SSI-1 | NO DATA | NO DATA | NO DATA |
| pyogenes | | | | |
| Streptococcus | MGAS10394 | NO DATA | NO DATA | NO DATA |
| pyogenes | | | | |
| Streptococcus | Manfredo (M5) | NO DATA | NO DATA | NO DATA |
| pyogenes | | | | |
| Streptococcus | SF370 (M1) | NO DATA | NO DATA | NO DATA |
| pyogenes | | | | |
| Streptococcus | 670 | NO DATA | NO DATA | NO DATA |
| pneumoniae | | | | |
| Streptococcus | R6 | NO DATA | NO DATA | NO DATA |
| pneumoniae | | | | |
| Streptococcus | TIGR4 | NO DATA | NO DATA | NO DATA |
| pneumoniae | | | | |
| Streptococcus | NCTC7868 | NO DATA | NO DATA | NO DATA |
| gordonii | | | | |
| Streptococcus | NCTC 12261 | NO DATA | NO DATA | NO DATA |
| mitis | | | | |
| Streptococcus | UA159 | NO DATA | NO DATA | NO DATA |
| mutans | | | | |

TABLE 6E
Base Compositions of Common Respiratory Pathogens for Bioagent
Identifying Amplicons Corresponding to Primer Pair Nos: 362, 363, and 367

| | | Primer 362 | Primer 363 | Primer 367 |
|---|---|---|---|---|
| Organism | Strain | [A G C T] | [A G C T] | [A G C T] |
| Klebsiella | MGH78578 | [21 33 22 16] | [16 34 26 26] | NO DATA |
| pneumoniae | | | | |
| Yersinia pestis | CO-92 Biovar | [20 34 18 20] | NO DATA | NO DATA |
| | Orientalis | | | |
| Yersinia pestis | KIM5 P12 (Biovar | [20 34 18 20] | NO DATA | NO DATA |
| | Mediaevalis) | | | |
| Yersinia pestis | 91001 | [20 34 18 20] | NO DATA | NO DATA |
| Haemophilus | KW20 | NO DATA | NO DATA | NO DATA |
| influenzae | | | | |
| Pseudomonas | PAO1 | [19 35 21 17] | [16 36 28 22] | NO DATA |
| aeruginosa | | | | |
| Pseudomonas | Pf0-1 | NO DATA | [18 35 26 23] | NO DATA |
| fluorescens | | | | |
| Pseudomonas | KT2440 | NO DATA | [16 35 28 23] | NO DATA |
| putida | | | | |
| Legionella | Philadelphia-1 | NO DATA | NO DATA | NO DATA |
| pneumophila | | | | |
| Francisella | schu 4 | NO DATA | NO DATA | NO DATA |
| tularensis | | | | |
| Bordetella | Tohama I | [20 31 24 17] | [15 34 32 21] | [26 25 34 19] |
| pertussis | | | | |
| Burkholderia | J2315 | [20 33 21 18] | [15 36 26 25] | [25 27 32 20] |
| cepacia | | | | |
| Burkholderia | K96243 | [19 34 19 20] | [15 37 28 22] | [25 27 32 20] |
| pseudomallei | | | | |
| Neisseria | FA 1090, ATCC 700825 | NO DATA | NO DATA | NO DATA |
| gonorrhoeae | | | | |
| Neisseria | MC58 (serogroup B) | NO DATA | NO DATA | NO DATA |
| meningitidis | | | | |
| Neisseria | serogroup C, FAM18 | NO DATA | NO DATA | NO DATA |
| meningitidis | | | | |
| Neisseria | Z2491 (serogroup A) | NO DATA | NO DATA | NO DATA |
| meningitidis | | | | |
| Chlamydophila | TW-183 | NO DATA | NO DATA | NO DATA |
| pneumoniae | | | | |
| Chlamydophila | AR39 | NO DATA | NO DATA | NO DATA |
| pneumoniae | | | | |
| Chlamydophila | CWL029 | NO DATA | NO DATA | NO DATA |
| pneumoniae | | | | |
| Chlamydophila | J138 | NO DATA | NO DATA | NO DATA |
| pneumoniae | | | | |
| Corynebacterium | NCTC13129 | NO DATA | NO DATA | NO DATA |
| diphtheriae | | | | |
| Mycobacterium | k10 | [19 34 23 16] | NO DATA | [24 26 35 19] |
| avium | | | | |
| Mycobacterium | 104 | [19 34 23 16] | NO DATA | [24 26 35 19] |
| avium | | | | |
| Mycobacterium | CSU#93 | [19 31 25 17] | NO DATA | [25 25 34 20] |
| tuberculosis | | | | |
| Mycobacterium | CDC 1551 | [19 31 24 18] | NO DATA | [25 25 34 20] |
| tuberculosis | | | | |
| Mycobacterium | H37Rv (lab strain) | [19 31 24 18] | NO DATA | [25 25 34 20] |
| tuberculosis | | | | |
| Mycoplasma | M129 | NO DATA | NO DATA | NO DATA |
| pneumoniae | | | | |
| Staphylococcus | MRSA252 | NO DATA | NO DATA | NO DATA |
| aureus | | | | |
| Staphylococcus | MSSA476 | NO DATA | NO DATA | NO DATA |
| aureus | | | | |
| Staphylococcus | COL | NO DATA | NO DATA | NO DATA |
| aureus | | | | |
| Staphylococcus | Mu50 | NO DATA | NO DATA | NO DATA |
| aureus | | | | |
| Staphylococcus | MW2 | NO DATA | NO DATA | NO DATA |
| aureus | | | | |
| Staphylococcus | N315 | NO DATA | NO DATA | NO DATA |
| aureus | | | | |
| Staphylococcus | NCTC 8325 | NO DATA | NO DATA | NO DATA |
| aureus | | | | |
| Streptococcus | NEM316 | NO DATA | NO DATA | NO DATA |
| agalactiae | | | | |
| Streptococcus | NC_002955 | NO DATA | NO DATA | NO DATA |
| equi | | | | |
| Streptococcus | MGAS8232 | NO DATA | NO DATA | NO DATA |
| pyogenes | | | | |
| Streptococcus | MGAS315 | NO DATA | NO DATA | NO DATA |
| pyogenes | | | | |
| Streptococcus | SSI-1 | NO DATA | NO DATA | NO DATA |
| pyogenes | | | | |
| Streptococcus | MGAS10394 | NO DATA | NO DATA | NO DATA |
| pyogenes | | | | |
| Streptococcus | Manfredo (M5) | NO DATA | NO DATA | NO DATA |
| pyogenes | | | | |
| Streptococcus | SF370 (M1) | NO DATA | NO DATA | NO DATA |
| pyogenes | | | | |
| Streptococcus | 670 | NO DATA | NO DATA | NO DATA |
| pneumoniae | | | | |
| Streptococcus | R6 | [20 30 19 23] | NO DATA | NO DATA |
| pneumoniae | | | | |
| Streptococcus | TIGR4 | [20 30 19 23] | NO DATA | NO DATA |
| pneumoniae | | | | |
| Streptococcus | NCTC7868 | NO DATA | NO DATA | NO DATA |
| gordonii | | | | |
| Streptococcus | NCTC 12261 | NO DATA | NO DATA | NO DATA |
| mitis | | | | |
| Streptococcus | UA159 | NO DATA | NO DATA | NO DATA |
| mutans | | | | |

Four sets of throat samples from military recruits at different military facilities taken at different time points were analyzed using the primers of the present invention. The first set was collected at a military training center from Nov. 1 to Dec. 20, 2002 during one of the most severe outbreaks of pneumonia associated with group A Streptococcus in the United States since 1968. During this outbreak, fifty-one throat swabs were taken from both healthy and hospitalized recruits and plated on blood agar for selection of putative group A Streptococcus colonies. A second set of 15 original patient specimens was taken during the height of this group A Streptococcus-associated respiratory disease outbreak. The third set were historical samples, including twenty-seven isolates of group A Streptococcus, from disease outbreaks at this and other military training facilities during previous years. The fourth set of samples was collected from five geographically separated military facilities in the continental U.S. in the winter immediately following the severe November/December 2002 outbreak.

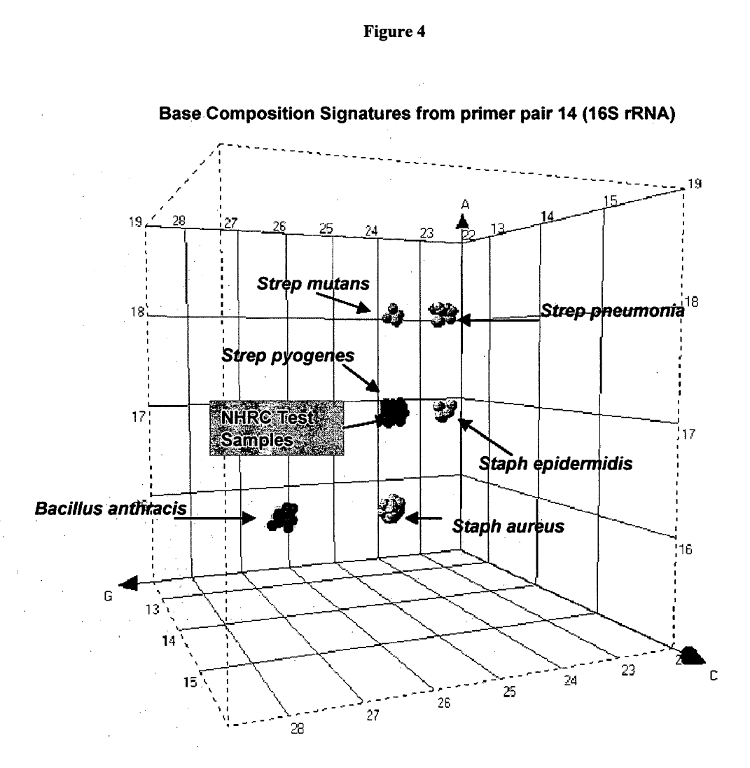

Pure colonies isolated from group A Streptococcus-selective media from all four collection periods were analyzed with the surveillance primer set. All samples showed base compositions that precisely matched the four completely sequenced strains of Streptococcus pyogenes. Shown in FIG. 4 is a 3D diagram of base composition (axes A, G and C) of bioagent identifying amplicons obtained with primer pair number 14 (a precursor of primer pair number 348 which targets 16S rRNA). The diagram indicates that the experimentally determined base compositions of the clinical samples closely match the base compositions expected for Streptococcus pyogenes and are distinct from the expected base compositions of other organisms.

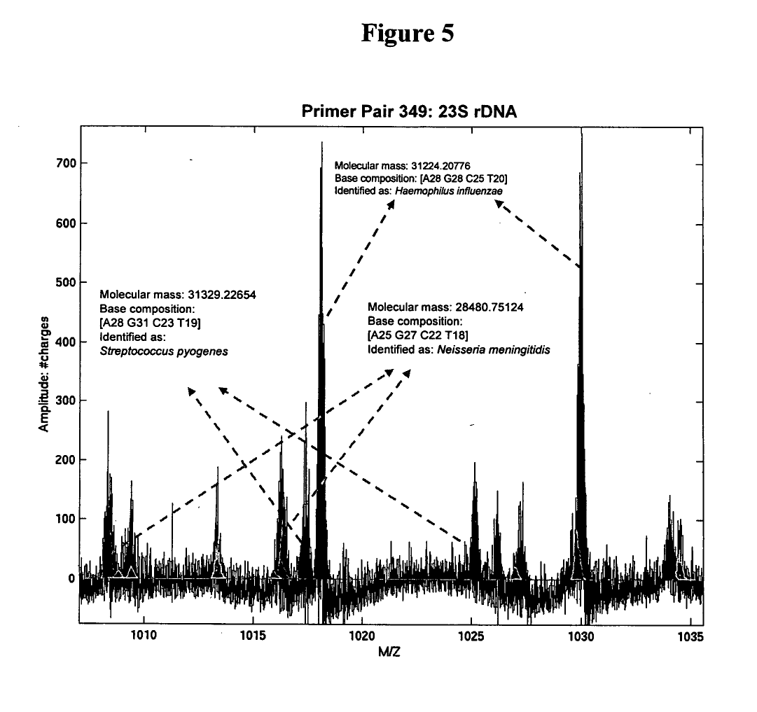

In addition to the identification of Streptococcus pyogenes, other potentially pathogenic organisms were identified concurrently. Mass spectral analysis of a sample whose nucleic acid was amplified by primer pair number 349 (SEQ ID NOs: 49 and 405) exhibited signals of bioagent identifying amplicons with molecular masses that were found to correspond to analogous base compositions of bioagent identifying amplicons of Streptococcus pyogenes (A27 G32 C24 T18), Neisseria meningitidis (A25 G27 C22 T18), and Haemophilus influenzae (A28 G28 C25 T20) (see FIG. 5 and Table 6B). These organisms were present in a ratio of 4:5:20 as determined by comparison of peak heights with peak height of an internal PCR calibration standard as described in commonly owned U.S. Patent Application Ser. No. 60/545,425 which is incorporated herein by reference in its entirety.

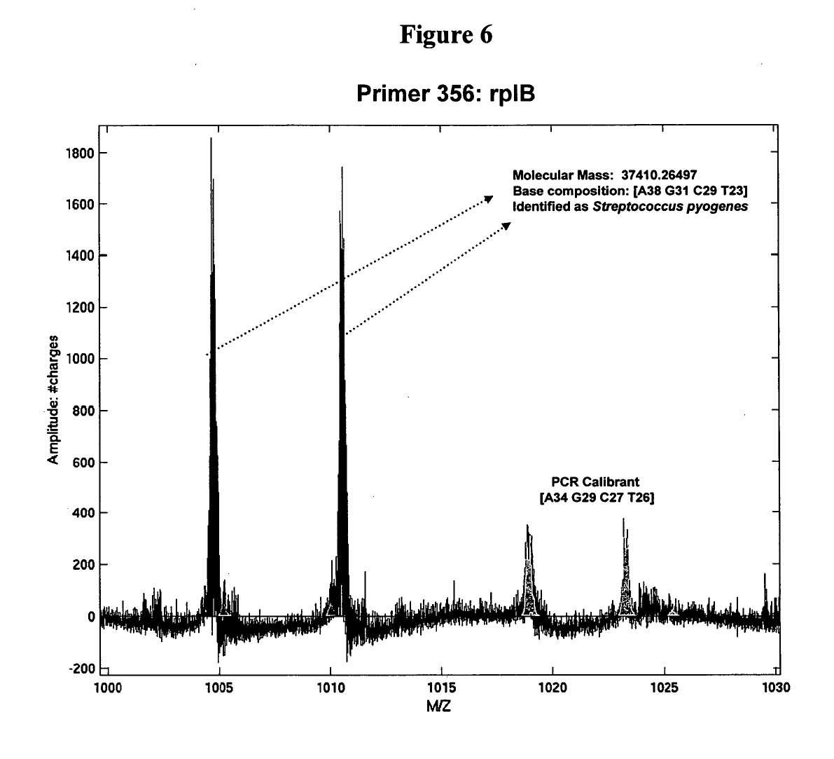

Since certain division-wide primers that target housekeeping genes are designed to provide coverage of specific divisions of bacteria to increase the confidence level for identification of bacterial species, they are not expected to yield bioagent identifying amplicons for organisms outside of the specific divisions. For example, primer pair number 356 (SEQ ID NOs: 232:592) primarily amplifies the nucleic acid of members of the classes Bacilli and Clostridia and is not expected to amplify proteobacteria such as Neisseria meningitidis and Haemophilus influenzae. As expected, analysis of the mass spectrum of amplification products obtained with primer pair number 356 does not indicate the presence of Neisseria meningitidis and Haemophilus influenzae but does indicate the presence of Streptococcus pyogenes (FIGS. 3 and 6, Table 6B). Thus, these primers or types of primers can confirm the absence of particular bioagents from a sample.

The 15 throat swabs from military recruits were found to contain a relatively small set of microbes in high abundance. The most common were Haemophilus influenza, Neisseria meningitides, and Streptococcus pyogenes. Staphylococcus epidermidis, Moraxella cattarhalis, Corynebacterium pseudodiphtheriticum, and Staphylococcus aureus were present in fewer samples. An equal number of samples from healthy volunteers from three different geographic locations, were identically analyzed. Results indicated that the healthy volunteers have bacterial flora dominated by multiple, commensal non-beta-hemolytic Streptococcal species, including the viridans group streptococci (S. parasangunis, S. vestibularis, S. mitis, S. oralis and S. pneumoniae; data not shown), and none of the organisms found in the military recruits were found in the healthy controls at concentrations detectable by mass spectrometry. Thus, the military recruits in the midst of a respiratory disease outbreak had a dramatically different microbial population than that experienced by the general population in the absence of epidemic disease.

### Example 8

### Drill-Down Analysis for Determination of emm-Type of Streptococcus pyogenes in Epidemic Surveillance

As a continuation of the epidemic surveillance investigation of Example 7, determination of sub-species characteristics (genotyping) of Streptococcus pyogenes, was carried out based on a strategy that generates strain-specific signatures according to the rationale of Multi-Locus Sequence Typing (MLST). In classic MLST analysis, internal fragments of several housekeeping genes are amplified and sequenced (Enright et al. Infection and Immunity, 2001, 69, 2416-2427). In classic MLST analysis, internal fragments of several housekeeping genes are amplified and sequenced. In the present investigation, bioagent identifying amplicons from housekeeping genes were produced using drill-down primers and analyzed by mass spectrometry. Since mass spectral analysis results in molecular mass, from which base composition can be determined, the challenge was to determine whether resolution of emm classification of strains of Streptococcus pyogenes could be determined.

An alignment was constructed of concatenated alleles of seven MLST housekeeping genes (glucose kinase (gki), glutamine transporter protein (gtr), glutamate racemase (murI), DNA mismatch repair protein (mutS), xanthine phosphoribosyl transferase (xpt), and acetyl-CoA acetyl transferase (yqiL)) from each of the 212 previously emm-typed strains of Streptococcus pyogenes. From this alignment, the number and location of primer pairs that would maximize strain identification via base composition was determined. As a result, 6 primer pairs were chosen as standard drill-down primers for determination of emm-type of Streptococcus pyogenes. These six primer pairs are displayed in Table 7. This drill-down set comprises primers with T modifications (note TMOD designation in primer names) which constitutes a functional improvement with regard to prevention of non-templated adenylation (vide supra) relative to originally selected primers which are displayed below in the same row.

TABLE 7
Group A Streptococcus Drill-Down Primer Pairs

| | | Forward | | Reverse | |
|---|---|---|---|---|---|
| | | Primer | | Primer | |
| Primer | | (SEQ | | (SEQ | Target |
| Pair No. | Forward Primer Name | ID NO:) | Reverse Primer Name | ID NO:) | Gene |
| 442 | SP101_SPET11_358_387_TMOD_F | 311 | SP101_SPET11_448_473_TMOD_R | 669 | gki |
| 80 | SP101_SPET11_358_387_F | 310 | SP101_SPET11_448_473_TMOD_R | 668 | gki |
| 443 | SP101_SPET11_600_629_TMOD_F | 314 | SP101_SPET11_686_714_TMOD_R | 671 | gtr |
| 81 | SP101_SPET11_600_629_F | 313 | SP101_SPET11_686_714_R | 670 | gtr |
| 426 | SP101_SPET11_1314_1336_TMOD_F | 278 | SP101_SPET11_1403_1431_TMOD_R | 633 | murI |
| 86 | SP101_SPET11_1314_1336_F | 277 | SP101_SPET11_1403_1431_R | 632 | murI |
| 430 | SP101_SPET11_1807_1835_TMOD_F | 286 | SP101_SPET11_1901_1927_TMOD_R | 641 | mutS |
| 90 | SP101_SPET11_1807_1835_F | 285 | SP101_SPET11_1901_1927_R | 640 | mutS |
| 438 | SP101_SPET11_3075_3103_TMOD_F | 302 | SP101_SPET11_3168_3196_TMOD_R | 657 | xpt |
| 96 | SP101_SPET11_3075_3103_F | 301 | SP101_SPET11_3168_3196_R | 656 | xpt |
| 441 | SP101_SPET11_3511_3535_TMOD_F | 309 | SP101_SPET11_3605_3629_TMOD_R | 664 | yqiL |
| 98 | SP101_SPET11_3511_3535_F | 308 | SP101_SPET11_3605_3629_R | 663 | yqiL |

The primers of Table 7 were used to produce bioagent identifying amplicons from nucleic acid present in the clinical samples. The bioagent identifying amplicons which were subsequently analyzed by mass spectrometry and base compositions corresponding to the molecular masses were calculated.

Of the 51 samples taken during the peak of the November/December 2002 epidemic (Table 8A-C rows 1-3), all except three samples were found to represent emm3, a Group A Streptococcus genotype previously associated with high respiratory virulence. The three outliers were from samples obtained from healthy individuals and probably represent non-epidemic strains. Archived samples (Tables 8A-C rows 5-13) from historical collections showed a greater heterogeneity of base compositions and emm types as would be expected from different epidemics occurring at different places and dates. The results of the mass spectrometry analysis and emm gene sequencing were found to be concordant for the epidemic and historical samples.

TABLE 8A
Base Composition Analysis of Bioagent Identifying Amplicons of Group A
Streptococcus samples from Six Military Installations Obtained
with Primer Pair Nos. 426 and 430

| | emm-type by | | | | murI | mutS |
|---|---|---|---|---|---|---|
| # of | Mass | emm-Gene | Location | | (Primer Pair | (Primer Pair |
| Instances | Spectrometry | Sequencing | (sample) | Year | No. 426) | No. 430) |
| 48 | 3 | 3 | MCRD San | 2002 | A39 G25 C20 T34 | A38 G27 C23 T33 |
| 2 | 6 | 6 | Diego | | A40 G24 C20 T34 | A38 G27 C23 T33 |
| 1 | 28 | 28 | (Cultured) | | A39 G25 C20 T34 | A38 G27 C23 T33 |
| 15 | 3 | ND | | | A39 G25 C20 T34 | A38 G27 C23 T33 |
| 6 | 3 | 3 | NHRC San | 2003 | A39 G25 C20 T34 | A38 G27 C23 T33 |
| 3 | 5, 58 | 5 | Diego- | | A40 G24 C20 T34 | A38 G27 C23 T33 |
| 6 | 6 | 6 | Archive | | A40 G24 C20 T34 | A38 G27 C23 T33 |
| 1 | 11 | 11 | (Cultured) | | A39 G25 C20 T34 | A38 G27 C23 T33 |
| 3 | 12 | 12 | | | A40 G24 C20 T34 | A38 G26 C24 T33 |
| 1 | 22 | 22 | | | A39 G25 C20 T34 | A38 G27 C23 T33 |
| 3 | 25, 75 | 75 | | | A39 G25 C20 T34 | A38 G27 C23 T33 |
| 4 | 44/61, 82, 9 | 44/61 | | | A40 G24 C20 T34 | A38 G26 C24 T33 |
| 2 | 53, 91 | 91 | | | A39 G25 C20 T34 | A38 G27 C23 T33 |
| 1 | 2 | 2 | Ft. | 2003 | A39 G25 C20 T34 | A38 G27 C24 T32 |
| 2 | 3 | 3 | Leonard | | A39 G25 C20 T34 | A38 G27 C23 T33 |
| 1 | 4 | 4 | Wood | | A39 G25 C20 T34 | A38 G27 C23 T33 |
| 1 | 6 | 6 | (Cultured) | | A40 G24 C20 T34 | A38 G27 C23 T33 |
| 11 | 25 or 75 | 75 | | | A39 G25 C20 T34 | A38 G27 C23 T33 |
| 1 | 25, 75, 33, | 75 | | | A39 G25 C20 T34 | A38 G27 C23 T33 |
| | 34, 4, 52, 84 | | | | | |
| 1 | 44/61 or 82 | 44/61 | | | A40 G24 C20 T34 | A38 G26 C24 T33 |
| | or 9 | | | | | |
| 2 | 5 or 58 | 5 | | | A40 G24 C20 T34 | A38 G27 C23 T33 |
| 3 | 1 | 1 | Ft. Sill | 2003 | A40 G24 C20 T34 | A38 G27 C23 T33 |
| 2 | 3 | 3 | (Cultured) | | A39 G25 C20 T34 | A38 G27 C23 T33 |
| 1 | 4 | 4 | | | A39 G25 C20 T34 | A38 G27 C23 T33 |
| 1 | 28 | 28 | | | A39 G25 C20 T34 | A38 G27 C23 T33 |
| 1 | 3 | 3 | Ft. | 2003 | A39 G25 C20 T34 | A38 G27 C23 T33 |
| 1 | 4 | 4 | Benning | | A39 G25 C20 T34 | A38 G27 C23 T33 |
| 3 | 6 | 6 | (Cultured) | | A40 G24 C20 T34 | A38 G27 C23 T33 |
| 1 | 11 | 11 | | | A39 G25 C20 T34 | A38 G27 C23 T33 |
| 1 | 13 | 94** | | | A40 G24 C20 T34 | A38 G27 C23 T33 |
| 1 | 44/61 or 82 | 82 | | | A40 G24 C20 T34 | A38 G26 C24 T33 |
| | or 9 | | | | | |
| 1 | 5 or 58 | 58 | | | A40 G24 C20 T34 | A38 G27 C23 T33 |
| 1 | 78 or 89 | 89 | | | A39 G25 C20 T34 | A38 G27 C23 T33 |
| 2 | 5 or 58 | ND | Lackland | 2003 | A40 G24 C20 T34 | A38 G27 C23 T33 |
| 1 | 2 | | AFB | | A39 G25 C20 T34 | A38 G27 C24 T32 |
| 1 | 81 or 90 | | (Throat | | A40 G24 C20 T34 | A38 G27 C23 T33 |
| 1 | 78 | | Swabs) | | A38 G26 C20 T34 | A38 G27 C23 T33 |
| 3*** | No detection | | | | No detection | No detection |
| 7 | 3 | ND | MCRD San | 2002 | A39 G25 C20 T34 | A38 G27 C23 T33 |
| 1 | 3 | ND | Diego | | No detection | A38 G27 C23 T33 |
| 1 | 3 | ND | (Throat | | No detection | No detection |
| 1 | 3 | ND | Swabs) | | No detection | No detection |
| 2 | 3 | ND | | | No detection | A38 G27 C23 T33 |
| 3 | No detection | ND | | | No detection | No detection |

TABLE 8B
Base Composition Analysis of Bioagent Identifying Amplicons of Group A
Streptococcus samples from Six Military Installations Obtained
with Primer Pair Nos. 438 and 441

| | emm-type by | | | | xpt | yqiL |
|---|---|---|---|---|---|---|
| # of | Mass | emm-Gene | Location | | (Primer Pair | (Primer Pair |
| Instances | Spectrometry | Sequencing | (sample) | Year | No. 438) | No. 441) |
| 48 | 3 | 3 | MCRD San | 2002 | A30 G36 C20 T36 | A40 G29 C19 T31 |
| 2 | 6 | 6 | Diego | | A30 G36 C20 T36 | A40 G29 C19 T31 |
| 1 | 28 | 28 | (Cultured) | | A30 G36 C20 T36 | A41 G28 C18 T32 |
| 15 | 3 | ND | | | A30 G36 C20 T36 | A40 G29 C19 T31 |
| 6 | 3 | 3 | NHRC San | 2003 | A30 G36 C20 T36 | A40 G29 C19 T31 |
| 3 | 5, 58 | 5 | Diego- | | A30 G36 C20 T36 | A40 G29 C19 T31 |
| 6 | 6 | 6 | Archive | | A30 G36 C20 T36 | A40 G29 C19 T31 |
| 1 | 11 | 11 | (Cultured) | | A30 G36 C20 T36 | A40 G29 C19 T31 |
| 3 | 12 | 12 | | | A30 G36 C19 T37 | A40 G29 C19 T31 |
| 1 | 22 | 22 | | | A30 G36 C20 T36 | A40 G29 C19 T31 |
| 3 | 25, 75 | 75 | | | A30 G36 C20 T36 | A40 G29 C19 T31 |
| 4 | 44/61, 82, 9 | 44/61 | | | A30 G36 C20 T36 | A41 G28 C19 T31 |
| 2 | 53, 91 | 91 | | | A30 G36 C19 T37 | A40 G29 C19 T31 |
| 1 | 2 | 2 | Ft. | 2003 | A30 G36 C20 T36 | A40 G29 C19 T31 |
| 2 | 3 | 3 | Leonard | | A30 G36 C20 T36 | A40 G29 C19 T31 |
| 1 | 4 | 4 | Wood | | A30 G36 C19 T37 | A41 G28 C19 T31 |
| 1 | 6 | 6 | (Cultured) | | A30 G36 C20 T36 | A40 G29 C19 T31 |
| 11 | 25 or 75 | 75 | | | A30 G36 C20 T36 | A40 G29 C19 T31 |
| 1 | 25, 75, 33, | 75 | | | A30 G36 C19 T37 | A40 G29 C19 T31 |
| | 34, 4, 52, 84 | | | | | |
| 1 | 44/61 or 82 | 44/61 | | | A30 G36 C20 T36 | A41 G28 C19 T31 |
| | or 9 | | | | | |
| 2 | 5 or 58 | 5 | | | A30 G36 C20 T36 | A40 G29 C19 T31 |
| 3 | 1 | 1 | Ft. Sill | 2003 | A30 G36 C19 T37 | A40 G29 C19 T31 |
| 2 | 3 | 3 | (Cultured) | | A30 G36 C20 T36 | A40 G29 C19 T31 |
| 1 | 4 | 4 | | | A30 G36 C19 T37 | A41 G28 C19 T31 |
| 1 | 28 | 28 | | | A30 G36 C20 T36 | A41 G28 C18 T32 |
| 1 | 3 | 3 | Ft. | 2003 | A30 G36 C20 T36 | A40 G29 C19 T31 |
| 1 | 4 | 4 | Benning | | A30 G36 C19 T37 | A41 G28 C19 T31 |
| 3 | 6 | 6 | (Cultured) | | A30 G36 C20 T36 | A40 G29 C19 T31 |
| 1 | 11 | 11 | | | A30 G36 C20 T36 | A40 G29 C19 T31 |
| 1 | 13 | 94** | | | A30 G36 C20 T36 | A41 G28 C19 T31 |
| 1 | 44/61 or 82 | 82 | | | A30 G36 C20 T36 | A41 G28 C19 T31 |
| | or 9 | | | | | |
| 1 | 5 or 58 | 58 | | | A30 G36 C20 T36 | A40 G29 C19 T31 |
| 1 | 78 or 89 | 89 | | | A30 G36 C20 T36 | A41 G28 C19 T31 |
| 2 | 5 or 58 | ND | Lackland | 2003 | A30 G36 C20 T36 | A40 G29 C19 T31 |
| 1 | 2 | | AFB | | A30 G36 C20 T36 | A40 G29 C19 T31 |
| 1 | 81 or 90 | | (Throat | | A30 G36 C20 T36 | A40 G29 C19 T31 |
| 1 | 78 | | Swabs) | | A30 G36 C20 T36 | A41 G28 C19 T31 |
| 3*** | No detection | | | | No detection | No detection |
| 7 | 3 | ND | MCRD San | 2002 | A30 G36 C20 T36 | A40 G29 C19 T31 |
| 1 | 3 | ND | Diego | | A30 G36 C20 T36 | A40 G29 C19 T31 |
| 1 | 3 | ND | (Throat | | A30 G36 C20 T36 | No detection |
| 1 | 3 | ND | Swabs) | | No detection | A40 G29 C19 T31 |
| 2 | 3 | ND | | | A30 G36 C20 T36 | A40 G29 C19 T31 |
| 3 | No detection | ND | | | No detection | No detection |

TABLE 8C
Base Composition Analysis of Bioagent Identifying Amplicons of Group A
Streptococcus samples from Six Military Installations Obtained
with Primer Pair Nos. 438 and 441

| | emm-type by | | | | gki | gtr |
|---|---|---|---|---|---|---|
| # of | Mass | emm-Gene | Location | | (Primer Pair | ((Primer Pair |
| Instances | Spectrometry | Sequencing | (sample) | Year | No. 442) | No. 443) |
| 48 | 3 | 3 | MCRD San | 2002 | A32 G35 C17 T32 | A39 G28 C16 T32 |
| 2 | 6 | 6 | Diego | | A31 G35 C17 T33 | A39 G28 C15 T33 |
| 1 | 28 | 28 | (Cultured) | | A30 G36 C17 T33 | A39 G28 C16 T32 |
| 15 | 3 | ND | | | A32 G35 C17 T32 | A39 G28 C16 T32 |
| 6 | 3 | 3 | NHRC San | 2003 | A32 G35 C17 T32 | A39 G28 C16 T32 |
| 3 | 5, 58 | 5 | Diego- | | A30 G36 C20 T30 | A39 G28 C15 T33 |
| 6 | 6 | 6 | Archive | | A31 G35 C17 T33 | A39 G28 C15 T33 |
| 1 | 11 | 11 | (Cultured) | | A30 G36 C20 T30 | A39 G28 C16 T32 |
| 3 | 12 | 12 | | | A31 G35 C17 T33 | A39 G28 C15 T33 |
| 1 | 22 | 22 | | | A31 G35 C17 T33 | A38 G29 C15 T33 |
| 3 | 25, 75 | 75 | | | A30 G36 C17 T33 | A39 G28 C15 T33 |
| 4 | 44/61, 82, 9 | 44/61 | | | A30 G36 C18 T32 | A39 G28 C15 T33 |
| 2 | 53, 91 | 91 | | | A32 G35 C17 T32 | A39 G28 C16 T32 |
| 1 | 2 | 2 | Ft. | 2003 | A30 G36 C17 T33 | A39 G28 C15 T33 |
| 2 | 3 | 3 | Leonard | | A32 G35 C17 T32 | A39 G28 C16 T32 |
| 1 | 4 | 4 | Wood | | A31 G35 C17 T33 | A39 G28 C15 T33 |
| 1 | 6 | 6 | (Cultured) | | A31 G35 C17 T33 | A39 G28 C15 T33 |
| 11 | 25 or 75 | 75 | | | A30 G36 C17 T33 | A39 G28 C15 T33 |
| 1 | 25, 75, 33, | 75 | | | A30 G36 C17 T33 | A39 G28 C15 T33 |
| | 34, 4, 52, 84 | | | | | |
| 1 | 44/61 or 82 | 44/61 | | | A30 G36 C18 T32 | A39 G28 C15 T33 |
| | or 9 | | | | | |
| 2 | 5 or 58 | 5 | | | A30 G36 C20 T30 | A39 G28 C15 T33 |
| 3 | 1 | 1 | Ft. Sill | 2003 | A30 G36 C18 T32 | A39 G28 C15 T33 |
| 2 | 3 | 3 | (Cultured) | | A32 G35 C17 T32 | A39 G28 C16 T32 |
| 1 | 4 | 4 | | | A31 G35 C17 T33 | A39 G28 C15 T33 |
| 1 | 28 | 28 | | | A30 G36 C17 T33 | A39 G28 C16 T32 |
| 1 | 3 | 3 | Ft. | 2003 | A32 G35 C17 T32 | A39 G28 C16 T32 |
| 1 | 4 | 4 | Benning | | A31 G35 C17 T33 | A39 G28 C15 T33 |
| 3 | 6 | 6 | (Cultured) | | A31 G35 C17 T33 | A39 G28 C15 T33 |
| 1 | 11 | 11 | | | A30 G36 C20 T30 | A39 G28 C16 T32 |
| 1 | 13 | 94** | | | A30 G36 C19 T31 | A39 G28 C15 T33 |
| 1 | 44/61 or 82 | 82 | | | A30 G36 C18 T32 | A39 G28 C15 T33 |
| | or 9 | | | | | |
| 1 | 5 or 58 | 58 | | | A30 G36 C20 T30 | A39 G28 C15 T33 |
| 1 | 78 or 89 | 89 | | | A30 G36 C18 T32 | A39 G28 C15 T33 |
| 2 | 5 or 58 | ND | Lackland | 2003 | A30 G36 C20 T30 | A39 G28 C15 T33 |
| 1 | 2 | | AFB | | A30 G36 C17 T33 | A39 G28 C15 T33 |
| 1 | 81 or 90 | | (Throat | | A30 G36 C17 T33 | A39 G28 C15 T33 |
| 1 | 78 | | Swabs) | | A30 G36 C18 T32 | A39 G28 C15 T33 |
| 3*** | No detection | | | | No detection | No detection |
| 7 | 3 | ND | MCRD San | 2002 | A32 G35 C17 T32 | A39 G28 C16 T32 |
| 1 | 3 | ND | Diego | | No detection | No detection |
| 1 | 3 | ND | (Throat | | A32 G35 C17 T32 | A39 G28 C16 T32 |
| 1 | 3 | ND | Swabs) | | A32 G35 C17 T32 | No detection |
| 2 | 3 | ND | | | A32 G35 C17 T32 | No detection |
| 3 | No detection | ND | | | No detection | No detection |

### Example 9

### Design of Calibrant Polynucleotides Based on Bioagent Identifying Amplicons for Identification of Species of Bacteria (Bacterial Bioagent Identifying Amplicons)

This example describes the design of 19 calibrant polynucleotides based on bacterial bioagent identifying amplicons corresponding to the primers of the broad surveillance set (Table 4) and the Bacillus anthracis drill-down set (Table 5).

Calibration sequences were designed to simulate bacterial bioagent identifying amplicons produced by the T modified primer pairs shown in Table 4 (primer names have the designation “TMOD”). The calibration sequences were chosen as a representative member of the section of bacterial genome from specific bacterial species which would be amplified by a given primer pair. The model bacterial species upon which the calibration sequences are based are also shown in Table 9. For example, the calibration sequence chosen to correspond to an amplicon produced by primer pair no. 361 is SEQ ID NO: 722. In Table 9, the forward (_F) or reverse (_R) primer name indicates the coordinates of an extraction representing a gene of a standard reference bacterial genome to which the primer hybridizes e.g.: the forward primer name 16S_EC_{—}713_{—}732_TMOD_F indicates that the forward primer hybridizes to residues 713-732 of the gene encoding 16S ribosomal RNA in an E. coli reference sequence (in this case, the reference sequence is an extraction consisting of residues 4033120-4034661 of the genomic sequence of E. coli K12 (GenBank gi number 16127994). Additional gene coordinate reference information is shown in Table 10. The designation “TMOD” in the primer names indicates that the 5′ end of the primer has been modified with a non-matched template T residue which prevents the PCR polymerase from adding non-templated adenosine residues to the 5′ end of the amplification product, an occurrence which may result in miscalculation of base composition from molecular mass data (vide supra).

The 19 calibration sequences described in Tables 9 and 10 were combined into a single calibration polynucleotide sequence (SEQ ID NO: 741—which is herein designated a “combination calibration polynucleotide”) which was then cloned into a pCR®-Blunt vector (Invitrogen, Carlsbad, Calif.). This combination calibration polynucleotide can be used in conjunction with the primers of Table 9 as an internal standard to produce calibration amplicons for use in determination of the quantity of any bacterial bioagent. Thus, for example, when the combination calibration polynucleotide vector is present in an amplification reaction mixture, a calibration amplicon based on primer pair 346 (16S rRNA) will be produced in an amplification reaction with primer pair 346 and a calibration amplicon based on primer pair 363 (rpoC) will be produced with primer pair 363. Coordinates of each of the 19 calibration sequences within the calibration polynucleotide (SEQ ID NO: 783) are indicated in Table 10.

TABLE 9
Bacterial Primer Pairs for Production of Bacterial Bioagent Identifying
Amplicons and Corresponding Representative Calibration Sequences

| | | Forward | | Reverse | Calibration | Calibration |
|---|---|---|---|---|---|---|
| | | Primer | | Primer | Sequence | Sequence |
| Primer | | (SEQ ID | | (SEQ | Model | (SEQ ID |
| Pair No. | Forward Primer Name | NO:) | Reverse Primer Name | ID NO:) | Species | NO:) |
| 361 | 16S_EC_1090_1111_2_TMOD_F | 5 | 16S_EC_1175_1196_TMOD_R | 370 | Bacillus | 764 |
| | | | | | anthracis | |
| 346 | 16S_EC_713_732_TMOD_F | 27 | 16S_EC_789_809_TMOD_R | 389 | Bacillus | 765 |
| | | | | | anthracis | |
| 347 | 16S_EC_785_806_TMOD_F | 30 | 16S_EC_880_897_TMOD_R | 392 | Bacillus | 766 |
| | | | | | anthracis | |
| 348 | 16S_EC_960_981_TMOD_F | 38 | 16S_EC_1054_1073_TMOD_R | 363 | Bacillus | 767 |
| | | | | | anthracis | |
| 349 | 23S_EC_1826_1843_TMOD_F | 49 | 23S_EC_1906_1924_TMOD_R | 405 | Bacillus | 768 |
| | | | | | anthracis | |
| 360 | 23S_EC_2646_2667_TMOD_F | 60 | 23S_EC_2745_2765_TMOD_R | 416 | Bacillus | 769 |
| | | | | | anthracis | |
| 350 | CAPC_BA_274_303_TMOD_F | 98 | CAPC_BA_349_376_TMOD_R | 452 | Bacillus | 770 |
| | | | | | anthracis | |
| 351 | CYA_BA_1353_1379_TMOD_F | 128 | CYA_BA_1448_1467_TMOD_R | 483 | Bacillus | 771 |
| | | | | | anthracis | |
| 352 | INFB_EC_1365_1393_TMOD_F | 161 | INFB_EC_1439_1467_TMOD_R | 516 | Bacillus | 772 |
| | | | | | anthracis | |
| 353 | LEF_BA_756_781_TMOD_F | 175 | LEF_BA_843_872_TMOD_R | 531 | Bacillus | 773 |
| | | | | | anthracis | |
| 356 | RPLB_EC_650_679_TMOD_F | 232 | RPLB_EC_739_762_TMOD_R | 592 | Clostridium | 774 |
| | | | | | botulinum | |
| 449 | RPLB_EC_690_710_F | 237 | RPLB_EC_737_758_R | 589 | Clostridium | 775 |
| | | | | | botulinum | |
| 359 | RPOB_EC_1845_1866_TMOD_F | 241 | RPOB_EC_1909_1929_TMOD_R | 597 | Yersinia | 776 |
| | | | | | Pestis | |
| 362 | RPOB_EC_3799_3821_TMOD_F | 245 | RPOB_EC_3862_3888_TMOD_R | 603 | Burkholderia | 777 |
| | | | | | mallei | |
| 363 | RPOC_EC_2146_2174_TMOD_F | 257 | RPOC_EC_2227_2245_TMOD_R | 621 | Burkholderia | 778 |
| | | | | | mallei | |
| 354 | RPOC_EC_2218_2241_TMOD_F | 262 | RPOC_EC_2313_2337_TMOD_R | 625 | Bacillus | 779 |
| | | | | | anthracis | |
| 355 | SSPE_BA_115_137_TMOD_F | 321 | SSPE_BA_197_222_TMOD_R | 687 | Bacillus | 780 |
| | | | | | anthracis | |
| 367 | TUFB_EC_957_979_TMOD_F | 345 | TUFB_EC_1034_1058_THOD_R | 701 | Burkholderia | 781 |
| | | | | | mallei | |
| 358 | VALS_EC_1105_1124_TMOD_F | 350 | VALS_EC_1195_1218_TMOD_R | 712 | Yersinia | 782 |
| | | | | | Pestis | |

TABLE 10
Primer Pair Gene Coordinate References and Calibration Polynucleotide
Sequence Coordinates within the Combination Calibration Polynucleotide

| | | | | Coordinates of Calibration |
|---|---|---|---|---|
| | | Reference GenBank GI No. of | | Sequence in Combination |
| Bacterial Gene | Gene Extraction Coordinates | Genomic (G) or Plasmid (P) | Primer Pair | Calibration Polynucleotide (SEQ |
| and Species | of Genomic or Plasmid Sequence | Sequence | No. | ID NO: 783) |
| 16S E. coli | 4033120 . . . 4034661 | 16127994 (G) | 346 | 16 . . . 109 |
| 16S E. coli | 4033120 . . . 4034661 | 16127994 (G) | 347 | 83 . . . 190 |
| 16S E. coli | 4033120 . . . 4034661 | 16127994 (G) | 348 | 246 . . . 353 |
| 16S E. coli | 4033120 . . . 4034661 | 16127994 (G) | 361 | 368 . . . 469 |
| 23S E. coli | 4166220 . . . 4169123 | 16127994 (G) | 349 | 743 . . . 837 |
| 23S E. coli | 4166220 . . . 4169123 | 16127994 (G) | 360 | 865 . . . 981 |
| rpoB E. coli. | 4178823 . . . 4182851 | 16127994 (G) | 359 | 1591 . . . 1672 |
| | (complement strand) | | | |
| rpoB E. coli | 4178823 . . . 4182851 | 16127994 (G) | 362 | 2081 . . . 2167 |
| | (complement strand) | | | |
| rpoC E. coli | 4182928 . . . 4187151 | 16127994 (G) | 354 | 1810 . . . 1926 |
| rpoC E. coli | 4182928 . . . 4187151 | 16127994 (G) | 363 | 2183 . . . 2279 |
| infB E. coli | 3313655 . . . 3310983 | 16127994 (G) | 352 | 1692 . . . 1791 |
| | (complement strand) | | | |
| tufB E. coli | 4173523 . . . 4174707 | 16127994 (G) | 367 | 2400 . . . 2498 |
| rplB E. coli | 3449001 . . . 3448180 | 16127994 (G) | 356 | 1945 . . . 2060 |
| rplB E. coli | 3449001 . . . 3448180 | 16127994 (G) | 449 | 1986 . . . 2055 |
| valS E. coli | 4481405 . . . 4478550 | 16127994 (G) | 358 | 1462 . . . 1572 |
| | (complement strand) | | | |
| capC | 56074 . . . 55628 | 6470151 (P) | 350 | 2517 . . . 2616 |
| B. anthracis | (complement strand) | | | |
| cya | 156626 . . . 154288 | 4894216 (P) | 351 | 1338 . . . 1449 |
| B. anthracis | (complement strand) | | | |
| lef | 127442 . . . 129921 | 4894216 (P) | 353 | 1121 . . . 1234 |
| B. anthracis | | | | |
| sspE | 226496 . . . 226783 | 30253828 (G) | 355 | 1007-1104 |
| B. anthracis | | | | |

### Example 10

### Use of a Calibration Polynucleotide for Determining the Quantity of Bacillus Anthracis in a Sample Containing a Mixture of Microbes

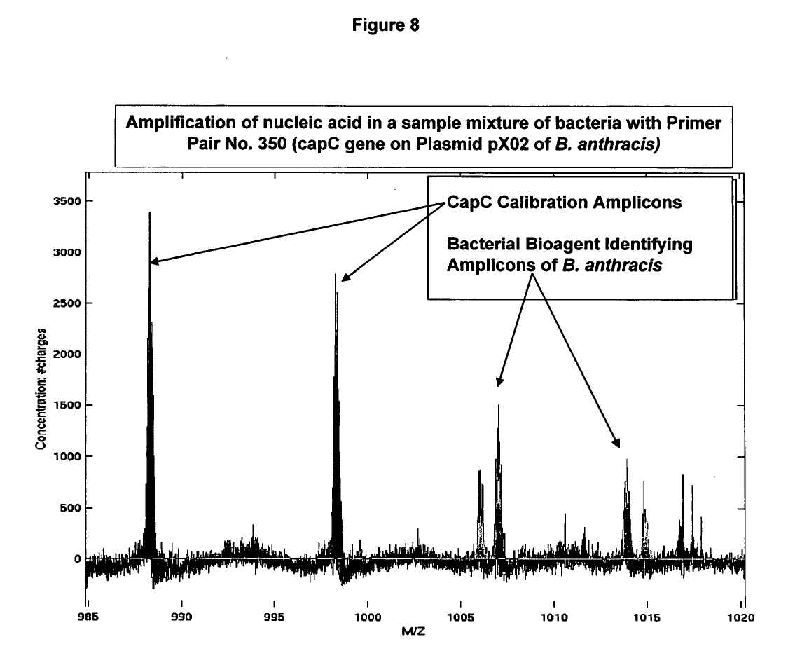

The process described in this example is shown in FIG. 7. The capC gene is a gene involved in capsule synthesis which resides on the pX02 plasmid of Bacillus anthracis. Primer pair number 350 (see Tables 9 and 10) was designed to identify Bacillus anthracis via production of a bacterial bioagent identifying amplicon. Known quantities of the combination calibration polynucleotide vector described in Example 3 were added to amplification mixtures containing bacterial bioagent nucleic acid from a mixture of microbes which included the Ames strain of Bacillus anthracis. Upon amplification of the bacterial bioagent nucleic acid and the combination calibration polynucleotide vector with primer pair no. 350, bacterial bioagent identifying amplicons and calibration amplicons were obtained and characterized by mass spectrometry. A mass spectrum measured for the amplification reaction is shown in FIG. 8). The molecular masses of the bioagent identifying amplicons provided the means for identification of the bioagent from which they were obtained (Ames strain of Bacillus anthracis) and the molecular masses of the calibration amplicons provided the means for their identification as well. The relationship between the abundance (peak height) of the calibration amplicon signals and the bacterial bioagent identifying amplicon signals provides the means of calculation of the copies of the pX02 plasmid of the Ames strain of Bacillus anthracis. Methods of calculating quantities of molecules based on internal calibration procedures are well known to those of ordinary skill in the art.

Averaging the results of 10 repetitions of the experiment described above, enabled a calculation that indicated that the quantity of Ames strain of Bacillus anthracis present in the sample corresponds to approximately 10 copies of pX02 plasmid.

### Example 11

### Drill-Down Genotyping of Campylobacter Species

A series of drill-down primers were designed as described in Example 1 with the objective of identification of different strains of Campylobacter jejuni. The primers are listed in Table 11 with the designation “CJST_CJ.” Housekeeping genes to which the primers hybridize and produce bioagent identifying amplicons include: tkt (transketolase), glyA (serine hydroxymethyltransferase), gltA (citrate synthase), aspA (aspartate ammonia lyase), glnA (glutamine synthase), pgm (phosphoglycerate mutase), and uncA (ATP synthetase alpha chain).

TABLE 11
Campylobacter Drill-down Primer Pairs

| Primer | | | | | |
|---|---|---|---|---|---|
| Pair | | Forward Primer | | Reverse Primer | Target |
| No. | Forward Primer Name | (SEQ ID NO:) | Reverse Primer Name | (SEQ ID NO:) | Gene |
| 1053 | CJST_CJ_1080_1110_F | 102 | CJST_CJ_1166_1198_R | 456 | gltA |
| 1064 | CJST_CJ_1680_1713_F | 107 | CJST_CJ_1795_1822_R | 461 | glyA |
| 1054 | CJST_CJ_2060_2090_F | 109 | CJST_CJ_2148_2174_R | 463 | pgm |
| 1049 | CJST_CJ_2636_2668_F | 113 | CJST_CJ_2753_2777_R | 467 | tkt |
| 1048 | CJST_CJ_360_394_F | 119 | CJST_CJ_442_476_R | 472 | aspA |
| 1047 | CJST_CJ_584_616_F | 121 | CJST_CJ_663_692_R | 474 | glnA |

The primers were used to amplify nucleic acid from 50 food product samples provided by the USDA, 25 of which contained Campylobacter jejuni and 25 of which contained Campylobacter coli. Primers used in this study were developed primarily for the discrimination of Campylobacter jejuni clonal complexes and for distinguishing Campylobacter jejuni from Campylobacter coli. Finer discrimination between Campylobacter coli types is also possible by using specific primers targeted to loci where closely-related Campylobacter coli isolates demonstrate polymorphisms between strains. The conclusions of the comparison of base composition analysis with sequence analysis are shown in Tables 12A-C.

TABLE 12A
Results of Base Composition Analysis of 50 Campylobacter Samples with
Drill-down MLST Primer Pair Nos: 1048 and 1047

| | | | | | | Base | Base |
|---|---|---|---|---|---|---|---|
| | | | | | | Composition of | Composition of |
| | | | MLST type or | | | Bioagent | Bioagent |
| | | | Clonal | MLST Type | | Identifying | Identifying |
| | | | Complex by | or Clonal | | Amplicon | Amplicon |
| | | | Base | Complex by | | Obtained with | Obtained with |
| | | Isolate | Composition | Sequence | | Primer Pair No: | Primer Pair |
| Group | Species | origin | analysis | analysis | Strain | 1048 (aspA) | No: 1047 (glnA) |
| J-1 | C. jejuni | Goose | ST 690/ | ST 991 | RM3673 | A30 G25 C16 T46 | A47 G21 C16 T25 |
| | | | 692/707/991 | | | | |
| J-2 | C. jejuni | Human | Complex | ST 356, | RM4192 | A30 G25 C16 T46 | A48 G21 C17 T23 |
| | | | 206/48/353 | complex | | | |
| | | | | 353 | | | |
| J-3 | C. jejuni | Human | Complex | ST 436 | RM4194 | A30 G25 C15 T47 | A48 G21 C18 T22 |
| | | | 354/179 | | | | |
| J-4 | C. jejuni | Human | Complex 257 | ST 257, | RM4197 | A30 G25 C16 T46 | A48 G21 C18 T22 |
| | | | | complex | | | |
| | | | | 257 | | | |
| J-5 | C. jejuni | Human | Complex 52 | ST 52, | RM277 | A30 G25 C16 T46 | A48 G21 C17 T23 |
| | | | | complex 52 | | | |
| J-6 | C. jejuni | Human | Complex 443 | ST 51, | RM4275 | A30 G25 C15 T47 | A48 G21 C17 T23 |
| | | | | complex | RM4279 | A30 G25 C15 T47 | A48 G21 C17 T23 |
| | | | | 443 | | | |
| J-7 | C. jejuni | Human | Complex 42 | ST 604, | RM1864 | A30 G25 C15 T47 | A48 G21 C18 T22 |
| | | | | complex 42 | | | |
| J-8 | C. jejuni | Human | Complex | ST 362, | RM3193 | A30 G25 C15 T47 | A48 G21 C18 T22 |
| | | | 42/49/362 | complex | | | |
| | | | | 362 | | | |
| J-9 | C. jejuni | Human | Complex | ST 147, | RM3203 | A30 G25 C15 T47 | A47 G21 C18 T23 |
| | | | 45/283 | Complex 45 | | | |
| | C. jejuni | Human | Consistent | ST 828 | RM4183 | A31 G27 C20 T39 | A48 G21 C16 T24 |
| C-1 | C. coli | Poultry | with 74 | ST 832 | RM1169 | A31 G27 C20 T39 | A48 G21 C16 T24 |
| | | | closely | ST 1056 | RM1857 | A31 G27 C20 T39 | A48 G21 C16 T24 |
| | | | related | ST 889 | RM1166 | A31 G27 C20 T39 | A48 G21 C16 T24 |
| | | | sequence | ST 829 | RM1182 | A31 G27 C20 T39 | A48 G21 C16 T24 |
| | | | types (none | ST 1050 | RM1518 | A31 G27 C20 T39 | A48 G21 C16 T24 |
| | | | belong to a | ST 1051 | RM1521 | A31 G27 C20 T39 | A48 G21 C16 T24 |
| | | | clonal | ST 1053 | RM1523 | A31 G27 C20 T39 | A48 G21 C16 T24 |
| | | | complex) | ST 1055 | RM1527 | A31 G27 C20 T39 | A48 G21 C16 T24 |
| | | | | ST 1017 | RM1529 | A31 G27 C20 T39 | A48 G21 C16 T24 |
| | | | | ST 860 | RM1840 | A31 G27 C20 T39 | A48 G21 C16 T24 |
| | | | | ST 1063 | RM2219 | A31 G27 C20 T39 | A48 G21 C16 T24 |
| | | | | ST 1066 | RM2241 | A31 G27 C20 T39 | A48 G21 C16 T24 |
| | | | | ST 1067 | RM2243 | A31 G27 C20 T39 | A48 G21 C16 T24 |
| | | | | ST 1068 | RM2439 | A31 G27 C20 T39 | A48 G21 C16 T24 |
| | | Swine | | ST 1016 | RM3230 | A31 G27 C20 T39 | A48 G21 C16 T24 |
| | | | | ST 1069 | RM3231 | A31 G27 C20 T39 | A48 G21 C16 T24 |
| | | | | ST 1061 | RM1904 | A31 G27 C20 T39 | A48 G21 C16 T24 |
| | | Unknown | | ST 825 | RM1534 | A31 G27 C20 T39 | A48 G21 C16 T24 |
| | | | | ST 901 | RM1505 | A31 G27 C20 T39 | A48 G21 C16 T24 |
| C-2 | C. coli | Human | ST 895 | ST 895 | RM1532 | A31 G27 C19 T40 | A48 G21 C16 T24 |
| C-3 | C. coli | Poultry | Consistent | ST 1064 | RM2223 | A31 G27 C20 T39 | A48 G21 C16 T24 |
| | | | with 63 | ST 1082 | RM1178 | A31 G27 C20 T39 | A48 G21 C16 T24 |
| | | | closely | ST 1054 | RM1525 | A31 G27 C20 T39 | A48 G21 C16 T24 |
| | | | related | ST 1049 | RM1517 | A31 G27 C20 T39 | A48 G21 C16 T24 |
| | | Marmoset | sequence | ST 891 | RM1531 | A31 G27 C20 T39 | A48 G21 C16 T24 |
| | | | types (none | | | | |
| | | | belong to a | | | | |
| | | | clonal | | | | |
| | | | complex) | | | | |

TABLE 12B
Results of Base Composition Analysis of 50 Campylobacter Samples with
Drill-down MLST Primer Pair Nos: 1053 and 1064

| | | | | | | Base | Base |
|---|---|---|---|---|---|---|---|
| | | | | | | Composition of | Composition of |
| | | | MLST type or | | | Bioagent | Bioagent |
| | | | Clonal | MLST Type | | Identifying | Identifying |
| | | | Complex by | or Clonal | | Amplicon | Amplicon |
| | | | Base | Complex by | | Obtained with | Obtained with |
| | | Isolate | Composition | Sequence | | Primer Pair | Primer Pair |
| Group | Species | origin | analysis | analysis | Strain | No: 1053 (gltA) | No: 1064 (glyA) |
| J-1 | C. jejuni | Goose | ST 690/ | ST 991 | RM3673 | A24 G25 C23 T47 | A40 G29 C29 T45 |
| | | | 692/707/991 | | | | |
| J-2 | C. jejuni | Human | Complex | ST 356, | RM4192 | A24 G25 C23 T47 | A40 G29 C29 T45 |
| | | | 206/48/353 | complex | | | |
| | | | | 353 | | | |
| J-3 | C. jejuni | Human | Complex | ST 436 | RM4194 | A24 G25 C23 T47 | A40 G29 C29 T45 |
| | | | 354/179 | | | | |
| J-4 | C. jejuni | Human | Complex 257 | ST 257, | RM4197 | A24 G25 C23 T47 | A40 G29 C29 T45 |
| | | | | complex | | | |
| | | | | 257 | | | |
| J-5 | C. jejuni | Human | Complex 52 | ST 52, | RM4277 | A24 G25 C23 T47 | A39 G30 C26 T48 |
| | | | | complex 52 | | | |
| J-6 | C. jejuni | Human | Complex 443 | ST 51, | RM4275 | A24 G25 C23 T47 | A39 G30 C28 T46 |
| | | | | complex | RM4279 | A24 G25 C23 T47 | A39 G30 C28 T46 |
| | | | | 443 | | | |
| J-7 | C. jejuni | Human | Complex 42 | ST 604, | RM1864 | A24 G25 C23 T47 | A39 G30 C26 T48 |
| | | | | complex 42 | | | |
| J-8 | C. jejuni | Human | Complex | ST 362, | RM3193 | A24 G25 C23 T47 | A38 G31 C28 T46 |
| | | | 42/49/362 | complex | | | |
| | | | | 362 | | | |
| J-9 | C. jejuni | Human | Complex | ST 147, | RM3203 | A24 G25 C23 T47 | A38 G31 C28 T46 |
| | | | 45/283 | Complex 45 | | | |
| | C. jejuni | Human | Consistent | ST 828 | RM4183 | A23 G24 C26 T46 | A39 G30 C27 T47 |
| C-1 | C. coli | | with 74 | ST 832 | RM1169 | A23 G24 C26 T46 | A39 G30 C27 T47 |
| | | | closely | ST 1056 | RM1857 | A23 G24 C26 T46 | A39 G30 C27 T47 |
| | | Poultry | related | ST 889 | RM1166 | A23 G24 C26 T46 | A39 G30 C27 T47 |
| | | | sequence | ST 829 | RM1182 | A23 G24 C26 T46 | A39 G30 C27 T47 |
| | | | types (none | ST 1050 | RM1518 | A23 G24 C26 T46 | A39 G30 C27 T47 |
| | | | belong to a | ST 1051 | RM1521 | A23 G24 C26 T46 | A39 G30 C27 T47 |
| | | | clonal | ST 1053 | RM1523 | A23 G24 C26 T46 | A39 G30 C27 T47 |
| | | | complex) | ST 1055 | RM1527 | A23 G24 C26 T46 | A39 G30 C27 T47 |
| | | | | ST 1017 | RM1529 | A23 G24 C26 T46 | A39 G30 C27 T47 |
| | | | | ST 860 | RM1840 | A23 G24 C26 T46 | A39 G30 C27 T47 |
| | | | | ST 1063 | RM2219 | A23 G24 C26 T46 | A39 G30 C27 T47 |
| | | | | ST 1066 | RM2241 | A23 G24 C26 T46 | A39 G30 C27 T47 |
| | | | | ST 1067 | RM2243 | A23 G24 C26 T46 | A39 G30 C27 T47 |
| | | | | ST 1068 | RM2439 | A23 G24 C26 T46 | A39 G30 C27 T47 |
| | | Swine | | ST 1016 | RM3230 | A23 G24 C26 T46 | A39 G30 C27 T47 |
| | | | | ST 1069 | RM3231 | A23 G24 C26 T46 | NO DATA |
| | | | | ST 1061 | RM1904 | A23 G24 C26 T46 | A39 G30 C27 T47 |
| | | Unknown | | ST 825 | RM1534 | A23 G24 C26 T46 | A39 G30 C27 T47 |
| | | | | ST 901 | RM1505 | A23 G24 C26 T46 | A39 G30 C27 T47 |
| C-2 | C. coli | Human | ST 895 | ST 895 | RM1532 | A23 G24 C26 T46 | A39 G30 C27 T47 |
| C-3 | C. coli | Poultry | Consistent | ST 1064 | RM2223 | A23 G24 C26 T46 | A39 G30 C27 T47 |
| | | | with 63 | ST 1082 | RM1178 | A23 G24 C26 T46 | A39 G30 C27 T47 |
| | | | closely | ST 1054 | RM1525 | A23 G24 C25 T47 | A39 G30 C27 T47 |
| | | | related | ST 1049 | RM1517 | A23 G24 C26 T46 | A39 G30 C27 T47 |
| | | Marmoset | sequence | ST 891 | RM1531 | A23 G24 C26 T46 | A39 G30 C27 T47 |
| | | | types (none | | | | |
| | | | belong to a | | | | |
| | | | clonal | | | | |
| | | | complex) | | | | |

TABLE 12C
Results of Base Composition Analysis of 50 Campylobacter Samples with
Drill-down MLST Primer Pair Nos: 1054 and 1049

| | | | | | | Base | Base |
|---|---|---|---|---|---|---|---|
| | | | | | | Composition of | Composition of |
| | | | MLST type or | | | Bioagent | Bioagent |
| | | | Clonal | MLST Type | | Identifying | Identifying |
| | | | Complex by | or Clonal | | Amplicon | Amplicon |
| | | | Base | Complex by | | Obtained with | Obtained with |
| | | Isolate | Composition | Sequence | | Primer Pair No: | Primer Pair |
| Group | Species | origin | analysis | analysis | Strain | 1054 (pgm) | No: 1049 (tkt) |
| J-1 | C. jejuni | Goose | ST 690/ | ST 991 | RM3673 | A26 G33 C18 T38 | A41 G28 C35 T38 |
| | | | 692/707/991 | | | | |
| J-2 | C. jejuni | Human | Complex | ST 356, | RM4192 | A26 G33 C19 T37 | A41 G28 C36 T37 |
| | | | 206/48/353 | complex | | | |
| | | | | 353 | | | |
| J-3 | C. jejuni | Human | Complex | ST 436 | RM4194 | A27 G32 C19 T37 | A42 G28 C36 T36 |
| | | | 354/179 | | | | |
| J-4 | C. jejuni | Human | Complex 257 | ST 257, | RM4197 | A27 G32 C19 T37 | A41 G29 C35 T37 |
| | | | | complex | | | |
| | | | | 257 | | | |
| J-5 | C. jejuni | Human | Complex 52 | ST 52, | RM4277 | A26 G33 C18 T38 | A41 G28 C36 T37 |
| | | | | complex 52 | | | |
| J-6 | C. jejuni | Human | Complex 443 | ST 51, | RM4275 | A27 G31 C19 T38 | A41 G28 C36 T37 |
| | | | | complex | RM4279 | A27 G31 C19 T38 | A41 G28 C36 T37 |
| | | | | 443 | | | |
| J-7 | C. jejuni | Human | Complex 42 | ST 604, | RM1864 | A27 G32 C19 T37 | A42 G28 C35 T37 |
| | | | | complex 42 | | | |
| J-8 | C. jejuni | Human | Complex | ST 362, | RM3193 | A26 G33 C19 T37 | A42 G28 C35 T37 |
| | | | 42/49/362 | complex | | | |
| | | | | 362 | | | |
| J-9 | C. jejuni | Human | Complex | ST 147, | RM3203 | A28 G31 C19 T37 | A43 G28 C36 T35 |
| | | | 45/283 | Complex 45 | | | |
| | C. jejuni | Human | Consistent | ST 828 | RM4183 | A27 G30 C19 T39 | A46 G28 C32 T36 |
| C-1 | C. coli | | with 74 | ST 832 | RM1169 | A27 G30 C19 T39 | A46 G28 C32 T36 |
| | | | closely | ST 1056 | RM1857 | A27 G30 C19 T39 | A46 G28 C32 T36 |
| | | Poultry | related | ST 889 | RM1166 | A27 G30 C19 T39 | A46 G28 C32 T36 |
| | | | sequence | ST 829 | RM1182 | A27 G30 C19 T39 | A46 G28 C32 T36 |
| | | | types (none | ST 1050 | RM1518 | A27 G30 C19 T39 | A46 G28 C32 T36 |
| | | | belong to a | ST 1051 | RM1521 | A27 G30 C19 T39 | A46 G28 C32 T36 |
| | | | clonal | ST 1053 | RM1523 | A27 G30 C19 T39 | A46 G28 C32 T36 |
| | | | complex) | ST 1055 | RM1527 | A27 G30 C19 T39 | A46 G28 C32 T36 |
| | | | | ST 1017 | RM1529 | A27 G30 C19 T39 | A46 G28 C32 T36 |
| | | | | ST 860 | RM1840 | A27 G30 C19 T39 | A46 G28 C32 T36 |
| | | | | ST 1063 | RM2219 | A27 G30 C19 T39 | A46 G28 C32 T36 |
| | | | | ST 1066 | RM2241 | A27 G30 C19 T39 | A46 G28 C32 T36 |
| | | | | ST 1067 | RM2243 | A27 G30 C19 T39 | A46 G28 C32 T36 |
| | | | | ST 1068 | RM2439 | A27 G30 C19 T39 | A46 G28 C32 T36 |
| | | Swine | | ST 1016 | RM3230 | A27 G30 C19 T39 | A46 G28 C32 T36 |
| | | | | ST 1069 | RM3231 | A27 G30 C19 T39 | A46 G28 C32 T36 |
| | | | | ST 1061 | RM1904 | A27 G30 C19 T39 | A46 G28 C32 T36 |
| | | Unknown | | ST 825 | RM1534 | A27 G30 C19 T39 | A46 G28 C32 T36 |
| | | | | ST 901 | RM1505 | A27 G30 C19 T39 | A46 G28 C32 T36 |
| C-2 | C. coli | Human | ST 895 | ST 895 | RM1532 | A27 G30 C19 T39 | A45 G29 C32 T36 |
| C-3 | C. coli | Poultry | Consistent | ST 1064 | RM2223 | A27 G30 C19 T39 | A45 G29 C32 T36 |
| | | | with 63 | ST 1082 | RM1178 | A27 G30 C19 T39 | A45 G29 C32 T36 |
| | | | closely | ST 1054 | RM1525 | A27 G30 C19 T39 | A45 G29 C32 T36 |
| | | | related | ST 1049 | RM1517 | A27 G30 C19 T39 | A45 G29 C32 T36 |
| | | Marmoset | sequence | ST 891 | RM1531 | A27 G30 C19 T39 | A45 G29 C32 T36 |
| | | | types (none | | | | |
| | | | belong to a | | | | |
| | | | clonal | | | | |
| | | | complex) | | | | |

The base composition analysis method was successful in identification of 12 different strain groups. Campylobacter jejuni and Campylobacter coli are generally differentiated by all loci. Ten clearly differentiated Campylobacter jejuni isolates and 2 major Campylobacter coli groups were identified even though the primers were designed for strain typing of Campylobacter jejuni. One isolate (RM4183) which was designated as Campylobacter jejuni was found to group with Campylobacter coli and also appears to actually be Campylobacter coil by full MLST sequencing.

### Example 12

### Identification of Acinetobacter baumannii Using Broad Range Survey and Division-Wide Primers in Epidemiological Surveillance

To test the capability of the broad range survey and division-wide primer sets of Table 4 in identification of Acinetobacter species, 183 clinical samples were obtained from individuals participating in, or in contact with individuals participating in Operation Iraqi Freedom (including US service personnel, US civilian patients at the Walter Reed Army Institute of Research (WRAIR), medical staff, Iraqi civilians and enemy prisoners). In addition, 34 environmental samples were obtained from hospitals in Iraq, Kuwait, Germany, the United States and the USNS Comfort, a hospital ship.

Upon amplification of nucleic acid obtained from the clinical samples, primer pairs 346-349, 360, 361, 354, 362 and 363 (Table 4) all produced bacterial bioagent amplicons which identified Acinetobacter baumannii in 215 of 217 samples. The organism Klebsiella pneumoniae was identified in the remaining two samples. In addition, 14 different strain types (containing single nucleotide polymorphisms relative to a reference strain of Acinetobacter baumannii) were identified and assigned arbitrary numbers from 1 to 14. Strain type 1 was found in 134 of the sample isolates and strains 3 and 7 were found in 46 and 9 of the isolates respectively.

The epidemiology of strain type 7 of Acinetobacter baumannii was investigated. Strain 7 was found in 4 patients and 5 environmental samples (from field hospitals in Iraq and Kuwait). The index patient infected with strain 7 was a pre-war patient who had a traumatic amputation in March of 2003 and was treated at a Kuwaiti hospital. The patient was subsequently transferred to a hospital in Germany and then to WRAIR. Two other patients from Kuwait infected with strain 7 were found to be non-infectious and were not further monitored. The fourth patient was diagnosed with a strain 7 infection in September of 2003 at WRAIR. Since the fourth patient was not related involved in Operation Iraqi Freedom, it was inferred that the fourth patient was the subject of a nosocomial infection acquired at WRAIR as a result of the spread of strain 7 from the index patient.

The epidemiology of strain type 3 of Acinetobacter baumannii was also investigated. Strain type 3 was found in 46 samples, all of which were from patients (US service members, Iraqi civilians and enemy prisoners) who were treated on the USNS Comfort hospital ship and subsequently returned to Iraq or Kuwait. The occurrence of strain type 3 in a single locale may provide evidence that at least some of the infections at that locale were a result of a nosocomial infections.

This example thus illustrates an embodiment of the present invention wherein the methods of analysis of bacterial bioagent identifying amplicons provide the means for epidemiological surveillance.

### Example 13

### Selection and Use of MLST Acinetobacter baumanii Drill-Down Primers

To combine the power of high-throughput mass spectrometric analysis of bioagent identifying amplicons with the sub-species characteristic resolving power provided by multi-locus sequence typing (MLST) such as the MLST methods of the MLST Databases at the Max-Planck Institute for Infectious Biology (web.mpiib-berlin.mpg.de/mlst/dbs/Mcatarrhalis/documents/primersCatarrhalis_html), an additional 21 primer pairs were selected based on analysis of housekeeping genes of the genus Acinetobacter. Genes to which the drill-down MLST analogue primers hybridize for production of bacterial bioagent identifying amplicons include anthranilate synthase component I (trpE), adenylate kinase (adk), adenine glycosylase (mutY), fumarate hydratase (fumC), and pyrophosphate phospho-hydratase (ppa). These 21 primer pairs are indicated with reference to sequence listings in Table 13. Primer pair numbers 1151-1154 hybridize to and amplify segments of trpE. Primer pair numbers 1155-1157 hybridize to and amplify segments of adk. Primer pair numbers 1158-1164 hybridize to and amplify segments of mutY. Primer pair numbers 1165-1170 hybridize to and amplify segments of fumC. Primer pair number 1171 hybridizes to and amplifies a segment of ppa. The primer names given in Table 13 indicates the coordinates to which the primers hybridize to a reference sequence which comprises a concatenation of the genes TrpE, efp (elongation factor p), adk, mutT, fumC, and ppa. For example, the forward primer of primer pair 1151 is named AB_MLST-11-OIF007_{—}62_{—}91_F because it hybridizes to the Acinetobacter MLST primer reference sequence of strain type 11 in sample 007 of Operation Iraqi Freedom (OIF) at positions 62 to 91.

TABLE 13
MLST Drill-Down Primers for Identification of Sub-species characteristics
(Strain Type) of Members of the Bacterial Genus Acinetobacter

| Primer | | Forward | | Reverse |
|---|---|---|---|---|
| Pair | | Primer | | Primer |
| No. | Forward Primer Name | (SEQ ID NO:) | Reverse Primer Name | (SEQ ID NO:) |
| 1151 | AB_MLST-11-OIF007_62_91_F | 83 | AB_MLST-11-OIF007_169_203_R | 426 |
| 1152 | AB_MLST-11-OIF007_185_214_F | 76 | AB_MLST-11-OIF007_291_324_R | 432 |
| 1153 | AB_MLST-11-OIF007_260_289_F | 79 | AB_MLST-11-OIF007_364_393_R | 434 |
| 1154 | AB_MLST-11-OIF007_206_239_F | 78 | AB_MLST-11-OIF007_318_344_R | 433 |
| 1155 | AB_MLST-11-OIF007_522_552_F | 80 | AB_MLST-11-OIF007_587_610_R | 435 |
| 1156 | AB_MLST-11-OIF007_547_571_F | 81 | AB_MLST-11-OIF007_656_686_R | 436 |
| 1157 | AB_MLST-11-OIF007_601_627_F | 82 | AB_MLST-11-OIF007_710_736_R | 437 |
| 1158 | AB_MLST-11- | 65 | AB_MLST-11-OIF007_1266_1296_R | 420 |
| | OIF007_1202_1225_F | | | |
| 1159 | AB_MLST-11- | 65 | AB_MLST-11-OIF007_1299_1316_R | 421 |
| | OIF007_1202_1225_F | | | |
| 1160 | AB_MLST-11- | 66 | AB_MLST-11-OIF007_1335_1362_R | 422 |
| | OIF007_1234_1264_F | | | |
| 1161 | AB_MLST-11- | 67 | AB_MLST-11-OIF007_1422_1448_R | 423 |
| | OIF007_1327_1356_F | | | |
| 1162 | AB_MLST-11- | 68 | AB_MLST-11-OIF007_1470_1494_R | 424 |
| | OIF007_1345_1369_F | | | |
| 1163 | AB_MLST-11- | 69 | AB_MLST-11-OIF007_1470_1494_R | 424 |
| | OIF007_1351_1375_F | | | |
| 1164 | AB_MLST-11- | 70 | AB_MLST-11-OIF007_1470_1494_R | 424 |
| | OIF007_1387_1412_F | | | |
| 1165 | AB_MLST-11- | 71 | AB_MLST-11-OIF007_1656_1680_R | 425 |
| | OIF007_1542_1569_F | | | |
| 1166 | AB_MLST-11- | 72 | AB_MLST-11-OIF007_1656_1680_R | 425 |
| | OIF007_1566_1593_F | | | |
| 1167 | AB_MLST-11- | 73 | AB_MLST-11-OIF007_1731_1757_R | 427 |
| | OIF007_1611_1638_F | | | |
| 1168 | AB_MLST-11- | 74 | AB_MLST-11-OIF007_1790_1821_R | 428 |
| | OIF007_1726_1752_F | | | |
| 1169 | AB_MLST-11- | 75 | AB_MLST-11-OIF007_1876_1909_R | 429 |
| | OIF007_1792_1826_F | | | |
| 1170 | AB_MLST-11- | 75 | AB_MLST-11-OIF007_1895_1927_R | 430 |
| | OIF007_1792_1826_F | | | |
| 1171 | AB_MLST-11- | 77 | AB_MLST-11-OIF007_2097_2118_R | 431 |
| | OIF007_1970_2002_F | | | |

Analysis of bioagent identifying amplicons obtained using the primers of Table 13 for over 200 samples from Operation Iraqi Freedom resulted in the identification of 50 distinct strain type clusters. The largest cluster, designated strain type 11 (ST11) includes 42 sample isolates, all of which were obtained from US service personnel and Iraqi civilians treated at the 28^{th} Combat Support Hospital in Baghdad. Several of these individuals were also treated on the hospital ship USNS Comfort. These observations are indicative of significant epidemiological correlation/linkage.

All of the sample isolates were tested against a broad panel of antibiotics to characterize their antibiotic resistance profiles. As an example of a representative result from antibiotic susceptibility testing, ST11 was found to consist of four different clusters of isolates, each with a varying degree of sensitivity/resistance to the various antibiotics tested which included penicillins, extended spectrum penicillins, cephalosporins, carbipenem, protein synthesis inhibitors, nucleic acid synthesis inhibitors, anti-metabolites, and anti-cell membrane antibiotics. Thus, the genotyping power of bacterial bioagent identifying amplicons, particularly drill-down bacterial bioagent identifying amplicons, has the potential to increase the understanding of the transmission of infections in combat casualties, to identify the source of infection in the environment, to track hospital transmission of nosocomial infections, and to rapidly characterize drug-resistance profiles which enable development of effective infection control measures on a time-scale previously not achievable.

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, internet web sites, and the like) cited in the present application is incorporated herein by reference in its entirety.

1.-30. (canceled)

31. A purified oligonucleotide primer pair comprising a forward primer and a reverse primer, said primer pair configured to generate an amplicon of between 54 consecutive nucleobases in length and 75 consecutive nucleobases in length from the sequence shown in GenBank accession number Y14051, said forward primer consisting of 15 to 24 consecutive nucleobases from SEQ ID NO: 183, and said reverse primer consisting of 15 to 27 consecutive nucleobases from SEQ ID NO: 538.

32-34. (canceled)

35. The purified oligonucleotide primer pair of claim 31 wherein the forward primer is SEQ ID NO: 183.

36. The purified oligonucleotide primer pair of claim 31 wherein the reverse primer is SEQ ID NO: 538.

37. The purified oligonucleotide primer pair of claim 31 wherein at least one of said forward primer or said reverse primer comprises at least one modified nucleobase.

38. The purified oligonucleotide primer pair of claim 37 wherein said modified nucleobase is a mass modified nucleobase.

39. The purified oligonucleotide primer pair of claim 37 wherein said mass modified nucleobase is 5-Iodo-C.

40. The purified oligonucleotide primer pair of claim 37 wherein said modified nucleobase is a universal nucleobase.

41. The purified oligonucleotide primer pair of claim 40 wherein said universal nucleobase is inosine.

42. The purified oligonucleotide primer pair of claim 31 wherein at least one of said forward primer or said reverse primer comprises a non-templated T residue at its 5′-end.

43. The purified oligonucleotide primer pair of claim 37 wherein said modified nucleobase comprises a molecular mass modifying tag.

44-53. (canceled)

54. A purified oligonucleotide pair, comprising a forward primer and a reverse primer, wherein said forward primer consists of 15 to 24 consecutive nucleobases selected from the sequence of SEQ ID NO: 183 and said reverse primer consists of 15 to 27 consecutive nucleobases selected from the sequence of SEQ ID NO: 538, which primer pair is configured to generate an amplicon between 54 and 100 consecutive nucleobases in length from the sequence shown in GenBank accession number Y14051.

55. The purified oligonucleotide primer pair of claim 54 wherein at least one of said forward primer or said reverse primer comprises at least one modified nucleobase.

56. The purified oligonucleotide primer pair of claim 55 wherein said modified nucleobase is a mass modified nucleobase.

57. The purified oligonucleotide primer pair of claim 55 wherein said mass modified nucleobase is 5-Iodo-C.

58. The purified oligonucleotide primer pair of claim 55 wherein said modified nucleobase is a universal nucleobase.

59. The purified oligonucleotide primer pair of claim 58 wherein said universal nucleobase is inosine.

60. The purified oligonucleotide primer pair of claim 54 wherein at least one of said forward primer or said reverse primer lacks a non-templated T residue at its 5′-end.

61. The purified oligonucleotide primer pair of claim 55 wherein said modified nucleobase comprises a molecular mass modifying tag.

62-65. (canceled)

66. A kit comprising a purified oligonucleotide primer pair and at least one additional purified oligonucleotide primer pair selected from Table 1.

67. A kit comprising a first primer pair as defined in claim 31, a second primer pair configured to identify a respiratory pathogen by generating an amplicon from a gene encoding TUFB, and a third primer pair configured to identify a respiratory pathogen by generating an amplicon from at least one of a gene encoding 16S rRNA, a gene encoding 23S rRNA, a gene encoding INFB, a gene encoding RPLB, a gene encoding RPOC, or a combination thereof.

68. The kit of claim 67 wherein said primer pair configured to generate an amplicon from a respiratory pathogen comprises primer pair no. 346, primer pair no. 361, primer pair no. 347, primer pair no. 348, primer pair no. 349, primer pair no. 360, primer pair no. 352, primer pair no. 356, primer pair no. 449, primer pair no. 354, primer pair no. 367 or a combination thereof.

69. The kit of claim 67 wherein said first primer pair comprises a forward primer and reverse primer that hybridize between residues 4507 and 4610 of accession number Y14051.

70. The kit of claim 69 wherein said first primer pair comprises a forward primer and reverse primer hybridize between residues 4507 and 4581 of accession number Y14051.

71. The kit of claim 70 wherein said first primer pair is SEQ ID NOS: 183:539.

72. The kit of claim 60 wherein said second primer pair is primer pair no. 367.


SEQUENCE LISTING (785 sequences)

SEQ ID NO: 1 - DNA, 30 bp, Artificial Sequence
  Primer
gtgagatgttgggttaagtcccgtaacgag

SEQ ID NO: 2 - DNA, 19 bp, Artificial Sequence
  Primer
atgttgggttaagtcccgc

SEQ ID NO: 3 - DNA, 25 bp, Artificial Sequence
  Primer
atgttgggttaagtcccgcaacgag

SEQ ID NO: 4 - DNA, 22 bp, Artificial Sequence
  Primer
ttaagtcccgcaacgagcgcaa

SEQ ID NO: 5 - DNA, 23 bp, Artificial Sequence
  Primer
tttaagtcccgcaacgagcgcaa

SEQ ID NO: 6 - DNA, 22 bp, Artificial Sequence
  Primer
ttaagtcccgcaacgatcgcaa

SEQ ID NO: 7 - DNA, 18 bp, Artificial Sequence
  Primer
tagtcccgcaacgagcgc

SEQ ID NO: 8 - DNA, 17 bp, Artificial Sequence
  Primer
caacgagcgcaaccctt

SEQ ID NO: 9 - DNA, 19 bp, Artificial Sequence
  Primer
caagtcatcatggccctta

SEQ ID NO: 10 - DNA, 20 bp, Artificial Sequence
  Primer
gctacacacgtgctacaatg

SEQ ID NO: 11 - DNA, 21 bp, Artificial Sequence
  Primer
cggattggagtctgcaactcg

SEQ ID NO: 12 - DNA, 22 bp, Artificial Sequence
  Primer
aagtcggaatcgctagtaatcg

SEQ ID NO: 13 - DNA, 21 bp, Artificial Sequence
  Primer
tacggtgaatacgttcccggg

SEQ ID NO: 14 - DNA, 21 bp, Artificial Sequence
  Primer
gccttgtacacacctcccgtc

SEQ ID NO: 15 - DNA, 19 bp, Artificial Sequence
  Primer
cttgtacacaccgcccgtc

SEQ ID NO: 16 - DNA, 22 bp, Artificial Sequence
  Primer
ttgtacacaccgcccgtcatac

SEQ ID NO: 17 - DNA, 25 bp, Artificial Sequence
  Primer
tgaacgctggtggcatgcttaacac

SEQ ID NO: 18 - DNA, 19 bp, Artificial Sequence
  Primer
cactggaactgagacacgg

SEQ ID NO: 19 - DNA, 26 bp, Artificial Sequence
  Primer
gtggcatgcctaatacatgcaagtcg

SEQ ID NO: 20 - DNA, 28 bp, Artificial Sequence
  Primer
tgagtgatgaaggccttagggttgtaaa

SEQ ID NO: 21 - DNA, 20 bp, Artificial Sequence
  Primer
taacacatgcaagtcgaacg

SEQ ID NO: 22 - DNA, 19 bp, Artificial Sequence
  Primer
ccagcagccgcggtaatac

SEQ ID NO: 23 - DNA, 20 bp, Artificial Sequence
  Primer
cggaattactgggcgtaaag

SEQ ID NO: 24 - DNA, 18 bp, Artificial Sequence
  Primer
gtgtagcggtgaaatgcg

SEQ ID NO: 25 - DNA, 27 bp, Artificial Sequence
  Primer
gagagtttgatcctggctcagaacgaa

SEQ ID NO: 26 - DNA, 20 bp, Artificial Sequence
  Primer
agaacaccgatggcgaaggc

SEQ ID NO: 27 - DNA, 21 bp, Artificial Sequence
  Primer
tagaacaccgatggcgaaggc

SEQ ID NO: 28 - DNA, 22 bp, Artificial Sequence
  Primer
gggagcaaacaggattagatac

SEQ ID NO: 29 - DNA, 22 bp, Artificial Sequence
  Primer
ggattagagaccctggtagtcc

SEQ ID NO: 30 - DNA, 23 bp, Artificial Sequence
  Primer
tggattagagaccctggtagtcc

SEQ ID NO: 31 - DNA, 26 bp, Artificial Sequence
  Primer
ggattagataccctggtagtccacgc

SEQ ID NO: 32 - DNA, 22 bp, Artificial Sequence
  Primer
tagataccctggtagtccacgc

SEQ ID NO: 33 - DNA, 22 bp, Artificial Sequence
  Primer
gataccctggtagtccacaccg

SEQ ID NO: 34 - DNA, 20 bp, Artificial Sequence
  Primer
agagtttgatcatggctcag

SEQ ID NO: 35 - DNA, 19 bp, Artificial Sequence
  Primer
accacgccgtaaacgatga

SEQ ID NO: 36 - DNA, 18 bp, Artificial Sequence
  Primer
aagcggtggagcatgtgg

SEQ ID NO: 37 - DNA, 22 bp, Artificial Sequence
  Primer
ttcgatgcaacgcgaagaacct

SEQ ID NO: 38 - DNA, 23 bp, Artificial Sequence
  Primer
tttcgatgcaacgcgaagaacct

SEQ ID NO: 39 - DNA, 17 bp, Artificial Sequence
  Primer
acgcgaagaaccttacc

SEQ ID NO: 40 - DNA, 17 bp, Artificial Sequence
  Primer
acgcgaagaaccttacc

SEQ ID NO: 41 - DNA, 20 bp, Artificial Sequence
  Primer
gcgaagaaccttaccaggtc

SEQ ID NO: 42 - DNA, 14 bp, Artificial Sequence
  Primer
cgaagaaccttacc

SEQ ID NO: 43 - DNA, 20 bp, Artificial Sequence
  Primer
tgcgcggaagatgtaacggg

SEQ ID NO: 44 - DNA, 22 bp, Artificial Sequence
  Primer
tgcatacaaacagtcggagcct

SEQ ID NO: 45 - DNA, 24 bp, Artificial Sequence
  Primer
aaactagataacagtagacatcac

SEQ ID NO: 46 - DNA, 19 bp, Artificial Sequence
  Primer
taccccaaaccgacacagg

SEQ ID NO: 47 - DNA, 19 bp, Artificial Sequence
  Primer
ccgtaacttcgggagaagg

SEQ ID NO: 48 - DNA, 18 bp, Artificial Sequence
  Primer
ctgacacctgcccggtgc

SEQ ID NO: 49 - DNA, 19 bp, Artificial Sequence
  Primer
tctgacacctgcccggtgc

SEQ ID NO: 50 - DNA, 16 bp, Artificial Sequence
  Primer
gacgcctgcccggtgc

SEQ ID NO: 51 - DNA, 19 bp, Artificial Sequence
  Primer
acctgcccagtgctggaag

SEQ ID NO: 52 - DNA, 21 bp, Artificial Sequence
  Primer
gggaactgaaacatctaagta

SEQ ID NO: 53 - DNA, 15 bp, Artificial Sequence
  Primer
ggtggatgccttggc

SEQ ID NO: 54 - DNA, 23 bp, Artificial Sequence
  Primer
aaggtactccggggataacaggc

SEQ ID NO: 55 - DNA, 22 bp, Artificial Sequence
  Primer
tagaacgtcgcgagacagttcg

SEQ ID NO: 56 - DNA, 18 bp, Artificial Sequence
  Primer
gacagttcggtccctatc

SEQ ID NO: 57 - DNA, 24 bp, Artificial Sequence
  Primer
ctgtccctagtacgagaggaccgg

SEQ ID NO: 58 - DNA, 25 bp, Artificial Sequence
  Primer
tctgtccctagtacgagaggaccgg

SEQ ID NO: 59 - DNA, 22 bp, Artificial Sequence
  Primer
ctgttcttagtacgagaggacc

SEQ ID NO: 60 - DNA, 23 bp, Artificial Sequence
  Primer
tctgttcttagtacgagaggacc

SEQ ID NO: 61 - DNA, 18 bp, Artificial Sequence
  Primer
ctagtacgagaggaccgg

SEQ ID NO: 62 - DNA, 17 bp, Artificial Sequence
  Primer
tagtacgagaggaccgg

SEQ ID NO: 63 - DNA, 26 bp, Artificial Sequence
  Primer
ggggagtgaaagagatcctgaaaccg

SEQ ID NO: 64 - DNA, 22 bp, Artificial Sequence
  Primer
cgagagggaaacaacccagacc

SEQ ID NO: 65 - DNA, 24 bp, Artificial Sequence
  Primer
tcgtgcccgcaatttgcataaagc

SEQ ID NO: 66 - DNA, 31 bp, Artificial Sequence
  Primer
ttgtagcacagcaaggcaaatttcctgaaac

SEQ ID NO: 67 - DNA, 30 bp, Artificial Sequence
  Primer
taggtttacgtcagtatggcgtgattatgg

SEQ ID NO: 68 - DNA, 25 bp, Artificial Sequence
  Primer
tcgtgattatggatggcaacgtgaa

SEQ ID NO: 69 - DNA, 25 bp, Artificial Sequence
  Primer
ttatggatggcaacgtgaaacgcgt

SEQ ID NO: 70 - DNA, 26 bp, Artificial Sequence
  Primer
tctttgccattgaagatgacttaagc

SEQ ID NO: 71 - DNA, 28 bp, Artificial Sequence
  Primer
tactagcggtaagcttaaacaagattgc

SEQ ID NO: 72 - DNA, 28 bp, Artificial Sequence
  Primer
ttgccaatgatattcgttggttagcaag

SEQ ID NO: 73 - DNA, 28 bp, Artificial Sequence
  Primer
tcggcgaaatccgtattcctgaaaatga

SEQ ID NO: 74 - DNA, 27 bp, Artificial Sequence
  Primer
taccactattaatgtcgctggtgcttc

SEQ ID NO: 75 - DNA, 35 bp, Artificial Sequence
  Primer
ttataacttactgcaatctattcagttgcttggtg

SEQ ID NO: 76 - DNA, 30 bp, Artificial Sequence
  Primer
tattgtttcaaatgtacaaggtgaagtgcg

SEQ ID NO: 77 - DNA, 33 bp, Artificial Sequence
  Primer
tggttatgtaccaaatactttgtctgaagatgg

SEQ ID NO: 78 - DNA, 34 bp, Artificial Sequence
  Primer
tgaagtgcgtgatgatatcgatgcacttgatgta

SEQ ID NO: 79 - DNA, 30 bp, Artificial Sequence
  Primer
tggaacgttatcaggtgccccaaaaattcg

SEQ ID NO: 80 - DNA, 31 bp, Artificial Sequence
  Primer
tcggtttagtaaaagaacgtattgctcaacc

SEQ ID NO: 81 - DNA, 25 bp, Artificial Sequence
  Primer
tcaacctgactgcgtgaatggttgt

SEQ ID NO: 82 - DNA, 27 bp, Artificial Sequence
  Primer
tcaagcagaagctttggaagaagaagg

SEQ ID NO: 83 - DNA, 30 bp, Artificial Sequence
  Primer
tgagattgctgaacatttaatgctgattga

SEQ ID NO: 84 - DNA, 29 bp, Artificial Sequence
  Primer
ttgcttaaagttggttttattggttggcg

SEQ ID NO: 85 - DNA, 34 bp, Artificial Sequence
  Primer
tcagttttaatgtctcgtatgatcgaatcaaaag

SEQ ID NO: 86 - DNA, 18 bp, Artificial Sequence
  Primer
gcacaacctgcggctgcg

SEQ ID NO: 87 - DNA, 24 bp, Artificial Sequence
  Primer
tctagtaataataggaccctcagc

SEQ ID NO: 88 - DNA, 15 bp, Artificial Sequence
  Primer
tatggctctactcaa

SEQ ID NO: 89 - DNA, 30 bp, Artificial Sequence
  Primer
tgagtcacttgaagttgatacaaatcctct

SEQ ID NO: 90 - DNA, 20 bp, Artificial Sequence
  Primer
gaatagcaattaatccaaat

SEQ ID NO: 91 - DNA, 24 bp, Artificial Sequence
  Primer
tcagttccgttatcgccattgcat

SEQ ID NO: 92 - DNA, 23 bp, Artificial Sequence
  Primer
tggaactattgcaactgctaatg

SEQ ID NO: 93 - DNA, 27 bp, Artificial Sequence
  Primer
tcactcttacatataaggaaggcgctc

SEQ ID NO: 94 - DNA, 30 bp, Artificial Sequence
  Primer
tcaggatggaaataaccaccaattcactac

SEQ ID NO: 95 - DNA, 28 bp, Artificial Sequence
  Primer
gttatttagcactcgtttttaatcagcc

SEQ ID NO: 96 - DNA, 20 bp, Artificial Sequence
  Primer
actcgtttttaatcagcccg

SEQ ID NO: 97 - DNA, 30 bp, Artificial Sequence
  Primer
gattattgttatcctgttatgccatttgag

SEQ ID NO: 98 - DNA, 31 bp, Artificial Sequence
  Primer
tgattattgttatcctgttatgccatttgag

SEQ ID NO: 99 - DNA, 21 bp, Artificial Sequence
  Primer
ttattgttatcctgttatgcc

SEQ ID NO: 100 - DNA, 21 bp, Artificial Sequence
  Primer
gttatcctgttatgccatttg

SEQ ID NO: 101 - DNA, 20 bp, Artificial Sequence
  Primer
ccgtggtattggagttattg

SEQ ID NO: 102 - DNA, 31 bp, Artificial Sequence
  Primer
ttgagggtatgcaccgtctttttgattcttt

SEQ ID NO: 103 - DNA, 32 bp, Artificial Sequence
  Primer
agttataaacacggctttcctatggcttatcc

SEQ ID NO: 104 - DNA, 31 bp, Artificial Sequence
  Primer
tggcttatccaaatttagatcgtggttttac

SEQ ID NO: 105 - DNA, 28 bp, Artificial Sequence
  Primer
ttatcgtttgtggagctagtgcttatgc

SEQ ID NO: 106 - DNA, 33 bp, Artificial Sequence
  Primer
tgctcgagtgattgactttgctaaatttagaga

SEQ ID NO: 107 - DNA, 34 bp, Artificial Sequence
  Primer
tgattttgctaaatttagagaaattgcggatgaa

SEQ ID NO: 108 - DNA, 31 bp, Artificial Sequence
  Primer
tcccaattaattctgccatttttccaggtat

SEQ ID NO: 109 - DNA, 31 bp, Artificial Sequence
  Primer
tcccggacttaatatcaatgaaaattgtgga

SEQ ID NO: 110 - DNA, 30 bp, Artificial Sequence
  Primer
tgcggatcgtttggtggttgtagatgaaaa

SEQ ID NO: 111 - DNA, 27 bp, Artificial Sequence
  Primer
tcgtttggtggtggtagatgaaaaagg

SEQ ID NO: 112 - DNA, 28 bp, Artificial Sequence
  Primer
tagatgaaaagggcgaagtggctaatgg

SEQ ID NO: 113 - DNA, 33 bp, Artificial Sequence
  Primer
tgcctagaagatcttaaaaatttccgccaactt

SEQ ID NO: 114 - DNA, 26 bp, Artificial Sequence
  Primer
tccccaggacaccctgaaatttcaac

SEQ ID NO: 115 - DNA, 31 bp, Artificial Sequence
  Primer
tggcatttcttatgaagcttgttctttagca

SEQ ID NO: 116 - DNA, 27 bp, Artificial Sequence
  Primer
tgaagcttgttctttagcaggacttca

SEQ ID NO: 117 - DNA, 27 bp, Artificial Sequence
  Primer
tttgattttacgccgtcctccaggtcg

SEQ ID NO: 118 - DNA, 34 bp, Artificial Sequence
  Primer
tcctgttatccctgaagtagttaatcaagtttgt

SEQ ID NO: 119 - DNA, 35 bp, Artificial Sequence
  Primer
tcctgttatccctgaagtagttaatcaagtttgtt

SEQ ID NO: 120 - DNA, 35 bp, Artificial Sequence
  Primer
taggcgaagatatacaaagagtattagaagctaga

SEQ ID NO: 121 - DNA, 33 bp, Artificial Sequence
  Primer
tccaggacaaatgtatgaaaaatgtccaagaag

SEQ ID NO: 122 - DNA, 34 bp, Artificial Sequence
  Primer
tgaaaaatgtccaagaagcatagcaaaaaaagca

SEQ ID NO: 123 - DNA, 26 bp, Artificial Sequence
  Primer
tcttatgccaagaggacagagtgagt

SEQ ID NO: 124 - DNA, 27 bp, Artificial Sequence
  Primer
tgtattaggggcatacagtcctcatcc

SEQ ID NO: 125 - DNA, 18 bp, Artificial Sequence
  Primer
gaaagagttcggattggg

SEQ ID NO: 126 - DNA, 22 bp, Artificial Sequence
  Primer
acaacgaagtacaatacaagac

SEQ ID NO: 127 - DNA, 27 bp, Artificial Sequence
  Primer
cgaagtacaatacaagacaaaagaagg

SEQ ID NO: 128 - DNA, 28 bp, Artificial Sequence
  Primer
tcgaagtacaatacaagacaaaagaagg

SEQ ID NO: 129 - DNA, 21 bp, Artificial Sequence
  Primer
acaatacaagacaaaagaagg

SEQ ID NO: 130 - DNA, 24 bp, Artificial Sequence
  Primer
caggtttagtaccagaacatgcag

SEQ ID NO: 131 - DNA, 20 bp, Artificial Sequence
  Primer
ggtttagtaccagaacatgc

SEQ ID NO: 132 - DNA, 22 bp, Artificial Sequence
  Primer
cggcgtacttcaacgacagcca

SEQ ID NO: 133 - DNA, 32 bp, Artificial Sequence
  Primer
ttatcagctagaccttttaggtaaagctaagc

SEQ ID NO: 134 - DNA, 32 bp, Artificial Sequence
  Primer
tccaaggtacactaaacttacttgagctaatg

SEQ ID NO: 135 - DNA, 32 bp, Artificial Sequence
  Primer
tcaaaaagccctaggtaaagagattccatatc

SEQ ID NO: 136 - DNA, 33 bp, Artificial Sequence
  Primer
tccgttcttacaaatagcaatagaacttgaagc

SEQ ID NO: 137 - DNA, 30 bp, Artificial Sequence
  Primer
tggagcttgaagctatcgctcttaaagatg

SEQ ID NO: 138 - DNA, 30 bp, Artificial Sequence
  Primer
tggaacttgaagctctcgctcttaaagatg

SEQ ID NO: 139 - DNA, 30 bp, Artificial Sequence
  Primer
tgggacttgaagctatcgctcttaaagatg

SEQ ID NO: 140 - DNA, 29 bp, Artificial Sequence
  Primer
tcttctcatcctatggctattatgcttgc

SEQ ID NO: 141 - DNA, 24 bp, Artificial Sequence
  Primer
ggtgaaagaagttgcctctaaagc

SEQ ID NO: 142 - DNA, 23 bp, Artificial Sequence
  Primer
atggacaaggttggcaaggaagg

SEQ ID NO: 143 - DNA, 26 bp, Artificial Sequence
  Primer
aaggaaggcgtgatcaccgttgaaga

SEQ ID NO: 144 - DNA, 19 bp, Artificial Sequence
  Primer
tggaagatctgggtcaggc

SEQ ID NO: 145 - DNA, 20 bp, Artificial Sequence
  Primer
tctgcccgtgtcgttggtga

SEQ ID NO: 146 - DNA, 25 bp, Artificial Sequence
  Primer
tccattgttcgtatggctcaagact

SEQ ID NO: 147 - DNA, 22 bp, Artificial Sequence
  Primer
tcaggtggcttacacggcgtag

SEQ ID NO: 148 - DNA, 28 bp, Artificial Sequence
  Primer
tctttcttgaatgctggtgtacgtatcg

SEQ ID NO: 149 - DNA, 27 bp, Artificial Sequence
  Primer
tcaacgaaggtaaaaaccatctcaacg

SEQ ID NO: 150 - DNA, 28 bp, Artificial Sequence
  Primer
tgttcgctgtttcacaaacaacattcca

SEQ ID NO: 151 - DNA, 28 bp, Artificial Sequence
  Primer
tacttacttgagaatccacaagctgcaa

SEQ ID NO: 152 - DNA, 22 bp, Artificial Sequence
  Primer
tggcgaacctggtgaacgaagc

SEQ ID NO: 153 - DNA, 22 bp, Artificial Sequence
  Primer
tagttgctcaaacagctgggct

SEQ ID NO: 154 - DNA, 28 bp, Artificial Sequence
  Primer
tcccggagcttttatgactaaagcagat

SEQ ID NO: 155 - DNA, 23 bp, Artificial Sequence
  Primer
tcgccgtggaaaaatcctacgct

SEQ ID NO: 156 - DNA, 29 bp, Artificial Sequence
  Primer
ttcctgaccgacccattattccctttatc

SEQ ID NO: 157 - DNA, 28 bp, Artificial Sequence
  Primer
tcctgaccgacccattattccctttatc

SEQ ID NO: 158 - DNA, 22 bp, Artificial Sequence
  Primer
gtcgtgaaaacgagctggaaga

SEQ ID NO: 159 - DNA, 21 bp, Artificial Sequence
  Primer
tgcgtttaccgcaatgcgtgc

SEQ ID NO: 160 - DNA, 29 bp, Artificial Sequence
  Primer
tgctcgtggtgcacaagtaacggatatta

SEQ ID NO: 161 - DNA, 30 bp, Artificial Sequence
  Primer
ttgctcgtggtgcacaagtaacggatatta

SEQ ID NO: 162 - DNA, 26 bp, Artificial Sequence
  Primer
cgtcagggtaaattccgtgaagttaa

SEQ ID NO: 163 - DNA, 24 bp, Artificial Sequence
  Primer
tggtaacagagccttataggcgca

SEQ ID NO: 164 - DNA, 25 bp, Artificial Sequence
  Primer
tggctccttggtatgactctgcttc

SEQ ID NO: 165 - DNA, 26 bp, Artificial Sequence
  Primer
tgctgaggcctggaccgattatttac

SEQ ID NO: 166 - DNA, 25 bp, Artificial Sequence
  Primer
ttatttacctgcactcccacaactg

SEQ ID NO: 167 - DNA, 22 bp, Artificial Sequence
  Primer
tccttgaccgcctttccgatac

SEQ ID NO: 168 - DNA, 20 bp, Artificial Sequence
  Primer
tgaggaccgtgtcgcgctca

SEQ ID NO: 169 - DNA, 25 bp, Artificial Sequence
  Primer
tcagaccatgctcgcagagaaactt

SEQ ID NO: 170 - DNA, 26 bp, Artificial Sequence
  Primer
tcagtatgtatccaccgtagccagtc

SEQ ID NO: 171 - DNA, 28 bp, Artificial Sequence
  Primer
tgggtgacattcatcaatttcatcgttc

SEQ ID NO: 172 - DNA, 17 bp, Artificial Sequence
  Primer
tcaagaagaaaaagagc

SEQ ID NO: 173 - DNA, 31 bp, Artificial Sequence
  Primer
caagaagaaaaagagcttctaaaaagaatac

SEQ ID NO: 174 - DNA, 26 bp, Artificial Sequence
  Primer
agcttttgcatattatatcgagccac

SEQ ID NO: 175 - DNA, 27 bp, Artificial Sequence
  Primer
tagcttttgcatattatatcgagccac

SEQ ID NO: 176 - DNA, 21 bp, Artificial Sequence
  Primer
cttttgcatattatatcgagc

SEQ ID NO: 177 - DNA, 19 bp, Artificial Sequence
  Primer
tttacagctttatgcaccg

SEQ ID NO: 178 - DNA, 17 bp, Artificial Sequence
  Primer
caacggatgctggcaag

SEQ ID NO: 179 - DNA, 24 bp, Artificial Sequence
  Primer
tgtagccgctaagcactaccatcc

SEQ ID NO: 180 - DNA, 23 bp, Artificial Sequence
  Primer
tggacggcatcacgattctctac

SEQ ID NO: 181 - DNA, 26 bp, Artificial Sequence
  Primer
tgaagtagaaatgactgaacgtccga

SEQ ID NO: 182 - DNA, 29 bp, Artificial Sequence
  Primer
taaaacaaactacggtaacattgatcgca

SEQ ID NO: 183 - DNA, 24 bp, Artificial Sequence
  Primer
tcaggtactgctatccaccctcaa

SEQ ID NO: 184 - DNA, 21 bp, Artificial Sequence
  Primer
tgtactgctatccaccctcaa

SEQ ID NO: 185 - DNA, 11 bp, Artificial Sequence
  Primer
tccaccctcaa

SEQ ID NO: 186 - DNA, 30 bp, Artificial Sequence
  Primer
tcaccaggttcaactcaaaaaatattaaca

SEQ ID NO: 187 - DNA, 27 bp, Artificial Sequence
  Primer
ttacacatatcgtgagcaatgaactga

SEQ ID NO: 188 - DNA, 30 bp, Artificial Sequence
  Primer
ttactccattattgcttggttacactttcc

SEQ ID NO: 189 - DNA, 25 bp, Artificial Sequence
  Primer
tacacaacaatggcggtaaagatgg

SEQ ID NO: 190 - DNA, 23 bp, Artificial Sequence
  Primer
tgcgcagctcttggtatcgagtt

SEQ ID NO: 191 - DNA, 27 bp, Artificial Sequence
  Primer
tgcctcgaagctgaatataaccaagtt

SEQ ID NO: 192 - DNA, 27 bp, Artificial Sequence
  Primer
tcaacggtaacttctatgttacttctg

SEQ ID NO: 193 - DNA, 27 bp, Artificial Sequence
  Primer
tcaagccgtacgtattattaggtgctg

SEQ ID NO: 194 - DNA, 27 bp, Artificial Sequence
  Primer
tccgtacgtattattaggtgctggtca

SEQ ID NO: 195 - DNA, 28 bp, Artificial Sequence
  Primer
tcgtacgtattattaggtgctggtcact

SEQ ID NO: 196 - DNA, 23 bp, Artificial Sequence
  Primer
tgttggtgctttctggcgcttaa

SEQ ID NO: 197 - DNA, 24 bp, Artificial Sequence
  Primer
tggtgctttctggcgcttaaacga

SEQ ID NO: 198 - DNA, 30 bp, Artificial Sequence
  Primer
tctactgattttggtaatcttgcagcacag

SEQ ID NO: 199 - DNA, 24 bp, Artificial Sequence
  Primer
tgcaagtggtacttcaacatgggg

SEQ ID NO: 200 - DNA, 31 bp, Artificial Sequence
  Primer
ttacaggaagtttaggtggtaatctaaaagg

SEQ ID NO: 201 - DNA, 20 bp, Artificial Sequence
  Primer
cagaatcaagttcccagggg

SEQ ID NO: 202 - DNA, 23 bp, Artificial Sequence
  Primer
agaatcaagttcccaggggttac

SEQ ID NO: 203 - DNA, 19 bp, Artificial Sequence
  Primer
aatctgctatttggtcagg

SEQ ID NO: 204 - DNA, 21 bp, Artificial Sequence
  Primer
gaaggatatacggttgatgtc

SEQ ID NO: 205 - DNA, 20 bp, Artificial Sequence
  Primer
tcctgaaaaatggagcacgg

SEQ ID NO: 206 - DNA, 19 bp, Artificial Sequence
  Primer
tggagcacggcttctgatc

SEQ ID NO: 207 - DNA, 25 bp, Artificial Sequence
  Primer
ggctcagccatttagttaccgctat

SEQ ID NO: 208 - DNA, 21 bp, Artificial Sequence
  Primer
tcagcgcgtacagtgggtgat

SEQ ID NO: 209 - DNA, 24 bp, Artificial Sequence
  Primer
tggtgactcggcatgttatgaagc

SEQ ID NO: 210 - DNA, 26 bp, Artificial Sequence
  Primer
ttataccggaaacttcccgaaaggag

SEQ ID NO: 211 - DNA, 26 bp, Artificial Sequence
  Primer
tgacatccggctcacgttattatggt

SEQ ID NO: 212 - DNA, 23 bp, Artificial Sequence
  Primer
tccggctcacgttattatggtac

SEQ ID NO: 213 - DNA, 23 bp, Artificial Sequence
  Primer
tgcaaaggaggtactcagaccat

SEQ ID NO: 214 - DNA, 22 bp, Artificial Sequence
  Primer
tgacatgcttgtccgttcaggc

SEQ ID NO: 215 - DNA, 26 bp, Artificial Sequence
  Primer
tggtacatgtgccttcattgatgctg

SEQ ID NO: 216 - DNA, 19 bp, Artificial Sequence
  Primer
tggcacggccatctccgtg

SEQ ID NO: 217 - DNA, 20 bp, Artificial Sequence
  Primer
tgcgggtagggagcttgagc

SEQ ID NO: 218 - DNA, 22 bp, Artificial Sequence
  Primer
tcctagaggaatggctgccacg

SEQ ID NO: 219 - DNA, 23 bp, Artificial Sequence
  Primer
taccccagggaaagtgccacaga

SEQ ID NO: 220 - DNA, 28 bp, Artificial Sequence
  Primer
taaaccccatcgggagcaagaccgaata

SEQ ID NO: 221 - DNA, 19 bp, Artificial Sequence
  Primer
gaggaaagtccatgctcgc

SEQ ID NO: 222 - DNA, 29 bp, Artificial Sequence
  Primer
taaggatagtgcaacagagatataccgcc

SEQ ID NO: 223 - DNA, 17 bp, Artificial Sequence
  Primer
gaggaaagtccgggctc

SEQ ID NO: 224 - DNA, 24 bp, Artificial Sequence
  Primer
tctaaatggtcgtgcagttgcgtg

SEQ ID NO: 225 - DNA, 30 bp, Artificial Sequence
  Primer
tggtaagagcgcaccggtaagttggtaaca

SEQ ID NO: 226 - DNA, 22 bp, Artificial Sequence
  Primer
taagagcgcaccggtaagttgg

SEQ ID NO: 227 - DNA, 23 bp, Artificial Sequence
  Primer
tgcataccggtaagttggcaaca

SEQ ID NO: 228 - DNA, 26 bp, Artificial Sequence
  Primer
tccaccaagagcaagatcaaataggc

SEQ ID NO: 229 - DNA, 19 bp, Artificial Sequence
  Primer
gaggaaagtccatgctcac

SEQ ID NO: 230 - DNA, 19 bp, Artificial Sequence
  Primer
tccgcggagttgactgggt

SEQ ID NO: 231 - DNA, 30 bp, Artificial Sequence
  Primer
gacctacagtaagaggttctgtaatgaacc

SEQ ID NO: 232 - DNA, 31 bp, Artificial Sequence
  Primer
tgacctacagtaagaggttctgtaatgaacc

SEQ ID NO: 233 - DNA, 30 bp, Artificial Sequence
  Primer
tgtaatgaaccctaatgaccatccacacgg

SEQ ID NO: 234 - DNA, 30 bp, Artificial Sequence
  Primer
taatgaaccctaatgaccatccacacggtg

SEQ ID NO: 235 - DNA, 23 bp, Artificial Sequence
  Primer
catccacacggtggtggtgaagg

SEQ ID NO: 236 - DNA, 24 bp, Artificial Sequence
  Primer
tcatccacacggtggtggtgaagg

SEQ ID NO: 237 - DNA, 21 bp, Artificial Sequence
  Primer
tccacacggtggtggtgaagg

SEQ ID NO: 238 - DNA, 18 bp, Artificial Sequence
  Primer
gaccacctcggcaaccgt

SEQ ID NO: 239 - DNA, 23 bp, Artificial Sequence
  Primer
tcagctgtcgcagttcatggacc

SEQ ID NO: 240 - DNA, 22 bp, Artificial Sequence
  Primer
tatcgctcaggcgaactccaac

SEQ ID NO: 241 - DNA, 23 bp, Artificial Sequence
  Primer
ttatcgctcaggcgaactccaac

SEQ ID NO: 242 - DNA, 23 bp, Artificial Sequence
  Primer
tcgttcctggaacacgatgacgc

SEQ ID NO: 243 - DNA, 29 bp, Artificial Sequence
  Primer
tcaacaacctcttggaggtaaagctcagt

SEQ ID NO: 244 - DNA, 29 bp, Artificial Sequence
  Primer
cttggaggtaagtctcattttggtgggca

SEQ ID NO: 245 - DNA, 24 bp, Artificial Sequence
  Primer
tgggcagcgtttcggcgaaatgga

SEQ ID NO: 246 - DNA, 23 bp, Artificial Sequence
  Primer
gggcagcgtttcggcgaaatgga

SEQ ID NO: 247 - DNA, 20 bp, Artificial Sequence
  Primer
cagcgtttcggcgaaatgga

SEQ ID NO: 248 - DNA, 28 bp, Artificial Sequence
  Primer
caaaacttattaggtaagcgtgttgact

SEQ ID NO: 249 - DNA, 25 bp, Artificial Sequence
  Primer
cgtgttgactattcggggcgttcag

SEQ ID NO: 250 - DNA, 27 bp, Artificial Sequence
  Primer
taagaagccggaaaccatcaactaccg

SEQ ID NO: 251 - DNA, 22 bp, Artificial Sequence
  Primer
acccagtgctgctgaaccgtgc

SEQ ID NO: 252 - DNA, 20 bp, Artificial Sequence
  Primer
cgccgacttcgacggtgacc

SEQ ID NO: 253 - DNA, 21 bp, Artificial Sequence
  Primer
tcgccgacttcgacggtgacc

SEQ ID NO: 254 - DNA, 21 bp, Artificial Sequence
  Primer
tggcccgaaagaagctgagcg

SEQ ID NO: 255 - DNA, 31 bp, Artificial Sequence
  Primer
tcaggagtcgttcaactcgatctacatgatg

SEQ ID NO: 256 - DNA, 29 bp, Artificial Sequence
  Primer
caggagtcgttcaactcgatctacatgat

SEQ ID NO: 257 - DNA, 30 bp, Artificial Sequence
  Primer
tcaggagtcgttcaactcgatctacatgat

SEQ ID NO: 258 - DNA, 19 bp, Artificial Sequence
  Primer
tgattccggtgcccgtggt

SEQ ID NO: 259 - DNA, 19 bp, Artificial Sequence
  Primer
tgattctggtgcccgtggt

SEQ ID NO: 260 - DNA, 24 bp, Artificial Sequence
  Primer
cttgctggtatgcgtggtctgatg

SEQ ID NO: 261 - DNA, 24 bp, Artificial Sequence
  Primer
ctggcaggtatgcgtggtctgatg

SEQ ID NO: 262 - DNA, 25 bp, Artificial Sequence
  Primer
tctggcaggtatgcgtggtctgatg

SEQ ID NO: 263 - DNA, 21 bp, Artificial Sequence
  Primer
tggtatgcgtggtctgatggc

SEQ ID NO: 264 - DNA, 24 bp, Artificial Sequence
  Primer
tgctcgtaagggtctggcggatac

SEQ ID NO: 265 - DNA, 26 bp, Artificial Sequence
  Primer
cgtcgtgtaattaaccgtaacaaccg

SEQ ID NO: 266 - DNA, 26 bp, Artificial Sequence
  Primer
cgtcgggtgattaaccgtaacaaccg

SEQ ID NO: 267 - DNA, 22 bp, Artificial Sequence
  Primer
tattggacaacggtcgtcgcgg

SEQ ID NO: 268 - DNA, 21 bp, Artificial Sequence
  Primer
tctggataacggtcgtcgcgg

SEQ ID NO: 269 - DNA, 27 bp, Artificial Sequence
  Primer
caaaggtaagcaaggacgtttccgtca

SEQ ID NO: 270 - DNA, 27 bp, Artificial Sequence
  Primer
caaaggtaagcaaggtcgtttccgtca

SEQ ID NO: 271 - DNA, 29 bp, Artificial Sequence
  Primer
aaccttaattggaaagaaacccaagaagt

SEQ ID NO: 272 - DNA, 30 bp, Artificial Sequence
  Primer
taaccttaattggaaagaaacccaagaagt

SEQ ID NO: 273 - DNA, 26 bp, Artificial Sequence
  Primer
caataccgcaacagcggtggcttggg

SEQ ID NO: 274 - DNA, 27 bp, Artificial Sequence
  Primer
tcaataccgcaacagcggtggcttggg

SEQ ID NO: 275 - DNA, 30 bp, Artificial Sequence
  Primer
gctggtgaaaataacccagatgtcgtcttc

SEQ ID NO: 276 - DNA, 31 bp, Artificial Sequence
  Primer
tgctggtgaaaataacccagatgtcgtcttc

SEQ ID NO: 277 - DNA, 23 bp, Artificial Sequence
  Primer
cgcaaaaaaatccagctattagc

SEQ ID NO: 278 - DNA, 24 bp, Artificial Sequence
  Primer
tcgcaaaaaaatccagctattagc

SEQ ID NO: 279 - DNA, 30 bp, Artificial Sequence
  Primer
cgagtatagctaaaaaaatagtttatgaca

SEQ ID NO: 280 - DNA, 31 bp, Artificial Sequence
  Primer
tcgagtatagctaaaaaaatagtttatgaca

SEQ ID NO: 281 - DNA, 29 bp, Artificial Sequence
  Primer
cctatattaatcgtttacagaaactggct

SEQ ID NO: 282 - DNA, 30 bp, Artificial Sequence
  Primer
tcctatattaatcgtttacagaaactggct

SEQ ID NO: 283 - DNA, 23 bp, Artificial Sequence
  Primer
ctggctaaaactttggcaacggt

SEQ ID NO: 284 - DNA, 24 bp, Artificial Sequence
  Primer
tctggctaaaactttggcaacggt

SEQ ID NO: 285 - DNA, 29 bp, Artificial Sequence
  Primer
atgattacaattcaagaaggtcgtcacgc

SEQ ID NO: 286 - DNA, 30 bp, Artificial Sequence
  Primer
tatgattacaattcaagaaggtcgtcacgc

SEQ ID NO: 287 - DNA, 25 bp, Artificial Sequence
  Primer
taacggttatcatggcccagatggg

SEQ ID NO: 288 - DNA, 26 bp, Artificial Sequence
  Primer
ttaacggttatcatggcccagatggg

SEQ ID NO: 289 - DNA, 28 bp, Artificial Sequence
  Primer
agcaggtggtgaaatcggccacatgatt

SEQ ID NO: 290 - DNA, 29 bp, Artificial Sequence
  Primer
tagcaggtggtgaaatcggccacatgatt

SEQ ID NO: 291 - DNA, 24 bp, Artificial Sequence
  Primer
cagagaccgttttatcctatcagc

SEQ ID NO: 292 - DNA, 25 bp, Artificial Sequence
  Primer
tcagagaccgttttatcctatcagc

SEQ ID NO: 293 - DNA, 25 bp, Artificial Sequence
  Primer
tctaaaacaccaggtcacccagaag

SEQ ID NO: 294 - DNA, 26 bp, Artificial Sequence
  Primer
ttctaaaacaccaggtcacccagaag

SEQ ID NO: 295 - DNA, 20 bp, Artificial Sequence
  Primer
atggccatggcagaagctca

SEQ ID NO: 296 - DNA, 21 bp, Artificial Sequence
  Primer
tatggccatggcagaagctca

SEQ ID NO: 297 - DNA, 30 bp, Artificial Sequence
  Primer
cttgtacttgtggctcacacggctgtttgg

SEQ ID NO: 298 - DNA, 31 bp, Artificial Sequence
  Primer
tcttgtacttgtggctcacacggctgtttgg

SEQ ID NO: 299 - DNA, 24 bp, Artificial Sequence
  Primer
accatgacagaaggcattttgaca

SEQ ID NO: 300 - DNA, 25 bp, Artificial Sequence
  Primer
taccatgacagaaggcattttgaca

SEQ ID NO: 301 - DNA, 29 bp, Artificial Sequence
  Primer
gatgactttttagctaatggtcaggcagc

SEQ ID NO: 302 - DNA, 30 bp, Artificial Sequence
  Primer
tgatgactttttagctaatggtcaggcagc

SEQ ID NO: 303 - DNA, 20 bp, Artificial Sequence
  Primer
tagctaatggtcaggcagcc

SEQ ID NO: 304 - DNA, 23 bp, Artificial Sequence
  Primer
gtcaaagtggcacgtttactggc

SEQ ID NO: 305 - DNA, 24 bp, Artificial Sequence
  Primer
tgtcaaagtggcacgtttactggc

SEQ ID NO: 306 - DNA, 18 bp, Artificial Sequence
  Primer
agcgtaaaggtgaacctt

SEQ ID NO: 307 - DNA, 19 bp, Artificial Sequence
  Primer
tagcgtaaaggtgaacctt

SEQ ID NO: 308 - DNA, 25 bp, Artificial Sequence
  Primer
gcttcaggaatcaatgatggagcag

SEQ ID NO: 309 - DNA, 26 bp, Artificial Sequence
  Primer
tgcttcaggaatcaatgatggagcag

SEQ ID NO: 310 - DNA, 30 bp, Artificial Sequence
  Primer
ggggattcagccatcaaagcagctattgac

SEQ ID NO: 311 - DNA, 31 bp, Artificial Sequence
  Primer
tggggattcagccatcaaagcagctattgac

SEQ ID NO: 312 - DNA, 22 bp, Artificial Sequence
  Primer
tcagccatcaaagcagctattg

SEQ ID NO: 313 - DNA, 30 bp, Artificial Sequence
  Primer
ccttacttcgaactatgaatcttttggaag

SEQ ID NO: 314 - DNA, 31 bp, Artificial Sequence
  Primer
tccttacttcgaactatgaatcttttggaag

SEQ ID NO: 315 - DNA, 27 bp, Artificial Sequence
  Primer
ggggattgatatcaccgataagaagaa

SEQ ID NO: 316 - DNA, 28 bp, Artificial Sequence
  Primer
tggggattgatatcaccgataagaagaa

SEQ ID NO: 317 - DNA, 26 bp, Artificial Sequence
  Primer
tcgccaatcaaaactaagggaatggc

SEQ ID NO: 318 - DNA, 27 bp, Artificial Sequence
  Primer
ttcgccaatcaaaactaagggaatggc

SEQ ID NO: 319 - DNA, 29 bp, Artificial Sequence
  Primer
gggcaacagcagcggattgcgattgcgcg

SEQ ID NO: 320 - DNA, 30 bp, Artificial Sequence
  Primer
tgggcaacagcagcggattgcgattgcgcg

SEQ ID NO: 321 - DNA, 24 bp, Artificial Sequence
  Primer
tcaagcaaacgcacaatcagaagc

SEQ ID NO: 322 - DNA, 23 bp, Artificial Sequence
  Primer
caagcaaacgcacaatcagaagc

SEQ ID NO: 323 - DNA, 17 bp, Artificial Sequence
  Primer
aacgcacaatcagaagc

SEQ ID NO: 324 - DNA, 31 bp, Artificial Sequence
  Primer
tgcacaatcagaagctaagaaagcgcaagct

SEQ ID NO: 325 - DNA, 23 bp, Artificial Sequence
  Primer
tgcaagcttctggtgctagcatt

SEQ ID NO: 326 - DNA, 19 bp, Artificial Sequence
  Primer
tgcttctggtgctagcatt

SEQ ID NO: 327 - DNA, 13 bp, Artificial Sequence
  Primer
tggtgctagcatt

SEQ ID NO: 328 - DNA, 27 bp, Artificial Sequence
  Primer
tgctagttatggtacagagtttgcgac

SEQ ID NO: 329 - DNA, 18 bp, Artificial Sequence
  Primer
tggtacagagtttgcgac

SEQ ID NO: 330 - DNA, 15 bp, Artificial Sequence
  Primer
tacagagtttgcgac

SEQ ID NO: 331 - DNA, 24 bp, Artificial Sequence
  Primer
tcgattaggcagcaacgaaagccg

SEQ ID NO: 332 - DNA, 23 bp, Artificial Sequence
  Primer
tcgacctttggcaggaactagac

SEQ ID NO: 333 - DNA, 26 bp, Artificial Sequence
  Primer
tcaaatgtacaaggtgaagtgcgtga

SEQ ID NO: 334 - DNA, 27 bp, Artificial Sequence
  Primer
tggatggcatggtgaaatggatatgtc

SEQ ID NO: 335 - DNA, 25 bp, Artificial Sequence
  Primer
atgtcgattgcaatccgtacttgtg

SEQ ID NO: 336 - DNA, 23 bp, Artificial Sequence
  Primer
gtgcatgcggatacagagcagag

SEQ ID NO: 337 - DNA, 20 bp, Artificial Sequence
  Primer
tgcaagcgcgaccacatacg

SEQ ID NO: 338 - DNA, 27 bp, Artificial Sequence
  Primer
gcactatgcacacgtagattgtcctgg

SEQ ID NO: 339 - DNA, 21 bp, Artificial Sequence
  Primer
ttgactgcccaggtcacgctg

SEQ ID NO: 340 - DNA, 21 bp, Artificial Sequence
  Primer
tagactgcccaggacacgctg

SEQ ID NO: 341 - DNA, 28 bp, Artificial Sequence
  Primer
tgcacgccgactatgttaagaacatgat

SEQ ID NO: 342 - DNA, 25 bp, Artificial Sequence
  Primer
tgatcactggtgctgctcagatgga

SEQ ID NO: 343 - DNA, 18 bp, Artificial Sequence
  Primer
aagacgacctgcacgggc

SEQ ID NO: 344 - DNA, 23 bp, Artificial Sequence
  Primer
ccacacgccgttcttcaacaact

SEQ ID NO: 345 - DNA, 24 bp, Artificial Sequence
  Primer
tccacacgccgttcttcaacaact

SEQ ID NO: 346 - DNA, 25 bp, Artificial Sequence
  Primer
aactaccgtcctcagttctacttcc

SEQ ID NO: 347 - DNA, 25 bp, Artificial Sequence
  Primer
aactaccgtccgcagttctacttcc

SEQ ID NO: 348 - DNA, 28 bp, Artificial Sequence
  Primer
ccacagttctacttccgtactactgacg

SEQ ID NO: 349 - DNA, 20 bp, Artificial Sequence
  Primer
cgtggcggcgtggttatcga

SEQ ID NO: 350 - DNA, 21 bp, Artificial Sequence
  Primer
tcgtggcggcgtggttatcga

SEQ ID NO: 351 - DNA, 24 bp, Artificial Sequence
  Primer
tatgctgaccgaccagtggtacgt

SEQ ID NO: 352 - DNA, 18 bp, Artificial Sequence
  Primer
cgacgcgctgcgcttcac

SEQ ID NO: 353 - DNA, 24 bp, Artificial Sequence
  Primer
cttctgcaacaagctgtggaacgc

SEQ ID NO: 354 - DNA, 19 bp, Artificial Sequence
  Primer
accgagcaaggagaccagc

SEQ ID NO: 355 - DNA, 28 bp, Artificial Sequence
  Primer
tcttgctctttcgtgagttcagtaaatg

SEQ ID NO: 356 - DNA, 26 bp, Artificial Sequence
  Primer
tcgatctggtttcatgctgtttcagt

SEQ ID NO: 357 - DNA, 20 bp, Artificial Sequence
  Primer
ttactcacccgtccgccgct

SEQ ID NO: 358 - DNA, 22 bp, Artificial Sequence
  Primer
tgttactcacccgtctgccact

SEQ ID NO: 359 - DNA, 17 bp, Artificial Sequence
  Primer
ttactcacccgtccgcc

SEQ ID NO: 360 - DNA, 20 bp, Artificial Sequence
  Primer
acaaccatgcaccacctgtc

SEQ ID NO: 361 - DNA, 22 bp, Artificial Sequence
  Primer
tacgcattactcacccgtccgc

SEQ ID NO: 362 - DNA, 20 bp, Artificial Sequence
  Primer
acgagctgacgacagccatg

SEQ ID NO: 363 - DNA, 21 bp, Artificial Sequence
  Primer
tacgagctgacgacagccatg

SEQ ID NO: 364 - DNA, 18 bp, Artificial Sequence
  Primer
acgacacgagctgacgac

SEQ ID NO: 365 - DNA, 12 bp, Artificial Sequence
  Primer
acacgagctgac

SEQ ID NO: 366 - DNA, 15 bp, Artificial Sequence
  Primer
tccccaccttcctcc

SEQ ID NO: 367 - DNA, 22 bp, Artificial Sequence
  Primer
gacgtcatccccaccttcctcc

SEQ ID NO: 368 - DNA, 21 bp, Artificial Sequence
  Primer
gacgtcatccccaccttcctc

SEQ ID NO: 369 - DNA, 22 bp, Artificial Sequence
  Primer
tgacgtcatccccaccttcctc

SEQ ID NO: 370 - DNA, 23 bp, Artificial Sequence
  Primer
ttgacgtcatccccaccttcctc

SEQ ID NO: 371 - DNA, 23 bp, Artificial Sequence
  Primer
ttgacgtcatccccaccttcctc

SEQ ID NO: 372 - DNA, 20 bp, Artificial Sequence
  Primer
tgacgtcatggccaccttcc

SEQ ID NO: 373 - DNA, 20 bp, Artificial Sequence
  Primer
tgacgtcatgcccaccttcc

SEQ ID NO: 374 - DNA, 20 bp, Artificial Sequence
  Primer
tgacgtcatccccaccttcc

SEQ ID NO: 375 - DNA, 21 bp, Artificial Sequence
  Primer
attgtagcacgtgtgtagccc

SEQ ID NO: 376 - DNA, 21 bp, Artificial Sequence
  Primer
cgagttgcagactgcgatccg

SEQ ID NO: 377 - DNA, 21 bp, Artificial Sequence
  Primer
cgagttgcagactgcgatccg

SEQ ID NO: 378 - DNA, 19 bp, Artificial Sequence
  Primer
gacgggcggtgtgtacaag

SEQ ID NO: 379 - DNA, 22 bp, Artificial Sequence
  Primer
accttgttacgacttcacccca

SEQ ID NO: 380 - DNA, 20 bp, Artificial Sequence
  Primer
ccttgttacgacttcacccc

SEQ ID NO: 381 - DNA, 20 bp, Artificial Sequence
  Primer
cacggctaccttgttacgac

SEQ ID NO: 382 - DNA, 17 bp, Artificial Sequence
  Primer
aaggaggtgatccagcc

SEQ ID NO: 383 - DNA, 17 bp, Artificial Sequence
  Primer
actgctgcctcccgtag

SEQ ID NO: 384 - DNA, 21 bp, Artificial Sequence
  Primer
cggctgctggcacgaagttag

SEQ ID NO: 385 - DNA, 20 bp, Artificial Sequence
  Primer
ctttacgcccagtaattccg

SEQ ID NO: 386 - DNA, 18 bp, Artificial Sequence
  Primer
cgcatttcaccgctacac

SEQ ID NO: 387 - DNA, 22 bp, Artificial Sequence
  Primer
gtatctaatcctgtttgctccc

SEQ ID NO: 388 - DNA, 21 bp, Artificial Sequence
  Primer
cgtggactaccagggtatcta

SEQ ID NO: 389 - DNA, 22 bp, Artificial Sequence
  Primer
tcgtggactaccagggtatcta

SEQ ID NO: 390 - DNA, 15 bp, Artificial Sequence
  Primer
cgtactccccaggcg

SEQ ID NO: 391 - DNA, 18 bp, Artificial Sequence
  Primer
ggccgtactccccaggcg

SEQ ID NO: 392 - DNA, 19 bp, Artificial Sequence
  Primer
tggccgtactccccaggcg

SEQ ID NO: 393 - DNA, 18 bp, Artificial Sequence
  Primer
gcgaccgtactccccagg

SEQ ID NO: 394 - DNA, 19 bp, Artificial Sequence
  Primer
gccttgcgaccgtactccc

SEQ ID NO: 395 - DNA, 21 bp, Artificial Sequence
  Primer
cccccgtcaattcctttgagt

SEQ ID NO: 396 - DNA, 19 bp, Artificial Sequence
  Primer
ggtaaggttcttcgcgttg

SEQ ID NO: 397 - DNA, 26 bp, Artificial Sequence
  Primer
tcgcaggcttacagaacgctctccta

SEQ ID NO: 398 - DNA, 20 bp, Artificial Sequence
  Primer
tcggactcgctttcgctacg

SEQ ID NO: 399 - DNA, 18 bp, Artificial Sequence
  Primer
gtgcgccctttctaactt

SEQ ID NO: 400 - DNA, 19 bp, Artificial Sequence
  Primer
tggctgcttctaagccaac

SEQ ID NO: 401 - DNA, 16 bp, Artificial Sequence
  Primer
gggtttccccattcgg

SEQ ID NO: 402 - DNA, 18 bp, Artificial Sequence
  Primer
ccttctcccgaagttacg

SEQ ID NO: 403 - DNA, 15 bp, Artificial Sequence
  Primer
caccgggcaggcgtc

SEQ ID NO: 404 - DNA, 19 bp, Artificial Sequence
  Primer
gaccgttatagttacggcc

SEQ ID NO: 405 - DNA, 20 bp, Artificial Sequence
  Primer
tgaccgttatagttacggcc

SEQ ID NO: 406 - DNA, 18 bp, Artificial Sequence
  Primer
tcgctaccttaggaccgt

SEQ ID NO: 407 - DNA, 21 bp, Artificial Sequence
  Primer
ccgacaaggaatttcgctacc

SEQ ID NO: 408 - DNA, 15 bp, Artificial Sequence
  Primer
ttcgctcgccgctac

SEQ ID NO: 409 - DNA, 22 bp, Artificial Sequence
  Primer
agccgacatcgaggtgccaaac

SEQ ID NO: 410 - DNA, 17 bp, Artificial Sequence
  Primer
ccggtcctctcgtacta

SEQ ID NO: 411 - DNA, 20 bp, Artificial Sequence
  Primer
agtccatcccggtcctctcg

SEQ ID NO: 412 - DNA, 22 bp, Artificial Sequence
  Primer
ttagatgctttcagcacttatc

SEQ ID NO: 413 - DNA, 20 bp, Artificial Sequence
  Primer
acttagatgctttcagcggt

SEQ ID NO: 414 - DNA, 18 bp, Artificial Sequence
  Primer
tgcttagatgctttcagc

SEQ ID NO: 415 - DNA, 21 bp, Artificial Sequence
  Primer
ttcgtgcttagatgctttcag

SEQ ID NO: 416 - DNA, 22 bp, Artificial Sequence
  Primer
tttcgtgcttagatgctttcag

SEQ ID NO: 417 - DNA, 24 bp, Artificial Sequence
  Primer
gtttcatgcttagatgctttcagc

SEQ ID NO: 418 - DNA, 21 bp, Artificial Sequence
  Primer
acaaaaggcacgccatcaccc

SEQ ID NO: 419 - DNA, 21 bp, Artificial Sequence
  Primer
acaaaaggtacgccgtcaccc

SEQ ID NO: 420 - DNA, 31 bp, Artificial Sequence
  Primer
taatgccgggtagtgcaatccattcttctag

SEQ ID NO: 421 - DNA, 18 bp, Artificial Sequence
  Primer
tgcacctgcggtcgagcg

SEQ ID NO: 422 - DNA, 28 bp, Artificial Sequence
  Primer
tgccatccataatcacgccatactgacg

SEQ ID NO: 423 - DNA, 27 bp, Artificial Sequence
  Primer
tgccagtttccacatttcacgttcgtg

SEQ ID NO: 424 - DNA, 25 bp, Artificial Sequence
  Primer
tcgcttgagtgtagtcatgattgcg

SEQ ID NO: 425 - DNA, 25 bp, Artificial Sequence
  Primer
tgagtcgggttcactttacctggca

SEQ ID NO: 426 - DNA, 35 bp, Artificial Sequence
  Primer
ttgtacatttgaaacaatatgcatgacatgtgaat

SEQ ID NO: 427 - DNA, 27 bp, Artificial Sequence
  Primer
taccggaagcaccagcgacattaatag

SEQ ID NO: 428 - DNA, 32 bp, Artificial Sequence
  Primer
tgcaactgaatagattgcagtaagttataagc

SEQ ID NO: 429 - DNA, 34 bp, Artificial Sequence
  Primer
tgaattatgcaagaagtgatcaattttctcacga

SEQ ID NO: 430 - DNA, 33 bp, Artificial Sequence
  Primer
tgccgtaactaacataagagaattatgcaagaa

SEQ ID NO: 431 - DNA, 22 bp, Artificial Sequence
  Primer
tgacggcatcgataccaccgtc

SEQ ID NO: 432 - DNA, 34 bp, Artificial Sequence
  Primer
tcacaggttctacttcatcaataatttccattgc

SEQ ID NO: 433 - DNA, 27 bp, Artificial Sequence
  Primer
tccgccaaaaactccccttttcacagg

SEQ ID NO: 434 - DNA, 30 bp, Artificial Sequence
  Primer
ttgcaatcgacatatccatttcaccatgcc

SEQ ID NO: 435 - DNA, 24 bp, Artificial Sequence
  Primer
ttctgcttgaggaatagtgcgtgg

SEQ ID NO: 436 - DNA, 31 bp, Artificial Sequence
  Primer
tacgttctacgatttcttcatcaggtacatc

SEQ ID NO: 437 - DNA, 27 bp, Artificial Sequence
  Primer
tacaacgtgataaacacgaccagaagc

SEQ ID NO: 438 - DNA, 28 bp, Artificial Sequence
  Primer
tccatattgttgcataaaacctgttggc

SEQ ID NO: 439 - DNA, 31 bp, Artificial Sequence
  Primer
tgagatgtcgaaaaaaacgttggcaaaatac

SEQ ID NO: 440 - DNA, 18 bp, Artificial Sequence
  Primer
acggcacgaggtagtcgc

SEQ ID NO: 441 - DNA, 23 bp, Artificial Sequence
  Primer
taaccatttcgcgtaagattcaa

SEQ ID NO: 442 - DNA, 28 bp, Artificial Sequence
  Primer
tcatgtgctaatgttactgctggatctg

SEQ ID NO: 443 - DNA, 14 bp, Artificial Sequence
  Primer
tgttactgctggat

SEQ ID NO: 444 - DNA, 17 bp, Artificial Sequence
  Primer
ttacttctaacccactc

SEQ ID NO: 445 - DNA, 21 bp, Artificial Sequence
  Primer
tgcgggctggttcaacaagag

SEQ ID NO: 446 - DNA, 19 bp, Artificial Sequence
  Primer
tgatgcgggctggttcaac

SEQ ID NO: 447 - DNA, 27 bp, Artificial Sequence
  Primer
tcctgttttatagccgccaagagtaag

SEQ ID NO: 448 - DNA, 28 bp, Artificial Sequence
  Primer
tcaaggttctcaccgtttaccttaggag

SEQ ID NO: 449 - DNA, 26 bp, Artificial Sequence
  Primer
tgaatcttgaaacaccatacgtaacg

SEQ ID NO: 450 - DNA, 21 bp, Artificial Sequence
  Primer
tgaatcttgaaacaccatacg

SEQ ID NO: 451 - DNA, 28 bp, Artificial Sequence
  Primer
gtaacccttgtctttgaattgtatttgc

SEQ ID NO: 452 - DNA, 29 bp, Artificial Sequence
  Primer
tgtaacccttgtctttgaattgtatttgc

SEQ ID NO: 453 - DNA, 20 bp, Artificial Sequence
  Primer
ggtaacccttgtctttgaat

SEQ ID NO: 454 - DNA, 18 bp, Artificial Sequence
  Primer
tggtaacccttgtctttg

SEQ ID NO: 455 - DNA, 34 bp, Artificial Sequence
  Primer
tcccttatttttctttctactaccttcggataat

SEQ ID NO: 456 - DNA, 33 bp, Artificial Sequence
  Primer
tcccctcatgtttaaatgatcaggataaaaagc

SEQ ID NO: 457 - DNA, 31 bp, Artificial Sequence
  Primer
tcggtttaagctctacatgatcgtaaggata

SEQ ID NO: 458 - DNA, 28 bp, Artificial Sequence
  Primer
tttgctcatgatctgcatgaagcataaa

SEQ ID NO: 459 - DNA, 29 bp, Artificial Sequence
  Primer
tgcaatgtgtgctatgtcagcaaaaagat

SEQ ID NO: 460 - DNA, 26 bp, Artificial Sequence
  Primer
tgagcgtgtggaaaaggacttggatg

SEQ ID NO: 461 - DNA, 28 bp, Artificial Sequence
  Primer
tatgtgtagttgagcttactacatgagc

SEQ ID NO: 462 - DNA, 31 bp, Artificial Sequence
  Primer
tggttcttacttgctttgcataaactttcca

SEQ ID NO: 463 - DNA, 27 bp, Artificial Sequence
  Primer
tcgatccgcatcaccatcaaaagcaaa

SEQ ID NO: 464 - DNA, 32 bp, Artificial Sequence
  Primer
tccacactggattgtaatttaccttgttcttt

SEQ ID NO: 465 - DNA, 31 bp, Artificial Sequence
  Primer
tctctttcaaagcaccattgctcattatagt

SEQ ID NO: 466 - DNA, 34 bp, Artificial Sequence
  Primer
tgaattctttcaaagcaccattgctcattatagt

SEQ ID NO: 467 - DNA, 25 bp, Artificial Sequence
  Primer
ttgctgccatagcaaagcctacagc

SEQ ID NO: 468 - DNA, 28 bp, Artificial Sequence
  Primer
tgtgctttttttgctgccatagcaaagc

SEQ ID NO: 469 - DNA, 34 bp, Artificial Sequence
  Primer
tgcttcaaaacgcatttttacattttcgttaaag

SEQ ID NO: 470 - DNA, 29 bp, Artificial Sequence
  Primer
tcctccttgtgcctcaaaacgcattttta

SEQ ID NO: 471 - DNA, 30 bp, Artificial Sequence
  Primer
tcaaagaacccgcacctaattcatcattta

SEQ ID NO: 472 - DNA, 35 bp, Artificial Sequence
  Primer
tcaactggttcaaaaacattaagttgtaattgtcc

SEQ ID NO: 473 - DNA, 35 bp, Artificial Sequence
  Primer
tacaactggttcaaaaacattaagctgtaattgtc

SEQ ID NO: 474 - DNA, 30 bp, Artificial Sequence
  Primer
ttcattttctggtccaaagtaagcagtatc

SEQ ID NO: 475 - DNA, 33 bp, Artificial Sequence
  Primer
tcccgaacaatgagttgtatcaactatttttac

SEQ ID NO: 476 - DNA, 25 bp, Artificial Sequence
  Primer
tgcctaacaaatcccgtctgagttc

SEQ ID NO: 477 - DNA, 26 bp, Artificial Sequence
  Primer
tgtcatcaagcaccccaaaatgaact

SEQ ID NO: 478 - DNA, 23 bp, Artificial Sequence
  Primer
ccacttttaataaggtttgtagc

SEQ ID NO: 479 - DNA, 19 bp, Artificial Sequence
  Primer
tgttgaccatgcttcttag

SEQ ID NO: 480 - DNA, 22 bp, Artificial Sequence
  Primer
cttctacatttttagccatcac

SEQ ID NO: 481 - DNA, 15 bp, Artificial Sequence
  Primer
cggcttcaagacccc

SEQ ID NO: 482 - DNA, 20 bp, Artificial Sequence
  Primer
tgttaacggcttcaagaccc

SEQ ID NO: 483 - DNA, 21 bp, Artificial Sequence
  Primer
ttgttaacggcttcaagaccc

SEQ ID NO: 484 - DNA, 28 bp, Artificial Sequence
  Primer
accacttttaataaggtttgtagctaac

SEQ ID NO: 485 - DNA, 20 bp, Artificial Sequence
  Primer
cgcggtcggctcgttgatga

SEQ ID NO: 486 - DNA, 29 bp, Artificial Sequence
  Primer
tcacctacagctttaaagccagcaaaatg

SEQ ID NO: 487 - DNA, 33 bp, Artificial Sequence
  Primer
tcttctgtaaagggtggtttattattcatccca

SEQ ID NO: 488 - DNA, 25 bp, Artificial Sequence
  Primer
tagccttggcaacatcagcaaaact

SEQ ID NO: 489 - DNA, 28 bp, Artificial Sequence
  Primer
ttggcgacggtatacccatagctttata

SEQ ID NO: 490 - DNA, 25 bp, Artificial Sequence
  Primer
tgaacatttgcgacggtatacccat

SEQ ID NO: 491 - DNA, 27 bp, Artificial Sequence
  Primer
tgtgaacatttgcgacggtatacccat

SEQ ID NO: 492 - DNA, 31 bp, Artificial Sequence
  Primer
tggtgggtatcttagcaatcattctaatagc

SEQ ID NO: 493 - DNA, 30 bp, Artificial Sequence
  Primer
tgcgatggtaggtatcttagcaatcattct

SEQ ID NO: 494 - DNA, 22 bp, Artificial Sequence
  Primer
caatctgctgacggatctgagc

SEQ ID NO: 495 - DNA, 23 bp, Artificial Sequence
  Primer
ttcaggtccatcgggttcatgcc

SEQ ID NO: 496 - DNA, 23 bp, Artificial Sequence
  Primer
ccgcggtcgaattgcatgccttc

SEQ ID NO: 497 - DNA, 20 bp, Artificial Sequence
  Primer
tagccgcggtcgaattgcat

SEQ ID NO: 498 - DNA, 24 bp, Artificial Sequence
  Primer
tcgaaccgaagttaccctgaccat

SEQ ID NO: 499 - DNA, 24 bp, Artificial Sequence
  Primer
tgccagcttagtcatacggacttc

SEQ ID NO: 500 - DNA, 30 bp, Artificial Sequence
  Primer
tattgcggatcaccatgatgatattcttgc

SEQ ID NO: 501 - DNA, 27 bp, Artificial Sequence
  Primer
tcgttgagatggtttttaccttcgttg

SEQ ID NO: 502 - DNA, 29 bp, Artificial Sequence
  Primer
tttgtgaaacagcgaacattttcttggta

SEQ ID NO: 503 - DNA, 22 bp, Artificial Sequence
  Primer
tcacgcgcatcatcaccagtca

SEQ ID NO: 504 - DNA, 27 bp, Artificial Sequence
  Primer
tcctgcaatatctaatgcactcttacg

SEQ ID NO: 505 - DNA, 27 bp, Artificial Sequence
  Primer
acctgcaatatctaatgcactcttacg

SEQ ID NO: 506 - DNA, 25 bp, Artificial Sequence
  Primer
ctttcgctttctcgaactcaaccat

SEQ ID NO: 507 - DNA, 22 bp, Artificial Sequence
  Primer
tagcccagctgtttgagcaact

SEQ ID NO: 508 - DNA, 32 bp, Artificial Sequence
  Primer
tccctaatagtagaaataactgcatcagtagc

SEQ ID NO: 509 - DNA, 32 bp, Artificial Sequence; identical to SEQ ID NO: 508
  Primer

SEQ ID NO: 510 - DNA, 23 bp, Artificial Sequence
  Primer
taggatttttccacggcggcatc

SEQ ID NO: 511 - DNA, 24 bp, Artificial Sequence
  Primer
tagccttttctccggcgtagatct

SEQ ID NO: 512 - DNA, 18 bp, Artificial Sequence
  Primer
catgatggtcacaaccgg

SEQ ID NO: 513 - DNA, 19 bp, Artificial Sequence
  Primer
tcggcatcacgccgtcgtc

SEQ ID NO: 514 - DNA, 29 bp, Artificial Sequence
  Primer
tgctgctttcgcatggttaattgcttcaa

SEQ ID NO: 515 - DNA, 30 bp, Artificial Sequence
  Primer
ttgctgctttcgcatggttaattgcttcaa

SEQ ID NO: 516 - DNA, 21 bp, Artificial Sequence
  Primer
aacttcgccttcggtcatgtt

SEQ ID NO: 517 - DNA, 25 bp, Artificial Sequence
  Primer
ttgcgttgcagattatctttaccaa

SEQ ID NO: 518 - DNA, 28 bp, Artificial Sequence
  Primer
tgttaagtgtgttgcggctgtctttatt

SEQ ID NO: 519 - DNA, 24 bp, Artificial Sequence
  Primer
tcacgcgacgagtgccatccattg

SEQ ID NO: 520 - DNA, 25 bp, Artificial Sequence
  Primer
tgacccaaagctgaaagctttactg

SEQ ID NO: 521 - DNA, 20 bp, Artificial Sequence
  Primer
ttttccagccatgcagcgac

SEQ ID NO: 522 - DNA, 27 bp, Artificial Sequence
  Primer
tccttctgatgcctgatggaccaggag

SEQ ID NO: 523 - DNA, 19 bp, Artificial Sequence
  Primer
tgtcactcccgacacgcca

SEQ ID NO: 524 - DNA, 27 bp, Artificial Sequence
  Primer
taaacgtccgataccaatggttcgctc

SEQ ID NO: 525 - DNA, 26 bp, Artificial Sequence
  Primer
tcaacaacacctccttattcccactc

SEQ ID NO: 526 - DNA, 17 bp, Artificial Sequence
  Primer
gaatatcaatttgtagc

SEQ ID NO: 527 - DNA, 31 bp, Artificial Sequence
  Primer
agataaagaatcacgaatatcaatttgtagc

SEQ ID NO: 528 - DNA, 23 bp, Artificial Sequence
  Primer
aggatagatttatttcttgttcg

SEQ ID NO: 529 - DNA, 30 bp, Artificial Sequence
  Primer
tcttccaaggatagatttatttcttgttcg

SEQ ID NO: 530 - DNA, 31 bp, Artificial Sequence
  Primer
ttcttccaaggatagatttatttcttgttcg

SEQ ID NO: 531 - DNA, 18 bp, Artificial Sequence
  Primer
tcttgacagcatccgttg

SEQ ID NO: 532 - DNA, 20 bp, Artificial Sequence
  Primer
cagataaagaatcgctccag

SEQ ID NO: 533 - DNA, 25 bp, Artificial Sequence
  Primer
tctcatcccgatattaccgccatga

SEQ ID NO: 534 - DNA, 23 bp, Artificial Sequence
  Primer
tggcaacagctcaacacctttgg

SEQ ID NO: 535 - DNA, 30 bp, Artificial Sequence
  Primer
tgatcctgaatgtttatatctttaacgcct

SEQ ID NO: 536 - DNA, 27 bp, Artificial Sequence
  Primer
tcccaatctaacttccacataccatct

SEQ ID NO: 537 - DNA, 27 bp, Artificial Sequence
  Primer
tggatagacgtcatatgaaggtgtgct

SEQ ID NO: 538 - DNA, 25 bp, Artificial Sequence
  Primer
tattcttcgttactcatgccataca

SEQ ID NO: 539 - DNA, 11 bp, Artificial Sequence
  Primer
tactcatgcca

SEQ ID NO: 540 - DNA, 11 bp, Artificial Sequence
  Primer
tattcttcgtt

SEQ ID NO: 541 - DNA, 29 bp, Artificial Sequence
  Primer
taaccaccccaagatttatctttttgcca

SEQ ID NO: 542 - DNA, 27 bp, Artificial Sequence
  Primer
tgtgatatggaggtgtagaaggtgtta

SEQ ID NO: 543 - DNA, 25 bp, Artificial Sequence
  Primer
gagctgcgccaacgaataaatcgtc

SEQ ID NO: 544 - DNA, 30 bp, Artificial Sequence
  Primer
tacgtcgcctttaacttggttatattcagc

SEQ ID NO: 545 - DNA, 28 bp, Artificial Sequence
  Primer
tgccgtaacatagaagttaccgttgatt

SEQ ID NO: 546 - DNA, 33 bp, Artificial Sequence
  Primer
tcgggcgtagtttttagtaattaaatcagaagt

SEQ ID NO: 547 - DNA, 28 bp, Artificial Sequence
  Primer
tcgtcgtatttatagtgaccagcaccta

SEQ ID NO: 548 - DNA, 28 bp, Artificial Sequence
  Primer
tcaacaccagcgttacctaaagtacctt

SEQ ID NO: 549 - DNA, 23 bp, Artificial Sequence
  Primer
tttaagcgccagaaagcaccaac

SEQ ID NO: 550 - DNA, 25 bp, Artificial Sequence
  Primer
tcgtttaagcgccagaaagcaccaa

SEQ ID NO: 551 - DNA, 27 bp, Artificial Sequence
  Primer
taagccagcaagagctgtatagttcca

SEQ ID NO: 552 - DNA, 22 bp, Artificial Sequence
  Primer
tacaggagcagcaggcttcaag

SEQ ID NO: 553 - DNA, 28 bp, Artificial Sequence
  Primer
tagcagcaaaagttatcacacctgcagt

SEQ ID NO: 554 - DNA, 31 bp, Artificial Sequence
  Primer
tggttgtagttcctgtagttgttgcattaac

SEQ ID NO: 555 - DNA, 25 bp, Artificial Sequence
  Primer
tcctgcagctctacctgctccatta

SEQ ID NO: 556 - DNA, 24 bp, Artificial Sequence
  Primer
ccctgtagtagaagaggtaaccac

SEQ ID NO: 557 - DNA, 20 bp, Artificial Sequence
  Primer
cctgtagtagaagaggtaac

SEQ ID NO: 558 - DNA, 19 bp, Artificial Sequence
  Primer
tgattatcagcggaagtag

SEQ ID NO: 559 - DNA, 18 bp, Artificial Sequence
  Primer
ccgtgctccatttttcag

SEQ ID NO: 560 - DNA, 20 bp, Artificial Sequence
  Primer
tcggataagctgccacaagg

SEQ ID NO: 561 - DNA, 20 bp, Artificial Sequence
  Primer
tcggataagctgccacaagg

SEQ ID NO: 562 - DNA, 28 bp, Artificial Sequence
  Primer
ttcccctgaccttcgattaaaggatagc

SEQ ID NO: 563 - DNA, 28 bp, Artificial Sequence
  Primer
ggtataacgcatcgcagcaaaagattta

SEQ ID NO: 564 - DNA, 22 bp, Artificial Sequence
  Primer
ttcggtataacgcatcgcagca

SEQ ID NO: 565 - DNA, 29 bp, Artificial Sequence
  Primer
tcgctcagcaataattcactataagccga

SEQ ID NO: 566 - DNA, 24 bp, Artificial Sequence
  Primer
taatgcgatactggcctgcaagtc

SEQ ID NO: 567 - DNA, 29 bp, Artificial Sequence
  Primer
tgtaaattccgcaaagactttggcattag

SEQ ID NO: 568 - DNA, 21 bp, Artificial Sequence
  Primer
tggtctgagtacctcctttgc

SEQ ID NO: 569 - DNA, 24 bp, Artificial Sequence
  Primer
tattggaaataccggcagcatctc

SEQ ID NO: 570 - DNA, 24 bp, Artificial Sequence
  Primer
ttcaagtgcttgctcaccattgtc

SEQ ID NO: 571 - DNA, 24 bp, Artificial Sequence
  Primer
tggctcataagacgcgcttgtaga

SEQ ID NO: 572 - DNA, 21 bp, Artificial Sequence
  Primer
tcgtttcaccctgtcatgccg

SEQ ID NO: 573 - DNA, 22 bp, Artificial Sequence
  Primer
tccgataagccggattctgtgc

SEQ ID NO: 574 - DNA, 23 bp, Artificial Sequence
  Primer
tgccgataagccggattctgtgc

SEQ ID NO: 575 - DNA, 27 bp, Artificial Sequence
  Primer
tctcttaccccaccctttcacccttac

SEQ ID NO: 576 - DNA, 20 bp, Artificial Sequence
  Primer
tgcctcgtgcaacccacccg

SEQ ID NO: 577 - DNA, 20 bp, Artificial Sequence
  Primer
tgcctcgcgcaacctacccg

SEQ ID NO: 578 - DNA, 22 bp, Artificial Sequence
  Primer
gtaagccatgttttgttccatc

SEQ ID NO: 579 - DNA, 25 bp, Artificial Sequence
  Primer
tttacctcgcctttccacccttacc

SEQ ID NO: 580 - DNA, 29 bp, Artificial Sequence
  Primer
tgctcttacctcaccgttccacccttacc

SEQ ID NO: 581 - DNA, 18 bp, Artificial Sequence
  Primer
ataagccgggttctgtcg

SEQ ID NO: 582 - DNA, 27 bp, Artificial Sequence
  Primer
tctatagagtccggactttcctcgtga

SEQ ID NO: 583 - DNA, 24 bp, Artificial Sequence
  Primer
tcaagcgatctacccgcattacaa

SEQ ID NO: 584 - DNA, 22 bp, Artificial Sequence
  Primer
ataagccatgttctgttccatc

SEQ ID NO: 585 - DNA, 27 bp, Artificial Sequence
  Primer
tgactttcctcccccttatcagtctcc

SEQ ID NO: 586 - DNA, 27 bp, Artificial Sequence
  Primer
ccaagtgctggtttaccccatggagta

SEQ ID NO: 587 - DNA, 22 bp, Artificial Sequence
  Primer
gtgctggtttaccccatggagt

SEQ ID NO: 588 - DNA, 23 bp, Artificial Sequence
  Primer
tgtgctggtttaccccatggagt

SEQ ID NO: 589 - DNA, 22 bp, Artificial Sequence
  Primer
tgtgctggtttaccccatggag

SEQ ID NO: 590 - DNA, 26 bp, Artificial Sequence
  Primer
tccaagtgctggtttaccccatggag

SEQ ID NO: 591 - DNA, 24 bp, Artificial Sequence
  Primer
tccaagtgctggtttaccccatgg

SEQ ID NO: 592 - DNA, 25 bp, Artificial Sequence
  Primer
ttccaagtgctggtttaccccatgg

SEQ ID NO: 593 - DNA, 29 bp, Artificial Sequence
  Primer
tgttttgtatccaagtgctggtttacccc

SEQ ID NO: 594 - DNA, 18 bp, Artificial Sequence
  Primer
ttcgctctcggcctggcc

SEQ ID NO: 595 - DNA, 20 bp, Artificial Sequence
  Primer
tcgtcgcggacttcgaagcc

SEQ ID NO: 596 - DNA, 21 bp, Artificial Sequence
  Primer
gctggattcgcctttgctacg

SEQ ID NO: 597 - DNA, 22 bp, Artificial Sequence
  Primer
tgctggattcgcctttgctacg

SEQ ID NO: 598 - DNA, 24 bp, Artificial Sequence
  Primer
ttgacgttgcatgttcgagcccat

SEQ ID NO: 599 - DNA, 30 bp, Artificial Sequence
  Primer
cgtataagctgcaccataagcttgtaatgc

SEQ ID NO: 600 - DNA, 30 bp, Artificial Sequence
  Primer
tttcttgaagagtatgagctgctccgtaag

SEQ ID NO: 601 - DNA, 24 bp, Artificial Sequence
  Primer
cgacttgacggttaacatttcctg

SEQ ID NO: 602 - DNA, 27 bp, Artificial Sequence
  Primer
gtccgacttgacggtcaacatttcctg

SEQ ID NO: 603 - DNA, 28 bp, Artificial Sequence
  Primer
tgtccgacttgacggtcaacatttcctg

SEQ ID NO: 604 - DNA, 28 bp, Artificial Sequence
  Primer
tgtccgacttgacggttagcatttcctg

SEQ ID NO: 605 - DNA, 28 bp, Artificial Sequence
  Primer
tgtccgacttgacggtcagcatttcctg

SEQ ID NO: 606 - DNA, 23 bp, Artificial Sequence
  Primer
tccagcaggttctgacggaaacg

SEQ ID NO: 607 - DNA, 26 bp, Artificial Sequence
  Primer
ttaccgagcaggttctgacggaaacg

SEQ ID NO: 608 - DNA, 24 bp, Artificial Sequence
  Primer
cgaacggccagagtagtcaacacg

SEQ ID NO: 609 - DNA, 24 bp, Artificial Sequence
  Primer
cgaacggcctgagtagtcaacacg

SEQ ID NO: 610 - DNA, 30 bp, Artificial Sequence
  Primer
tcaagcgccatctctttcggtaatccacat

SEQ ID NO: 611 - DNA, 30 bp, Artificial Sequence
  Primer
tcaagcgccatttcttttggtaaaccacat

SEQ ID NO: 612 - DNA, 30 bp, Artificial Sequence
  Primer
attcaagagccatttcttttggtaaaccac

SEQ ID NO: 613 - DNA, 21 bp, Artificial Sequence
  Primer
gttcaaatgcctggataccca

SEQ ID NO: 614 - DNA, 19 bp, Artificial Sequence
  Primer
gagcatcagcgtgcgtgct

SEQ ID NO: 615 - DNA, 20 bp, Artificial Sequence
  Primer
tgagcatcagcgtgcgtgct

SEQ ID NO: 616 - DNA, 21 bp, Artificial Sequence
  Primer
acgcgggcatgcagagatgcc

SEQ ID NO: 617 - DNA, 20 bp, Artificial Sequence
  Primer
ggcgcttgtacttaccgcac

SEQ ID NO: 618 - DNA, 22 bp, Artificial Sequence
  Primer
ttggccatcagaccacgcatac

SEQ ID NO: 619 - DNA, 22 bp, Artificial Sequence
  Primer
ttggccatcaggccacgcatac

SEQ ID NO: 620 - DNA, 19 bp, Artificial Sequence
  Primer
acgccatcaggccacgcat

SEQ ID NO: 621 - DNA, 20 bp, Artificial Sequence
  Primer
tacgccatcaggccacgcat

SEQ ID NO: 622 - DNA, 20 bp, Artificial Sequence
  Primer
ttacgccatcaggccacgca

SEQ ID NO: 623 - DNA, 25 bp, Artificial Sequence
  Primer
cgcaccatgcgtagagatgaagtac

SEQ ID NO: 624 - DNA, 25 bp, Artificial Sequence
  Primer
cgcaccgtgggttgagatgaagtac

SEQ ID NO: 625 - DNA, 26 bp, Artificial Sequence
  Primer
tcgcaccgtgggttgagatgaagtac

SEQ ID NO: 626 - DNA, 24 bp, Artificial Sequence
  Primer
tgctagacctttacgtgcaccgtg

SEQ ID NO: 627 - DNA, 24 bp, Artificial Sequence
  Primer
tactagacgacgggtcaggtaacc

SEQ ID NO: 628 - DNA, 25 bp, Artificial Sequence
  Primer
gtttttcgttgcgtacgatgatgtc

SEQ ID NO: 629 - DNA, 27 bp, Artificial Sequence
  Primer
acgtttttcgttttgaacgataatgct

SEQ ID NO: 630 - DNA, 27 bp, Artificial Sequence
  Primer
gaccccaacctggccttttgtcgttga

SEQ ID NO: 631 - DNA, 28 bp, Artificial Sequence
  Primer
tgaccccaacctggccttttgtcgttga

SEQ ID NO: 632 - DNA, 29 bp, Artificial Sequence
  Primer
aaactatttttttagctatactcgaacac

SEQ ID NO: 633 - DNA, 30 bp, Artificial Sequence
  Primer
taaactatttttttagctatactcgaacac

SEQ ID NO: 634 - DNA, 30 bp, Artificial Sequence
  Primer
ggataattggtcgtaacaagggatagtgag

SEQ ID NO: 635 - DNA, 31 bp, Artificial Sequence
  Primer
tggataattggtcgtaacaagggatagtgag

SEQ ID NO: 636 - DNA, 26 bp, Artificial Sequence
  Primer
atatgattatcattgaactgcggccg

SEQ ID NO: 637 - DNA, 27 bp, Artificial Sequence
  Primer
tatatgattatcattgaactgcggccg

SEQ ID NO: 638 - DNA, 28 bp, Artificial Sequence
  Primer
gcgtgacgaccttcttgaattgtaatca

SEQ ID NO: 639 - DNA, 29 bp, Artificial Sequence
  Primer
tgcgtgacgaccttcttgaattgtaatca

SEQ ID NO: 640 - DNA, 27 bp, Artificial Sequence
  Primer
ttggacctgtaatcagctgaatactgg

SEQ ID NO: 641 - DNA, 28 bp, Artificial Sequence
  Primer
tttggacctgtaatcagctgaatactgg

SEQ ID NO: 642 - DNA, 22 bp, Artificial Sequence
  Primer
attgcccagaaatcaaatcatc

SEQ ID NO: 643 - DNA, 23 bp, Artificial Sequence
  Primer
tattgcccagaaatcaaatcatc

SEQ ID NO: 644 - DNA, 26 bp, Artificial Sequence
  Primer
tgtggccgatttcaccacctgctcct

SEQ ID NO: 645 - DNA, 27 bp, Artificial Sequence
  Primer
ttgtggccgatttcaccacctgctcct

SEQ ID NO: 646 - DNA, 23 bp, Artificial Sequence
  Primer
tctgggtgacctggtgttttaga

SEQ ID NO: 647 - DNA, 24 bp, Artificial Sequence
  Primer
ttctgggtgacctggtgttttaga

SEQ ID NO: 648 - DNA, 28 bp, Artificial Sequence
  Primer
agctgctagatgagcttctgccatggcc

SEQ ID NO: 649 - DNA, 29 bp, Artificial Sequence
  Primer
tagctgctagatgagcttctgccatggcc

SEQ ID NO: 650 - DNA, 28 bp, Artificial Sequence
  Primer
ccataaggtcaccgtcaccattcaaagc

SEQ ID NO: 651 - DNA, 29 bp, Artificial Sequence
  Primer
tccataaggtcaccgtcaccattcaaagc

SEQ ID NO: 652 - DNA, 23 bp, Artificial Sequence
  Primer
ggaatttaccagcgatagacacc

SEQ ID NO: 653 - DNA, 24 bp, Artificial Sequence
  Primer
tggaatttaccagcgatagacacc

SEQ ID NO: 654 - DNA, 26 bp, Artificial Sequence
  Primer
tgccactttgacaactcctgttgctg

SEQ ID NO: 655 - DNA, 27 bp, Artificial Sequence
  Primer
ttgccactttgacaactcctgttgctg

SEQ ID NO: 656 - DNA, 29 bp, Artificial Sequence
  Primer
aatcgacgaccatcttggaaagatttctc

SEQ ID NO: 657 - DNA, 30 bp, Artificial Sequence
  Primer
taatcgacgaccatcttggaaagatttctc

SEQ ID NO: 658 - DNA, 25 bp, Artificial Sequence
  Primer
tcgacgaccatcttggaaagatttc

SEQ ID NO: 659 - DNA, 27 bp, Artificial Sequence
  Primer
ccagcagttactgtcccctcatctttg

SEQ ID NO: 660 - DNA, 28 bp, Artificial Sequence
  Primer
tccagcagttactgtcccctcatctttg

SEQ ID NO: 661 - DNA, 26 bp, Artificial Sequence
  Primer
gctgctttgatggctgaatccccttc

SEQ ID NO: 662 - DNA, 27 bp, Artificial Sequence
  Primer
tgctgctttgatggctgaatccccttc

SEQ ID NO: 663 - DNA, 25 bp, Artificial Sequence
  Primer
gggtctacacctgcacttgcataac

SEQ ID NO: 664 - DNA, 26 bp, Artificial Sequence
  Primer
tgggtctacacctgcacttgcataac

SEQ ID NO: 665 - DNA, 19 bp, Artificial Sequence
  Primer
atcccctgcttctgctgcc

SEQ ID NO: 666 - DNA, 20 bp, Artificial Sequence
  Primer
tatcccctgcttctgctgcc

SEQ ID NO: 667 - DNA, 24 bp, Artificial Sequence
  Primer
taccttttccacaacagaatcagc

SEQ ID NO: 668 - DNA, 26 bp, Artificial Sequence
  Primer
ccaaccttttccacaacagaatcagc

SEQ ID NO: 669 - DNA, 27 bp, Artificial Sequence
  Primer
tccaaccttttccacaacagaatcagc

SEQ ID NO: 670 - DNA, 29 bp, Artificial Sequence
  Primer
cccattttttcacgcatgctgaaaatatc

SEQ ID NO: 671 - DNA, 30 bp, Artificial Sequence
  Primer
tcccattttttcacgcatgctgaaaatatc

SEQ ID NO: 672 - DNA, 29 bp, Artificial Sequence
  Primer
gattggcgataaagtgatattttctaaaa

SEQ ID NO: 673 - DNA, 30 bp, Artificial Sequence
  Primer
tgattggcgataaagtgatattttctaaaa

SEQ ID NO: 674 - DNA, 26 bp, Artificial Sequence
  Primer
gcccaccagaaagactagcaggataa

SEQ ID NO: 675 - DNA, 27 bp, Artificial Sequence
  Primer
tgcccaccagaaagactagcaggataa

SEQ ID NO: 676 - DNA, 25 bp, Artificial Sequence
  Primer
cctacccaacgttcaccaagggcag

SEQ ID NO: 677 - DNA, 26 bp, Artificial Sequence
  Primer
tcctacccaacgttcaccaagggcag

SEQ ID NO: 678 - DNA, 25 bp, Artificial Sequence
  Primer
catgacagccaagacctcacccacc

SEQ ID NO: 679 - DNA, 26 bp, Artificial Sequence
  Primer
tcatgacagccaagacctcacccacc

SEQ ID NO: 680 - DNA, 15 bp, Artificial Sequence
  Primer
tgtgctttgaatgct

SEQ ID NO: 681 - DNA, 20 bp, Artificial Sequence
  Primer
tcatttgtgctttgaatgct

SEQ ID NO: 682 - DNA, 29 bp, Artificial Sequence
  Primer
tcataactagcatttgtgctttgaatgct

SEQ ID NO: 683 - DNA, 27 bp, Artificial Sequence
  Primer
ttgcacgtctgtttcagttgcaaattc

SEQ ID NO: 684 - DNA, 27 bp, Artificial Sequence
  Primer
ttgcacgtctgtttcagttgcaaattc

SEQ ID NO: 685 - DNA, 20 bp, Artificial Sequence
  Primer
tctgtttcagttgcaaattc

SEQ ID NO: 686 - DNA, 26 bp, Artificial Sequence
  Primer
tgcacgtctgtttcagttgcaaattc

SEQ ID NO: 687 - DNA, 27 bp, Artificial Sequence
  Primer
ttgcacgtctgtttcagttgcaaattc

SEQ ID NO: 688 - DNA, 30 bp, Artificial Sequence
  Primer
tttcacagcatgcacgtctgtttcagttgc

SEQ ID NO: 689 - DNA, 26 bp, Artificial Sequence
  Primer
ttgtgattgttttgcagctgattgtg

SEQ ID NO: 690 - DNA, 13 bp, Artificial Sequence
  Primer
tgcagctgattgt

SEQ ID NO: 691 - DNA, 22 bp, Artificial Sequence
  Primer
tgattgttttgcagctgattgt

SEQ ID NO: 692 - DNA, 26 bp, Artificial Sequence
  Primer
ttcaaaaccttgctctcgccaaacaa

SEQ ID NO: 693 - DNA, 26 bp, Artificial Sequence
  Primer
tacatcgtttcgcccaagatcaatca

SEQ ID NO: 694 - DNA, 27 bp, Artificial Sequence
  Primer
tcctcttttcacaggctctacttcatc

SEQ ID NO: 695 - DNA, 30 bp, Artificial Sequence
  Primer
tatttgggtttcattccactcagattctgg

SEQ ID NO: 696 - DNA, 24 bp, Artificial Sequence
  Primer
tgcgcgagcttttatttgggtttc

SEQ ID NO: 697 - DNA, 23 bp, Artificial Sequence
  Primer
ttcaaaatgcggaggcgtatgtg

SEQ ID NO: 698 - DNA, 20 bp, Artificial Sequence
  Primer
tgcccaggtacaacctgcat

SEQ ID NO: 699 - DNA, 30 bp, Artificial Sequence
  Primer
tccaggcattaccatttctactccttctgg

SEQ ID NO: 700 - DNA, 25 bp, Artificial Sequence
  Primer
ggcatcaccatttccttgtccttcg

SEQ ID NO: 701 - DNA, 26 bp, Artificial Sequence
  Primer
tggcatcaccatttccttgtccttcg

SEQ ID NO: 702 - DNA, 24 bp, Artificial Sequence
  Primer
gttgtcaccaggcattaccatttc

SEQ ID NO: 703 - DNA, 24 bp, Artificial Sequence
  Primer
gttgtcgccaggcataaccatttc

SEQ ID NO: 704 - DNA, 21 bp, Artificial Sequence
  Primer
gccgtccatttgagcagcacc

SEQ ID NO: 705 - DNA, 21 bp, Artificial Sequence
  Primer
gccgtccatctgagcagcacc

SEQ ID NO: 706 - DNA, 26 bp, Artificial Sequence
  Primer
tatagcaccatccatctgagcggcac

SEQ ID NO: 707 - DNA, 24 bp, Artificial Sequence
  Primer
tatgtgctcacgagtttgcggcat

SEQ ID NO: 708 - DNA, 26 bp, Artificial Sequence
  Primer
tggatgtgctcacgagtctgtggcat

SEQ ID NO: 709 - DNA, 19 bp, Artificial Sequence
  Primer
gcgctccacgtcttcacgc

SEQ ID NO: 710 - DNA, 20 bp, Artificial Sequence
  Primer
acgaactggatgtcgccgtt

SEQ ID NO: 711 - DNA, 24 bp, Artificial Sequence
  Primer
cggtacgaactggatgtcgccgtt

SEQ ID NO: 712 - DNA, 25 bp, Artificial Sequence
  Primer
tcggtacgaactggatgtcgccgtt

SEQ ID NO: 713 - DNA, 27 bp, Artificial Sequence
  Primer
ttcgcgcatccaggagaagtacatgtt

SEQ ID NO: 714 - DNA, 24 bp, Artificial Sequence
  Primer
gcgttccacagcttgttgcagaag

SEQ ID NO: 715 - DNA, 23 bp, Artificial Sequence
  Primer
tcgcagttcatcagcacgaagcg

SEQ ID NO: 716 - DNA, 23 bp, Artificial Sequence
  Primer
tataacgcacatcgtcagggtga

SEQ ID NO: 717 - DNA, 24 bp, Artificial Sequence
  Primer
caagcggtttgcctcaaatagtca

SEQ ID NO: 718 - DNA, 19 bp, Artificial Sequence
  Primer
tggcacgagcctgacctgt

SEQ ID NO: 719 - DNA, 1542 bp, Artificial Sequence
  Escherichia coli
aaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 720 - DNA, 2904 bp, Artificial Sequence
  Escherichia coli
ggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgcagcgatgcgttgagctaaccggtactaatgaaccgtgaggcttaacctt

SEQ ID NO: 721 - DNA, 447 bp, Artificial Sequence
  Bacillus anthracis
atgtttggatcagatttatatattgcattagtattaggagttacactgagccttatttttacagaaagaacaggtattttacctgcaggtttagttgtacctggttatttagcactcgtttttaatcagcccgtatttatgttggttgttttatttatcagtattttaacatatgtaatcgttacgtatggtgtttcaagattcatgattttatatggccgtagaaaatttgcggcaacgctaattacaggtatttgtttaaaacttttatttgattattgttatcctgttatgccatttgagatttttgaattccgtggtattggagttattgttccaggattaattgcaaatacaattcaaagacaagggttaccattaacaattggaactacaattttgttaagtggtgcaacatttgcaatcatgaatatttattacttattt

SEQ ID NO: 722 - DNA, 2339 bp, Artificial Sequence
  Bacillus anthracis
atgactagaaataaatttatacctaataagtttagtattatatccttttcagtattactatttgctatatcctcctcacaggctatagaagtaaatgctatgaatgaacattacactgagagtgatattaaaagaaaccataaaactgaaaaaaataaaactgaaaaagaaaaatttaaagacagtattaataacttagttaaaacagaatttaccaatgaaactttagataaaatacagcagacacaagacttattaaaaaagatacctaaggatgtacttgaaatttatagtgaattaggaggagaaatctattttacagatatagatttagtagaacataaggagttacaagatttaagtgaagaagagaaaaatagtatgaatagtagaggtgaaaaagttccgtttgcatcccgttttgtatttgaaaagaaaagggaaacacctaaattaattataaatatcaaagattatgcaattaatagtgaacaaagtaaagaagtatattatgaaattggaaaggggatttctcttgatattataagtaaggataaatctctagatccagagtttttaaatttaattaagagtttaagcgatgatagtgatagtagcgaccttttatttagtcaaaaatttaaagagaagctagaattgaataataaaagtatagatataaattttataaaagaaaatttaactgaatttcagcatgcgttttctttagcgttttcttattattttgcacctgaccatagaacggtattagagttatatgcccccgacatgtttgagtatatgaataagttagaaaaagggggatttgagaaaataagtgaaagtttgaagaaagaaggtgtggaaaaagataggattgatgtgctgaaaggagaaaaagcacttaaagcttcaggtttagtaccagaacatgcagatgcttttaaaaaaattgctagagaattaaatacatatattctttttaggcctgttaataagttagctacaaaccttattaaaagtggtgtggctacaaagggattgaatgttcatggaaagagttcggattggggccctgtagctggatacataccatttgatcaagatttatctaagaagcatggtcaacaattagctgtcgagaaaggaaatttagaaaataaaaaatcaattacagagcatgaaggtgaaataggtaaaataccattaaagttagaccatttaagaatagaagagttaaaggaaaatgggataattttgaagggtaaaaaagaaattgataatggtaaaaaatattatttgttagaatcgaataatcaggtatatgaatttagaattagcgatgaaaacaacgaagtacaatacaagacaaaagaaggtaaaattactgttttaggggaaaaattcaattggagaaatatagaagtgatggctaaaaatgtagaaggggtcttgaagccgttaacagctgactatgatttatttgcacttgccccaagtttaacagaaataaaaaaacaaataccacaaaaagaatgggataaagtagttaacaccccaaattcattagaaaagcaaaaaggtgttactaatttattgattaaatatggaattgagaggaaaccggattcaactaagggaactttatcaaattggcaaaaacaaatgcttgatcgtttgaatgaagcagtcaaatatacaggatatacagggggggatgtggttaaccatggcacagagcaagataatgaagagtttcctgaaaaagataacgaaatttttataattaatccagaaggtgaatttatattaactaaaaattgggagatgacaggtagatttatagaaaaaaacattacgggaaaagattatttatattattttaaccgttcttataataaaatagctcctggtaataaagcttatattgagtggactgatccgattacaaaagccaaaataaataccatccctacgtcagcagagtttataaaaaacttatccagtatcagaagatcttcaaatgtaggagtttataaagatagtggcgacaaagacgaatttgcaaaaaaagaaagcgtgaaaaaaattgcaggatatttgtcagactattacaattcagcaaatcatattttttctcaggaaaaaaagcgtaaaatatcaatatttcgtggaatccaagcctataatgaaattgaaaatgttctaaaatctaaacaaatagcaccagaatacaaaaattattttcaatatttaaaggaaaggattaccaatcaagttcaattgcttctaacacatcaaaaatctaatattgaatttaaattattgtataaacaatt

SEQ ID NO: 723 - DNA, 1917 bp, Artificial Sequence
  Escherichia coli
atgggtaaaataattggtatcgacctgggtactaccaactcttgtgtagcgattatggatggcaccactcctcgcgtgctggagaacgccgaaggcgatcgcaccacgccttctatcattgcctatacccaggatggtgaaactctagttggtcagccggctaaacgtcaggcagtgacgaacccgcaaaacactctgtttgcgattaaacgcctgattggtcgccgcttccaggacgaagaagtacagcgtgatgtttccatcatgccgttcaaaattattgctgctgataacggcgacgcatgggtcgaagttaaaggccagaaaatggcaccgccgcagatttctgctgaagtgctgaaaaaaatgaagaaaaccgctgaagattacctgggtgaaccggtaactgaagctgttatcaccgtaccggcatactttaacgatgctcagcgtcaggcaaccaaagacgcaggccgtatcgctggtctggaagtaaaacgtatcatcaacgaaccgaccgcagctgcgctggcttacggtctggacaaaggcactggcaaccgtactatcgcggtttatgacctgggtggtggtactttcgatatttctattatcgaaatcgacgaagttgacggcgaaaaaaccttcgaagttctggcaaccaacggtgatacccacctggggggtgaagacttcgacagccgtctgatcaactatctggttgaagaattcaagaaagatcagggcattgacctgcgcaacgatccgctggcaatgcagcgcctgaaagaagcggcagaaaaagcgaaaatcgaactgtcttccgctcagcagaccgacgttaacctgccatacatcactgcagacgcgaccggtccgaaacacatgaacatcaaagtgactcgtgcgaaactggaaagcctggttgaagatctggtaaaccgttccattgagccgctgaaagttgcactgcaggacgctggcctgtccgtatctgatatcgacgacgttatcctcgttggtggtcagactcgtatgccaatggttcagaagaaagttgctgagttctttggtaaagagccgcgtaaagacgttaacccggacgaagctgtagcaatcggtgctgctgttcagggtggtgttctgactggtgacgtaaaagacgtactgctgctggacgttaccccgctgtctctgggtatcgaaaccatgggcggtgtgatgacgacgctgatcgcgaaaaacaccactatcccgaccaagcacagccaggtgttctctaccgctgaagacaaccagtctgcggtaaccatccatgtgctgcagggtgaacgtaaacgtgcggctgataacaaatctctgggtcagttcaacctagatggtatcaacccggcaccgcgcggcatgccgcagatcgaagttaccttcgatatcgatgctgacggtatcctgcacgtttccgcgaaagataaaaacagcggtaaagagcagaagatcaccatcaaggcttcttctggtctgaacgaagatgaaatccagaaaatggtacgcgacgcagaagctaacgccgaagctgaccgtaagtttgaagagctggtacagactcgcaaccagggcgaccatctgctgcacagcacccgtaagcaggttgaagaagcaggcgacaaactgccggctgacgacaaaactgctatcgagtctgcgctgactgcactggaaactgctctgaaaggtgaagacaaagccgctatcgaagcgaaaatgcaggaactggcacaggtttcccagaaactgatggaaatcgcccagcagcaacatgcccagcagcagactgccggtgctgatgcttctgcaaacaacgcgaaagatgacgatgttgtcgacgctgaatttgaagaagtcaaagacaaaaaataa

SEQ ID NO: 724 - DNA, 1647 bp, Artificial Sequence
  Escherichia coli
atggcagctaaagacgtaaaattcggtaacgacgctcgtgtgaaaatgctgcgcggcgtaaacgtactggcagatgcagtgaaagttaccctcggtccaaaaggccgtaacgtagttctggataaatctttcggtgcaccgaccatcaccaaagatggtgtttccgttgctcgtgaaatcgaactggaagacaagttcgaaaatatgggtgcgcagatggtgaaagaagttgcctctaaagcaaacgacgctgcaggcgacggtaccaccactgcaaccgtactggctcaggctatcatcactgaaggtctgaaagctgttgctgcgggcatgaacccgatggacctgaaacgtggtatcgacaaagcggttaccgctgcagttgaagaactgaaagcgctgtccgtaccatgctctgactctaaagcgattgctcaggttggtaccatctccgctaactccgacgaaaccgtaggtaaactgatcgctgaagcgatggacaaagtcggtaaagaaggcgttatcaccgttgaagacggtaccggtctgcaggacgaactggacgtggttgaaggtatgcagttcgaccgtggctacctgtctccttacttcatcaacaagccggaaactggcgcagtagaactggaaagcccgttcatcctgctggctgacaagaaaatctccaacatccgcgaaatgctgccggttctggaagctgttgccaaagcaggcaaaccgctgctgatcatcgctgaagatgtagaaggcgaagcgctggcaactctggttgttaacaccatgcgtggcatcgtgaaagtcgctgcggttaaagcaccgggcttcggcgatcgtcgtaaagctatgctgcaggatatcgcaaccctgactggcggtaccgtgatctctgaagagatcggtatggagctggaaaaagcaaccctggaagacctgggtcaggctaaacgtgttgtgatcaacaaagacaccaccactatcatcgatggcgtgggtgaagaagctgcaatccagggccgtgttgctcagatccgtcagcagattgaagaagcaacttctgactacgaccgtgaaaaactgcaggaacgcgtagcgaaactggcaggcggcgttgcagttatcaaagtgggtgctgctaccgaagttgaaatgaaagagaaaaaagcacgcgttgaagatgccctgcacgcgacccgtgctgcggtagaagaaggcgtggttgctggtggtggtgttgcgctgatccgcgtagcgtctaaactggctgacctgcgtggtcagaacgaagaccagaacgtgggtatcaaagttgcactgcgtgcaatggaagctccgctgcgtcagatcgtattgaactgcggcgaagaaccgtctgttgttgctaacaccgttaaaggcggcgacggcaactacggttacaacgcagcaaccgaagaatacggcaacatgatcgacatgggtatcctggatccaaccaaagtaactcgttctgctctgcagtacgcagcttctgtggctggcctgatgatcaccaccgaatgcatggttaccgacctgccgaaaaacgatgcagctgacttaggcgctgctggcggtatgggcggcatgggtggcatgggcggcatgatgtaa

SEQ ID NO: 725 - DNA, 1935 bp, Artificial Sequence
  Escherichia coli
atggcgaaaaacctaatactctggctggtcattgccgttgtgctgatgtcagtattccagagctttgggcccagcgagtctaatggccgtaaggtggattactctaccttcctacaagaggtcaataacgaccaggttcgtgaagcgcgtatcaacggacgtgaaatcaacgttaccaagaaagatagtaaccgttataccacttacattccggttcaggatccgaaattactggataacctgttgaccaagaacgtcaaggttgtcggtgaaccgcctgaagaaccaagcctgctggcttctatcttcatctcctggttcccgatgctgttgctgattggtgtctggatcttcttcatgcgtcaaatgcagggcggcggtggcaaaggtgccatgtcgtttggtaagagcaaagcgcgcatgctgacggaagatcagatcaaaacgacctttgctgacgttgcgggctgcgacgaagcaaaagaagaagttgctgaactggttgagtatctgcgcgagccgagccgcttccagaaactcggcggtaagatcccgaaaggcgtcttgatggtcggtcctccgggtaccggtaaaacgctgctggcgaaagcgattgcaggcgaagcgaaagttccgttctttactatctccggttctgacttcgtagaaatgttcgtcggtgtgggtgcatcccgtgttcgtgacatgttcgaacaggcgaagaaagcggcaccgtgcatcatctttatcgatgaaatcgacgccgtaggccgccagcgtggcgctggtctgggcggtggtcacgatgaacgtgaacagactctgaaccagatgctggttgagatggatggcttcgaaggtaacgaaggtatcatcgttatcgccgcgactaaccgtccggacgttctcgacccggccctgctgcgtcctggccgtttcgaccgtcaggttgtggtcggcttgccagatgttcgcggtcgtgagcagatcctgaaagttcacatgcgtcgcgtaccattggcacccgatatcgacgcggcaatcattgcccgtggtactcctggtttctccggtgctgacctggcgaacctggtgaacgaagcggcactgttcgctgctcgtggcaacaaacgcgttgtgtcgatggttgagttcgagaaagcgaaagacaaaatcatgatgggtgcggaacgtcgctccatggtgatgacggaagcgcagaaagaatcgacggcttaccacgaagcgggtcatgcgattatcggtcgcctggtgccggaacacgatccggtgcacaaagtgacgattatcccacgcggtcgtgcgctgggtgtgactttcttcttgcctgagggcgacgcaatcagcgccagccgtcagaaactggaaagccagatttctacgctgtacggtggtcgtctggcagaagagatcatctacgggccggaacatgtatctaccggtgcgtccaacgatattaaagttgcgaccaacctggcacgtaacatggtgactcagtggggcttctctgagaaattgggtccactgctgtacgcggaagaagaaggtgaagtgttcctcggccgtagcgtagcgaaagcgaaacatatgtccgatgaaactgcacgtatcatcgaccaggaagtgaaagcactgattgagcgtaactataatcgtgcgcgtcagcttctgaccgacaatatggatattctgcatgcgatgaaagatgctctcatgaaatatgagactatcgacgcaccgcagattgatgacctgatggcacgtcgcgatgtacgtccgccagcgggctgggaagaaccaggcgcttctaacaattctggcgacaatggtagtccaaaggctcctcgtccggttgatgaaccgcgtacgccgaacccgggtaacaccatgtcagagcagttaggcgacaagtaa

SEQ ID NO: 726 - DNA, 2673 bp, Artificial Sequence
  Escherichia coli
atgacagatgtaacgattaaaacgctggccgcagagcgacagacctccgtggaacgcctggtacagcaatttgctgatgcaggtatccggaagtctgctgacgactctgtgtctgcacaagagaaacagactttgattgaccacctgaatcagaaaaattcaggcccggacaaattgacgctgcaacgtaaaacacgcagcacccttaacattcctggtaccggtggaaaaagcaaatcggtacaaatcgaagtccgcaagaaacgcacctttgtgaaacgcgatccgcaagaggctgaacgccttgcagcggaagagcaagcgcagcgtgaagcggaagagcaagcccgtcgtgaggcagaagaatcggctaaacgcgaggcgcaacaaaaagctgaacgtgaggccgcagaacaagctaagcgtgaagctgctgaacaagcgaaacgtgaagctgcggaaaaagacaaagtgagcaatcaacaagacgatatgactaaaaacgcccaggctgaaaaagcccgccgtgagcaggaagctgcagagctcaagcgtaaagctgaagaagaagcgcgtcgtaaactcgaagaagaagcacgtcgcgttgctgaagaagcacgtcgtatggcggaagaaaacaaatggactgataacgcggaaccgactgaagattccagcgattatcacgtcactacttctcaacatgctcgccaggcagaagacgaaagcgatcgtgaagtcgaaggcggccgtggccgtggtcgtaacgcgaaagcagcgcgtccgaagaaaggcaacaaacacgctgaatcaaaagctgatcgtgaagaagcacgcgcagcagtacgtggcggtaaaggcggaaaacgtaaaggttcttcgctgcagcaaggcttccagaagcctgctcaggccgttaaccgtgacgttgtgatcggcgaaactatcaccgttggcgaactggcgaacaagatggcggttaaaggctctcaggtcatcaaagcgatgatgaaactgggcgcaatggcaaccatcaaccaggttatcgatcaggaaaccgcacagctggttgctgaagagatgggccataaagttatcctgcgtcgtgaaaacgagctggaagaggcggtaatgagcgaccgtgacacgggtgctgcggctgaaccgcgcgcgccggttgtgaccatcatgggtcacgttgaccacggtaaaacctctctgctggactacattcgttcaacgaaagtggcctctggcgaagcgggcggcattacccagcacattggtgcataccacgttgaaactgaaaacggcatgatcaccttcctggacaccccggggcacgccgcgtttacttcaatgcgtgctcgtggtgcgcaggcaacggacatcgtagtcctggttgttgctgccgacgacggtgtgatgccgcagaccatcgaagcaatccagcacgcgaaagcggcgcaggtaccggtggtggttgcagtgaacaagatcgataaaccagaagctgatccggatcgcgttaagaacgaactctcccagtacggcatcctgccggaagagtggggcggtgaaagccagttcgtacacgtatctgcgaaagcgggtaccggtatcgatgaactgctggacgctatcctgctgcaggcggaagttctggagctgaaagcggtacgtaaaggtatggcgagcggtgcggttatcgaatccttcctcgataaaggtcgtggtccggttgctaccgttctggtacgtgaaggtactctgcacaagggcgatatcgttctgtgtggcttcgaatacggtcgtgttcgtgcgatgcgtaacgaactgggtcaggaagtgctggaagcgggtccgtccattccggtggaaatcctcggcctgtccggcgtaccggctgcgggtgatgaagttaccgttgtacgtgacgagaagaaagcgcgtgaagttgcactctatcgtcagggtaaattccgcgaagttaaactggcgcgtcagcagaaatctaaactcgagaacatgttcgccaacatgaccgaaggcgaagttcacgaagtgaatatcgtcctgaaggcagacgtacagggttctgtcgaagcgatctccgactccttgctgaaactgtctactgacgaagttaaagtgaagatcatcggttctggcgtaggtggtatcaccgaaaccgacgccaccctggctgcggcgtccaacgccatcctggttggctttaacgtacgtgctgatgcctctgcacgtaaagtgattgaagcggaaagcctggatctgcgttactactccgtcatctataacctgattgacgaagtgaaagcggcgatgagcggtatgctgtctccggaactgaaacagcagattatcggtctggcggaagttcgtgacgtgttcaaatcgccgaaatttggtgccatcgcaggctgtatggttaccgaaggtgtggttaaacgtcacaacccgatccgcgttctgcgtgacaacgtggttatctacgaaggcgagctggagtccctgcgccgcttcaaagatgacgttaacgaagtccgtaacggtatggaatgtggtatcggcgttaagaactacaacgacgtccgcactggcgatgtgatcgaagtattcgaaatcatcgagatccaacgtaccattgcttaa

SEQ ID NO: 727 - DNA, 2480 bp, Artificial Sequence
  Bacillus anthracis
atgaatataaaaaaagaatttataaaagtaattagtatgtcatgtttagtaacagcaattactttgagtggtcccgtctttatcccccttgtacagggggcgggcggtcatggtgatgtaggtatgcacgtaaaagagaaagagaaaaataaagatgagaataagagaaaagatgaagaacgaaataaaacacaggaagagcatttaaaggaaatcatgaaacacattgtaaaaatagaagtaaaaggggaggaagctgttaaaaaagaggcagcagaaaagctacttgagaaagtaccatctgatgttttagagatgtataaagcaattggaggaaagatatatattgtggatggtgatattacaaaacatatatctttagaagcattatctgaagataagaaaaaaataaaagacatttatgggaaagatgctttattacatgaacattatgtatatgcaaaagaaggatatgaacccgtacttgtaatccaatcttcggaagattatgtagaaaatactgaaaaggcactgaacgtttattatgaaataggtaagatattatcaagggatattttaagtaaaattaatcaaccatatcagaaatttttagatgtattaaataccattaaaaatgcatctgattcagatggacaagatcttttatttactaatcagcttaaggaacatcccacagacttttctgtagaattcttggaacaaaatagcaatgaggtacaagaagtatttgcgaaagcttttgcatattatatcgagccacagcatcgtgatgttttacagctttatgcaccggaagcttttaattacatggataaatttaacgaacaagaaataaatctatccttggaagaacttaaagatcaacggatgctgtcaagatatgaaaaatgggaaaagataaaacagcactatcaacactggagcgattctttatctgaagaaggaagaggacttttaaaaaagctgcagattcctattgagccaaagaaagatgacataattcattctttatctcaagaagaaaaagagcttctaaaaagaatacaaattgatagtagtgattttttatctactgaggaaaaagagtttttaaaaaagctacaaattgatattcgtgattctttatctgaagaagaaaaagagcttttaaatagaatacaggtggatagtagtaatcctttatctgaaaaagaaaaagagtttttaaaaaagctgaaacttgatattcaaccatatgatattaatcaaaggttgcaagatacaggagggttaattgatagtccgtcaattaatcttgatgtaagaaagcagtataaaagggatattcaaaatattgatgctttattacatcaatccattggaagtaccttgtacaataaaatttatttgtatgaaaatatgaatatcaataaccttacagcaaccctaggtgcggatttagttgattccactgataatactaaaattaatagaggtattttcaatgaattcaaaaaaaatttcaaatatagtatttctagtaactatatgattgttgatataaatgaaaggcctgcattagataatgagcgtttgaaatggagaatccaattatcaccagatactcgagcaggatatttagaaaatggaaagcttatattacaaagaaacatcggtctggaaataaaggatgtacaaataattaagcaatccgaaaaagaatatataaggattgatgcgaaagtagtgccaaagagtaaaatagatacaaaaattcaagaagcacagttaaatataaatcaggaatggaataaagcattagggttaccaaaatatacaaagcttattacattcaacgtgcataatagatatgcatccaatattgtagaaagtgcttatttaatattgaatgaatggaaaaataatattcaaagtgatcttataaaaaaggtaacaaattacttagttgatggtaatggaagatttgtttttaccgatattactctccctaatatagctgaacaatatacacatcaagatgagatatatgagcaagttcattcaaaagggttatatgttccagaatcccgttctatattactccatggaccttcaaaaggtgtagaattaaggaatgatagtgagggttttatacacgaatttggacatgctgtggatgattatgctggatatctattagataagaaccaatctgatttagttacaaattctaaaaaattcattgatatttttaaggaagaagggagtaatttaacttcgtatgggagaacaaatgaagcggaattttttgcagaagcctttaggttaatgcattctacggaccatgctgaacgtttaaaagttcaaaaaaatgctccgaaaactttccaatttattaacgatcagattaagttcattattaactcataagtaatgtattaaaaattttcaaatggatttaataataataataataataa

SEQ ID NO: 728 - DNA, 2295 bp, Artificial Sequence
  Bacillus anthracis
atgaaaaaacgaaaagtgttaataccattaatggcattgtctacgatattagtttcaagcacaggtaatttagaggtgattcaggcagaagttaaacaggagaaccggttattaaatgaatcagaatcaagttcccaggggttactaggatactattttagtgatttgaattttcaagcacccatggtggttacctcttctactacaggggatttatctattcctagttctgagttagaaaatattccatcggaaaaccaatattttcaatctgctatttggtcaggatttatcaaagttaagaagagtgatgaatatacatttgctacttccgctgataatcatgtaacaatgtgggtagatgaccaagaagtgattaataaagcttctaattctaacaaaatcagattagaaaaaggaagattatatcaaataaaaattcaatatcaacgagaaaatcctactgaaaaaggattggatttcaagttgtactggaccgattctcaaaataaaaaagaagtgatttctagtgataacttacaattgccagaattaaaacaaaaatcttcgaactcaagaaaaaagcgaagtacaagtgctggacctacggttccagaccgtgacaatgatggaatccctgattcattagaggtagaaggatatacggttgatgtcaaaaataaaagaacttttctttcaccatggatttctaatattcatgaaaagaaaggattaaccaaatataaatcatctcctgaaaaatggagcacggcttctgatccgtacagtgatttcgaaaaggttacaggacggattgataagaatgtatcaccagaggcaagacacccccttgtggcagcttatccgattgtacatgtagatatggagaatattattctctcaaaaaatgaggatcaatccacacagaatactgatagtcaaacgagaacaataagtaaaaatacttctacaagtaggacacatactagtgaagtacatggaaatgcagaagtgcatgcgtcgttctttgatattggtgggagtgtatctgcaggatttagtaattcgaattcaagtacggtcgcaattgatcattcactatctctagcaggggaaagaacttgggctgaaacaatgggtttaaataccgctgatacagcaagattaaatgccaatattagatatgtaaatactgggacggctccaatctacaacgtgttaccaacgacttcgttagtgttaggaaaaaatcaaacactcgcgacaattaaagctaaggaaaaccaattaagtcaaatacttgcacctaataattattatccttctaaaaacttggcgccaatcgcattaaatgcacaagacgatttcagttctactccaattacaatgaattacaatcaatttcttgagttagaaaaaacgaaacaattaagattagatacggatcaagtatatgggaatatagcaacatacaattttgaaaatggaagagtgagggtggatacaggctcgaactggagtgaagtgttaccgcaaattcaagaaacaactgcacgtatcatttttaatggaaaagatttaaatctggtagaaaggcggatagcggcggttaatcctagtgatccattagaaacgactaaaccggatatgacattaaaagaagcccttaaaatagcatttggatttaacgaaccgaatggaaacttacaatatcaagggaaagacataaccgaatttgattttaatttcgatcaacaaacatctcaaaatatcaagaatcagttagcggaattaaacgcaactaacatatatactgtattagataaaatcaaattaaatgcaaaaatgaatattttaataagagataaacgttttcattatgatagaaataacatagcagttggggcggatgagtcagtagttaaggaggctcatagagaagtaattaattcgtcaacagagggattattgttaaatattgataaggatataagaaaaatattatcaggttatattgtagaaattgaagatactgaagggcttaaagaagttataaatgacagatatgatatgttgaatatttctagtttacggcaagatggaaaaacatttatagattttaaaaaatataatgataaattaccgttatatataagtaatcccaattataaggtaaatgtatatgctgttactaaagaaaacactattattaatcctagtgagaatggggatactagtaccaacgggatcaagaaaattttaatcttttctaaaaaaggctatgagataggataa

SEQ ID NO: 729 - DNA, 822 bp, Artificial Sequence
  Escherichia coli
atggcagttgttaaatgtaaaccgacatctccgggtcgtcgccacgtagttaaagtggttaaccctgagctgcacaagggcaaaccttttgctccgttgctggaaaaaaacagcaaatccggtggtcgtaacaacaatggccgtatcaccactcgtcatatcggtggtggccacaagcaggcttaccgtattgttgacttcaaacgcaacaaagacggtatcccggcagttgttgaacgtcttgagtacgatccgaaccgttccgcgaacatcgcgctggttctgtacaaagacggtgaacgccgttacatcctggcccctaaaggcctgaaagctggcgaccagattcagtctggcgttgatgctgcaatcaaaccaggtaacaccctgccgatgcgcaacatcccggttggttctactgttcataacgtagaaatgaaaccaggtaaaggcggtcagctggcacgttccgctggtacttacgttcagatcgttgctcgtgatggtgcttatgtcaccctgcgtctgcgttctggtgaaatgcgtaaagtagaagcagactgccgtgcaactctgggcgaagttggcaatgctgagcatatgctgcgcgttctgggtaaagcaggtgctgcacgctggcgtggtgttcgtccgaccgttcgcggtaccgcgatgaacccggtagaccacccacatggtggtggtgaaggtcgtaactttggtaagcacccggtaactccgtggggcgttcagaccaaaggtaagaagacccgcagcaacaagcgtactgataaattcatcgtacgtcgccgtagcaaataa

SEQ ID NO: 730 - DNA, 4029 bp, Artificial Sequence
  Escherichia coli
atggtttactcctataccgagaaaaaacgtattcgtaaggattttggtaaacgtccacaagttctggatgtaccttatctcctttctatccagcttgactcgtttcagaaatttatcgagcaagatcctgaagggcagtatggtctggaagctgctttccgttccgtattcccgattcagagctacagcggtaattccgagctgcaatacgtcagctaccgccttggcgaaccggtgtttgacgtccaggaatgtcaaatccgtggcgtgacctattccgcaccgctgcgcgttaaactgcgtctggtgatctatgagcgcgaagcgccggaaggcaccgtaaaagacattaaagaacaagaagtctacatgggcgaaattccgctcatgacagacaacggtacctttgttatcaacggtactgagcgtgttatcgtttcccagctgcaccgtagtccgggcgtcttctttgactccgacaaaggtaaaacccactcttcgggtaaagtgctgtataacgcgcgtatcatcccttaccgtggttcctggctggacttcgaattcgatccgaaggacaacctgttcgtacgtatcgaccgtcgccgtaaactgcctgcgaccatcattctgcgcgccctgaactacaccacagagcagatcctcgacctgttctttgaaaaagttatctttgaaatccgtgataacaagctgcagatggaactggtgccggaacgcctgcgtggtgaaaccgcatcttttgacatcgaagctaacggtaaagtgtacgtagaaaaaggccgccgtatcactgcgcgccacattcgccagctggaaaaagacgacgtcaaactgatcgaagtcccggttgagtacatcgcaggtaaagtggttgctaaagactatattgatgagtctaccggcgagctgatctgcgcagcgaacatggagctgagcctggatctgctggctaagctgagccagtctggtcacaagcgtatcgaaacgctgttcaccaacgatctggatcacggcccatatatctctgaaaccttacgtgtcgacccaactaacgaccgtctgagcgcactggtagaaatctaccgcatgatgcgccctggcgagccgccgactcgtgaagcagctgaaagcctgttcgagaacctgttcttctccgaagaccgttatgacttgtctgcggttggtcgtatgaagttcaaccgttctctgctgcgcgaagaaatcgaaggttccggtatcctgagcaaagacgacatcattgatgttatgaaaaagctcatcgatatccgtaacggtaaaggcgaagtcgatgatatcgaccacctcggcaaccgtcgtatccgttccgttggcgaaatggcggaaaaccagttccgcgttggcctggtacgtgtagagcgtgcggtgaaagagcgtctgtctctgggcgatctggataccctgatgccacaggatatgatcaacgccaagccgatttccgcagcagtgaaagagttcttcggttccagccagctgtctcagtttatggaccagaacaacccgctgtctgagattacgcacaaacgtcgtatctccgcactcggcccaggcggtctgacccgtgaacgtgcaggcttcgaagttcgagacgtacacccgactcactacggtcgcgtatgtccaatcgaaacccctgaaggtccgaacatcggtctgatcaactctctgtccgtgtacgcacagactaacgaatacggcttccttgagactccgtatcgtaaagtgaccgacggtgttgtaactgacgaaattcactacctgtctgctatcgaagaaggcaactacgttatcgcccaggcgaactccaacttggatgaagaaggccacttcgtagaagacctggtaacttgccgtagcaaaggcgaatccagcttgttcagccgcgaccaggttgactacatggacgtatccacccagcaggtggtatccgtcggtgcgtccctgatcccgttcctggaacacgatgacgccaaccgtgcattgatgggtgcgaacatgcaacgtcaggccgttccgactctgcgcgctgataagccgctggttggtactggtatggaacgtgctgttgccgttgactccggtgtaactgcggtagctaaacgtggtggtgtcgttcagtacgtggatgcttcccgtatcgttatcaaagttaacgaagacgagatgtatccgggtgaagcaggtatcgacatctacaacctgaccaaatacacccgttctaaccagaacacctgtatcaaccagatgccgtgtgtgtctctgggtgaaccggttgaacgtggcgacgtgctggcagacggtccgtccaccgacctcggtgaactggcgcttggtcagaacatgcgcgtagcgttcatgccgtggaatggttacaacttcgaagactccatcctcgtatccgagcgtgttgttcaggaagaccgtttcaccaccatccacattcaggaactggcgtgtgtgtcccgtgacaccaagctgggtccggaagagatcaccgctgacatcccgaacgtgggtgaagctgcgctctccaaactggatgaatccggtatcgtttacattggtgcggaagtgaccggtggcgacattctggttggtaaggtaacgccgaaaggtgaaactcagctgaccccagaagaaaaactgctgcgtgcgatcttcggtgagaaagcctctgacgttaaagactcttctctgcgcgtaccaaacggtgtatccggtacggttatcgacgttcaggtctttactcgcgatggcgtagaaaaagacaaacgtgcgctggaaatcgaagaaatgcagctcaaacaggcgaagaaagacctgtctgaagaactgcagatcctcgaagcgggtctgttcagccgtatccgtgctgtgctggtagccggtggcgttgaagctgagaagctcgacaaactgccgcgcgatcgctggctggagctgggcctgacagacgaagagaaacaaaatcagctggaacagctggctgagcagtatgacgaactgaaacacgagttcgagaagaaactcgaagcgaaacgccgcaaaatcacccagggcgacgatctggcaccgggcgtgctgaagattgttaaggtatatctggcggttaaacgccgtatccagcctggtgacaagatggcaggtcgtcacggtaacaagggtgtaatttctaagatcaacccgatcgaagatatgccttacgatgaaaacggtacgccggtagacatcgtactgaacccgctgggcgtaccgtctcgtatgaacatcggtcagatcctcgaaacccacctgggtatggctgcgaaaggtatcggcgacaagatcaacgccatgctgaaacagcagcaagaagtcgcgaaactgcgcgaattcatccagcgtgcgtacgatctgggcgctgacgttcgtcagaaagttgacctgagtaccttcagcgatgaagaagttatgcgtctggctgaaaacctgcgcaaaggtatgccaatcgcaacgccggtgttcgacggtgcgaaagaagcagaaattaaagagctgctgaaacttggcgacctgccgacttccggtcagatccgcctgtacgatggtcgcactggtgaacagttcgagcgtccggtaaccgttggttacatgtacatgctgaaactgaaccacctggtcgacgacaagatgcacgcgcgttccaccggttcttacagcctggttactcagcagccgctgggtggtaaggcacagttcggtggtcagcgtttcggggagatggaagtgtgggcgctggaagcatacggcgcagcatacaccctgcaggaaatgctcaccgttaagtctgatgacgtgaacggtcgtaccaagatgtataaaaacatcgtggacggcaaccatcagatggagccgggcatgccagaatccttcaacgtattgttgaaagagattcgttcgctgggtatcaacatcgaactggaagacgagtaa

SEQ ID NO: 731 - DNA, 4224 bp, Artificial Sequence
  Escherichia coli
gtgaaagatttattaaagtttctgaaagcgcagactaaaaccgaagagtttgatgcgatcaaaattgctctggcttcgccagacatgatccgttcatggtctttcggtgaagttaaaaagccggaaaccatcaactaccgtacgttcaaaccagaacgtgacggccttttctgcgcccgtatctttgggccggtaaaagattacgagtgcctgtgcggtaagtacaagcgcctgaaacaccgtggcgtcatctgtgagaagtgcggcgttgaagtgacccagactaaagtacgccgtgagcgtatgggccacatcgaactggcttccccgactgcgcacatctggttcctgaaatcgctgccgtcccgtatcggtctgctgctcgatatgccgctgcgcgatatcgaacgcgtactgtactttgaatcctatgtggttatcgaaggcggtatgaccaacctggaacgtcagcagatcctgactgaagagcagtatctggacgcgctggaagagttcggtgacgaattcgacgcgaagatgggggcggaagcaatccaggctctgctgaagagcatggatctggagcaagagtgcgaacagctgcgtgaagagctgaacgaaaccaactccgaaaccaagcgtaaaaagctgaccaagcgtatcaaactgctggaagcgttcgttcagtctggtaacaaaccagagtggatgatcctgaccgttctgccggtactgccgccagatctgcgtccgctggttccgctggatggtggtcgtttcgcgacttctgacctgaacgatctgtatcgtcgcgtcattaaccgtaacaaccgtctgaaacgtctgctggatctggctgcgccggacatcatcgtacgtaacgaaaaacgtatgctgcaggaagcggtagacgccctgctggataacggtcgtcgcggtcgtgcgatcaccggttctaacaagcgtcctctgaaatctttggccgacatgatcaaaggtaaacagggtcgtttccgtcagaacctgctcggtaagcgtgttgactactccggtcgttctgtaatcaccgtaggtccatacctgcgtctgcatcagtgcggtctgccgaagaaaatggcactggagctgttcaaaccgttcatctacggcaagctggaactgcgtggtcttgctaccaccattaaagctgcgaagaaaatggttgagcgcgaagaagctgtcgtttgggatatcctggacgaagttatccgcgaacacccggtactgctgaaccgtgcaccgactctgcaccgtctgggtatccaggcatttgaaccggtactgatcgaaggtaaagctatccagctgcacccgctggtttgtgcggcatataacgccgacttcgatggtgaccagatggctgttcacgtaccgctgacgctggaagcccagctggaagcgcgtgcgctgatgatgtctaccaacaacatcctgtccccggcgaacggcgaaccaatcatcgttccgtctcaggacgttgtactgggtctgtactacatgacccgtgactgtgttaacgccaaaggcgaaggcatggtgctgactggcccgaaagaagcagaacgtctgtatcgctctggtctggcttctctgcatgcgcgcgttaaagtgcgtatcaccgagtatgaaaaagatgctaacggtgaattagtagcgaaaaccagcctgaaagacacgactgttggccgtgccattctgtggatgattgtaccgaaaggtctgccttactccatcgtcaaccaggcgctgggtaaaaaagcaatctccaaaatgctgaacacctgctaccgcattctcggtctgaaaccgaccgttatttttgcggaccagatcatgtacaccggcttcgcctatgcagcgcgttctggtgcatctgttggtatcgatgacatggtcatcccggagaagaaacacgaaatcatctccgaggcagaagcagaagttgctgaaattcaggagcagttccagtctggtctggtaactgcgggcgaacgctacaacaaagttatcgatatctgggctgcggcgaacgatcgtgtatccaaagcgatgatggataacctgcaaactgaaaccgtgattaaccgtgacggtcaggaagagaagcaggtttccttcaacagcatctacatgatggccgactccggtgcgcgtggttctgcggcacagattcgtcagcttgctggtatgcgtggtctgatggcgaagccggatggctccatcatcgaaacgccaatcaccgcgaacttccgtgaaggtctgaacgtactccagtacttcatctccacccacggtgctcgtaaaggtctggcggataccgcactgaaaactgcgaactccggttacctgactcgtcgtctggttgacgtggcgcaggacctggtggttaccgaagacgattgtggtacccatgaaggtatcatgatgactccggttatcgagggtggtgacgttaaagagccgctgcgcgatcgcgtactgggtcgtgtaactgctgaagacgttctgaagccgggtactgctgatatcctcgttccgcgcaacacgctgctgcacgaacagtggtgtgacctgctggaagagaactctgtcgacgcggttaaagtacgttctgttgtatcttgtgacaccgactttggtgtatgtgcgcactgctacggtcgtgacctggcgcgtggccacatcatcaacaagggtgaagcaatcggtgttatcgcggcacagtccatcggtgaaccgggtacacagctgaccatgcgtacgttccacatcggtggtgcggcatctcgtgcggctgctgaatccagcatccaagtgaaaaacaaaggtagcatcaagctcagcaacgtgaagtcggttgtgaactccagcggtaaactggttatcacttcccgtaatactgaactgaaactgatcgacgaattcggtcgtactaaagaaagctacaaagtaccttacggtgcggtactggcgaaaggcgatggcgaacaggttgctggcggcgaaaccgttgcaaactgggacccgcacaccatgccggttatcaccgaagtaagcggttttgtacgctttactgacatgatcgacggccagaccattacgcgtcagaccgacgaactgaccggtctgtcttcgctggtggttctggattccgcagaacgtaccgcaggtggtaaagatctgcgtccggcactgaaaatcgttgatgctcagggtaacgacgttctgatcccaggtaccgatatgccagcgcagtacttcctgccgggtaaagcgattgttcagctggaagatggcgtacagatcagctctggtgacaccctggcgcgtattccgcaggaatccggcggtaccaaggacatcaccggtggtctgccgcgcgttgcggacctgttcgaagcacgtcgtccgaaagagccggcaatcctggctgaaatcagcggtatcgtttccttcggtaaagaaaccaaaggtaaacgtcgtctggttatcaccccggtagacggtagcgatccgtacgaagagatgattccgaaatggcgtcagctcaacgtgttcgaaggtgaacgtgtagaacgtggtgacgtaatttccgacggtccggaagcgccgcacgacattctgcgtctgcgtggtgttcatgctgttactcgttacatcgttaacgaagtacaggacgtataccgtctgcagggcgttaagattaacgataaacacatcgaagttatcgttcgtcagatgctgcgtaaagctaccatcgttaacgcgggtagctccgacttcctggaaggcgaacaggttgaatactctcgcgtcaagatcgcaaaccgcgaactggaagcgaacggcaaagtgggtgcaacttactcccgcgatctgctgggtatcaccaaagcgtctctggcaaccgagtccttcatctccgcggcatcgttccaggagaccactcgcgtgctgaccgaagcagccgttgcgggcaaacgcgacgaactgcgcggcctgaaagagaacgttatcgtgggtcgtctgatcccggcaggtaccggttacgcgtaccaccaggatcgtatgcgtcgccgtgctgcgggtgaagctccggctgcaccgcaggtgactgcagaagacgcatctgccagcctggcagaactgctgaacgcaggtctgggcggttctgataacgagtaa

SEQ ID NO: 732 - DNA, 3734 bp, Artificial Sequence
  Concatenation of S. pyogenes genes
aaccttaattggaaagaaacccaagaagtcggttccgttgttgaaaaagaattgggcattccttttgccattgacaatgatgccaatgtggctgcccttggtgaacgttgggtaggtgctggtgaaaataacccagatgtcgtcttcatgacacttggaacaggtgtcggtggaggcattattgctgatggtaacttgattcatggtgttgcaggagcaggtggtgaaatcggccacatgattgttgagccagaaaatggctttgcttgtacttgtggctcacacggctgtttggaaacagtagcttcagcaacaggagttgtcaaagtggcacgtttactggcagaagcctacgaaggggattcagccatcaaagcagctattgacaatggtgaaggtgttaccagtaaagacattttcatggcggctgaagcaggggattcctttgctgattctgttgtggaaaaggttggttactaccttggccttgcttcagcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaccttacttcgaaccatgaatcttttggaagtaccaactaagggacagattaggtttgaggggattgatattaccgataagaagaatgatattttcagcatgcgtgaaaaaatgggaatggttttccagcagtttaacctctttcccaatatgactattttagaaaatatcactttatcgccaatcaaaactaagggaatggctaaagcagaggctgacaaaacagccttgagcttgttggacaaagttggattatcagaaaaagccaaggcttatcctgctagtctttctggtgggcaacagcagcggattgcgattgcgcgtggactggctatggatccagatgttttactctttgatgaaccaacttcagctctagacccagaaatggtgggtgaggtcttggctgtcatgcaagatttggctaaatctgggatgactatggttattnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngcatgcaataccgcaacagcggtggcttgggaagaagtaaaagcagctttagatattcctgttttaggagttgtcttaccgggggcaagcgcagctattaaatcaacgacaaaaggccaggttggggtcatcggaaccccaatgacagtggcttcagacatttatcgcaaaaaaatccagctattagcaccatctattcaagtaaggagtcttgcttgcccgaagtttgtaccgattgtggaatcaaatgagatgtgttcgagtatagctaaaaaaatagtttatgacagtctagcaccattagtcggtaaaatagatacccttgtactaggatgtactcactatcccttgttacgaccaattatccaaaatgttatggggccatctgttaagctgattgacagtggagcagaatgcgtccgagatatctctgtcttannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacctatattaatcgtttacagaaactggctaaaattttggcaacggtagatgttttgcaaagtttagcagtcgttgctgaaaccaatcattatatccggccgcagttcaatgataatcatgtgattacaattcaagaaggtcgtcacgcggttgttgaaaaggttatgggagtgcaggaatacattcccaatagtatctcttttgaccaacagaccagtattcagctgattacaggtccaaatatgagtggtaagtcgacttatatgagacagctggccttaacggttatcatggcccagatgggttcatttgtggctgctgatcatgttgatttacctttatttgatgcgatttttacgcgtattggggctgctgatgatttgatttctgggcaatcaacctttannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctatggcctatgttctttggaatcacttcatgaacatcaatcccaaaacaagccgtaattggtcaaacagagaccgttttatcctatcagcaggtcatggaagtgccatgctttatagcttgttacacttagctggttatgatttatctgtagaagatttaaagaacttccgtcaatggggttctaaaacaccaggtcacccagaagtgaaccacacagacggtgtcgaagcaaccacaggacctcttggtcaagggatcgcaaatgccgttgggatggccatggcagaagctcatctagcagctaaatttaacaaaccaggctttgacatcgttgatcactacacatttgctttgaatggtgacggtgaccttatggaaggggtcagccaagaagcagcaagtatggcaggacatttaaaacttgggaaattggtcttgctatatgattcaaacgacannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngagagaatattctaaaggtagataattttttaactcatcaagttgattaccggttgatgaaagcaattggtaaagtgtttgctcaaaaatatgctgaggctggcattacaaaagtggttacaatcgaagcttcaggtattgcaccagccgtatacgctgcagaagcaatggatgttcctatgatttttgcgaaaaaacataaaaacattaccatgacagaaggcattttgacagcagaagtttattctttcactaaacaagtgacgagcacggtgtctatcgctggtaaattcctatctaaagaagacaaggttttgattattgatgactttttagctaatggtcaggcagccaaaggcttgattgagattattggtcaagcaggggcacaagtcgtcggcgttggtattgtgattgagaaatctttccaagatggtcgtcgattgattnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttgcagctgatagccaacgtaaagcccaacttgccatagaaaaaggtcgtttcaaagaagagattgcacctgtcactattcctcagcgtaaaggtgaacctttactcgttgatcaagatgaataccctaaatttggaacgacagtggataagttagcaaagttacgccctgcttttatcaaagatgaggggacagtaactgctggtaatgcttcaggaatcaatgatggagcagcggcaattttattgatgagtaaagaaaaagctgaagaattagggctccctattttagctaaaatcactagttatgcaagtgcaggtgtagacccaagtattatgggctgcggaccaatacctgctacgaaaaaggctcttgcaaaggctcagctgacaattgatgacattgatttgattgaagcaaacgaagcttttgc

SEQ ID NO: 733 - DNA, 288 bp, Artificial Sequence
  Bacillus anthracis
atgagtaaaaaacaacaaggttataacaaggcaacttctggtgctagcattcaaagcacaaatgctagttatggtacagagtttgcgactgaaacaaatgtacaagcagtaaaacaagcaaacgcacaatcagaagctaagaaagcgcaagcttctggtgctagcattcaaagcacaaatgctagttatggtacagaatttgcaactgaaacagacgtgcatgctgtgaaaaaacaaaatgcacaatcagctgcaaaacaatcacaatcttctagttcaaatcagtaa

SEQ ID NO: 734 - DNA, 1185 bp, Artificial Sequence
  Escherichia coli
atgtctaaagaaaagtttgaacgtacaaaaccgcacgttaacgtcggtactatcggccacgttgaccatggtaaaacaacgctgaccgctgcaatcactaccgtactggctaaaacctacggcggtgctgctcgcgcattcgaccagatcgataacgcgccggaagaaaaagctcgtggtatcaccatcaacacttctcacgttgaatacgacaccccgacccgtcactacgcacacgtagactgcccggggcacgccgactatgttaaaaacatgatcaccggtgctgcgcagatggacggcgcgatcctggtagttgctgcgactgacggcccgatgccgcagactcgtgagcacatcctgctgggtcgtcaggtaggcgttccgtacatcatcgtgttcctgaacaaatgcgacatggttgatgacgaagagctgctggaactggttgaaatggaagttcgtgaacttctgtctcagtacgacttcccgggcgacgacactccgatcgttcgtggttctgctctgaaagcgctggaaggcgacgcagagtgggaagcgaaaatcctggaactggctggcttcctggattcttacattccggaaccagagcgtgcgattgacaagccgttcctgctgccgatcgaagacgtattctccatctccggtcgtggtaccgttgttaccggtcgtgtagaacgcggtatcatcaaagttggtgaagaagttgaaatcgttggtatcaaagagactcagaagtctacctgtactggcgttgaaatgttccgcaaactgctggacgaaggccgtgctggtgagaacgtaggtgttctgctgcgtggtatcaaacgtgaagaaatcgaacgtggtcaggtactggctaagccgggcaccatcaagccgcacaccaagttcgaatctgaagtgtacattctgtccaaagatgaaggcggccgtcatactccgttcttcaaaggctaccgtccgcagttctacttccgtactactgacgtgactggtaccatcgaactgccggaaggcgtagagatggtaatgccgggcgacaacatcaaaatggttgttaccctgatccacccgatcgcgatggacgacggtctgcgtttcgcaatccgtgaaggcggccgtaccgttggcgcgggcgttgtagcaaaagttctgagctaa

SEQ ID NO: 735 - DNA, 2856 bp, Artificial Sequence
  Escherichia coli
atggaaaagacatataacccacaagatatcgaacagccgctttacgagcactgggaaaagcagggctactttaagcctaatggcgatgaaagccaggaaagtttctgcatcatgatcccgccgccgaacgtcaccggcagtttgcatatgggtcacgccttccagcaaaccatcatggataccatgatccgctatcagcgcatgcagggcaaaaacaccctgtggcaggtcggtactgaccacgccgggatcgctacccagatggtcgttgagcgcaagattgccgcagaagaaggtaaaacccgtcacgactacggccgcgaagctttcatcgacaaaatctgggaatggaaagcggaatctggcggcaccattacccgtcagatgcgccgtctcggcaactccgtcgactgggagcgtgaacgcttcaccatggacgaaggcctgtccaatgcggtgaaagaagttttcgttcgtctgtataaagaagacctgatttaccgtggcaaacgcctggtaaactgggatccgaaactgcgcaccgctatctctgacctggaagtcgaaaaccgcgaatcgaaaggttcgatgtggcacatccgctatccgctggctgacggtgcgaaaaccgcagacggtaaagattatctggtggtcgcgactacccgtccagaaaccctgctgggcgatactggcgtagccgttaacccggaagatccgcgttacaaagatctgattggcaaatatgtcattctgccgctggttaaccgtcgtattccgatcgttggcgacgaacacgccgacatggaaaaaggcaccggctgcgtgaaaatcactccggcgcacgactttaacgactatgaagtgggtaaacgtcacgccctgccgatgatcaacatcctgacctttgacggcgatatccgtgaaagcgcccaggtgttcgataccaaaggtaacgaatctgacgtttattccagcgaaatccctgcagagttccagaaactggagcgttttgctgcacgtaaagcagtcgttgccgcagttgacgcgcttggcctgctggaagaaattaaaccgcacgacctgaccgttccttacggcgaccgtggcggcgtagttatcgaaccaatgctgaccgaccagtggtacgtgcgtgccgatgtcctggcgaaaccggcggttgaagcggttgagaacggcgacattcagttcgtaccgaagcagtacgaaaacatgtacttctcctggatgcgcgatattcaggactggtgtatctctcgtcagttgtggtggggtcaccgtatcccggcatggtatgacgaagcgggtaacgtttatgttggccgcaacgaagacgaagtgcgtaaagaaaataacctcggtgctgatgttgtcctgcgtcaggacgaagacgttctcgatacctggttctcttctgcgctgtggaccttctctacccttggctggccggaaaataccgacgccctgcgtcagttccacccaaccagcgtgatggtatctggtttcgacatcattttcttctggattgcccgcatgatcatgatgaccatgcacttcatcaaagatgaaaatggcaaaccgcaggtgccgttccacaccgtttacatgaccggcctgattcgtgatgacgaaggccagaagatgtccaaatccaagggtaacgttatcgacccactggatatggttgacggtatttcgctgccagaactgctggaaaaacgtaccggcaatatgatgcagccgcagctggcggacaaaatccgtaagcgcaccgagaagcagttcccgaacggtattgagccgcacggtactgacgcgctgcgcttcaccctggcggcgctggcgtctaccggtcgtgacatcaactgggatatgaagcgtctggaaggttaccgtaacttctgtaacaagctgtggaacgccagccgctttgtgctgatgaacacagaaggtcaggattgcggcttcaacggcggcgaaatgacgctgtcgctggcggaccgctggattctggcggagttcaaccagaccatcaaagcgtaccgcgaagcgctggacagcttccgcttcgatatcgccgcaggcattctgtatgagttcacctggaaccagttctgtgactggtatctcgagctgaccaagccggtaatgaacggtggcaccgaagcagaactgcgcggtactcgccatacgctggtgactgtactggaaggtctgctgcgcctcgcgcatccgatcattccgttcatcaccgaaaccatctggcagcgtgtgaaagtactttgcggtatcactgccgacaccatcatgctgcagccgttcccgcagtacgatgcatctcaggttgatgaagccgcactggccgacaccgaatggctgaaacaggcgatcgttgcggtacgtaacatccgtgcagaaatgaacatcgcgccgggcaaaccgctggagctgctgctgcgtggttgcagcgcggatgcagaacgtcgcgtaaatgaaaaccgtggcttcctgcaaaccctggcgcgtctggaaagtatcaccgtgctgcctgccgatgacaaaggtccggtttccgttacgaagatcatcgacggtgcagagctgctgatcccgatggctggcctcatcaacaaagaagatgagctggcgcgtctggcgaaagaagtggcgaagattgaaggtgaaatcagccgtatcgagaacaaactggcgaacgaaggctttgtcgcccgcgcaccggaagcggtcatcgcgaaagagcgtgagaagctggaaggctatgcggaagcgaaagcgaaactgattgaacagcaggctgttatcgccgcgctgtaa

SEQ ID NO: 736 - DNA, 1770 bp, Artificial Sequence
  Escherichia coli
atgcgtacagaatattgtggacagctccgtttgtcccacgtggggcagcaggtgactctgtgtggttgggtcaaccgtcgtcgtgatcttggtagcctgatcttcatcgatatgcgcgaccgcgaaggtatcgtgcaggtatttttcgatccggatcgtgcggacgcgttaaagctggcctctgaactgcgtaatgagttctgcattcaggtcacgggcaccgtacgtgcgcgtgacgaaaaaaatattaaccgcgatatggcgaccggcgaaatcgaagtgctggcgtcctcgctgactatcatcaaccgcgcagatgttctgccgcttgactctaaccacgtcaacaccgaagaagcgcgtctgaaataccgctacctcgacctgcgtcgtccggaaatggctcagcgcctgaaaacccgcgctaaaatcaccagcctggtgcgccgttttatggatgaccacggcttcctcgacatcgaaactccgatgctgaccaaagccacgccggaaggcgcgcgtgactacctggtgccttctcgtgtgcacaaaggtaaattctacgcactgccgcaatccccgcagttgttcaaacagctgctgatgatgtccggttttgaccgttactatcagatcgttaaatgcttccgtgacgaagacctgcgtgctgaccgtcagcctgaatttactcagatcgatgtggaaacttctttcatgaccgcgccgcaagtgcgtgaagtgatggaagcgctggtgcgtcatctgtggctggaagtgaagggtgtggatctgggcgatttcccggtaatgacctttgcggaagcagaacgccgttatggttctgataaaccggatctgcgtaacccgatggaactgactgacgttgctgatctgctgaaatctgttgagtttgctgtatttgcaggtccggcgaacgatccgaaaggtcgcgtagcggctctgcgcgttccgggcggcgcatcgctgacccgtaagcagatcgacgaatacggtaacttcgttaaaatctacggcgcgaaaggtctggcttacatcaaagttaacgaacgcgcgaaaggtctggaaggtatcaacagcccggtagcgaagttccttaatgcagaaatcatcgaagacatcctggatcgtactgccgcgcaagatggcgatatgattttcttcggtgccgacaacaagaaaattgttgccgacgcgatgggtgcactgcgcctgaaagtgggtaaagaccttggtctgaccgacgaaagcaaatgggcaccgctgtgggttatcgacttcccgatgtttgaagacgacggtgaaggcggcctgacggcaatgcaccatccgttcacctcaccgaaagatatgacggctgcagaactgaaagctgcaccggaaaatgcggtggcgaacgcttacgatatggtcatcaatggttacgaagtgggcggtggttcagtacgtatccataatggtgatatgcagcagacggtgtttggtattctgggtatcaacgaagaggaacagcgcgagaaattcggcttcctgctcgacgctctgaaatacggtactccgccgcacgcaggtctggcattcggtcttgaccgtctgaccatgctgctgaccggcaccgacaatatccgtgacgttatcgccttcccgaaaaccacggcggcagcgtgtctgatgactgaagcaccgagctttgctaacccgactgcactggctgagctgagcattcaggttgtgaagaaggctgagaataac

SEQ ID NO: 737 - DNA, 3699 bp, Artificial Sequence
  Yersinia pestis
atgatggttttccagccaatcagtgagtttctcttgataaggaatgcgggaatgtctatgtattttaataaaataatttcatttaatattatttcacgaatagttatttgtatctttttgatatgtggaatgttcatggctggggcttcagaaaaatatgatgctaacgcaccgcaacaggtccagccttattctgtctcttcatctgcatttgaaaatctccatcctaataatgaaatggagagttcaatcaatcccttttccgcatcggatacagaaagaaatgctgcaataatagatcgcgccaataaggagcaggagactgaagcggtgaataagatgataagcaccggggccaggttagctgcatcaggcagggcatctgatgttgctcactcaatggtgggcgatgcggttaatcaagaaatcaaacagtggttaaatcgattcggtacggctcaagttaatctgaattttgacaaaaatttttcgctaaaagaaagctctcttgattggctggctccttggtatgactctgcttcattcctcttttttagtcagttaggtattcgcaataaagacagccgcaacacacttaaccttggcgtcgggatacgtacattggagaacggttggctgtacggacttaatactttttatgataatgatttgaccggccacaaccaccgtatcggtcttggtgccgaggcctggaccgattatttacagttggctgccaatgggtattttcgcctcaatggatggcactcgtcgcgtgatttctccgactataaagagcgcccagccactgggggggatttgcgcgcgaatgcttatttacctgcactcccacaactgggggggaagttgatgtatgagcaatacaccggtgagcgtgttgctttattgtgcccagaaaacccccagctaggctgggggttcagtaaagctttcagctttgggtcagttataaaaaccccttttgatttgttaaaacagtttgcggtctggcaactgcaaatgttcaacaagaaatcaaaagggggtcccaatgagggatgaaaagagcttagcgcacacccgatggaactgtaaatatcatatagtttttgcgccgaagtaccgaaggcaggtgttctacagggaaaaacgcagagcgattggcagtattttaagaaaactgtgcgaatggaaaaacgtgaatatcctggaagcagaatactgtgtggatcacatccatatgcttctggagatcccgcccaagatgagtgtctcgggatttatggggtacctgaagggaaagagcagtctgatgctttatgagcagtttggcgatttgaagttcaaataccgtaacagggagttttggtgtcgagggtattacgttgatacggtagggaaaaacacggccaggatacaagaatacataaagcaccaattggaagaggataaaatgggtgagcaactctcgatcccgtatcccggtagcccgtttacgggccgtaagtaatccatagatgcaaatgtcagatcgcgatgcgcctgttagggcgcggctggtaacagagccttataggcgcatatgaaaaacctccggctatgccggaggatatttattttggtaaagataatctgcaacgcaacccttatgccgtgactgccgggatcaattacacccccgtgcctctactcactgtcggggtagatcagcgtatggggaaaagcagtaagcatgaaacacagtggaacctccaaatgaactatcgcctgggcgagagttttcagtcgcaacttagcccttcagcggtggcgggaacacgtctactggctgagagccgctataaccttgtcgatcgtaacaataatatcgtgttggagtatcagaaacagcaggtggttaaactgacattatcgccagcaactatctccggcctgccgggtcaggtttatcaggtgaacgcacaagtacaaggggcatctgctgtaagggaaattgtctggagtgatgccgaactgattgccgctggcggcacattaacaccactgagtaccacacaattcaacttggttttaccgccttataaacgcacagcacaagtgagtcgggtaacggacgacctgacagccaacttttattcgcttagtgcgctcgcggttgatcaccaaggaaacagatctaactcattcacattgagcgtcaccgttcagcagcctcagttgacattaacggcggccgtcattggtgatggcgcaccggctagtgggaaaactgcaatcaccgttgagttcaccgttgctgattttgaggggaaacccttagccgggcaggaagtggtgataaccaccaataatggtgcgctaccgaataaaatcacggaaaagacagatgcaaatggcgtcgcgcgcattgcattaaccaatacgacagatggcgtgacggtagtcacagcagaagtggaggggcaacggcaaagtgttgatacccactttgttaagggtactatcgcggcggataaatccactctggctgcggtaccgacatctatcatcgctgatggtctaatggcttcaaccatcacgttggagttgaaggatacctatggggacccgcaggctggcgcgaatgtggcttttgacacaaccttaggcaatatgggcgttatcacggatcacaatgacggcacttatagcgcaccattgaccagtaccacgttgggggtagcaacagtaacggtgaaagtggatggggctgcgttcagtgtgccgagtgtgacggttaatttcacggcagatcctattccagatgctggccgctccagtttcaccgtctccacaccggatatcttggctgatggcacgatgagttccacattatcctttgtccctgtcgataagaatggccattttatcagtgggatgcagggcttgagttttactcaaaacggtgtgccggtgagtattagccccattaccgagcagccagatagctataccgcgacggtggttgggaataccgccggtgatgtcacaatcacgcctctggttgataccctgatactgagtacattgcagaaaaaaatatccctattcccggtacctacgctgaccggtattctggttaacgggcaaaatttcgctacggataaagggttcccgaaaacgatctttaaaaacgccacattccagttacagatggataacgatgttgctaataatactcagtatgagtggtcgtcgtcattcacacccaatgtatcggttaacgatcagggtcaggtgacgattacctaccaaacctatagcgaagtggctgtgacggcgaaaagtaaaaaattcccaagttattcggtgagttatcggttctacccaaatcggtggatatacgatggcggcacttcgctggtatcgagtatcgaggccagcagacaatgccaaggttcagatatgtctgcggttcttgaatcctcacgtgcaaccaacggaacgcgtgcgcctgacgggacattgtggggcgagtgggggagcttgaccgcgtatagttctgattggcaatctggcgaatattgggtcaaaaggaccagcacggattttgaaaccatgaatatgaacactggcctgctgcaaccagggcctgcatacttggcgttcccgctctgtgcgctgtcaatataaccagataacagatagcaataagaacag

SEQ ID NO: 738 - DNA, 3891 bp, Artificial Sequence
  Clostridium botulinum
atgcaatttgttaataaacaatttaattataaagatcctgtaaatggtgttgatattgcttatataaaaattccaaatgtaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtaagagggataataacttctaaaactaaatcattagataaaggatacaataaggcattaaatgatttatgtatcaaagttaataattgggacttgttttttagtccttcagaagataattttactaatgatctaaataaaggagaagaaattacatctgatactaatatagaagcagcagaagaaaatattagtttagatttaatacaacaatattatttaacctttaattttgataatgaacctgaaaatatttcaatagaaaatctttcaagtgacattataggccaattagaacttatgcctaatatagaaagatttcctaatggaaaaaagtatgagttagataaatatactatgttccattatcttcgtgctcaagaatttgaacatggtaaatctaggattgctttaacaaattctgttaacgaagcattattaaatcctagtcgtgtttatacatttttttcttcagactatgtaaagaaagttaataaagctacggaggcagctatgtttttaggctgggtagaacaattagtatatgattttaccgatgaaactagcgaagtaagtactacggataaaattgcggatataactataattattccatatataggacctgctttaaatataggtaatatgttatataaagatgattttgtaggtgctttaatattttcaggagctgttattctgttagaatttataccagagattgcaatacctgtattaggtacttttgcacttgtatcatatattgcgaataaggttctaaccgttcaaacaatagataatgctttaagtaaaagaaatgaaaaatgggatgaggtctataaatatatagtaacaaattggttagcaaaggttaatacacagattgatctaataagaaaaaaaatgaaagaagctttagaaaatcaagcagaagcaacaaaggctataataaactatcagtataatcaatatactgaggaagagaaaaataatattaattttaatattgatgatttaagttcgaaacttaatgagtctataaataaagctatgattaatataaataaatttttgaatcaatgctctgtttcatatttaatgaattctatgatcccttatggtgttaaacggttagaagattttgatgctagtcttaaagatgcattattaaagtatatatatgataatagaggaactttaattggtcaagtagatagattaaaagataaagttaataatacacttagtacagatataccttttcagctttccaaatacgtagataatcaaagattattatctacatttactgaatatattaagaatattattaatacttctatattgaatttaagatatgaaagtaatcatttaatagacttatctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtattttaaaagacttttggggtgattatttacaatatgataaaccatactatatgttaaatttatatgatccaaataaatatgtcgatgtaaataatgtaggtattagaggttatatgtatcttaaagggcctagaggtagcgtaatgactacaaacatttatttaaattcaagtttgtatagggggacaaaatttattataaaaaaatatgcttctggaaataaagataatattgttagaaataatgatcgtgtatatattaatgtagtagttaaaaataaagaatataggttagctactaatgcatcacaggcaggcgtagaaaaaatactaagtgcattagaaatacctgatgtaggaaatctaagtcaagtagtagtaatgaagtcaaaaaatgatcaaggaataacaaataaatgcaaaatgaatttacaagataataatgggaatgatataggctttataggatttcatcagtttaataatatagctaaactagtagcaagtaattggtataatagacaaatagaaagatctagtaggactttgggttgctcatgggaatttattcctgtagatgatggatggggagaaaggccactgtaa

SEQ ID NO: 739 - DNA, 9047 bp, Artificial Sequence
  Staphylococcus aureus
aagcttcttgtgaaaaaatatcttcaatcttatgaaagatctccacattatcgctaacttttgagacaagtgtttttccaatttcacccgcttccccatttgcaccacgatacaattgattattaataataagcccagcaccaatacctttatgtatacttaaagcaataagattattgtaggataaattatgattaaaattacgttcatataacgctgaaagattcgcttcattttcaactacgactggaacattagtaatttcttttattttcttagcaattgaaattccttcagtttcatggaatggtaaatatgtcacatgctgctcattatccacaactccatgtatagaaacagacacacctaatagtccgttataagtatcaagtttctcctgaatatcaatatgtttttttattatgcttaatatactactaaccttttcatcaggtaaatcataagattcatgcttaatgacattaccatcaaaataattgtacatcacttcaacagaactataagttaaatccaaagaaataaaataaccataaagatgattaaccttcagaagaataggttttctaccaccactcttcgtgctatcaccctcaccaacctcattaacaagagatttatactttaacttattcaaaatactagaaatcgttgccttattaactcttttaagatatttggacgcgaaatattatgatggtataaatttcccttagcactcttttttcattatcatttatattttattttccataattgcctaccccataagttattttgaatttagtttatagtttatcatgtatttttataccaactattttaacataaattaaattaaaccttagaacaacttaaaacctcacttaggttatcatcttcgttcttttattttttttattatttcaataaattacgtatttccaatatgacgattttttatgcaaagtcgttgtatgtttttcaaatttttgacatttatgcaatctaaatagccaattagggaatttttaacattttttatttgtttgatattatacttaatgtatcttaaatagaaagaggtatgcatatggatttcactggtgttattacaagcattattgatttaatcaagacttgcattcaggctttcggttaattttttcaactaaaaaacagaggaaatattcaacgacttgattgtttcctctgttttctatgtattgttgtaaacacaacaattttattttttattcaatatatttctcaattcttctatttcatcttgtgatagatcttctttttctacaaagtttaagacaagtgaattgaaaccgcctttgtatactttattgataaagtttttagatgttttatattttatatcactttcttctacaagagagtaatattgaaaaattttattgtcttttttacgatctataaatccctttttatacaatctcgttataagtgtacgaatggtttttggactccagtccttttgcatttgtatttcttctattatattattcgcacttgcatattttttcatccaaatgatattcataacttcccattctgcagatgatatttcatacgttttattatccattatattaattccatttcttttaaaatacgctcagaaatttgttgtgctttttcgccattcgcattgtcttcgccttttaaatgtgtagcaaaataatacgtattatctttcgtttcaacataacctacgaaccatccatttgcttctttgtgattcacgattcctgttccagttttacctacatatttataagtatctttttgtttcaaagtcatactattttcaactttttcaatagccttattatcaaaatgcatgttatgttgtttcatatttttcaacaaattaacctgttctattgcagaaatttttaatgaagattcattccaataattttcattccctgatatttcttcattaccatattcaattagatctaaataagatttaacctcatcttgtcttaaatgtttgtttaaattttcgtaataccaatttactgaatatttcattgaagaatttaaattttgatcttggttccattctttaaatggatattgatgtttatcccattgttgttcagtatgatttaatgagagtaaattttggtcgaatgccattaacgctaaataaattttgtaagtagaattaggtgaatatcgttgtttactttctggttcattataaatagaataagcttgctcccgttcattataaagcacaaaacttccatcaaatcctttgaaatacggagctagttgatttaattttttatatgatacatttgtttcatatttgtcttgttgaacatgtgcagatagtaacggtgcttgtattaaaagcgatatactacatacaatatacgcaacaatacgcttgtttcgattaggtttaggcattgaatcataaagtgcaatatacttaacacgttctttaatatttgaattaaaacctagtaaatattgtgctgccacattatttatgtgctgagattttaaaatagagcattttaatatcgattcaccataacgtatatgttcatggcgattcaaaatttttaaaacgtttctatcacatactttttcacagtcattgtccatcattgttttacttatatatagtgcaggattaaaccagaatatcattttaaaaacaacataaagctggttgaatattaagtcatgacttttcacatgtgatagttcatgtagaataatatattcaatttctttgtcattcatggtttcgactacgacagttggtagtacaatttgggatttcactaaaccaaataccatcggattatcaatgtttgaactataactaattgttatatgctttttgtagaactgcatcttactttgacatactttaagtcgttcattaagatatgacgattccaatgacgaacttttaataacatcaatttgtcggaatgccttaatcatataaaataagcacaacaaactaccaaatacccatatcaaaagaatcatatacgttatatttgaggtctcaaactgattaacattaattgctaagtctttcgtaacagatgattgttgaccatctaacatatgactaaccgaagaagtcgtgtcagatacatttcgattcatcatatcttttgaaaatgtaaaattcgatattttgtaaaatggtattaatggaattaacgtggagacgagcactaataaccaaatcttatgtgacataatattttgagtatattttatatagagcattctcactaaaaaaattacacatatcgtgagcaatgaactgattatacttaacattaaaaaagatgataacaccttctacacctccatatcacaaaaattataacattattttgacataaatactacatttgtaatatactacaaatgtagtcttatataaggaggatattgatgaaaaagataaaaattgttccacttattttaatagttgtagttgtcgggtttggtatatatttttatgcttcaaaagataaagaaattaataatactattgatgcaattgaagataaaaatttcaaacaagtttataaagatagcagttatatttctaaaagcgataatggtgaagtagaaatgactgaacgtccgataaaaatatataatagtttaggcgttaaagatataaacattcaggatcgtaaaataaaaaaagtatctaaaaataaaaaacgagtagatgctcaatataaaattaaaacaaactacggtaacattgatcgcaacgttcaatttaattttgttaaagaagatggtatgtggaagttagattgggatcatagcgtcattattccaggaatgcagaaagaccaaagcatacatattgaaaatttaaaatcagaacgtggtaaaattttagaccgaaacaatgtggaattggccaatacaggaacacatatgagattaggcatcgttccaaagaatgtatctaaaaaagattataaagcaatcgctaaagaactaagtatttctgaagactatatcaacaacaaatggatcaaaattgggtacaagatgataccttcgttccactttaaaaccgttaaaaaaatggatgaatatttaagtgatttcgcaaaaaaatttcatcttacaactaatgaaacagaaagtcgtaactatcctctagaaaaagcgacttcacatctattaggttatgttggtcccattaactctgaagaattaaaacaaaaagaatataaaggctataaagatgatgcagttattggtaaaaagggactcgaaaaactttacgataaaaagctccaacatgaagatggctatcgtgtcacaatcgttgacgataatagcaatacaatcgcacatacattaatagagaaaaagaaaaaagatggcaaagatattcaactaactattgatgctaaagttcaaaagagtatttataacaacatgaaaaatgattatggctcaggtactgctatccaccctcaaacaggtgaattattagcacttgtaagcacaccttcatatgacgtctatccatttatgtatggcatgagtaacgaagaatataataaattaaccgaagataaaaaagaacctctgctcaacaagttccagattacaacttcaccaggttcaactcaaaaaatattaacagcaatgattgggttaaataacaaaacattagacgataaaacaagttataaaatcgatggtaaaggttggcaaaaagataaatcttggggtggttacaacgttacaagatatgaagtggtaaatggtaatatcgacttaaaacaagcaatagaatcatcagataacattttctttgctagagtagcactcgaattaggcagtaagaaatttgaaaaaggcatgaaaaaactaggtgttggtgaagatataccaagtgattatccattttataatgctcaaatttcaaacaaaaatttagataatgaaatattattagctgattcaggttacggacaaggtgaaatactgattaacccagtacagatcctttcaatctatagcgcattagaaaataatggcaatattaacgcacctcacttattaaaagacacgaaaaacaaagtttggaagaaaaatattatttccaaagaaaatatcaatctattaaatgatggtatgcaacaagtcgtaaataaaacacataaagaagatatttatagatcttatgcaaacttaattggcaaatccggtactgcagaactcaaaatgaaacaaggagaaagtggcagacaaattgggtggtttatatcatatgataaagataatccaaacatgatgatggctattaatgttaaagatgtacaagataaaggaatggctagctacaatgccaaaatctcaggtaaagtgtatgatgagctatatgagaacggtaataaaaaatacgatatagatgaataacaaaacagtgaagcaatccgtaacgatggttgcttcactgttttattatgaattattaataagtgctgttacttctcccttaaatacaatttcttcattttcattgtatgttgaaagtgacactgtaacgagtccattttctttttttatggatttcttatttgtaatttcagcgataacgtacaatgtattacctgggtatacaggtttaataaatttaacgttattcatttgtgttcctgctacaacttcttctccgtatttaccttcttctacccataatttaaatgatattgaaagtgtatgcatgccagatgcaatgatacctttaaatctactttgttctgctttttctttatctatatgcatatattgaggatcaaaagttgttgcaaattggataatttcttcttctgtaatatgaaggctttttgttttgaatgtttctcctactataaaatcatcgtatttcatatatgtctctctttcttattcaaattaattttttagtatgtaacatgttaaaggtaagtctaccgtcactgaaacgtaagactcacctctaactttctattgagacaaatgcaccattttatctgcattgtctgtaaagataccatcaactccccaattagcaagttggtttgcacgtgctggtttgtttacagtccatacgttcaattcataacccgcttcttttaccatttttacttttgctttagtaagtttggcatcttcagtgtttactattttagcattacagtaatctaaaagtgttctccagtcttcacgaaacgaagttgtatggaatataactgctctgttatattatggcatgatttcttctgcaagtttaacaagcacaacattaaagcttaaatgagctcttcttgattctgatttaagtttgttaattgttcttccacttgcttaaccatacttttagaaagtgctagtccattcggtccagtaataccttttaattctacatttaaattcatattatattcatttgctatttttactacatcatcgaaagttggcaaatgttcatctttgaatttttcaccaaaccaagatcctgcagaagcatctttaatttcatcataattcaattcagttatttccccggacatatttgtagtccgttctaaataatcatcatgaatgataatcagttgttcatcttttgtaattgcaacatctaactccaaccagtttataccttctacttctgaagcagctttaaatgatgcaattgtattttccggagctttactaggtaatcctctatgtccatatacagttagcatattacctctccttgcattttatttttttaattaacgtaactgtattatcacattaatcgcacttttatttccattaaaaagagatgaatatcataaataaagaagtcgatagattcgtattgattatggagttaatctacgtctcatctcatttttaaaaaatcatttatgactcccaagctccattttgtaatcaagtctagtttttctgtaccccttatctgcaattttacttaggattgcttttaacttaccccttatcagaattttactgagaactgcttttaacgatcacctcttatctgcaattttgcctagaactgcttttaacgtacctcttatctgcaattttactgagaactgcttttaacttaccccttatcagcaattttgcatggaattgcttttaacgtacctcttatctgcaattttactgagaactgcttttaacaaacctcttatctgcaattttacttagaactgcttttaagctacctcttatcctgtaattttactgagaactgcttttaacaaacctcttatctgcaattttacttagaactgcttttaacaaacctcttatctgcaattttacttagaattgcttttactattcctcttattagtataatctcagtaagaatgcgtataaaaatgaaaattacaaccgattttgtaagtctggacgcctgagggaatagtatgtgcgagagactaatggctcgagccatacccctaggcaagcatgcacgtacaaaatcgtaagataaaaaaataagcatatcactgtaaactttaaaaaatcagtttagtgatatgcttatttatttcgagttaggatttatgtcccaagctcatcaagcacaatcggccactagtttatttctctatcttatatgttctgatatggtcttctatactgtataagtatacttttgaatatggatcttgtgtcaattcacgttcgaaatcaaattcttgattatcaaatgctgttaaagaatgtttcgtattcttcgactgataattgctctctagattctagcatatttaagtgtttctctttatctaatgctttgtcatatcctttaacgattgaaccactaaagatttctcctactgctcctgaaccataactaaatagacatacttctcttctggttggaatgtgtggttctgtaataacgaaattaaacttaagtataatgatcctgtataaatgttaccaacatctctattccataatacggttctgttgcaaagttgaatttatagtataattttaacaaaaaggagtcttctgtatgaactatttcagatataaacaatttaacaaggatgttatcactgtagccgttggctactatctaagatatacattgagttatcgtgatatatctgaaatattaagggaacgtggtgtaaacgttcatcattcaacggtctaccgttgggttcaagaatatgccccaattttgtatcaaatttggaagaaaaagcataaaaaagcttattacaaatggcgtattgatgagacgtacatcaaaataaaaggaaaatggagctatttatatcgtgccattgatgcagagggacatacattagatatttggttgcgtaagcaacgagataatcattcagcatatgcgtttatcaaacgtctcattaaacaatttggtaaacctcaaaaggtaattacagatcaggcaccttcaacgaaggtagcaatggctaaagtaattaaagcttttaaacttaaacctgactgtcattgtacatcgaaatatctgaataacctcattgagcaagatcaccgtcatattaaagtaagaaagacaaggtatcaaagtatcaatacagcaaagaatactttaaaaggtattgaatgtatttacgctctatataaaaagaaccgcaggtctcttcagatctacggattttcgccatgccacgaaattagcatcatgctagcaagttaagcgaacactgacatgataaattagtggttagctatatttttttactttgcaacagaaccgaaaataatctcttcaatttatttttatatgaatcctgtgactcaagattgtaatatctaaagatttcagttcatcatagacaatgttcttttcaacattttttatagcaaattgattaaataaattctctaatttctcccgtttgatttcactaccatagattatattatcattgatatagtcaatgaataatgtacaaattatcactcataacagt

SEQ ID NO: 740 - DNA, 1832 bp, Artificial Sequence
  Acinetobacter baumanii
cgcgtgtacgtaatactggtgaagccccacgtcctaagacgatgtttgagccaggtgaagaattacttgttattgacggtccattcacagactttaaaggtgtggtggaagaggttcaatacgataagtcacgtttaacgttgacgattaacgtgtttaatcgaccaactcaggttgaactcgaatttcgccaagtcgaaaaaacgatttaatcttaattggttgaaaagcgcccgattttatcgggcattgttgttgtaacagtattgttacgtagaaattggggagcctaacggcgtttgtacccagaggtatttaaaatggctaagaagattgacggctatatcaagctgcaagttccagctggtaaagcaaatccatctccaccgattggtcctgcactaggtcaacgtggtgtaaacatcatggcgttctgtaaagaattccaaaaacaatttaaagttcctacggctaaactcttaccagacttaccaagttttacgggcggcttggtgggttatttgggctacgatgctgtccgctacatcgagccacgtttaaagaatgtacctgcggctgatccgattacgctgccagatttatggttgatgctctcaaagacagtcattgtttttgacaatcttaaagatacgctatttttaattgtgcatgcggatacagagcagagtaatgcttatgaagacgctcaacaaaaattagatcaattagaacagttgttggcgactccagttagtttgcaagcgcgaccacatacgcctccgcattttgaatcaattactggtaaagcaaaattcttagagacggtagagaaggttaaagaatatattcgtgcaggcgatgtgatgcaggttgtacctgggcagcgtatggtttctgattttgatggagaagctttacaggtttaccgtgcattacgtcatttaaatccatcaccttatctattcttggttcaaggacaaacgattactgataaaaaaccatttcatattgttggttcatcaccggaaattttatctcgtttagaaaatggtattgctacagttcgacctttggcaggaactagaccgcgcggtaaaactaaagaagaagatatagcattagaaaaagatttgctttctgatgaaaaagagattgctgaacatttaatgctgattgatcttgggcgaaacgatgtagggcgcgtatcaaaaataggtaaggtccaagttacggatcaaatggtgatcgagcgctattcacatgtcatgcatattgtttcaaatgtacaaggtgaagtgcgtgatgatatcgatgcacttgatgtatttaaagccacctttccggcaggaacgttatcaggtgccccaaaaattcgtgcaatggaaattattgatgaagtagagcctgtgaaaagaggagtttttggcggggctgttggttatttgggatggcatggtgaaatggatatgtcgattgcaatccgtacttgtgttatccgtgacaaaaaggtgtatgtacaggctggtgcagggctagttgctgactcaaatccagaatctgagtggaatgaaacccaaataaaagctcgcgcagtgatcaaagcggttgaattatcatcaaacggattgattttatgagtttttagcggtttttttaaaaaaaccgcttgcatcgtttggaagttttgctaaactgcacaccgttccgatacgaaacgtacgaaacactaagaaacgccggcatagctcagttggtagagcaactgacttgtaatcagtaggtccacagttcgaatccgtgtgccggcaccattttaaagtgttaagttaatt

SEQ ID NO: 741 - DNA, 382 bp, Artificial Sequence
  Acinetobacter baumanii
cacaatgacattgcaagcaattgctcaatgtcaaaaatctggtggtacatgtgccttcattgatgctgagcacgccctagaccctcaatatgcacgcaagcttggtgtagatattgataacctacttgtttcacaacccgacaatggtgagcaagcacttgaaattgctgacatgcttgtccgttcaggcgcaattgatttaatcgttgtggactcggtagctgcacttacccctaaagcagaaatcgaaggtgagatgggtgactctcatatgggtctacaagcgcgtcttatgagccaggcacttcgtaaaattacgggtaatgctaaacgttcaaactgtatggttatcttcattaaccagattcgtatgaaaattggt

SEQ ID NO: 742 - DNA, 344 bp, Artificial Sequence
  Acinetobacter baumanii
aaatctgcccgtgtcgttggtgacgtaatcggtaaatatcacccgcatggtgactcagctgtttatgaaaccattgttcgtatggctcaagactttagcttacgttatttattggttgatggtcagggtaacttcggttcgatcgatggcgatagcgccgcggcaatgcgttataccgaagtccgtatgactaagctggcacatgagcttcttgcagatttagaaaaagacacagttgactgggaagataactacgacggttcggaacgtatccctgaagtacttccgacacgtgttccaaacttgttaatcaacggtgctgcgggtatcgccgtaggtatggc

SEQ ID NO: 743 - DNA, 909 bp, Artificial Sequence
  Acinetobacter baumanii
gataatagctataaagtttcaggtggcttacacggcgtaggtgtttctgttgttaacgcactttcaagtaaattgcatctaacaatttaccgtgctggtcaaatccatgagcaagaatatcatcatggtgatccgcaatatccattgcgtgtgattggtgaaacggataataccggaacaactgtacgtttttggccaagtgcagaaacattcagtcaaaccatttttaatgttgaaattctagcacgccgtttacgtgagctttctttcttgaatgctggtgtacgtatcgttttacgtgatgaacgtattaaccttgagcatgtgtatgactatgaaggcggtttatctgagtttgtaaaatacatcaacgaaggtaaaaaccatctcaacgaaatcttccatttcacagctgatgctgacaacggtattgctgtagaagttgcattgcaatggaacgatagttaccaagaaaatgttcgctgtttcacaaacaacattccacaaaaagatggtggtacgcacttagcaggtttccgcgcagctttaacacgtggcttaaaccagtatctggaaaatgaaaatattctcaagaaagaaaaagtgaatgtgactggtgatgatgcgcgtgaaggtttaacagcgattatttctgttaaggttcctgatccaaaattctcgtctcagacaaaagaaaaattggtatcaagtgaggtaaaaccagcggtagagcaagcaatgaacaaagagttctctgcttacttacttgagaatccacaagctgcaaaatcaattgcaggcaagattattgatgctgcacgcgcacgtgatgctgcacgtaaagcacgtgaaatgacacgccgtaagagtgcattagatattgcaggtttgcctggtaaattggctgat

SEQ ID NO: 744 - DNA, 1430 bp, Artificial Sequence
  Acinetobacter baumanii
aacttgctctttcgtgagttcagtaaatgacttttcttgtatatggtagagtttaggtatcgaaactctgccattttttttatctatgttatattttccgcagtttttgaattttgactatttgaggcaaaccgcttggcgaccccattttggtacaacaccgcacttcatttattaaaaccgttttatcgttggcggataaaaagacgtgcagaaagtctagaattatatcagcaggaatgtctggaaagatttgggccatttgaagcaccgaagaatgtaaaagcgatctggtttcatgctgtttcagtcggggaaaccaatgctgcacagcccttaattgaatattacctaaaacttgggcagccagtcttagtgaccaataccacgaaaacaggtcaggctcgtgccaagtcactttttctaaaagaaccatatttagatttatttcaagccgtttatttgcctgtagaccagaagcctcttttaaaaaaattttttgagttatatcagccaaagcttttagcactggttgaaactgaactctggccaaatttaatcgatcaagccaaattacagcatgtaccttgtttgctgcttaatgctcggttgtcagaaaaatctgcaaaaggatatggcaaagtctcgggtttaaccgcaggtatgttaaaacagctggactgggtgttagctcaagatagtgcaactcgtcagcgttatgttgagcttggtttagacgaacacaaaagtcaggtcgttggtaatattaagtttgatattcatgcgccagaggcttttattaaacaagctgcccaattgcatcagcaatggtatctggaaaatcggcaggttgtgacgattgccagtacacatgcacccgaagaacaacaaattttggaagcactcgcaccttatttaaattcagatcgtgagttggtgtgtattgtggtgcctcgtcatcctgagcgtttcgatgaagtatttgaaatttgccaaaatttaaatttaattacgcatcgtagaagtatgggccaaagtattcatgccagcacgcaagtttatctcgctgacagtatgggtgagctctggttatggtatgccttaagtcaggtgtgttttgtaggcggttctttaaatgagccgggtggggggcataatattttagaacctatggttttaaatgtacctactgtagttggaccgcgttattttaactttcaaacgattgtcgatgagttcattgatgaaaatgctgtgcttattgctcaagatgcgcagcaggtcgttgatatctggttagcatgtctggcagaacttgaggcgactgaacagttagtgatacaggcgcataaagtgttgcaacgtaatcaaggttccttacaaaaacatatcggggtgattaatcgctatctggccgaaaaatcatgaat

SEQ ID NO: 745 - DNA, 3609 bp, Artificial Sequence
  Concatenation of C. jejuni genes
atgataggtgaagatatacaaagagtattagaagctagaaaattgattttagagatcaatttgggtggaactgctattggaacaggaattaattctcatcctgattatccgaaggttgtagaaagaaaaataagagaagtgacaggttttgaatatactgtggctgaggatttaatcgaggcgactcaagatacgggagcttatgtacaaatttcaggtgttttaaaacgtgttgcaacaaaactttctaaagtatgtaatgacttaagacttttaagtagtggtccaaaatgtggtcttaatgagattaatcttccaaaaatgcaaccaggtagttctatcatgccaggtaaggtaaatcctgttattcctgaagtagttaatcaagtttgttattttgttattggagcagacgtaactgtaacttttgcttgtgagggtggacaattacaacttaatgtttttgaaccagttgtannnnnnnnnnnnnnnnnnnnnnnnnnnnnnnnnnnnnnnnnnnnnnnnnngatccttttacggctgatcctactatcatagtattttgtgatgtgtatgatatttacaaaggacaaatgtatgaaaaatgtccaagaagcatagcaaaaaaagcaatagaacaccttaaaaatagtggcatagctgatactgcttactttggaccagaaaatgaattctttgtttttgatagtgtaaaaatagttgatactactcattgttctaagtatgaagttgataccgaagaaggagagtggaatgatgatagagaatttaccgatagctacaatactggacacaggccaagaaacaaaggtggatattttccagttcagccaattgattctttagtagatattcgttctgaaatggttcaaacccttgaaaaagtaggtcttaaaacttttgttcatcatcatgaagttgcacaaggacaagctgaaataggagtaaattttggcacgcttgtagaagcagctgacaatgttnnnnnnnnnnnnnnnnnnnnnnnnnnnnnnnnnnnnnnnnnnnnnnnnnngaaatgaaaaaacgttcttttatccatgaaggtatgcaccgtctttttgattctttccctgataatgctcatcctatggcagttttacaaggtgctgtttcatcgcttagtgctttttatcctgatcatttaaacatgaatgtaaaagaagaatatatggaaatggcagctagaatagtagctaaaatccctactatagtggccaccgcttatagatataaacacggctttcctatggcttatccaaatttagatcgtggttttacagaaaatttcttatatatgttaagaacctatccttacgatcatgtagagcttaaacctatagaagtaaaagcacttgatacagtttttatgcttcatgcagatcatgagcaaaatgcttcaacttcaacagttcgtnnnnnnnnnnnnnnnnnnnnnnnnnnnnnnnnnnnnnnnnnnnnnnnnnnggcggacatttaactcatggtgcaaaagtaagttcttcgggcaaaatgtatgaaagctgtttttacggcgtagaacttgatggaagaattgattatgaaaaagtaagagaaattgctaaaaaagaaaaaccaaaacttatagtttgtggagctagtgcttatgcaagagtgattgattttgctaaatttagagaaattgctgatgaaataggtgcctatctttttgctgatatagcacatattgcaggtcttgttgtggcaggtgagcatccaagtccttttccatacgctcatgtagtaagctcaactacacataaaactttgcgtggcccaagaggtggtattattatgacaaatgatgaagagcttgctaaaaaaattaattctgccatttttccaggtattcaaggtggtcctttgatgcatgtaattgctgcaaaagcagtaggatttaaatttaatcttagcgatgagtggaaagtttatgcaaaacaagtaagaactaatnnnnnnnnnnnnnnnnnnnnnnnnnnnnnnnnnnnnnnnnnnnnnnnnnnggacttaatatcaatgaaaattgtggagctttacatcctgcaaatttagctgctgaggtaaagcgtttgcgtgcagatgtgggctttgcttttgatggtgatgcagatcgtttggtggttgtagatgaaaaaggcgaagtggctaatggggatagtttattgggcgtattggcactttatcttaaagaacaaggtaaattacaatcaagtgttgtggctactataatgagtaatggtgctttaaaagagtttttaaataaacatggtatagaacttgatacttgtaatgttggcgataaatatgtgcttgaaaaactaaaagccaatggtggaaattttggtggagaacaaagtgggcatattatttttagcgattatgcaaaaactggagatggtttgatagccgccttgcaatttagtgctttaatgctttctaagaaaaaatctgcaagttctattttgggtcaagttaaaccttatcctcagcttttaaccaatnnnnnnnnnnnnnnnnnnnnnnnnnnnnnnnnnnnnnnnnnnnnnnnnnnttacatttaagcggctatgacttaagcttagaagatcttaaaaatttccgccaacttcattctaaaacccctggacaccctgaaatttcaactcttggagtagaaatcgctacaggccctttaggacaaggcgttgccaatgctgtaggctttgctatggcagcaaaaaaagcacaaaatttgctaggcagtgatttaatcgatcataaaatttattgtctttgcggagatggggatttacaagaaggcatttcttatgaagcttgttctttagcaggacttcacaaacttgataacttcatacttatttatgatagcaacaatatctctatagaaggcgatgtaggtttagcctttaacgaaaatgtaaaaatgcgttttgaagcacaaggatttgaagttttaagtataaatggacacgattatgaagaaatcaataaagccttagaacaagctaaannnnnnnnnnnnnnnnnnnnnnnnnnnnnnnnnnnnnnnnnnnnnnnnnngccggtgcttcagatcctgcggctttacagtacttagctccatatactggtgtaaccatgggtgaattttttagagacaacgcaaaacatgctttaattgtttatgatgatttgagcaagcatgctgtagcttatcgcgaaatgtctttgattttacgtcgtcctccaggtcgtgaagcttatccaggtgatgttttttaccttcattcaagattgcttgaaagagcaagcaagctaaatgatgaattaggtgctggttctttgacggcattgccgataattgaaacacaagcaggagatgtttctgcttatattccaactaatgttatttcaattacagatggacaaattttcttagaaactgatttatttaactcaggaattcgtcctgcaattaatgttggtttatcagtatctcgtgtaggtggggctgctcaaattaaagctacaaaacaagtttcaggcacattaagacttgaccttgctcaa

SEQ ID NO: 746 - DNA, 1214 bp, Artificial Sequence
  Bordetella pertussis
ttcgggatgggcatgggcgtcgcacacgacgtcgaacaccgtgccggtttcatccagtgcgggttcctgctcgaccgtggccacgcgcaggcccgaactgcgggcgatgtcgccgtcgtcgggctgcgtgcggccatcgagcaggcgcagcagcgaagatttgcccgcgccgttgcgcccgatcaggccgatgcgctcgccttcctggatggcgaaatcggcgtggtccagcaaggggtggtggccgtaggccagttgaacgtcggtaagcgtaataagggaagcgagggccatggacagacagagtgatgcgtattggcctgtattgtcgcaggaacgcgcggggcgggcagcgcgcggccgggagccggggcgccgactgtactaaaatgccgctcgcagggcagatcgggcaatcgcgggggatgcaaatccttcgaggaaggtccggactccacagggcgggatagcggctaacggccgtccggcgacgctggcgggcttgcccgccggaaaagccgaggaacagggccacagagacgagtctgtcatgagggcgcgcctggcgcgcaccggcacggccatctccgtgccgcgccgtccggaaacgggcggcggcatgacagggtgaaacgcggcaacctctatccggagcaacatcaaataggcatgcgtacggccgtaaggccgggaagggcggctccgtccaagcatgcgggtaggtggctggagcggtccagcaatggttcgccaagaggaatgattgcccgccggggaaacccggcgtacagaatccggcctatagatctgctctgcactgcattttcatgacagccggccggaatccggccggctggcttacggcgccctacgtaaaacaatgaaagcacgcgcccaggccctacttctgatgaatgacatttaatagtcggcgcaagctgctgatttatcagcaagttttccgcgcgccgtttttacaattggctctaagtccttgttgtgattgaaaaaattgcaaccacggccttgacccatgaagttgcttcccgtagagtgggaaaaagtaggaatttgtgcaactaagtggggcaaacgggtgttccagggaagcagcgcactcacgttggatgctaaggggcggatctcgattccgacccggcatcgtgacgcgctcatggaccgtgccgaaggccggttgaccctgacccgtcatc

SEQ ID NO: 747 - DNA, 925 bp, Artificial Sequence
  Burkholderia mallei
gaccgatccggcaagccgcggcttcgcgccgctgccggtgctgggcgtgcccggctggtggccggcgaacgcgtcgccggccttctacgacgatccgcaggtgtttcgccgcggccgccgacgcgcaccggatgtcctcgcccgtgccgcgtgacggacgacgcttgcctcggcgcgcccgcgcggcgccgcgcaccgccggtgcgcgctttcgcgcaagccgccgccgcggcaagggcgtcgcgccgagcggatgccgctgtacggtgctagaatcccgctcgcaaagcaggctaggcagtcgcggctttcgcggttcgccgcgaagggcgaggaaagtccggactccaacagggcagggtgatggctaacggccatccgtggcgacacgcggaacagggcaacagaaagcaaaccgccgatggcccggcgcaagccgggatcaggcaagggtgaaacggtgcggtaagagcgcaccgcggctgcggcgacgcagaccggcacggtaacctccacccggagcaattccaagtaggcggacgcgcatcttcggatgcaggacggtgcccccgtctcgttcgcgggtaggaagcttgagcgcgtcagcaatggcgcgcctagaggaatggctgccacgggccgcgcgccttcgggcgtgcggttcgcacagaatccggcttatcggcccgctttgccgcccgatgacgaaaggccggcgcccgatgcggcgccggccttttttccgtttcgcggtccgcgcgcacggccgcttgacggacgaagcgcgttacgcggcgacgatttcgaacgaatgcgacagctcggccgtcttggcgatcatgatcgacgccgaacagtatttgtcgtgcgacagattgatcgcgcgctcgacggtcgcggggttcaggttgcggcccgtcaccgt

SEQ ID NO: 748 - DNA, 713 bp, Artificial Sequence
  Bacillus subtilis
gttcttaacgttcgggtaatcgctgcagatcttgaatctgtagaggaaagtccatgctcgcacggtgctgagatgcccgtagtgttcgtgcctagcgaagtcataagctagggcagtctttagaggctgacggcaggaaaaaagcctacgtcttcggatatggctgagtatccttgaaagtgccacagtgacgaagtctcactagaaatggtgagagtggaacgcggtaaacccctcgagcgagaaacccaaattttggtaggggaaccttcttaacggaattcaacggagagaaggacagaatgctttctgtagatagatgattgccgcctgagtacgaggtgatgagccgtttgcagtacgatggaacaaaacatggcttacagaacgttagaccacttacatttaaaatgatgaaaacaagctctcccgtataaggagagcttttatcttgaaaagagaaaagttttaaaagacagggtgatacgatgaagaagtatacactaattgcaacggcgccgatgggcattgaagctgttgtcgcaaaggaagtacgagatttaggatacgaatgcaaggttgataacgggaaagttatttttgaaggtgatgcacttgccatctgccgtgcgaacctttggcttagaacagccgaccgcataaaggttcaggttgcttcttttaaagcgaaaacatttgatgaactgtttgaa

SEQ ID NO: 749 - DNA, 828 bp, Artificial Sequence
  Clostridium perfringens
aaaacaagttctttttcatataatgatgtataagtaatacttagggtggagaattatgtggtccaattttggaaatagatttaatggagactgctttaaagacttaaggagagatttcaatagattaatgagaaattttaagagaaatgcttgtaaaagatgtcttattaataattgctatttcagaaatgccttaaagtggggggctgtaggtggcatattaaccttccttataataagtcaaataggagttcctttagcaattgtttgtattggaatagtagcaatatttgtgatttgtaataaatggtagaaaaaataatttgaaaaaaataagtatatatgttaatattaatcttgcgagtaagccagacaatcgctgctagtcttagaactaggagaggaaagtccgagctccatagggcaggatgctggataacgtccagtggaggtgactctaaggatagtgcaacagaaataaaccgcctagatttatctaggtaagggtggaaaggtgaggtaagagctcaccagggtataggtgactatactgctatgtaaaccccatctggagcaagaccaaataggaggacatataggggctgcccgtcccgtcctcgggtgtgtcgcttgagcctatcggcaacggtaggcctagatagatgattgtcaaatacagaactcggcttatagacttatctcgtatttataaaacactaggttataatacctaagtgtttttttattttacaaaaaaatatactgatagtttctttccttattaactttaatcttacagtattaatttaattttattggatatatcta

SEQ ID NO: 750 - DNA, 777 bp, Artificial Sequence
  Escherichia coli
gaagctgaccagacagtcgccgcttcgtcgtcgtcctcttcgggggagacgggcggaggggaggaaagtccgggctccatagggcagggtgccaggtaacgcctgggggggaaacccacgaccagtgcaacagagagcaaaccgccgatggcccgcgcaagcgggatcaggtaagggtgaaagggtgcggtaagagcgcaccgcgcggctggtaacagtccgtggcacggtaaactccacccggagcaaggccaaataggggttcataaggtacggcccgtactgaacccgggtaggctgcttgagccagtgagcgattgctggcctagatgaatgactgtccacgacagaacccggcttatcggtcagtttcacctgatttacgtaaaaacccgcttcggcgggtttttgcttttggaggggcagaaagatgaatgactgtccacgacgctatacccaaaagaaagcggcttatcggtcagtttcacctggtttacgtaaaaacccgcttcggcgggtttttgcttttggaggggcagaaagatgaatgactgtccacgacactatacccaaaagaaagcggcttatcggtcagtttcacctgttttacgtaaaaacccgcttcggcgggtttttacttttggaggggcagaaagatgaatgactgtccacgacactatacccaaaagaaagcggcttatcggtcagttttacctgatgtacgtaataaaccgttccggcgggtttcagattgttgagtgcgctttattcatgccg

SEQ ID NO: 751 - DNA, 834 bp, Artificial Sequence
  Rickettsia prowazekii
taataaattaatttattcatatcaaagtttgctaatagtgtatatgtttttaagtactacattatttatacattaggaaaaaaatagtagcaatgttaaattaagatatctttctttaaagaattgacattatggaatttgttattataattgtaattatattgtgtgtacaataattacaataaattttcccctcagaacctaacaagctaattgaaattcttttaacatattattgactaatttaagaaaagctaccataatctaaatggtcgtgcagttgcgtgatgataatcacgaggaaagtccggactctatagaggtatggtgccggttaacatccggcagagtattattactttagggctagtaccacagaaaatataccgccgagtatttcggtaagggtgaaaaggtgtggtaagagcacaccggtaagttggcaacaagttacgcatggttaaccccaccaagagcaagatcaaataggcattacagaatttaaatatttatttaagttcctgggttacctctaatcggattgtaatgcgggtagatcgcttgaggtaaacggtaacgtttatcctagataaataactgcaatgaattaatattcatacagaatccggcttatagaccagatgagcaggtattacatgtgttaataccgcagagttattgcgagtaactgaaaaaagtatggcaatctagaaaaataatcagattctgtggatttttagtgttccctcgcaatgacgaaaataatacacgtatagattacagcatgagatgatactaatacttactgtaacataatttaatgaaaaagttata

SEQ ID NO: 752 - DNA, 783 bp, Artificial Sequence
  Staphylococcus aureus
tgatattttgggtaatcgctatattatatagaggaaagtccatgctcacacagtctgagatgattgtagtgttcgtgcttgatgaaacaataaatcaaggcattaatttgacggcaatgaaatatcctaagtctttcgatatggatagagtaatttgaaagtgccacagtgacgtagcttttatagaaatataaaaggtggaacgcggtaaacccctcgagtgagcaatccaaatttggtaggagcacttgtttaacggaattcaacgtataaacgagacacacttcgcgaaatgaagtggtgtagacagatggttatcacctgagtaccagtgtgactagtgcacgtgatgagtacgatggaacagaacatggcttatagaaatatcactactagtttagctctcctagatgatggagagcttttttcatgaaaagaacacttaaaattaacaccttgtcttgatataatgacactgccttgttttaaaatagtaagcggatgcgttaatgtatcagcgattaaatttgttggaaatgtataaaaaacacaagctaagaataaaatacctgtataaaaggagaatcatatatgtttcaattacttgcagtttgtccgatgggattagaagctgttgttgctagggaaattcaagaattaggctatgaaacaaatgttgaaaatggtcgtatattttttgaaggagacgcaagtgcaattgtaaaggcaaatttatggttgcgcacagcagaccgaatcaaaattgttgttggacgttttaac

SEQ ID NO: 753 - DNA, 1086 bp, Artificial Sequence
  Vibrio cholerae
gtgaaatcggtctggcgatcgagatgggctgtgatgcagaagacatcgcactgaccattcatgctcacccaacgctgcatgagtctgtgggtctggcggctgaagtattcgaaggcaccatcactgacctgccaaacgccaaagcgaaaaagaaaaagtaatttctgattcactggtttggttaaaaagtgtttgattgaagaaaccgctgcttgcagcggttttttttcgcttttgattcccaattgaatgcaaagtaagcagttaaaccgagatttaacgcgctggcggattgtttctggactctgctcacacttctcgctacaatccgccgcggagttgactgggtagtcgctgccttattgacgtcccttggcctatcgccagggagactgataagggggaggaaagtccgggctccatagagcagggtgccaggtaacgcctggggggcgcaagcctacgacaagtgcaacagagagcaaaccgccgatggcctctccttcgggatgggatcaggtaagggtgaaagggtgcggtaagagcgcaccgtgcgactggcaacagttcgtagcagggtaaactccacccggagcaagaccaaataggcctccacatagcgttgctcgcgttaggaggcgggtaggttgcttgagccagtgagtgattgctggcctagaggaatggctactaccgcgcaagcggaacagaacccggcttatacgtcgactccacctattgcagacccatcatagccttgtgttatggtgggttttttgctttttgctgactcaggtaacagcaaaaccattaaacttagcgcaaagtcacgtcctgtatacgcttatggctagcggtagtgtctgaaatcggtacactaacccatatgacgggcatgcccagcacaacgagaaaatcatgactgcttctttccaacacatctctgtattattgaacgaatcgattgaaggattggcgatcaagccggatggtatctacatcgatggcacctttggccgcggtggtcacagtcgcaccattctcgctcagttaggcccagaaggtcgtctgtacagcattgaccgt

SEQ ID NO: 754 - DNA, 369 bp, Artificial Sequence
  Coxiella burnetii
gagttaaccggagtatccatcgtgacttaccaacacatcaaagttcccagccaaggtgaaaaaatcaccgttaataaagccgttttagaagttcctgaccgacccattattccctttatcgaaggagatgggattggcattgatatcgcgcccgtcatgaaaaacgtggtcgatgccgccgtggaaaaatcctacgctggaaagcgaaaaattgaatggatggagatctacgccggagaaaaggctacgaaagtgtatggcaaagacaattggctgcctgatgagacactcgaagccattaaagaataccaagtggccattaaaggtcccttaaccacgccggtggggggtggcatacgttcgctcaat

SEQ ID NO: 755 - DNA, 1317 bp, Artificial Sequence
  Acinetobacter baumannii
attacgacggaaattgtaagtaattttgtcaataatttacttgattaaaattatcaagcacttggaaagtctatcaagtgtttgtatgattcaaatgtgaatagcttaaaaataatactggggtaaaaaaatatctcaggggccaataaatttaggctgagcttgaacaacaattgttatctctggaggatatccatgaaattgagtcgtattgcacttgctactatgcttgttgctgctccattagctgctgctaatgctggcgtaacagttactccattattgcttggttacactttccaagacagccaacacaacaatggcggtaaagatggtaacttaactaacggtcctgagttacaagacgatttattcgttggcgcagctcttggtatcgagttaactccatggttaggtttcgaagctgaatataaccaagttaaaggcgacgtagacggcgcttctgctggtgctgaatataaacaaaaacaaatcaacggtaacttctatgttacttctgatttaattactaaaaactacgacagcaaaatcaagccgtacgtattattaggtgctggtcactataaatacgactttgatggcgtaaaccgtggtacacgtggtacttctgaagaaggtactttaggtaacgctggtgttggtgctttctggcgcttaaacgacgctttatctcttcgtactgaagctcgtgctacttataatgctgatgaagagttctggaactatacagctcttgctggcttaaacgtagttcttggtggtcacttgaagcctgctgctcctgtagtagaagttgctccagttgaaccaactccagttgctccacaaccacaagagttaactgaagaccttaacatggaacttcgtgtgttctttgatactaacaaatcaaacatcaaagaccaatacaagccagaaattgctaaagttgctgaaaaattatctgaataccctaacgctactgcacgtatcgaaggtcacacagataacactggtccacgtaagttgaacgaacgtttatctttagctcgtgctaactctgttaaatcagctcttgtaaacgaatacaacgttgatgcttctcgtttgtctactcaaggtttcgcttgggatcaaccgattgctgacaacaaaactaaagaaggtcgtgctatgaaccgtcgtgtattcgcgacaatcactggtagccgtactgtagtagttcaacctggtcaagaagcggcagctcctgcagcagctcaataatttgagttcttgaacagtaaaaaagcgactcgttagagtcgcttttttatg

SEQ ID NO: 756 - DNA, 4932 bp, Artificial Sequence
  Rickettsia prowazekii
atggctcaaaaaccaaattttctaaaaaaaataatttccgcaggattggtaactgcttccacggctactatagtagctggtttctctggtgtagcaatgggtgctgctatgcaatataataggacaacaaatgcagcagctacaacctttgatggtataggctttgatcaagctgctggtgctaatattcctgtcgctccaaattcagttattactgctaatgctaataatcctattacttttaatactccaaacggtcatttaaatagtttatttttggatactgcaaatgatttagcagtaacaattaatgaggatactaccttaggatttataacaaatattgctcagcaggctaagttctttaattttactgttgctgctggtaaaattcttaacataacagggcagggtattactgttcaagaagcttctaatacaataaatgctcaaaatgctcttacaaaagtgcatggtggcgctgctattaacgctaatgatcttagcgggctaggatcaataacctttgctgctgcgccttctgtattagaatttaatttaataaatcctacaactcaagaagctcctcttacacttggtgctaattctaaaatagttaatggtggtaatgggacattaaatattactaatggatttattcaggtttcagataacacttttgctggtattaagaccattaatatcgatgattgtcaaggtttaatgtttaattctactcctgatgccgctaatactttaaatttacaagtaggtggtaatactattaattttaatggaatagacggtactggtaaattagtattagtcagtaagaatggtgctgctaccgaatttaatgttacaggaactttaggtggtaatctaaaaggtattattgaattgaacactgcagcagtagctggtaaacttatctctcaaggaggtgctgctaatgcagtaataggtacagataatggagcaggtagagctgcaggatttattgttagtgttgataatggtaatgcagcaacaatttctggacaagtttatgctaaaaacatggtgatacaaagtgctaatgcaggtggacaagtcacttttgaacacatagttgatgttggtttaggcggtaccaccaactttaaaactgcagattctaaagttataataacagaaaactcaaactttggttctactaattttggtaatcttgacacacagattgtagtccctgatactaagattcttaaaggtaacttcataggtgatgtaaaaaataacggtaatactgcaggtgtgattacttttaatgctaatggtgctttagtaagtgctagtactgatccaaatattgcagtaacaaatattaatgcaattgaagcagaaggggccggggttgtagaattatcaggaatacatattgcagaattacgtttagggaatggtggctctatctttaaacttgctgatggcacagtaattaatggtccagttaaccaaaatgctcttatgaataataatgctcttgcagctggttctattcagttagatgggagtgctataattaccggtgatataggtaacggtggtgttaatgctgcgttacaacacattactttagctaacgatgcttcaaaaatattagcactcgatggcgcaaatattatcggggctaatgttggtggtgcaattcattttcaagctaacggtggtactattaaattaacaaatactcaaaataatattgtagttaattttgatttagatataactactgataaaacaggtgttgttgatgcaagtagtttaacaaataatcaaactttaactattaatggtagtatcggtactgttgtagctaatactaaaacacttgcacaattaaacatcgggtcaagtaaaacaatattaaatgctggcgatgtcgctattaacgagttagttatagaaaataatggttcagtacaacttaatcacaatacttacttaataacaaaaactatcaatgctgcaaaccaaggtcaaataatcgttgccgctgatcctcttaatactaatactactcttgctgatggtacaaatttaggtagtgcagaaaatccactttctactattcattttgccactaaagctgctaatgctgactctatattaaatgtaggtaaaggagtaaatttatatgctaataatattactactaacgatgctaatgtaggttctttacactttaggtctggtggtacaagtatagtaagtggtacagttggtggacagcaaggtcataagcttaataatttaatattagataatggtactactgttaagtttttaggtgatacaacatttaatggtggtactaaaattgaaggtaaatccatcttgcaaattagcaataattatactactgatcatgttgaatctgctgataatactggtacattagaatttgttaacactgatcctataaccgtaacattaaataaacaaggtgcttattttggtgttttaaaacaagtaattatttctggtccaggtaacatagtatttaatgagataggtaatgtaggaattgtacatggtatagcagctaattcaatttcttttgaaaatgcaagtttaggtacatctttattcttacctagtggtactccattagatgttttaacaattaaaagtaccgtaggtaatggtacagtagataattttaatgctcctattgtagttgtatcaggtattgatagtatgatcaataacggtcaaatcatcggtgataaaaagaatattatagctctatcgcttggaagtgataacagtattactgttaatgctaatacattatattcaggtatcagaactacaaaaaataatcaaggtactgtgacacttagtggtggtatgcctaataatcctggtacaatttatggtttaggtttagagaatggtagtccaaagttaaaacaagtgacatttactacagattataacaacttaggtagtattattgcaaataatgtaacaattaatgattatgtaactcttactacaggaggtatagcagggacagattttgacgctaaaattactcttggaagtgttaacggtaacgctaacgtaaggtttgttgatagtacattttctgatcctagaagtatgattgttgctactcaagctaataagggtactgtaacttatttaggtaatgcattagttagtaatatcggtagtttagatactcctgtagcttctgttagatttacaggtaatgatagtggggcaggattacaaggcaatatttattcacaaaatatagattttggtacttataatttaactattctaaattctaatgtcattttaggtggtggtactactgctattaatggtgaaatcgatcttctgacaaataatttaatatttgcaaatggtacttcaacatggggtgataatacttctattagtacaacgttaaatgtatcaagcggtaatataggtcaagtagtcattgccgaagatgctcaagttaacgcaacaactacaggaactacaaccattaaaatacaagataatgctaatgcaaatttcagtggcacacaagcttatactttaattcaaggtggtgctagatttaatggtactttaggagctcctaactttgctgtaacaggaagtaatattttcgtaaaatatgaactaatacgtgattctaaccaggattatgtattaacacgtactaacgatgtattaaacgtagttacaacagctgttggaaatagtgcaattgcaaatgcacctggtgtaagtcagaacatttctagatgcttagaatcaacaaatacagcagcttataataatatgcttttagctaaagatccttctgatgttgcaacatttgtaggagctattgctacagatacaagtgcggctgtaactacagtaaacttaaatgatacacaaaaaactcaagatctacttagtaataggctaggtacacttagatatctaagtaatgctgaaacttctgatgttgctggatctgcaacaggtgcagtgtcttcaggtgatgaagcggaagtatcttatggtgtatgggctaaacctttctataacattgcagaacaagacaaaaaaggtggtatagctggttataaagcaaaaactactggggttgtagttggtttagatactctcgctagcgataacctaatgattggggcagctattgggatcactaaaactgatataaaacaccaagattataagaaaggtgataaaactgatattaatggtttatcattctctctatatggttcccaacagcttgttaagaatttctttgctcaaggtaatgcaatctttaccttaaacaaagtcaaaagtaaaagtcagcgttacttcttcgagtctaatggtaagatgagcaagcaaattgctgctggtaattacgataacatgacatttggtggtaatttaatatttggttatgattataatgcaatgccaaatgtattagtaactccaatggcaggacttagctacttaaaatcttctaatgaaaattataaagaaaccggtacaacagttgcaaataagcgcattaatagcaaatttagtgatagagtcgatttaatagtaggggctaaagtagctggtagtactgtgaatataactgatattgtgatatatccggaaattcattcttttgtggtgcacaaagtaaatggtaaattatctaactctcagtctatgttagatggacaaactgctccatttatcagtcaacctgatagaactgctaaaacgtcttataatataggcttaagtgcaaacataaaatctgatgctaagatggagtatggtatcggttatgattttaattctgcaagtaaatatactgcacatcaaggtactttaaaagtacgtgtaaacttctaa

SEQ ID NO: 757 - DNA, 1311 bp, Artificial Sequence
  Rickettsia prowazekii
atgactaatggcaataataataacttagaatttgcagaattaaaaattagaggtaaactatttaagttacctatacttaaagcaagtatcggtaaagatgtaatcgatataagtagggtatctgcggaagccgattactttacttatgatccgggttttatgtctactgcttcttgtcaatctactatcacatatatagacggtgataaaggcatattatggtatcgaggatatgatattaaagacttagctgagaaaagtgattttttagaagtggcatatttgatgatttatggggagctaccaagtagtgatcagtattgtaattttactaaaaaggttgctcatcattcattagtgaatgaaagattacactatttatttcaaaccttttgtagttcttctcatcctatggctattatgcttgcagctgttggttctctttcagcattctatcctgatttattaaattttaatgaaacagactatgaacttaccgctattagaatgattgctaagatacctactatcgctgcaatgtcttataaatattctatagggcaaccgtttatttatcctgataattcattagattttaccgaaaattttctacatatgatgtttgcaactccttgtactaaatataaagtaaatccaataataaaaaatgctcttaataagatatttatcttacatgcagaccatgagcagaatgcttctacttcaacagttcggattgctggctcatcaggagctaatccttttgcatgtattagcactggtattgcatcactttgggggcctgctcacggcggggctaatgaagcagtgataaatatgcttaaagaaattggcagttctgagaatattcctaaatatgtagctaaagctaaagataagaatgatccatttaggttaatgggttttggtcatcgagtatataaaagctatgacccgcgtgccgcagtacttaaagaaacttgtaaagaagtattaaatgaattaggtcagttagacaataatccgctgttacaaatagcaatagaacttgaagctctcgctcttaaagatgaatattttattgaaagaaaattatatccaaatgttgatttttattcaggcattatctataaagctatgggtataccgtcgcaaatgttcactgtactttttgcaatagcaagaaccgtaggttggatggcacaatggaaagaaatgcacgaagatcctgaacaaaaaatcagtagacctagacagctttacactggttatgtacatagagagtataagtgtattgtagaaagaaagtga

SEQ ID NO: 758 - DNA, 882 bp, Artificial Sequence
  Vibrio cholerae
atgttcggattaggacacaactcaaaagagatatcgatgagtcatattggtactaaattcattcttgctgaaaaatttacctttgatcccctaagcaatactctgattgacaaagaagatagtgaagagatcattcgattaggcagcaacgaaagccgaattctttggctgctggcccaacgtccaaacgaggtgatttctcgcaatgatttgcatgactttgtttggcgagagcaaggttttgaagtcgatgattccagcttaacccaagccatttcgactctgcgcaaaatgctcaaagattcgacaaagtccccacaatacgtcaaaacggttccgaaacgcggttaccaattgatcgcccgagtggaaacggttgaagaagagatggctcgcgaaagcgaagctgctcatgacatctctcagccagaatctgtcaatgaatacgcagagtcaagcagtgtgccttcatcagccactgtagtgaacacaccgcagccagccaatgttgtgacgaataaatcggctccaaacttggggaatcgactgcttattctgatagcggtcttacttcccctcgcagtattactgctcactaacccgagccaaaccagctttaaacccctaacggttgtcgatggcgtagccgtcaatatgccgaataaccaccctgatctttcaaactggctaccgtcaatcgaactgtgcgttaaaaaatacaatgaaaagcatactggtgggctcaagccgatagaagtcattgccacaggtggacaaaataaccagttaacgctgaattacattcacagccctgaagtttcaggggaaaacataaccttacgcatcgttgctaaccctaacgatgccatcaaagtgtgtgag

SEQ ID NO: 759 - DNA, 1095 bp, Artificial Sequence
  Francisella tularensis
atgcttaaagttggttttattggttggcgcggaatggtcggctcagttttaatgtctcgtatgatcgaatcaaaagattttgattgtattttgccaacgtttttttcgacatctcaggtagggcagctgccaacaggttttatgcaacaatatggagcgttacaagatgcctatagtatcgaccaactaagtagtatggatatacttctaagttgccaaggtggtgaatataccaaagaaatacaccacaaattaagagaagccggctggcaaggtttctggatagacgctgcatcgacactacgcttagacaaagatagtactctagttctagaccctctaaatcacgatcaaataattaatgctattgataatggtaaaaaagattttatcggtagtaattgtactgttagtctaatgtcactagctatagctggactactcaaagaagatcttgttgaatgggttaactctagtacttatcaagcaatttcaggagcgggtgccgcagcaatgcaagaactacttcaacaaacaagccttttaagcaaaattgataatagagatgaagatattctaattagagaaaaaattctcagagaattatcaaaagactcatcaaaaatccctcaacaaaaaactgtacaaactttggcttataatctattaccttggatagatgttggtatgcctagtggacaaacaaaagaagagtacaaagcagctacagaacttaataaaattctagatactaaaaaaacaatccctgtcgatggtatatgtgtcagagtaccaagtctaagatcacactctcaagcattaacagtcaaacttagacagaaattaacaattgaagaaattaagcaaaaaatatctcaaggtaatgaatgggttaaagtaatagataataacaaagaagatactttaaaatacctaacacctcaagctaattcaggaactcttgatattgctataggtcgtatcaaatcatcgttattagctgatgatatatttcattgtttcacagtaggtgatcagctattatggggagctgctgagccccttagaagagttttaaatattattaaaatataa

SEQ ID NO: 760 - DNA, 1020 bp, Artificial Sequence
  Francisella tularensis
atgaataaaaaaatcttagtaacaggtggtgtaggctatataggtagtcatacagtggtagaacttcttgatagagattatcaagttgtggtggtagataatctttcaaatagcaaagtatctgtaatagacagggttaaaaaaatcacaaataaagattttgatttttatcagctagaccttttaggtaaagctaagctaacaaaagtttttcaagagtatgatatttatgctgtaattcattttgctggctttaaagctgtaggtgagagtgttgaaaaaccgttagagtattatcataacaatatccaaggtacactaaacttacttgagctaatgcaagagtataaagtttataattttgtctttagttcatcggcgactgtatatgggatgaataataaaccaccctttacagaagatatgcctctaagtacaactaacccatacggtgcaactaagctaatgttagaagacattttgcgagatttgcaaaatgctaataataattttaatattacatgtcttagatattttaatccagtcggcgcccatagtagtgggatgataggagaggatccacagggtatacctaataacctcatgccttatgtcgcgcaagtaggtgctggtaaactagctaaacttagtatctttggtggtgactatgagactatagatggtacaggagtgagagactatatacatgttgtagatttagcaataggtcatatattagcgttagaaaaattatcacaagataagcctagctggagagcttataatcttggttctggaaatggctattctgtattagagattgtcaaagcttatcaaaaagccctaggtaaagagattccatatcagatagtagctaggagagccggtgatattgcagcgagttttgctgatgttgccaaggctaaaagagagttgggttttgagacacaaaagactatagatgatatttgtgatgatatgcttaaatggcaaaagtacgcaaaagagaataatatctag

SEQ ID NO: 761 - DNA, 840 bp, Artificial Sequence
  Shigella flexneri
cgccccctggctgatgccgtgacagcatggttcccggaaaacaaacaatctgatgtatcacagatatggcatgcttttgaacatgaagagcacgccaacaccttttccgcgttccttgaccgcctttccgataccgtctctgcacgcaatacctccggattccgtgaacaggtcgctgcatggctggaaaaactcagtgcctctgcggagcttcgacagcagtctttcgctgttgctgctgatgccactgagagctgtgaggaccgtgtcgcgctcacatggaacaatctccggaaaaccctcctggtccatcaggcatcagaaggccttttcgataatgataccggcgctctgctctccctgggcagggaaatgttccgcctcgaaattctggaggacattgcccgggataaagtcagaactctccattttgtggatgagatagaagtctacctggccttccagaccatgctcgcagagaaacttcagctctccactgccgtgaaggaaatgcgtttctatggcgtgtcgggagtgacagcaaatgacctccgcactgccgaagccatggtcagaagccgtgaagagaatgaatttacggactggttctccctctggggaccatggcatgctgtactgaagcgtacggaagctgaccgctgggcgcaggcagaagagcagaaatatgagatgctggagaatgagtaccctcagagggtggctgaccggctgaaagcatcaggtctgagcggtgatgcggatgcggagagggaagccggtgcacaggtgatgcgtgagactgaacagcagatttaccgtcagctgactgacgaggtactg

SEQ ID NO: 762 - DNA, 503 bp, Artificial Sequence
  Campylobacter jejuni
aaaaacttaaaaaaagctgttttttacttgatattgttttttaaatatgctaaaattaggcgtttcaattaaaacaaaggagcttttatgactaaagcagatttcatttcattagttgctcaaacagctgggctaacaaaaaaagacgctactactgctactgatgcagttatttctactattactgatgttttagctaaaggtgatagcatcagttttattggttttggtactttttcaactcaagaaagagctgctagagaagctagagtaccaagcacaggaaaaacaatcaaagttcctgctacaagagttgcaaaatttaaggtaggtaaaaaccttaaagaagctgttgcaaaagcaagcggcaaaaagaaaaaataaaacttcggctagataaattctagccttctttcttaattaattcagatattttgtatcttttatttactttatttttttaaacttttataaaactatcattattaaacaaaaaaggatat

SEQ ID NO: 763 - DNA, 2118 bp, Artificial Sequence
  Concatenation of A. baumannii genes
cgcgcggtaaaactaaagaagaagatatagcattagaaaaagatttgctgtctgatgaaaaagagattgctgaacatttaatgctgattgatcttgggcgaaacgatgtagggcgtgtatcgaaaataggtaaagtccaagtcacggatcaaatggtgatcgagcgttattcacatgtcatgcatattgtttcaaatgtacaaggtgaagtgcgtgatgatatcgatgcacttgatgtatttaaagccacctttccagcaggaacgttatcaggtgccccaaaaattcgtgcaatggaaattattgatgaagtagaacctgtgaaaaggggagtttttggcggggctgttggttatttgggatggcatggtgaaatggatatgtcgattgcaatccgtacttgtgttatccgtgataaaaaggtgtatgtacaggctggtgcagggnnnnnnnnnnnnnnnnnnnnnnnnnnnnnnnnnnnnnnnnggaatctggcggtttagtttcagatgaactcattatcggtttagtaaaagaacgtattgctcaacctgactgcgtgaatggttgtattttcgacggcttcccacgcactattcctcaagcagaagctttggaaaaagaagggatcagcattgatcatgtaattgaaattgatgtacctgatgaagaaatcgtaaaacgtctttctggtcgtcgtcagcatcctgcttctggtcgtgtttatcacgttgtatacaatccacctaaagtggaaggtaaagatgatgtcacaggnnnnnnnnnnnnnnnnnnnnnnnnnnnnnnnnnnnnnnnncgttcaaccgtgtaaaattacgtaaccttaaaactggtaaagttttagaaaaaacttttaaatctggtgatactttagaagctgctgacatcgtagaagtagaaatgaactacctatacaacgatggcgaaatgtggcacttcatggacccagaaagcttcgaacaaattgcagctgacaaaactgcaatgggtgatgctgctaaatggttaaaagacgactcaaatgaaacatgtacaatcatgttattcaacggcgttcctttaaacgtaaatgcacctaacttcgttgtattgaaagttgttgaaactgatccgggcgtacgtggtgatacttctggtggtnnnnnnnnnnnnnnnnnnnnnnnnnnnnnnnnnnnnnnnntcgtgcccgyaatttgcataaagctgccggccttgtagcacagcaaggcaaatttcctgaaactctagaagaatggattgcactacccggcattggtcgctcgaccgcaggtgcactcatgtctttaggtttacgtcagtatggcgtgattatggatggcaacgtgaaacgcgttttagcccgtttctttgccattgaagatgacttaagcaaaccacagcacgaacgtgaaatgtggaaactggctgaagagctttgtcccacccaacgcaatcatgactacactcaagcgannnnnnnnnnnnnnnnnnnnnnnnnnnnnnnnnnnnnnnnttaaaaacactagcggtaagcttaaacaagattgccaatgatattcgttggttagcaagtggtccacgttgcggcttcggcgaaatccgtattcctgaaaatgaacctggttcaagtatcatgccaggtaaagtgaacccgactcaaagtgaagccatgaccatggttgttgctcaagtacttggcaacgataccactattaatgtcgctggtgcttctggtaacttcgagctcaatgtatttatgccagtgattgcttataacttactgcaatctattcagttgcttggtgatgcatgtaatagttttaatgatcactgtgcagtagggatcgagccaaatcgtgagaaaattgatcatttcttgcataattctcttatgttagttacggcannnnnnnnnnnnnnnnnnnnnnnnnnnnnnnnnnnnnnnncccggttatgtaccaaatactttgtctgaagatggtgacccattagacgtacttgttgtaactccacatcctgttgctgccggttctgtaattcgttgccgcccagtgggcaaattaaacatggaagacgacggtggtatcgatgccnnnn

SEQ ID NO: 764 - DNA, 276 bp, Acinetobacter baumannii
atgagcgagctaggcttaaaaagcagtggcaagccaaaaaaatcagcgcgtacagtgggtgatgtacttggtaaataccacccacatggtgactcggcatgttatgaagccatggtactcatggctcagccatttagttaccgctatcctttaatcgaaggtcaggggaactggggttcacctgatgatcctaaatcttttgctgcgatgcgttataccgaagccaaactctcggcttatagtgaattattgctgagcgaattaggtcagggcact

SEQ ID NO: 765 - DNA, 9610 bp, Yersinia pestis
tgtaacgaacggtgcaatagtgatccacacccaacgcctgaaatcagatccagggggtaatctgctctcctgattcaggagagtttatggtcacttttgagacagttatggaaattaaaatcctgcacaagcagggaatgagtagccgggcgattgccagagaactggggatctcccgcaataccgttaaacgttatttgcaggcaaaatctgagccgccaaaatatacgccgcgacctgctgttgcttcactcctggatgaataccgggattatattcgtcaacgcatcgccgatgctcatccttacaaaatcccggcaacggtaatcgctcgcgagatcagagaccagggatatcgtggcggaatgaccattctcagggcattcattcgttctctctcggttcctcaggagcaggagcctgccgttcggttcgaaactgaacccggacgacagatgcaggttgactggggcactatgcgtaatggtcgctcaccgcttcacgtgttcgttgctgttctcggatacagccgaatgctgtacatcgaattcactgacaatatgcgttatgacacgctggagacctgccatcgtaatgcgttccgcttctttggtggtgtgccgcgcgaagtgttgtatgacaatatgaaaactgtggttctgcaacgtgacgcatatcagaccggtcagcaccggttccatccttcgctgtggcagttcggcaaggagatgggcttctctccccgactgtgtcgccccttcagggcacagactaaaggtaaggtggaacggatggtgcagtacacccgtaacagtttttacatcccactaatgactcgcctgcgcccgatggggatcactgtcgatgttgaaacagccaaccgccacggtctgcgctggctgcacgatgtcgctaaccaacgaaagcatgaaacaatccaggcccgtccctgcgatcgctggctcgaagagcagcagtccatgctggcactgcctccggagaaaaaagagtatgacgtgcatcttgatgaaaatctggtgaacttcgacaaacaccccctgcatcatccactctccatctacgactcattctgcagaggagtggcgtgatgatggaactgcaacatcaacgactgatggcgctcgccgggcagttgcaactggaaagccttataagcgcagcgcctgcgctgtcacaacaggcagtagaccaggaatggagttatatggacttcctggagcatctgcttcatgaagaaaaactggcacgtcatcaacgtaaacaggcgatgtatacccgaatggcagccttcccggcggtgaaaacgttcgaagagtatgacttcacattcgccaccggagcaccgcagaagcaactccagtcgttacgctcactcagcttcatagaacgtaatgaaaatatcgtattactggggccatcaggtgtggggaaaacccatctggcaatagcgatgggctatgaagcagtccgtgcaggtatcaaagttcgcttcacaacagcagcagatctgttacttcagttatctacggcacaacgtcagggccgttataaaacgacgcttcagcgtggagtaatggccccccgcctgctcatcattgatgaaataggctatctgccgttcagtcaggaagaagcaaagctgttcttccaggtcatcgctaaacgttacgaaaagagcgcaatgatcctgacatccaatctgccgttcgggcagtgggatcaaacgttcgccggtgatgcagcactgacctcagcgatgctggaccgtatcttacaccactcacatgtcgttcaaatcaaaggagaaagctatcgactcagacagaaacgaaaggccggggttatagcagaagctaatcctgagtaaaacggtggatcaatattgggccgttggtggagatataagtggatcacttttcatccgtcgttgacaccctgatgaattcacgtgttcacgcctgaataacaagaatgccggagatacgcagtcatattttttacacaattctctaatcccgacaaggtcgtaggtcgttataggaaaattcttagcaccattccggaacaatcagaacagcaggccatgaacgactgacaacattacgaatataaaaaacgcacccgggccagacattccccctactgattaaaccagccggacttgtccacggaacggtctttttaaaccgacacacagtctgagtacagatacatgtcacgatgatgcaggattagcggaagagtgtgagcacgtttccgggaactgtggtgaaccatagctcaatattcgagtgagggcataccggaaacgcgctcagattcgttgtaacgcgattttccgtaccgggcaattttttcagttgttttttcgtttcatgtcgtcagaaacgttctgagcgcgtttccggcatctgatgctacgcaaaccatccccatggtcagttgacagccggaaacacgcgggtgtcgttttagcgtatcgacgggacggcgtcgagaagcacaaaaaacagatgttgtactcagtcagttgttttacagacagcactgcggcagattgaaaaagtaccgtactttcaggaatgtccagaaaccatgtgtcagacttcgttctcccccttccgggtgaatttttttgtcatccgttcaggaatctctttataacgattactccatttcaggattttttatgtggcgtttactacaggcaggatattcaaaggcaaaaaaatcccccggaacaggcggaacccggacagggggagaacgaatcgctaaataattttcgtagttgtatttcccatcgttgctactgcaacgggatgaatttgccgcagtttatcctgtaaaacaatcctgatttactcacactccacatatcactgacggagcacaacggaatagtgaacaaacaacaacaaactgcgctgaatatggcgcgatttatcagaagccagagcctgatactgcttgaaaaactggatgctctggatgccgacgagcaggcggccatgtgtgaacgactgcacgaactcgcggaagaactccagaacagcatccaggctcgctttgaagccgaaagtgaaacaggaacataacgaagctcccggagacggtcacagcttgtctgtgaacggatgccgggagcagacaagcccgtcagggcgcgtcagcgggttttagcgggtgtcggggcgcagccatgacccagtcacgtagcgatagcggagtgtatactggcttagtcatgcggcatcagtgcggattgtatgaaaagtgcaccatgtacggtgtgaaatgccgcacagatgcgtaaggagaacatgcagatgccgatgctcttccgcttcctcgctcactgactcgctgcgctcggtcgttcggctgcggcgagcggtgtctgctcactcaaaagcggtgatactgttatccacacaatcaggggataacgccggaaagaacatgtgagcaaaaaacgaagaccccagaaaaggccgcgccggaggcgctttttccataggctccgcccccctgacgagcatcacaaaaatcgacgctcaagtcagaggtggcgaaacccgacaggacttaaagataccaggcgtttccccccggaagctccctcgtgcgctctcctgttccgaccctgccgcttaccggatacctctccgcctttctcccttcgggaagcgtggcgctttctcatagctcacgctgttggtatctcagttcggtgtaggtcgttcgctccaagctgggctgtgtgcacgaaccccccgttcagcccgaccactgcgccttatccggtaactatcgtcttgagtccaacccggtaagacacgactttacgccactggcagcagccattggtaactgaaaagtggatttagatacgcagaactcttgaagttgaagccttatcgcggctacactgaaaggacagcatttggtatctgtgctccacttaagccagctaccacaggttagaaagcctgagaaacttctaaccttcgaaagaacccacgcctgagaacgtgggttttttcgtttacaggcagcagattacgcgcagaaaaaaaggatctcaagaagatcctttgatcttttctactgaattgcgctcccgatcagttcagcagaagattatgatggggttctatgggtattgctgcggtaacacccatgttacttgaggttgtatgtagtctgtgtagaattatacacataaggcttaaactgctcttttttttcaatatgcaattggaagttcattgactacataaatagattattccaaataatttatttatgtaagaacaggatgggagggggaatgatctcaaagttattttgcttggctctcatatttttatcatcaagtggccttgcagaaaaaaacacatatacagcaaaagacatcttgcaaaacctagaattaaatacctttggcaattcattgtctcatggcatctatgggaaacagacaaccttcaagcaaaccgagtttacaaatattaaaagcaacaccaaaaaacacattgcacttatcaataaagacaactcatggatgatatcattaaaaatactaggaattaagagagatgagtatactgtctgttttgaagatttctctctaataagaccgccaacatatgtagccatacatcctctacttataaaaaaagtaaaatctggaaactttatagtagtgaaagaaataaagaaatctatccctggttgcactgtatattatcattaatagcaagcccctcattattatgaggggctcatggttattttaacaatccactatcgatatctttttgcaccagagcgccctctcgtttacgtctgtcagacattccatcaacaatattattaaaagcatttacaaggccattccagtcttttgcgataactttattccatactgtgggagcagttctggataacttaaaccctttttgatatccaatagacaccagtgctgtacgggttctcaacggtaaatcgctgaaccgaagaccgatattagcgtcattgaaaagaccttcaatcttatgtgagaatttatcaatataaatattagataagagatgagcttcattatcagaaagcgtcagaggtgctgttctcactttatcataagcctccttccctcgaagcatataatacccatcaagtctatctgcaatatactgagggacaccgtcattcaataaatcctgtttgcttcgctgaccaaggtcaaccccggaaccgaatgtaacaccggtactgttaaaataatcgctactaggattagacggaaaatgacttgtcggattaaacccttcaaaaccattactggagaaaatatcgtggtcaacaatatttaccgaacgacgtaaaaattccttcagttgactaatattgtcaaagttaatgacagtgttgtccgctaggacgatgcgatttcggttattattcagaatgtcttcgttctctttcttatcgagatgttcaatagattcggcaatcgttccctcaagaaccatgacacggtagactttcacaccgtctttttcctgacctgtttcaacagttattttctgttcgtaagacacggtcccttcagtttttgaaattttactttcctggcggatcttatttgaatattcactgtctttctccatctccgtatcaatcggaaaccccataatgtacatcagtttaaaattactccggccaggcagatccacataatgtggtaatgcaattgtaatcgaattagcttcaaaatttggtctgtaactgcttaatgtacttccggaaaagagaaaagccggaacaccacctgaaccattcactaccattgtatctgacataaaaattcctctttaacacataaaaaaacaataagttaaaaaaaaatactgtacataaaaccactgtttttatgtacagtaataaaattacgccgctttattttctctgtcaataatatgaaatttcatttttgtgatctgaatcactcttataaaaatcaggaagggaagattcgcagcagaaaaacagcaccgggtaacatcagaaaaaaacagaaaggagataacgtgagcaaaacaaaatctggtcgccaccgactgagcaaaacagacaaacgcctgctggctgcacttgtcgttgccggatacgaagaacggacagcccgtgacctcatccagaaacacgtttacacactgacacaggccgacctgcgccatctggtcagtgaaatcagtaacggtgtgggacagtcacaggcctacgatgcgatttaccaggcgagacgcattcgtctcgcccgtaaatacctgagcggaaaaaaaccggaaggggtggaaccccgggaagggcaggaacgggaagatttaccataactcccgttatcagtaccatcggctcaacgctcgttgtcggatctgaaaaattcgctcaaaagatcatatttccctggatattttccaccgtttcttatgtgagcaaagtcacataattctgtcagacgacgagaaaacggatatcgattattgtttaatatttttacattattaaaaatgaaattagataatcagatacaaataatatgttttcgttcatgcagagagattaagggtgtctaatgaagaaaagttctattgtggcaaccattataactattctgtccgggagtgctaatgcagcatcatctcagttaataccaaatatatcccctgacagctttacagttgcagcctccaccgggatgctgagtggaaagtctcatgaaatgctttatgacgcagaaacaggaagaaagatcagccagttagactggaagatcaaaaatgtcgctatcctgaaaggtgatatatcctgggatccatactcatttctgaccctgaatgccagggggtggacgtctctggcttccgggtcaggtaatatggatgactacgactggatgaatgaaaatcaatctgagtggacagatcactcatctcatcctgctacaaatgttaatcatgccaatgaatatgacctcaatgtgaaaggctggttactccaggatgagaattataaagcaggtataacagcaggatatcaggaaacacgtttcagttggacagctacaggtggttcatatagttataataatggagcttataccggaaacttcccgaaaggagtgcgggtaataggttataaccagcgcttttctatgccatatattggacttgcaggccagtatcgcattaatgattttgagttaaatgcattatttaaattcagcgactgggttcgggcacatgataatgatgagcactatatgagagatcttactttccgtgagaagacatccggctcacgttattatggtaccgtaattaacgctggatattatgtcacacctaatgccaaagtctttgcggaatttacatacagtaaatatgatgagggcaaaggaggtactcagaccattgataagaatagtggagattctgtctctattggcggagatgctgccggtatttccaataaaaattatactgtgacggcgggtctgcaatatcgcttctgaaaaatacagatcatatctctcttttcatcctcccctagcggggaggatgtctgtggaaaggaggttggtgtttgaccaaccttcagatgtgtgaaaaatcacctttttcaccataatgacggggcgctcattctgttgttttgccttgacattctccacgtctttcagggcatggagaaggtcaaattagacatggaacgctactctccttcctgtaggaagctcaacatccaagcttaatttgcctcccattgcttcaacgtaacgctttaacgtcgccagctttaaatcatttccgcgctgctccagctttgttactgctggctggcttatacccatcgcctcagcaacttgtttttgtgataactggagttcttcacgcatcatctgcaagccgacctcaagaatcatctcatctgccatttctttaattcgtgtctggctttcaggtgaacgactggcaatcacctcatctaatgttctcattacttgctctccagtgtgttcagatgtgctgtaaattcatcctcagctatacgcaccagtttttcataaaaccgcttatcattacttttatctcctgcacaaagaacgatagcccgacgaatcggatcgaacgcataaaaggctcttatcggacggccagaaaactgaacgcgaagctctttcatatttttgtaccgagaacctttcacggtatcggcatatggcctgggtaactcaggtccgtaaacctgtagctttttcaaatcagccaaaaccttttcctgaagagcgtcttcttgctcatttagccagtcgtcaaatcgctggctaaaaagtaccatccacatgctcaaccctataacctgtagcttaccccactaacaatataacctacgagttatattttcaagaaaagctggctatttaacataacggcaatttgtacgcaccactgaaatgcgttcagcgcgatcacggcaacagacaggcaaaaatagcaacaaacctcccgaaaaaccgccgcgatcgcgcctgataaattttaaccttatgcatatctatgcagccaggcgaatcacgaacgaattgcctgcctgatgtaactgaaacgggtgttttttcctgatttggtgggcgtggaagacggaacatgaacgggaaaacagaattcatgccagatgagcgcgatctggcaattaaggcaaaacacagcaacaaagacacgccagaatcgcgcccggatatgttttaacgcgattttcagactcagacaaattcagcagaatgctactccattcaccgggctgatggtgaatacatgcgtatccaggatgagtacatttctggctctgccacagctctgtctgttggcagctttcgcctgtccggaaacctgcttaaaacgctcccgaaaggcctctgaaccagaaagcaacaaaacacaggccattaagtaaatcgcgttaaaacacgtctgatggattgctgcaaaaaaaagtccctaatggagcagggactgttaaacccagtgaatagcgtctaaattaaagtaagaatacgaccaggtactcttcagaaaagagattaatccaccgcacagaataatcaacagtaaaaacaaacaaccctgattttttatttttctttttttcgataaaaacaaaattaaagaaataattaatcagaacattccttaacttcagggcattgcctgtgttccattttgtgattagtctgaaacttccgaaggtggataacacccggtatttttttgctcacataaagcccctccttcaggcagaggggctttttctttgccaccacataaaaaaggccctcacaggaggtgttctgtgagggcgtatgataaggactgaatcgatggttaatatgtctagtcctgacttttgcatctccgaatataaaaccctgtttaacggcatgcaaaaccaaaaaataaaaatgtgacatcgcaatgccagataatattgacgcatgagggaatgcgtaccccgacccctg

SEQ ID NO: 766 - DNA, 102 bp, Artificial Sequence
  Calibration Polynucleotide
tttaagtcccgcaacgagcgcaacccttgatcttagttgtttagttgggcactctaaggtgactgccggtgacaaaccggaggaaggtggggatgacgtcaa

SEQ ID NO: 767 - DNA, 94 bp, Artificial Sequence
  Calibration Polynucleotide
tagaacaccgatggcgaaggcgactttctggtctgtaactgacactgagaaagcgtggggagcaaacaggattagataccctggtagtccacga

SEQ ID NO: 768 - DNA, 108 bp, Artificial Sequence
  Calibration Polynucleotide
tggattagagaccctggtagtccacgccgtaaacgatgagtgctaagtgttagaggcctttagtgctgaagttaacgcattaagcactccgcctggggagtacggcca

SEQ ID NO: 769 - DNA, 108 bp, Artificial Sequence
  Calibration Polynucleotide
tttcgatgcaacgcgaagaaccttaccaggtcttgacatcctctgacaaccctagcttctccttcgggagcagagtgacaggtggtgcatggctgtcgtcagctcgta

SEQ ID NO: 770 - DNA, 95 bp, Artificial Sequence
  Calibration Polynucleotide
tctgacacctgcccggtgctggaaggttaaggagaggggttagcgtaactctgaactgaagccccagtaaacggcggccgtaactataacggtca

SEQ ID NO: 771 - DNA, 117 bp, Artificial Sequence
  Calibration Polynucleotide
tctgttcttagtacgagaggaccgggatggacgcaccggtaccagttgttctgccaagggcatagctgggtagctatgtgcggaagggataagtgctgaaagcatctaagcacgaaa

SEQ ID NO: 772 - DNA, 100 bp, Artificial Sequence
  Calibration Polynucleotide
tgattattgttatcctgttatgccatttgagatttttgagtggtattggagttattgttccaggattaattgcaaatacaattcaaagacaagggttaca

SEQ ID NO: 773 - DNA, 112 bp, Artificial Sequence
  Calibration Polynucleotide
tcgaagtacaatacaagacaaaagaaggtaaaattactgttttaggggaaaaattcaagaaatatagaagtgatggctaaaaatgtagaaggggtcttgaagccgttaacaa

SEQ ID NO: 774 - DNA, 100 bp, Artificial Sequence
  Calibration Polynucleotide
ttgctcgtggtgcacaagtaacggatattacaatcattgttgttgcagctgatgacggcgtaataaacagttgaagcaattaaccatgcgaaagcagcaa

SEQ ID NO: 775 - DNA, 114 bp, Artificial Sequence
  Calibration Polynucleotide
tagcttttgcatattatatcgagccacagcatcgtgatgttttacagctttatgcaccggaagcttttaatggataaatttaacgaacaagaaataaatctatccttggaagaa

SEQ ID NO: 776 - DNA, 116 bp, Artificial Sequence
  Calibration Polynucleotide
tgacctacagtaagaggttctgtaatgaaccctaatgaccatccacacggtggtggtgaaggtagatctcctatcggaaagtccacgtactccatggggtaaaccagcacttggaa

SEQ ID NO: 777 - DNA, 70 bp, Artificial Sequence
  Calibration Polynucleotide
tccacacggtggtggtgaaggtagatctcctatcggaaagtccacgtactccatggggtaaaccagcaca

SEQ ID NO: 778 - DNA, 82 bp, Artificial Sequence
  Calibration Polynucleotide
ttatcgctcaggcgaactccaacctggatgatgaaggccgctttttagaaggtgacttgtcgtagcaaaggcgaatccagca

SEQ ID NO: 779 - DNA, 87 bp, Artificial Sequence
  Calibration Polynucleotide
tgggcagcgtttcggcgaaatggaagtggctcgaagcgtatggcgcttcgtacgtgctgcaggaaatgttgaccgtcaagtcggaca

SEQ ID NO: 780 - DNA, 97 bp, Artificial Sequence
  Calibration Polynucleotide
tcaggagtcgttcaactcgatctacatgatggccgaccgcccggggttcggcggtgcagattcgtcagctggccggcatgcgtggcctgatggcgta

SEQ ID NO: 781 - DNA, 117 bp, Artificial Sequence
  Calibration Polynucleotide
tctggcaggtatgcgtggtctgatggccaatccatctggtcgtatcatcgaacttccaatcaagtttccgtgaaggtttaacagtacttgagtacttcatctcaacccacggtgcga

SEQ ID NO: 782 - DNA, 98 bp, Artificial Sequence
  Calibration Polynucleotide
tcaagcaaacgcacaatcagaagctaagaaagcgcaagcttctggaaagcacaaatgctagttatggtacagaatttgcaactgaaacagacgtgcaa

SEQ ID NO: 783 - DNA, 99 bp, Artificial Sequence
  Calibration Polynucleotide
tccacacgccgttcttcaacaactaccgtgttctacttccgtacgacggacgtgacgggctcgatcgagctgccgaaggacaaggaaatggtgatgcca

SEQ ID NO: 784 - DNA, 111 bp, Artificial Sequence
  Calibration Sequence
tcgtggcggcgtggttatcgaacccatgctgaccgatcaatggtacgtgcacaccgccccccaaagtcgcgattgaagccgtagagaacggcgacatccagttcgtaccga

SEQ ID NO: 785 - DNA, 2100 bp, Artificial Sequence
  Combination Calibration Polynucleotide
gaagtagagatatggaggaacaccagtggcgaaggcgactttctggtctgtaactgacactgagaaagcgtggggagcaaacaggattagataccctggtagtccacgccgtaaacgatgagtgctaagtgttagaggcctttagtgctgaagttaacgcattaagcactccgcctggggagtacggccgcaaggctgaaactcaaaggaattgacggggcacaagcggtggagcatgtggtttaattcgaagcaacgcgaagaaccttaccaggtcttgacatcctctgacaaccctagcttctccttcgggagcagagtgacaggtggtgcatggttgtcgtcagctcgtgtcgtgagatgttgggttaagtcccgcaacgagcgcaacccttgatcttagttgtttagttgggcactctaaggtgactgccggtgacaaaccggaggaaggtggggatgacgtcaaatcatcatgccccagtaccgtgagggaaaggtgaaaagcaccccggaaggggagtgaaagagatcctgaaaccgtgtgccatagtcagagcccgttaacgggtgatggcgtgccttttgtagaatgaaccggcgagttataagatccgtagtcaaaagggaaacagcccagaccgccagctaaggtcccaaagtgtgtattgaaaaggatgtggagttgcttagacaactaggatgttggcttagaagcagccaccatttaaagagtatagggggtgacacctgcccggtgctggaaggttaaggagaggggttagcgtaactctgaactgaagccccagtaaacggcggccgtaactataacggtcctaaggtagcgaaagaaatttgagaggagctgtccttagtacgagaggaccgggatggacgcaccggtaccagttgttctgccaagggcatagctgggtagctatgtgcggaagggataagtgctgaaagcatctaagcatgaagcccccctcaagatgagagcagtaaaacaagcaaacgcacaatcagaagctaagaaagcgcaagcttctggaaagcacaaatgctagttatggtacagaatttgcaactgaaacagacgtgcatgctgtgaaatttgcgaaagcttttgcatattatatcgagccacagcatcgtgatgttttacagctttatgcaccggaagcttttaatggataaatttaacgaacaagaaataaatctatccttggaagaacttaaagatcaacggatgctggcaagatatgaaaaataagataaaacagcactatcaacactggagcgattctttatctgaagaaggaagagcgatgaaaacaacgaagtacaatacaagacaaaagaaggtaaaattactgttttaggggaaaaattcaagaaatatagaagtgatggctaaaaatgtagaaggggtcttgaagccgttaacagctgttatggcgaccgtggcggcgtggttatcgaacccatgctgaccgatcaatggtacgtgcacaccgccccccaaagtcgcgattgaagccgtagagaacggcgagatccagttcgtccctaaacagtacggcaacttcgttatcgctcaggcgaactccaacctggatgatgaaggccgctttttagaaggtgacttgtcgtagcaaaggcgaatcaagcctgtttagccacaactatgcgtgctcgtggtgcacaagtaacggatattacaatcattgttgttgcagctgatgacggcgtaataaacagttgaagcgattaaccatgcgaaagcagcaggagtaccaactttactcagcttgctggtatgcgtggtctgatggccaatccatctggtcgtatcatcgaacttccaatcaagtttccgtgaaggtttaacagtacttgagtacttcatctctacgcatggtgcgcgtaaaggtcatgggagtaagacctacagtaagaggttctgtaatgaaccctaatgaccatccacacggtggtggtgaaggtagatctcctatcggaaagtccacgtactccatggggtaaaccagcacttggatacaaaacaagcgcagttcggcggccagcgcttcggtgaaa